(19) United States
(12) Patent Application Publication
(10) Pub. No.: US 2016/0229866 A1
(43) Pub. Date: Aug. 11, 2016

(54) HEPATITIS C VIRUS INHIBITORS

(71) Applicant: IDENIX PHARMACEUTICALS INC., Cambridge, MA (US)

(72) Inventors: Cyril B. Dousson, Canet (FR); Jean-Laurent Paparin, Vendermian (FR)

(73) Assignee: Idenix Pharmaceuticals Inc., Cambridge, MA (US)

(21) Appl. No.: 15/022,755

(22) PCT Filed: Sep. 19, 2014
(86) PCT No.: PCT/US14/56519

§ 371 Date: Mar. 17, 2016

Related U.S. Application Data
(60) Provisional application No. 61/960,538, filed on Sep. 20, 2013.
(60) Provisional application No. 62/016,503, filed on Jun. 24, 2014.

(51) Int. Cl. C07D 495/04; A61K 31/4184; A61K 31/675; C07F 9/6561; A61K 45/06

(52) U.S. Cl. CPC C07D 495/04; C07F 9/6561; A61K 45/06; A61K 31/675; A61K 31/4184

(57) ABSTRACT

Provided herein are hepatitis C virus inhibitor compounds, for example, of any of Formulae I to XIV, Ilia to XlVa, Illb to XlVb, IIIc to XIVc, Hid to XlVd, Ille to XlVe, IA to IAe, IIA to IIAe, IB, IIB to IIBd, IIIB to IIIBd, IC to ICc, ID to IDd, and IE to lEc.pharmaceutical compositions comprising the compounds, and processes of preparation thereof. Also provided are methods of their use for treating an HCV infection.

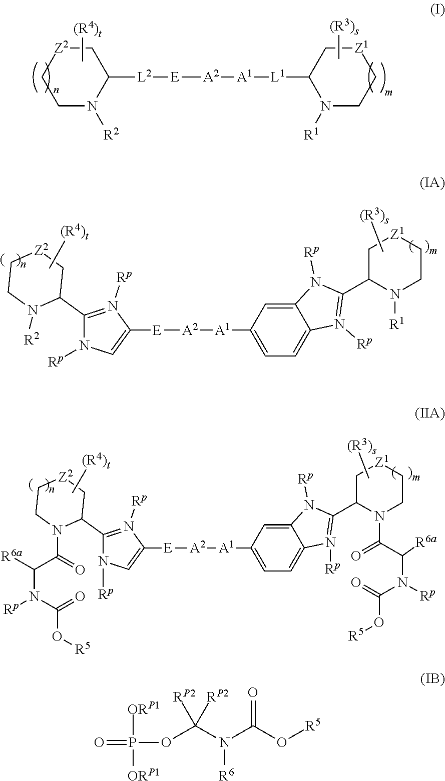

### CROSS REFERENCE TO RELATED APPLICATIONS

The present application claims the benefit of priority of U.S. Provisional Application No. 61/960,538, filed Sep. 20, 2013, and U.S. Provisional Application No. 62/016,503, filed Jun. 24, 2014, the content of each of which is hereby incorporated by reference in its entirety.

### FIELD

Provided herein are hepatitis C virus inhibitor compounds, pharmaceutical compositions comprising the compounds, and preparation thereof. Also provided are methods of their use for treating an HCV infection.

### BACKGROUND

Hepatitis C virus (HCV) is known to cause at least 80% of post transfusion hepatitis and a substantial proportion of sporadic acute hepatitis (Kuo et al., Science 1989, 244, 362-364; Thomas, Curr. Top. Microbiol. Immunol. 2000, 25-41). Preliminary evidence also implicates HCV in many cases of “idiopathic” chronic hepatitis, “cryptogenic” cirrhosis, and probably hepatocellular carcinoma unrelated to other hepatitis viruses, such as hepatitis B virus (Di Besceglie et al., Scientific American 1999, October, 80-85; Boyer et al., J. Hepatol. 2000, 32, 98-112).

HCV is an enveloped virus containing a positive-sense single-stranded RNA genome of approximately 9.4 kb (Kato et al., Proc. Natl. Acad. Sci. USA 1990, 87, 9524-9528; Kato, Acta Medica Okayama 2001, 55, 133-159). The viral genome consists of a 5′ untranslated region (UTR), a long open reading frame encoding a polyprotein precursor of approximately 3011 amino acids, and a short 3′ UTR. The 5′ UTR is the most highly conserved part of the HCV genome and is important for the initiation and control of polyprotein translation. Translation of the HCV genome is initiated by a cap-independent mechanism known as an internal ribosome entry. This mechanism involves the binding of ribosomes to an RNA sequence known as the internal ribosome entry site (IRES). An RNA pseudoknot structure has recently been determined to be an essential structural element of the HCV IRES. Viral structural proteins include a nucleocapsid core protein (C) and two envelope glycoproteins, E1 and E2. HCV also encodes two proteinases, a zinc-dependent metalloproteinase encoded by the NS2-NS3 region and a serine proteinase encoded in the NS3 region. These proteinases are required for cleavage of specific regions of the precursor polyprotein into mature peptides. The carboxyl half of nonstructural protein 5, NS5B, contains the RNA-dependent RNA polymerase. The function of the remaining nonstructural proteins, NS4A and NS4B, and that of NS5A (the amino-terminal half of nonstructural protein 5) remain unknown.

Presently, the most effective HCV therapy employs a combination of alpha-interferon and ribavirin, leading to sustained efficacy in about 40% of patients (Poynard et al., Lancet 1998, 352, 1426-1432). Recent clinical results demonstrate that pegylated alpha-interferon is superior to unmodified alpha-interferon as monotherapy. However, even with experimental therapeutic regimens involving combinations of pegylated alpha-interferon and ribavirin, a substantial fraction of patients do not have a sustained reduction in viral load (Manns et al., Lancet 2001, 358, 958-965; Fried et al., N. Engl. J. Med. 2002, 347, 975-982; Hadziyannis et al., Ann. Intern. Med. 2004, 140, 346-355). Thus, there is a clear and unmet need to develop effective therapeutics for the treatment of HCV infections.

### SUMMARY OF THE DISCLOSURE

Provided herein is a compound of Formula I:

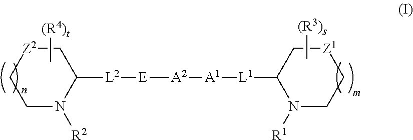

or a single enantiomer, a racemic mixture, a diastereomer, a mixture of diastereomers, or an isotopic variant thereof; or a pharmaceutically acceptable salt or solvate thereof;

A¹, A², and E are each independently (a) a bond; or (b) C₁₋₆ alkylene, C₂₋₆ alkenylene, C₂₋₆ alkynylene, C₂₋₂₀ cycloalkylene, C₆₋₂₀ arylene, heteroarylene, or heterocyclylene;

L¹ and L² are each independently (a) a bond; (b) C₁₋₆ alkylene, C₂₋₆ alkenylene, C₂₋₆ alkynylene, C₃₋₇cycloalkylene, C₆₋₁₄ arylene, heteroarylene, or heterocyclylene; or (c) —C(O)—, —C(O)O—, —C(O)NR^{1a}—, —C(═NR^{1a})NR^{1c}—, —O—, —OC(O)O—, —OC(O)NR^{1a}—, —OC(═NR^{1a})NR^{1c}—, —OP(O)(OR^{1a})—, —OP(O)(OR^{1a})O—, —NR—, —NR^{1a}C(O)NR^{1c}—, —NR^{1a}C(═NR^{b})NR^{1c}—, —NR^{1a}S(O)NR^{1c}—, —NR^{1a}S(O)₂NR^{1c}—, —S—, —S(O)—, —S(O)₂—, —S(O)NR^{1a}—, or —S(O)₂NR^{1a}—; wherein at least one of L¹ and L² is heteroarylene or heterocyclylene, which is substituted with —C₁₋₆ alkylene-OP(O)(OR^{P1})₂ (in one embodiment, —CH₂—OP(O)(OR^{P1})₂), or —C₁₋₆ alkylene-O-linked amino acid or a derivative thereof (in one embodiment, —CH₂—OC(O)C(R^{aa})₂NR^{1b}R^{1c});

Z¹ and Z² are each independently a bond, —O—, —S—, —S(O)—, —S(O₂)—, or —N(R^{N})—;

R¹ and R² are each independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (c) —C(O)R^{a}, —C(O)CH(N(R^{1c})C(O)OR^{1b})R^{1a}, —C(O)OR^{1a}, —C(O)NR^{1b}R^{1c}, —C(NR^{1a})NR^{1b}R^{1c}, —P(O)(OR^{1a})R^{1d}, —CH₂P(O)(OR^{1a})R^{1d}, —S(O)R^{1a}, —S(O)₂R^{1a}, —S(O)NR^{1b}R^{1c}, or —S(O)₂NR^{1b}R^{1c}; (d) —C₁₋₆ alkylene-OP(O)(OR^{P1})₂, in one embodiment, —CH₂—OP(O)(OR^{P1})₂, or (e) —C₁₋₆ alkylene-O-linked amino acid or a derivative thereof, in one embodiment, —CH₂—OC(O)C(R^{aa})₂NR^{1b}R^{1c};

each R³ and R⁴ is independently (a) cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —SR^{1a}, —S(O)R^{1a}, —S(O)₂R^{1a}, —S(O)NR^{1b}R^{1c}, or —S(O)₂NR^{1b}R^{1c}; or

two R³ or two R⁴ that are attached to the same ring are linked together to form a bond, —O—, —NR^{N}—, —S—, C₁₋₆ alkylene, C₁₋₆ heteroalkylene, C₂₋₆ alkenylene, or C₂₋₆ heteroalkenylene;

each R^{N} is independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (c) —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1d}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —S(O)R^{1a}, —S(O)₂R^{1a}, —S(O)NR^{1b}R^{1c}, or —S(O)₂NR^{1b}R^{1c}; (d) —C₁₋₆ alkylene-OP(O)(OR^{P1})₂, in one embodiment, —CH₂—OP(O)(OR^{P1})₂, or (e) —C₁₋₆ alkylene-O-linked amino acid or a derivative thereof, in one embodiment, —CH₂—OC(O)C(R^{aa})₂NR^{1b}R^{1c};

each R^{P1} is independently (a) hydrogen; (b) a monovalent cation, in one embodiment, Na⁺ or K⁺, in another embodiment, Li⁺, Rb⁺, or Cs+; or (c) C₁₋₆ alkyl, C₂₋₆ alkenyl, C₂₋₆ alkynyl, C₃₋₇cycloalkyl, C₆₋₁₄ aryl, C₇₋₁₅ aralkyl, heteroaryl, or heterocyclyl; or two R^{P1} together are a divalent cation, in one embodiment, Mg²⁺ or Ca²⁺;

each R^{1a}, R^{1b}, R^{1c}, and R^{1d} is independently hydrogen, C₁₋₆ alkyl, C₂₋₆ alkenyl, C₂₋₆ alkynyl, C₃₋₇cycloalkyl, C₆₋₁₄ aryl, C₇₋₁₅ aralkyl, heteroaryl, or heterocyclyl; or R^{1a} and R^{1c} together with the C and N atoms to which they are attached form heterocyclyl; or R^{1b} and R^{1c} together with the N atom to which they are attached form heterocyclyl;

each R^{aa} independently is a side chain of a naturally occurring or non-naturally occurring amino acid; in one embodiment, each R^{aa} independently is hydrogen, C₁₋₆ alkyl, heteroalkyl, C₆₋₁₄ aryl-C₁₋₆ alkyl and heteroaryl-C₁₋₆ alkyl;

each m and n is independently an integer of 1, 2, 3, or 4; and

each s and t is independently an integer of 0, 1, 2, 3, 4, 5, 6, or 7;

with the proviso that the compound is neither

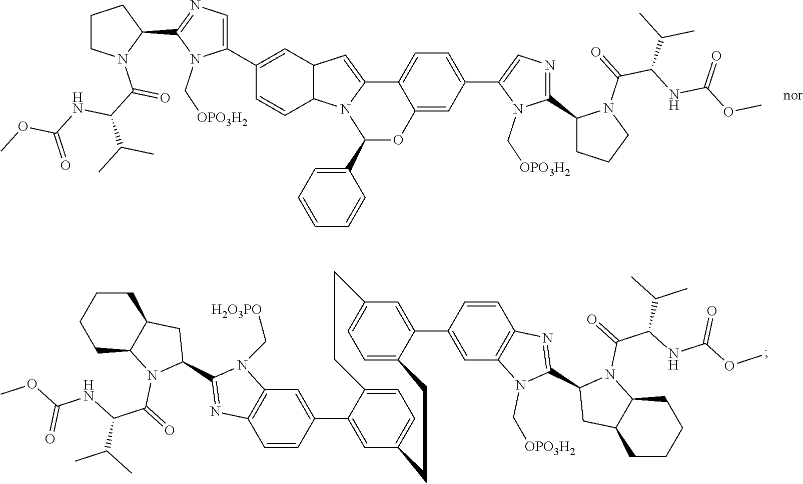

wherein each alkyl, alkylene, heteroalkylene, alkenyl, alkenylene, heteroalkenylene, alkynyl, alkynylene, cycloalkyl, cycloalkylene, aryl, arylene, aralkyl, heteroaryl, heteroarylene, heterocyclyl, and heterocyclylene is optionally substituted with one or more, in one embodiment, one, two, three, or four, substituents Q, where each Q is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is further optionally substituted with one or more, in one embodiment, one, two, three, or four, substituents Q^{a}; or (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OP(O)(OR^{e})₂, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —P(O)(OR^{a})R^{d}, —CH₂P(O)(OR^{a})R^{d}, —CH₂OP(O)(OR^{e})₂, —CH₂OC(O)C(R^{a})₂NR^{b}R^{c}, —SR^{a}, —S(O)R^{a}, —S(O)₂R^{a}, —S(O)NR^{b}R^{c}, or —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a}; and each R^{e} is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iv) two R^{e} together are a divalent cation (e.g., Mg²⁺ or Ca²⁺);

wherein each Q^{a} is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{f}, —C(O)OR^{f}, —C(O)NR^{g}R^{h}, —C(NR^{f})NR^{g}R^{h}, —OR^{f}, —OC(O)R^{f}, —OC(O)OR^{f}, —OC(O)NR^{g}R^{h}, —OC(═NR)NR^{g}R^{h}, —OP(O)(ORⁿ)₂, —OS(O)R^{f}, —OS(O)₂R^{f}, —OS(O)NR^{g}R^{h}, —OS(O)₂NR^{g}R^{h}, —NR^{g}R^{h}, —NR^{f}C(O)R^{k}, —NR^{f}C(O)OR^{k}, —NR^{f}C(O)NR^{g}R^{h}, —NR^{f}C(═NR^{k})NR^{g}R^{h}, —NR^{f}S(O)R^{k}, —NR^{f}S(O)₂R^{k}, —NR^{f}S(O)NR^{g}R^{h}, —NR^{f}S(O)₂NR^{g}R^{h}, —P(O)(OR^{f})R^{k}, —CH₂P(O)(OR^{f})R^{k}, —SR^{f}, —S(O)R^{f}, —S(O)₂R, —S(O)NR^{g}R^{h}, or —S(O)₂NR^{g}R^{h}; wherein each R^{f}, R^{g}, R^{h}, and R^{k} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{g} and R^{h} together with the N atom to which they are attached form heterocyclyl; and each Rⁿ is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iv) two Rⁿ together are a divalent cation (e.g., Mg²⁺ or Ca²⁺).

In certain embodiments of the compound of Formula I, or single enantiomer, racemic mixture, diastereomer, mixture of diastereomers, or isotopic variant thereof; or pharmaceutically acceptable salt or solvate thereof;

A¹, A², and E are each independently (a) a bond; or (b) C₁₋₆ alkylene, C₂₋₆ alkenylene, C₂₋₆ alkynylene, C₂₋₂₀ cycloalkylene, C₆₋₂₀ arylene, heteroarylene, or heterocyclylene;

L¹ and L² are each independently (a) a bond; (b) C₁₋₆ alkylene, C₂₋₆ alkenylene, C₂₋₆ alkynylene, C₃₋₇ cycloalkylene, C₆₋₁₄ arylene, heteroarylene, or heterocyclylene; or (c) —C(O)—, —C(O)O—, —C(O)NR^{a}—, —C(═NR^{1a})NR^{1c}—, —O—, —OC(O)O—, —OC(O)NR^{1a}—, —OC(═NR^{1a})NR^{1c}—, —OP(O)(OR^{1a})—, —OP(O)(OR^{1a})O—, —NR^{1a}—, —NR^{1a}C(O)NR^{1c}—, —NR^{1a}C(═NR^{1b})NR^{1c}—, —NR^{1a}S(O)NR^{1c}—, —NR^{1a}S(O)₂NR^{1c}—, —S—, —S(O)—, —S(O)₂—, —S(O)NR^{1a}—, or —S(O)₂NR^{1a}—; wherein at least one of L¹ and L² is heteroarylene or heterocyclylene, which is substituted with —C₁₋₆ alkylene-OP(O)(OR^{P1})₂, in one embodiment, —CH₂—OP(O)(OR^{P1})₂;

Z¹ and Z² are each independently a bond, —O—, —S—, —S(O)—, —S(O₂)—, or —N(R^{N})—;

R¹ and R² are each independently (a) hydrogen; (b) C₁₋₆ alkyl, C₂₋₆ alkenyl, C₂₋₆ alkynyl, C₃₋₇cycloalkyl, C₆₋₁₄ aryl, C₇₋₁₅ aralkyl, heteroaryl, or heterocyclyl; (c) —C(O)R^{1a}, —C(O)CH(N(R^{1c})C(O)OR^{1b})R^{1a}, —C(O)OR^{1a}, —C(O)NR^{1b}R^{1c}, —C(NR^{1a})NR^{1b}R^{1a}, —P(O)(OR^{1a})R^{1d}, —CH₂P(O)(OR^{1a})R^{1d}, —S(O)R^{1a}, —S(O)₂R^{1a}, —S(O)NR^{1b}R^{1c}, or —S(O)₂NR^{1b}R^{1c}; or (d) —C₁₋₆ alkylene-OP(O)(OR^{P1})₂, in one embodiment, —CH₂—OP(O)(OR^{P1})₂;

each R³ and R⁴ is independently (a) cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —SR^{1a}, —S(O)R^{1a}, —S(O)₂R^{1a}, —S(O)NR^{1b}R^{1c}, or —S(O)₂NR^{1b}R^{1c}; or

two R³ or two R⁴ that are attached to the same ring are linked together to form a bond, —O—, —NR^{N}—, —S—, C₁₋₆ alkylene, C₁₋₆ heteroalkylene, C₂₋₆ alkenylene, or C₂₋₆ heteroalkenylene;

each R^{N} is independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (c) —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —S(O)R^{1a}, —S(O)₂R^{1a}, —S(O)NR^{1b}R^{1c}, or —S(O)₂NR^{b}R^{1c}; or (d) —C₁₋₆ alkylene-OP(O)(OR^{P1})₂, in one embodiment, —CH₂—OP(O)(OR^{P1})₂;

each R^{P1} is independently (a) hydrogen; (b) a monovalent cation, in one embodiment, Na⁺ or K⁺; or (c) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or two R^{P1} together are a divalent cation, in one embodiment, Mg²⁺ or Ca²⁺;

each R^{1a}, R^{1b}, R^{1c}, and R^{1d} is independently hydrogen, C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or R^{1a} and R^{1c} together with the C and N atoms to which they are attached form heterocyclyl; or R^{1b} and R^{1c} together with the N atom to which they are attached form heterocyclyl;

each m and n is independently an integer of 1, 2, 3, or 4; and

each s and t is independently an integer of 0, 1, 2, 3, 4, 5, 6, or 7;

with the proviso that the compound is neither

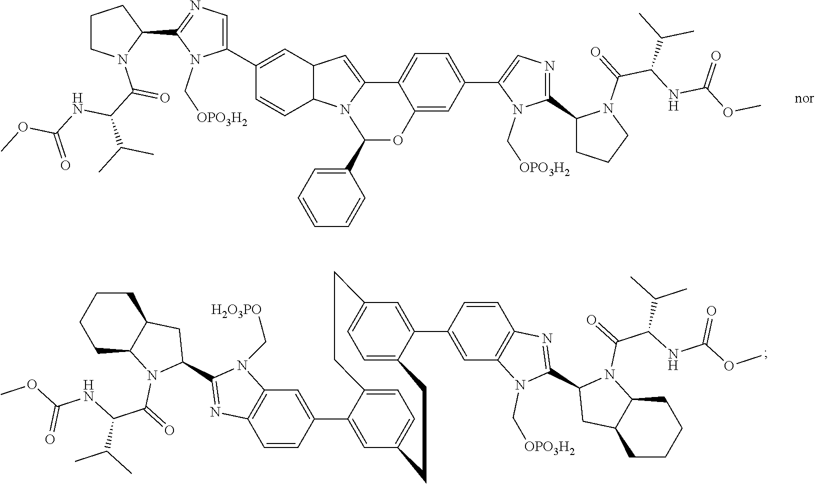

wherein each alkyl, alkylene, heteroalkylene, alkenyl, alkenylene, heteroalkenylene, alkynyl, alkynylene, cycloalkyl, cycloalkylene, aryl, arylene, aralkyl, heteroaryl, heteroarylene, heterocyclyl, and heterocyclylene is optionally substituted with one or more, in one embodiment, one, two, three, or four, substituents Q, where each Q is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is further optionally substituted with one or more, in one embodiment, one, two, three, or four, substituents Q^{a}; or (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OP(O)(OR^{e})₂, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —P(O)(OR^{a})R^{d}, —CH₂P(O)(OR^{a})R^{d}, —SR^{a}, —S(O)R^{a}, —S(O)₂R^{a}, —S(O)NR^{b}R^{c}, or —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and Re together with the N atom to which they are attached form heterocyclyl, optionally substituted with one or more, in one embodiment, one, two, three, or four, substituents Q^{a}; and each R^{e} is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iv) two R^{e} together are a divalent cation (e.g., Mg²⁺ or Ca²⁺); wherein each Q^{a} is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{f}, —C(O)OR^{f}, —C(O)NR^{g}R^{h}, —C(NR^{f})NR^{g}R^{h}, —OR^{f}, —OC(O)R^{f}, —OC(O)OR^{f}, —OC(O)NR^{g}R^{h}, —OC(═NR)NR^{g}R^{h}, —OP(O)(ORⁿ)₂, —OS(O)R^{f}, —OS(O)₂R^{f}, —OS(O)NR^{g}R^{h}, —OS(O)₂NR^{g}R^{h}, NR^{g}R—NR^{f}C(O)R^{k}, —NR^{f}C(O)OR^{k}, —NR^{f}C(O)NR^{g}R^{h}, —NR^{f}C(═NR^{k})NR^{g}R^{h}, —NR^{f}S(O)R^{k}, —NR^{f}S(O)₂R^{k}, —NR^{f}S(O)NR^{g}R^{h}, —NR^{f}S(O)₂NR^{g}R^{h}, —P(O)(OR^{f})R^{k}, —CH₂P(O)(OR^{f})R^{k}, —SR^{f}, —S(O)R^{f}, —S(O)₂R^{f}, —S(O)NR^{g}R^{h}, or —S(O)₂NR^{g}R^{h}; wherein each R^{f}, R^{g}, R^{h}, and R^{k} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{g} and R^{h} together with the N atom to which they are attached form heterocyclyl; and each Rⁿ is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iv) two Rⁿ together are a divalent cation (e.g., Mg²⁺ or Ca²⁺).

Also provided herein is a compound of Formula IA:

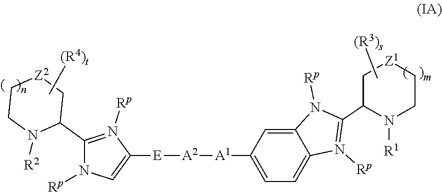

or a single enantiomer, a racemic mixture, a diastereomer, a mixture of diastereomers, or an isotopic variant thereof; or a pharmaceutically acceptable salt or solvate thereof;

A¹, A², and E are each independently (a) a bond; or (b) C₁₋₆ alkylene, C₂₋₆ alkenylene, C₂₋₆ alkynylene, C₂₋₂₀ cycloalkylene, C₆₋₂₀ arylene, heteroarylene; or heterocyclylene;

Z¹ and Z² are each independently a bond, —O—, —S—, —S(O)—, —S(O₂)—, or —N(R^{N})—;

R¹ and R² are each independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (c) —C(O)R^{1a}, —C(O)CH(N(R^{1c})C(O)OR^{1b})R^{1a}, —C(O)OR^{1a}, —C(O)NR^{1b}R^{1c}, —C(NR^{1a})NR^{1b}R^{1c}, —P(O)(OR^{1a})R^{1d}, —CH₂P(O)(OR^{1a})R^{1d}, —S(O)R^{1a}, —S(O)₂R^{1a}, —S(O)NR^{1b}R^{1c}, or —S(O)₂NR^{1b}R^{1c}; (d) —C₁₋₆ alkylene-OP(O)(OR^{P1})₂, in one embodiment, —CH₂—OP(O)(OR^{P1})₂, or (e) —C₁₋₆ alkylene-O-linked amino acid or a derivative thereof, in one embodiment, —CH₂—OC(O)C(R^{aa})₂NR^{1b}R^{1c};

each R³ and R⁴ is independently (a) cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —SR^{1a}, —S(O)R^{1a}, —S(O)₂R^{1a}, —S(O)NR^{1b}R^{1c}, or —S(O)₂NR^{1b}R^{1c}; or

two R³ or two R⁴ that are attached to the same ring are linked together to form a bond, —O—, —NR^{N}—, —S—, C₁₋₆ alkylene, C₁₋₆ heteroalkylene, C₂₋₆ alkenylene, or C₂₋₆ heteroalkenylene;

each R^{N} is independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (c) —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1a}, —OS(O)R^{1a}, —OS(O)₂R^{1a}, —OS(O)NR^{1b}R^{a}C, —OS(O)₂NR^{1b}R^{1c}, —NR^{1b}R^{1c}, —NR^{1a}C(O)R^{d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —S(O)R^{1a}, —S(O)₂R^{1a}, —S(O)NR^{1b}R^{1c}, or —S(O)₂NR^{1b}R^{1c}; (d) —C₁₋₆ alkylene-OP(O)(OR^{P1})₂, in one embodiment, —CH₂—OP(O)(OR^{P1})₂, or (e) —C₁₋₆ alkylene-O-linked amino acid or a derivative thereof, in one embodiment, —CH₂—OC(O)C(R^{aa})₂NR^{1b}R^{1c};

each R^{P} is independently absent, hydrogen, —C₁₋₆ alkylene-OP(O)(OR^{P1})₂ (in one embodiment, —CH₂—OP(O)(OR^{P1})₂), or —C₁₋₆ alkylene-O-linked amino acid or a derivative thereof (in one embodiment, —CH₂—OC(O)C(R^{aa})₂NR^{1b}R^{1c}); wherein, when the two R^{P} groups attached to the imidazolylene are neither absent nor hydrogen, the imidazolyene group carries a positive charge; and when the two RP groups attached to the benzimidazolylene are neither absent nor hydrogen, the benzimidazolylene group carries a positive charge; and wherein at least one of the R^{P} groups is neither absent nor hydrogen;

each R^{P1} is independently (a) hydrogen; (b) a monovalent cation, in one embodiment, Na⁺ or K⁺, in another embodiment, Li⁺, Rb⁺, or Cs⁺; or (c) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or two R^{P1} together are a divalent cation, in one embodiment, Mg²⁺ or Ca²⁺;

each R^{1a}, R^{1b}, R^{1c}, and R^{1d} is independently hydrogen, C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or R^{1a} and R^{1c} together with the C and N atoms to which they are attached form heterocyclyl; or R^{1b} and R^{1c} together with the N atom to which they are attached form heterocyclyl;

each R^{aa} independently is a side chain of a naturally occurring or non-naturally occurring amino acid; in one embodiment, each R^{aa} independently is hydrogen, C₁₋₆ alkyl, heteroalkyl, C₆₋₁₄ aryl-C₁₋₆alkyl and heteroaryl-C₁₋₆alkyl;

each m and n is independently an integer of 1, 2, 3, or 4; and

each s and t is independently an integer of 0, 1, 2, 3, 4, 5, 6, or 7;

wherein each alkyl, alkylene, heteroalkylene, alkenyl, alkenylene, heteroalkenylene, alkynyl, alkynylene, cycloalkyl, cycloalkylene, aryl, arylene, aralkyl, heteroaryl, heteroarylene, imidazolylene, benzimidazolyl, heterocyclyl, and heterocyclylene is optionally substituted with one or more, in one embodiment, one, two, three, or four, substituents Q, where each Q is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is further optionally substituted with one or more, in one embodiment, one, two, three, or four, substituents Q^{a}; or (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OP(O)(OR^{e})₂, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —P(O)(OR^{a})R^{d}, —CH₂P(O)(OR^{a})R^{d}, —CH₂OP(O)(OR^{e})₂, —CH₂OC(O)C(R^{a})₂NR^{b}R^{c}, —SR^{a}, —S(O)R^{a}, —S(O)₂R^{a}, —S(O)NR^{b}R^{c}, or —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a}; and each R^{e} is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₅ aralkyl, heteroaryl, or heterocyclyl, each of which is optionally substituted with one or more, in one embodiment, one, two, three, or four, substituents Q^{a}; or (iv) two R^{e} together are a divalent cation (e.g., Mg²⁺ or Ca²⁺);

wherein each Q^{a} is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{f}, —C(O)OR^{f}, —C(O)NR^{g}R^{h}, —C(NR^{f})NR^{g}R^{h}, —OR^{f}, —OC(O)R^{f}, —OC(O)OR^{f}, —OC(O)NR^{g}R^{h}, —OC(═NR)NR^{g}R^{h}, —OP(O)(ORⁿ)₂, —OS(O)R^{f}, —OS(O)₂R^{f}, —OS(O)NR^{g}R^{h}, —OS(O)₂NR^{g}R^{h}, —NR^{g}R^{h}, —NR^{f}C(O)R^{k}, —NR^{f}C(O)OR^{k}, —NR^{f}C(O)NR^{g}R^{h}, —NR^{f}C(═NR^{k})NR^{g}R^{h}, —NR^{f}S(O)R^{k}, —NR^{f}S(O)₂R^{k}, —NR^{f}S(O)NR^{g}R^{h}, —NR^{f}S(O)₂NR^{g}R^{h}, —P(O)(OR^{f})R^{k}, —CH₂P(O)(OR^{f})R^{k}, —SR^{f}, —S(O)R^{f}, —S(O)₂R, —S(O)NR^{g}R^{h}, or —S(O)₂NR^{g}R^{h}; wherein each R^{f}, R^{g}, R^{h}, and R^{k} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{g} and R^{h} together with the N atom to which they are attached form heterocyclyl; and each Rⁿ is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iv) two Rⁿ together are a divalent cation (e.g., Mg²⁺ or Ca²⁺).

In certain embodiments of the compound of Formula IA, or single enantiomer, racemic mixture, diastereomer, mixture of diastereomers, or isotopic variant thereof; or pharmaceutically acceptable salt or solvate thereof;

A¹, A², and E are each independently (a) a bond; or (b) C₁₋₆ alkylene, C₂₋₆ alkenylene, C₂₋₆ alkynylene, C₂₋₂₀ cycloalkylene, C₆₋₂₀ arylene, heteroarylene; or heterocyclylene;

Z¹ and Z² are each independently a bond, —O—, —S—, —S(O)—, —S(O₂)—, or —N(R^{N})—;

R¹ and R² are each independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (c) —C(O)R^{a}, —C(O)CH(N(R^{1c})C(O)OR^{1b})R^{1a}, —C(O)OR^{a}, —C(O)NR^{1b}R^{1c}, —C(NR^{a})NR^{1b}R^{1c}, —P(O)(OR^{1a})R^{1d}, —CH₂P(O)(OR^{a})R^{1d}, —S(O)R^{1a}, —S(O)₂R^{1a}, —S(O)NR^{1b}R^{1c}, or —S(O)₂NR^{1b}R^{1c}; or (d) —C₁₋₆ alkylene-OP(O)(OR^{P1})₂, in one embodiment, —CH₂—OP(O)(OR^{P1})₂;

each R³ and R⁴ is independently (a) cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —SR^{1a}, —S(O)R^{1a}, —S(O)₂R^{1a}, —S(O)NR^{1b}R^{1c}, or —S(O)₂NR^{1b}R^{1c}; or

two R³ or two R⁴ that are attached to the same ring are linked together to form a bond, —O—, —NR^{N}—, —S—, C₁₋₆ alkylene, C₁₋₆ heteroalkylene, C₂₋₆ alkenylene, or C₂₋₆ heteroalkenylene;

each R^{N} is independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (c) —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{d}, —CH₂P(O)(OR^{1a})R^{1d}, —S(O)R^{1a}, —S(O)₂R^{1a}, —S(O)NR^{1b}R^{1c}, or —S(O)₂NR^{1b}R^{1c}; or (d) —C₁₋₆ alkylene-OP(O)(OR^{P1})₂, in one embodiment, —CH₂—OP(O)(OR^{P1})₂;

each R^{P} is independently absent, hydrogen, or —C₁₋₆ alkylene-OP(O)(OR^{P1})₂, in one embodiment, —CH₂—OP(O)(OR^{P1})₂; wherein, when the two R^{P} groups attached to the imidazolylene are neither absent nor hydrogen, the imidazolyene group carries a positive charge; and when the two R^{P} groups attached to the benzimidazolylene are neither absent nor hydrogen, the benzimidazolylene group carries a positive charge; and wherein at least one of the R^{P} groups is neither absent nor hydrogen;

each R^{P1} is independently (a) hydrogen; (b) a monovalent cation, in one embodiment, Na⁺ or K⁺; or (c) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or two R^{P1} together are a divalent cation, in one embodiment, Mg²⁺ or Ca²⁺;

each R^{1a}, R^{1b}, R^{1c}, and R^{1d} is independently hydrogen, C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or R^{1a} and R^{1c} together with the C and N atoms to which they are attached form heterocyclyl; or R^{1b} and R^{1c} together with the N atom to which they are attached form heterocyclyl;

each m and n is independently an integer of 1, 2, 3, or 4; and

each s and t is independently an integer of 0, 1, 2, 3, 4, 5, 6, or 7;

wherein each alkyl, alkylene, heteroalkylene, alkenyl, alkenylene, heteroalkenylene, alkynyl, alkynylene, cycloalkyl, cycloalkylene, aryl, arylene, aralkyl, heteroaryl, heteroarylene, imidazolylene, benzimidazolyl, heterocyclyl, and heterocyclylene is optionally substituted with one or more, in one embodiment, one, two, three, or four, substituents Q, where each Q is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is further optionally substituted with one or more, in one embodiment, one, two, three, or four, substituents Q^{a}; or (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OP(O)(OR^{e})₂, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —P(O)(OR^{a})R^{d}, —CH₂P(O)(OR^{a})R^{d}, —SR^{a}, —S(O)R^{a}, —S(O)₂R^{a}, —S(O)NR^{b}R^{c}, or —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a}; and each R^{e} is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iv) two R^{e} together are a divalent cation (e.g., Mg²⁺ or Ca²⁺);

wherein each Q^{a} is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{f}, —C(O)OR^{f}, —C(O)NR^{g}R^{h}, —C(NR^{f})NR^{g}R^{h}, —OR^{f}, —OC(O)R^{f}, —OC(O)OR^{f}, —OC(O)NR^{g}R^{h}, —OC(═NR^{f})NR^{g}R^{h}, —OP(O)(ORⁿ)₂, —OS(O)R^{f}, —OS(O)₂R^{f}, —OS(O)NR^{g}R^{h}, —OS(O)₂NR^{g}R^{h}, —NR^{g}R^{h}, —NR^{f}C(O)R^{k}, —NR^{f}C(O)OR^{k}, —NR^{f}C(O)NR^{g}R^{h}, —NR^{f}C(═NR^{k})NR^{g}R^{h}, —NR^{f}S(O)R^{k}, —NR^{f}S(O)₂R^{k}, —NR^{f}S(O)NR^{g}R^{h}, —NR^{f}S(O)₂NR^{g}R^{h}, —P(O)(OR^{f})R^{k}, —CH₂P(O)(OR^{f})R^{k}, —SR^{f}, —S(O)R^{f}, —S(O)₂R, —S(O)NR^{g}R^{h}, or —S(O)₂NR^{g}R^{h}; wherein each R^{f}, R^{g}, R^{h}, and R^{k} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{g} and R^{h} together with the N atom to which they are attached form heterocyclyl; and each Rⁿ is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iv) two Rⁿ together are a divalent cation (e.g., Mg²⁺ or Ca²⁺).

Additionally, provided herein is a compound of Formula IIA:

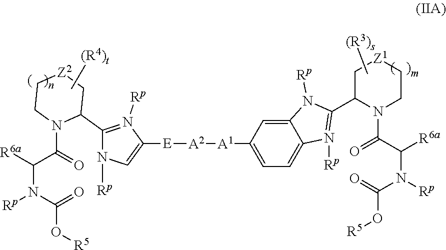

or a single enantiomer, a racemic mixture, a diastereomer, a mixture of diastereomers, or an isotopic variant thereof; or a pharmaceutically acceptable salt or solvate thereof; wherein:

A¹, A², and E are each independently (a) a bond; or (b) C₁₋₆ alkylene, C₂₋₆ alkenylene, C₂₋₆ alkynylene, C₂₋₂₀ cycloalkylene, C₆₋₂₀ arylene, heteroarylene; or heterocyclylene;

Z¹ and Z² are each independently a bond, —O—, —S—, —S(O)—, —S(O₂)—, or —N(R^{N})—;

each R³ and R⁴ is independently (a) cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —SR^{1a}, —S(O)R^{1a}, —S(O)₂R^{1a}, —S(O)NR^{1b}R^{1c}, or —S(O)₂NR^{1b}R^{1c}; or

two R³ or two R⁴ that are attached to the same ring are linked together to form a bond, —O—, —NR^{N}—, —S—, C₁₋₆ alkylene, C₁₋₆ heteroalkylene, C₂₋₆ alkenylene, or C₂₋₆ heteroalkenylene;

each R⁵ is independently C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl;

each R^{6a} is independently hydrogen, C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl;

each R^{N} is independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (c) —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R¹, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —S(O)R^{1a}, —S(O)₂R^{1a}, —S(O)NR^{1b}R^{1c}, or —S(O)₂NR^{1b}R^{1c}; (d) —C₁₋₆ alkylene-OP(O)(OR^{P1})₂, in one embodiment, —CH₂—OP(O)(OR^{P1})₂; or (e) —C₁₋₆ alkylene-O-linked amino acid or a derivative thereof, in one embodiment, —CH₂—OC(O)C(R^{aa})₂NR^{1b}R^{1c};

each R^{P} is independently absent, hydrogen, —C₁₋₆alkylene-OP(O)(OR^{P1})₂ (in one embodiment, —CH₂—OP(O)(OR^{P1})₂), or —C₁₋₆ alkylene-O-linked amino acid or a derivative thereof; (in one embodiment, —CH₂—OC(O)C(R^{aa})₂NR^{1b}R^{1c}); wherein, when the two R^{P} groups attached to the imidazolylene are neither absent nor hydrogen, the imidazolyene group carries a positive charge; and when the two R^{P} groups attached to the benzimidazolylene are neither absent nor hydrogen, the benzimidazolylene group carries a positive charge; with the proviso that at least one of the R^{P} groups is neither absent nor hydrogen;

each R^{P1} is independently (a) hydrogen; (b) a monovalent cation, in one embodiment, Na⁺ or K⁺, in another embodiment, Li⁺, Rb⁺, or Cs⁺; or (c) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or two R^{P1} together are a divalent cation, in one embodiment, Mg²⁺ or Ca²⁺;

each R^{1a}, R^{1b}, R^{1c}, and R^{1d} is independently hydrogen, C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or R^{1a} and R^{1c} together with the C and N atoms to which they are attached form heterocyclyl; or R^{1b} and R^{1c} together with the N atom to which they are attached form heterocyclyl;

each R^{aa} independently is a side chain of a naturally occurring or non-naturally occurring amino acid; in one embodiment, each R^{aa} independently is hydrogen, C₁₋₆ alkyl, heteroalkyl, C₆₋₁₄ aryl-C₁₋₆ alkyl and heteroaryl-C₁₋₆ alkyl;

each m and n is independently an integer of 1, 2, 3, or 4; and

each s and t is independently an integer of 0, 1, 2, 3, 4, 5, 6, or 7;

wherein each alkyl, alkylene, heteroalkylene, alkenyl, alkenylene, heteroalkenylene, alkynyl, alkynylene, cycloalkyl, cycloalkylene, aryl, arylene, aralkyl, heteroarylene, heteroaryl, imidazolylene, heterocyclyl, and heterocyclylene is optionally substituted with one or more, in one embodiment, one, two, three, or four, substituents Q, where each Q is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OP(O)(OR^{e})₂, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —P(O)(OR^{a})R^{d}, —CH₂P(O)(OR^{a})R^{d}, —SR^{a}, —S(O)R^{a}, —S(O)₂R^{a}, —S(O)NR^{b}R^{c}, or —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a}; and each R^{e} is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iv) two R^{e} together are a divalent cation (e.g., Mg²⁺ or Ca²⁺);

wherein each Q^{a} is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; and (c) —C(O)R^{f}, —C(O)OR^{f}, —C(O)NR^{g}R^{h}, —C(NR^{f})NR^{g}R^{h}, —OR^{f}, —OC(O)R^{f}, —OC(O)OR^{f}, —OC(O)NR^{g}R^{h}, —OC(═NR^{f})NR^{g}R^{h}, —OP(O)(ORⁿ)₂, —OS(O)R^{f}, —OS(O)₂R^{f}, —OS(O)NR^{g}R^{h}, —OS(O)₂NR^{g}R^{h}, —NR^{g}R^{h}, —NR^{f}C(O)R^{k}, —NR^{f}C(O)OR^{k}, —NR^{f}C(O)NR^{g}R^{h}, —NR^{f}C(═NR^{k})NR^{g}R^{h}, —NR^{f}S(O)R^{k}, —NR^{f}S(O)₂R^{k}, —NR^{f}S(O)NR^{g}R^{h}, —NR^{f}S(O)₂NR^{g}R^{h}, —P(O)(OR^{f})R^{k}, —CH₂P(O)(OR^{f})R^{k}, —SR^{f}, —S(O)R^{f}, —S(O)₂R, —S(O)NR^{g}R^{h}, or —S(O)₂NR^{g}R^{h}; wherein each R^{f}, R^{g}, R^{h}, and R^{k} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{g} and R^{h} together with the N atom to which they are attached form heterocyclyl; and each Rⁿ is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iv) two Rⁿ together are a divalent cation (e.g., Mg²⁺ or Ca²⁺).

In certain embodiments of the compound of Formula IIA, or single enantiomer, racemic mixture, diastereomer, mixture of diastereomers, or isotopic variant thereof; or pharmaceutically acceptable salt or solvate thereof;

A¹, A², and E are each independently (a) a bond; or (b) C₁₋₆ alkylene, C₂₋₆ alkenylene, C₂₋₆ alkynylene, C₂₋₂₀ cycloalkylene, C₆₋₂₀ arylene, heteroarylene; or heterocyclylene;

Z¹ and Z² are each independently a bond, —O—, —S—, —S(O)—, —S(O₂)—, or —N(R^{N})—;

each R³ and R⁴ is independently (a) cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1e}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —SR^{1a}, —S(O)R^{1a}, —S(O)₂R^{1a}, —S(O)NR^{1b}R^{1c}, or —S(O)₂NR^{1b}R^{1a}; or

two R³ or two R⁴ that are attached to the same ring are linked together to form a bond, —O—, —NR^{N}—, —S—, C₁₋₆ alkylene, C₁₋₆ heteroalkylene, C₂₋₆ alkenylene, or C₂₋₆ heteroalkenylene;

each R⁵ is independently C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl;

each R^{6a} is independently hydrogen, C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl;

each R^{N} is independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (c) —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —S(O)R^{1a}, —S(O)₂R^{1a}, —S(O)NR^{1b}R^{1c}, or —S(O)₂NR^{1b}R^{1c}; or (d) —C₁₋₆ alkylene-OP(O)(OR^{P1})₂, in one embodiment, —CH₂—OP(O)(OR^{P1})₂;

each R^{P} is independently absent, hydrogen, or —C₁₋₆ alkylene-OP(O)(OR^{P1})₂; in one embodiment, —CH₂—OP(O)(OR^{P1})₂; wherein, when the two R^{P} groups attached to the imidazolylene are neither absent nor hydrogen, the imidazolyene group carries a positive charge; and when the two R^{P} groups attached to the benzimidazolylene are neither absent nor hydrogen, the benzimidazolylene group carries a positive charge; with the proviso that at least one of the R^{P} groups is neither absent nor hydrogen;

each R^{P1} is independently (a) hydrogen; (b) a monovalent cation, in one embodiment, Na⁺ or K⁺; or (c) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or two R^{P1} together are a divalent cation, in one embodiment, Mg²⁺ or Ca²⁺;

each R¹, R^{1b}, R^{1c}, and R^{1d} is independently hydrogen, C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or R^{1a} and R^{1c} together with the C and N atoms to which they are attached form heterocyclyl; or R^{1b} and R^{1c} together with the N atom to which they are attached form heterocyclyl;

each m and n is independently an integer of 1, 2, 3, or 4; and

each s and t is independently an integer of 0, 1, 2, 3, 4, 5, 6, or 7;

wherein each alkyl, alkylene, heteroalkylene, alkenyl, alkenylene, heteroalkenylene, alkynyl, alkynylene, cycloalkyl, cycloalkylene, aryl, arylene, aralkyl, heteroarylene, heteroaryl, imidazolylene, heterocyclyl, and heterocyclylene is optionally substituted with one or more, in one embodiment, one, two, three, or four, substituents Q, where each Q is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OP(O)(OR^{e})₂, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —P(O)(OR^{a})R^{d}, —CH₂P(O)(OR^{a})R^{d}, —SR^{a}, —S(O)R^{a}, —S(O)₂R^{a}, —S(O)NR^{b}R^{c}, or —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and Re together with the N atom to which they are attached form heterocyclyl, optionally substituted with one or more, in one embodiment, one, two, three, or four, substituents Q^{a}; and each R^{e} is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iv) two R^{e} together are a divalent cation (e.g., Mg²⁺ or Ca²⁺);

wherein each Q^{a} is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; and (c) —C(O)R^{f}, —C(O)OR^{f}, —C(O)NR^{g}R^{h}, —C(NR^{f})NR^{g}R^{h}, —OR^{f}, —OC(O)R^{f}, —OC(O)OR^{f}, —OC(O)NR^{g}R^{h}, —OC(═NR^{f})NR^{g}R^{h}, —OP(O)(ORⁿ)₂, —OS(O)R^{f}, —OS(O)₂R^{f}, —OS(O)NR^{g}R^{h}, —OS(O)₂NR^{g}R^{h}, —NR^{g}R^{h}, —NR^{f}C(O)R^{k}, —NR^{f}C(O)OR^{k}, —NR^{f}C(O)NR^{g}R^{h}, —NR^{f}C(═NR^{k})NR^{g}R^{h}, —NR^{f}S(O)R^{k}, —NR^{f}S(O)₂R^{k}, —NR^{f}S(O)NR^{g}R^{h}, —NR^{f}S(O)₂NR^{g}R^{h}, —P(O)(OR^{f})R^{k}, —CH₂P(O)(OR^{f})R^{k}, —SR^{f}, —S(O)R^{f}, —S(O)₂R, —S(O)NR^{g}R^{h}, and —S(O)₂NR^{g}R^{h}; wherein each R^{f}, R^{g}, R^{h}, and R^{k} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{g} and R^{h} together with the N atom to which they are attached form heterocyclyl; and each Rⁿ is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iv) two Rⁿ together are a divalent cation (e.g., Mg²⁺ or Ca²⁺).

Provided herein is a compound of Formula IB:

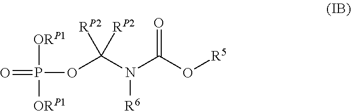

or a single enantiomer, a racemic mixture, a diastereomer, a mixture of diastereomers, or an isotopic variant thereof; or a pharmaceutically acceptable salt or solvate thereof;

R⁵ is C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl;

R⁶ is (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —CHR^{6a}C(O)R^{6b};

R^{6a} is hydrogen, C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl;

R^{6b} is:

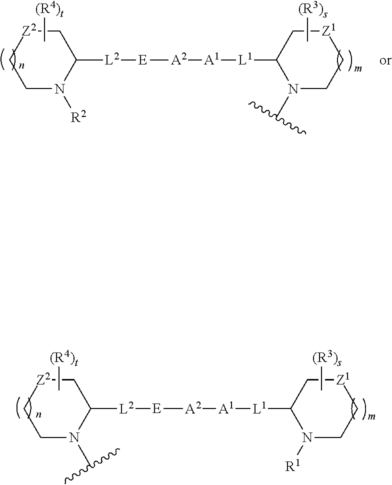

wherein:
A¹, A², and E are each independently (a) a bond; or (b) C₁₋₆ alkylene, C₂₋₆ alkenylene, C₂₋₆ alkynylene, C₂₋₂₀ cycloalkylene, C₆₋₂₀ arylene, heteroarylene; or heterocyclylene;
L¹ and L² are each independently (a) a bond; (b) C₁₋₆ alkylene, C₂₋₆ alkenylene, C₂₋₆ alkynylene, C₃₋₇cycloalkylene, C₆₋₁₄ arylene, heteroarylene, or heterocyclylene; or (c) —C(O)—, —C(O)O—, —C(O)NR^{a}—, —C(═NR^{1a})NR^{1c}—, —O—, —OC(O)O—, —OC(O)NR^{1a}—, —OC(═NR^{1a})NR^{1c}—, —OP(O)(OR^{1a})—, —OP(O)(OR^{1a})O—, —NR^{a}—, —NR^{1a}C(O)NR^{1c}—, —NR^{1a}C(═NR^{1b})NR^{1c}—, —NR^{1a}S(O)NR^{1c}—, —NR^{1a}S(O)₂NR^{1a}—, —S—, —S(O)—, —S(O)₂—, —S(O)NR^{1a}—, or —S(O)₂NR^{a}—;
Z¹ and Z² are each independently a bond, —O—, —S—, —S(O)—, —S(O₂)—, or —N(R^{N})—;
R¹ and R² are each independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (c) —C(O)R^{1a}, —C(O)CH(N(R^{1c})C(O)OR^{1b})R^{1a}, —C(O)OR^{1a}, —C(O)NR^{1b}R^{1c}, —C(NR^{1a})NR^{1b}R^{1c}, —P(O)(OR^{1a})R^{1d}, —CH₂P(O)(OR^{1a})R^{1d}, —S(O)R^{a}, —S(O)₂R^{1a}, —S(O)NR^{b}R^{1c}, or —S(O)₂NR^{1b}R^{1c}; (d) —C₁₋₆ alkylene-OP(O)(OR^{P1})₂, in one embodiment, —CH₂—OP(O)(OR^{P1})₂; or (e) —C₁₋₆ alkylene-O-linked amino acid or a derivative thereof, in one embodiment, —CH₂—OC(O)C(R^{aa})₂NR^{1b}R^{1c};
each R³ and R⁴ is independently (a) cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R¹, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{a})R^{1d}, —CH₂P(O)(OR^{1a})R^{1d}, —SR, —S(O)R^{1a}, —S(O)₂R^{1a}, —S(O)NR^{1b}R^{1c}, or —S(O)₂NR^{1b}R^{1c}; or
two R³ or two R⁴ that are attached to the same ring are linked together to form a bond, —O—, —NR^{N}—, —S—, C₁₋₆ alkylene, C₁₋₆ heteroalkylene, C₂₋₆ alkenylene, or C₂₋₆ heteroalkenylene;
each R^{N} is independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (c) —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1c}(O)OR^{1d}, —NR^{1a}C(O)NR^{1b}R^{1c}, —NR^{a}C(═NR^{1d})NR^{1b}R^{1c}, —NR^{1a}S(O)R^{d}, —NR^{1a}S(O)₂R^{d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{a})R^{1d}, —S(O)R^{1a}, —S(O)₂R^{a}, —S(O)NR^{1b}R^{1c}, or —S(O)₂NR^{1b}R^{1c}; (d) —C₁₋₆ alkylene-OP(O)(OR^{P1})₂, in one embodiment, —CH₂—OP(O)(OR^{P1})₂; or (e) —C₁₋₆ alkylene-O-linked amino acid or a derivative thereof, in one embodiment, —CH₂—OC(O)C(R^{aa})₂NR^{1b}R^{1c};
each m and n is independently an integer of 1, 2, 3, or 4; and
each s and t is independently an integer of 0, 1, 2, 3, 4, 5, 6, or 7;

each R^{P1} is independently (a) hydrogen; (b) a monovalent cation, in one embodiment, Na⁺ or K⁺, in another embodiment, Li⁺, Rb⁺, or Cs⁺; (c) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (d) two R^{P1} together are a divalent cation, in one embodiment, Mg²⁺ or Ca²⁺; and

each R^{P2} is independently (a) hydrogen, cyano, halo, or nitro; or (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl;

each R^{1a}, R^{1b}, R^{1c}, and R^{1d} is independently hydrogen, C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or R^{1a} and R^{1c} together with the C and N atoms to which they are attached form heterocyclyl; or R^{1b} and R^{1c} together with the N atom to which they are attached form heterocyclyl;

each R^{aa} independently is a side chain of a naturally occurring or non-naturally occurring amino acid; in one embodiment, each R^{aa} independently is hydrogen, C₁₋₆ alkyl, heteroalkyl, C₆₋₁₄ aryl-C₁₋₆ alkyl and heteroaryl-C₁₋₆ alkyl;

wherein each alkyl, alkylene, heteroalkylene, alkenyl, alkenylene, heteroalkenylene, alkynyl, alkynylene, cycloalkyl, cycloalkylene, aryl, arylene, aralkyl, heteroaryl, heteroarylene, heterocyclyl, and heterocyclylene is optionally substituted with one or more, in one embodiment, one, two, three, or four, substituents Q, where each Q is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OP(O)(OR^{e})₂, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —P(O)(OR^{a})R^{d}, —CH₂P(O)(OR^{a})R^{d}, —CH₂OP(O)(OR^{e})₂, —CH₂OC(O)C(R^{a})₂NR^{b}R^{c}, —SR^{a}, —S(O)R^{a}, —S(O)₂R^{a}, —S(O)NR^{b}R^{c}, or —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a}; and each R^{e} is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iv) two R^{e} together are a divalent cation (e.g., Mg²⁺ or Ca²⁺);

wherein each Q^{a} is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; and (c) —C(O)R^{f}, —C(O)OR^{f}, —C(O)NR^{g}R^{h}, —C(NR^{f})NR^{g}R^{h}, —OR^{f}, —OC(O)R^{f}, —OC(O)OR^{f}, —OC(O)NR^{g}R^{h}, —OC(═NR^{f})NR^{g}R^{h}, —OP(O)(ORⁿ)₂, —OS(O)R^{f}, —OS(O)₂R^{f}, —OS(O)NR^{g}R^{h}, —OS(O)₂NR^{g}R^{h}, —NR^{g}R^{h}, —NR^{f}C(O)R^{k}, —NR^{f}C(O)OR^{k}, —NR^{f}C(O)NR^{g}R^{h}, —NR^{f}C(═NR^{k})NR^{g}R^{h}, —NR^{f}S(O)R^{k}, —NR^{f}S(O)₂R^{k}, —NR^{f}S(O)NR^{g}R^{h}, —NR^{f}S(O)₂NR^{g}R^{h}, —P(O)(OR^{f})R^{k}, —CH₂P(O)(OR^{f})R^{k}, —SR^{f}, —S(O)R^{f}, —S(O)₂R, —S(O)NR^{g}R^{h}, or —S(O)₂NR^{g}R^{h}; wherein each R^{f}, R^{g}, R^{h}, and R^{k} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{g} and R^{h} together with the N atom to which they are attached form heterocyclyl; and each Rⁿ is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iv) two Rⁿ together are a divalent cation (e.g., Mg²⁺ or Ca²⁺).

In certain embodiments of the compound of Formula IB, or single enantiomer, racemic mixture, diastereomer, mixture of diastereomers, or isotopic variant thereof; or pharmaceutically acceptable salt or solvate thereof;

R⁵ is C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl;

R⁶ is (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —CHR^{6a}C(O)R^{6b};

R^{6a} is hydrogen, C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl;

R^{6b} is:

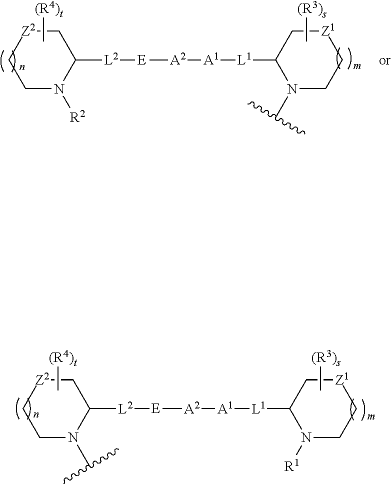

wherein:
A¹, A², and E are each independently (a) a bond; or (b) C₁₋₆ alkylene, C₂₋₆ alkenylene, C₂₋₆ alkynylene, C₂₋₂₀ cycloalkylene, C₆₋₂₀ arylene, heteroarylene; or heterocyclylene;
L¹ and L² are each independently (a) a bond; (b) C₁₋₆ alkylene, C₂₋₆ alkenylene, C₂₋₆ alkynylene, C₃₋₇ cycloalkylene, C₆₋₁₄ arylene, heteroarylene, or heterocyclylene; or (c) —C(O)—, —C(O)O—, —C(O)NR^{1a}—, —C(═NR^{1a})NR^{1c}—, —O—, —OC(O)O—, —OC(O)NR^{1a}—, —OC(═NR^{1a})NR^{1c}—, —OP(O)(OR^{1a})—, —OP(O)(OR^{1a})O—, —NR^{1a}—, —NR^{1a}C(O)NR^{1c}—, —NR^{1a}C(═NR^{b})NR^{1c}—, —NR^{1a}S(O)NR^{1c}—, —NR^{1a}S(O)₂NR^{1a}—, —S—, —S(O)—, —S(O)₂—, —S(O)NR^{1c}—, or —S(O)₂NR^{a}—;
Z¹ and Z² are each independently a bond, —O—, —S—, —S(O)—, —S(O₂)—, or —N(R^{N})—;
R¹ and R² are each independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (c) —C(O)R^{1a}, —C(O)CH(N(R^{1c})C(O)OR^{1b})R^{1a}, —C(O)OR^{1a}, —C(O)NR^{1b}R^{1c}, —C(NR^{1a})NR^{1b}R^{1c}, —P(O)(OR^{1a})R^{1d}, —CH₂P(O)(OR^{1a})R^{1d}, —S(O)R^{1a}, —S(O)₂R^{1a}, —S(O)NR^{1b}R^{1c}, or —S(O)₂NR^{1b}R^{1c}; or (d) —C₁₋₆ alkylene-OP(O)(OR^{P1})₂, in one embodiment, —CH₂—OP(O)(OR^{P1})₂;
each R³ and R⁴ is independently (a) cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —SR^{a}, —S(O)R^{1a}, —S(O)₂R^{1a}, —S(O)NR^{1b}R^{1c}, or —S(O)₂NR^{1b}R^{1c}; or
two R³ or two R⁴ that are attached to the same ring are linked together to form a bond, —O—, —NR^{N}—, —S—, C₁₋₆ alkylene, C₁₋₆ heteroalkylene, C₂₋₆ alkenylene, or C₂₋₆ heteroalkenylene;
each R^{N} is independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (c) —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{a})R^{1d}, —S(O)R^{1a}, —S(O)₂R^{a}, —S(O)NR^{1b}R^{1c}, or —S(O)₂NR^{1b}R^{1e}; or (d) —C₁₋₆ alkylene-OP(O)(OR^{P1})₂, in one embodiment, —CH₂—OP(O)(OR^{P1})₂;
each m and n is independently an integer of 1, 2, 3, or 4; and
each s and t is independently an integer of 0, 1, 2, 3, 4, 5, 6, or 7;

each R^{P1} is independently (a) hydrogen; (b) a monovalent cation, in one embodiment, Na⁺ or K⁺; (c) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (d) two R^{P1} together are a divalent cation, in one embodiment, Mg²⁺ or Ca²⁺; and

each R^{P2} is independently (a) hydrogen, cyano, halo, or nitro; or (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl;

each R^{1a}, R^{1b}, R^{1c}, and R^{1d} is independently hydrogen, C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or R^{1a} and R^{1e} together with the C and N atoms to which they are attached form heterocyclyl; or R^{1b} and R^{1c} together with the N atom to which they are attached form heterocyclyl;

wherein each alkyl, alkylene, heteroalkylene, alkenyl, alkenylene, heteroalkenylene, alkynyl, alkynylene, cycloalkyl, cycloalkylene, aryl, arylene, aralkyl, heteroaryl, heteroarylene, heterocyclyl, and heterocyclylene is optionally substituted with one or more, in one embodiment, one, two, three, or four, substituents Q, where each Q is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OP(O)(OR^{e})₂, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —P(O)(OR^{a})R^{d}, —CH₂P(O)(OR^{a})R^{d}, —SR^{a}, —S(O)R^{a}, —S(O)₂R^{a}, —S(O)NR^{b}R^{c}, or —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and Re together with the N atom to which they are attached form heterocyclyl, optionally substituted with one or more, in one embodiment, one, two, three, or four, substituents Q^{a}; and each R^{e} is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iv) two R^{e} together are a divalent cation (e.g., Mg²⁺ or Ca²⁺);

wherein each Q^{a} is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; and (c) —C(O)R^{f}, —C(O)OR^{f}, —C(O)NR^{g}R^{h},

—C(NR^{f})NR^{g}R^{h}, —OR^{f}, —OC(O)R^{f}, —OC(O)OR^{f}, —OC(O)NR^{g}R^{h}, —OC(═NR^{f})NR^{g}R^{h}, —OP(O)(ORⁿ)₂, —OS(O)R^{f}, —OS(O)₂R^{f}, —OS(O)NR^{g}R^{h}, —OS(O)₂NR^{g}R^{h}, —NR^{g}R^{h}, —NR^{f}C(O)R^{k}, —NR^{f}C(O)OR^{k}, —NR^{f}C(O)NR^{g}R^{h}, —NR^{f}C(═NR^{k})NR^{g}R^{h}, —NR^{f}S(O)R^{k}, —NR^{f}S(O)₂R^{k}, —NR^{f}S(O)NR^{g}R^{h}, —NR^{f}S(O)₂NR^{g}R^{h}, —P(O)(OR^{f})R^{k}, —CH₂P(O)(OR^{f})R^{k}, —SR^{f}, —S(O)R^{f}, —S(O)₂R, —S(O)NR^{g}R^{h}, or —S(O)₂NR^{g}R^{h}; wherein each R^{f}, R^{g}, R^{h}, and R^{k} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{g} and R^{h} together with the N atom to which they are attached form heterocyclyl; and each Rⁿ is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iv) two Rⁿ together are a divalent cation (e.g., Mg²⁺ or Ca²⁺).

Further provided herein are pharmaceutical compositions comprising a compound disclosed herein, e.g., a compound of any of Formulae disclosed herein, including Formulae I to XIV, IIIa to XIVa, IIIb to XIVb, IIIc to XIVc, IIId to XIVd, IIIe to XIVe, IA to IAe, IIA to IIAe, IB, IIB to IIBd, IIIB to IIIBd, IC to ICc, ID to IDd, and IE to IEc, including a single enantiomer, a racemic mixture, a diastereomer, a mixture of diastereomers, or an isotopic variant thereof; or a pharmaceutically acceptable salt or solvate thereof; and optionally one or more pharmaceutically acceptable excipients or carriers.

Provided herein is a method for treating or preventing an HCV infection in a subject, which comprises administering to the subject a compound disclosed herein, e.g., a compound of any of Formulae disclosed herein, including Formulae I to XIV, IIIa to XIVa, IIIb to XIVb, IIIc to XIVc, IIId to XIVd, IIIe to XIVe, IA to IAe, IIA to IIAe, IB, IIB to IIBd, IIIB to IIIBd, IC to ICc, ID to IDd, and IE to IEc, including a single enantiomer, a racemic mixture, a diastereomer, a mixture of diastereomers, or an isotopic variant thereof; or a pharmaceutically acceptable salt or solvate thereof.

Provided herein is a method for treating, preventing, or ameliorating one or more symptoms of a liver disease or disorder associated with an HCV infection in a subject, comprising administering to the subject a compound disclosed herein, e.g., a compound of any of Formulae disclosed herein, including Formulae I to XIV, IIIa to XIVa, IIIb to XIVb, IIIc to XIVc, IIId to XIVd, IIIe to XIVe, IA to IAe, IIA to IIAe, IB, IIB to IIBd, IIIB to IIIBd, IC to ICc, ID to IDd, and IE to IEc, including a single enantiomer, a racemic mixture, a diastereomer, a mixture of diastereomers, or an isotopic variant thereof; or a pharmaceutically acceptable salt or solvate thereof.

Provided herein is a method for inhibiting replication of a virus in a host, comprising contacting the host with a compound disclosed herein, e.g., a compound of any of Formulae disclosed herein, including Formulae I to XIV, IIIa to XIVa, IIIb to XIVb, IIIc to XIVc, IIId to XIVd, IIIe to XIVe, IA to IAe, IIA to IIAe, IB, IIB to IIBd, IIIB to IIIBd, IC to ICc, ID to IDd, and IE to IEc, including a single enantiomer, a racemic mixture, a diastereomer, a mixture of diastereomers, or an isotopic variant thereof; or a pharmaceutically acceptable salt or solvate thereof.

### DETAILED DESCRIPTION

To facilitate understanding of the disclosure set forth herein, a number of terms are defined below.

Generally, the nomenclature used herein and the laboratory procedures in organic chemistry, medicinal chemistry, and pharmacology described herein are those well known and commonly employed in the art. Unless defined otherwise, all technical and scientific terms used herein generally have the same meaning as commonly understood by one of ordinary skill in the art to which this disclosure belongs.

The term “subject” refers to an animal, including, but not limited to, a primate (e.g., human), cow, pig, sheep, goat, horse, dog, cat, rabbit, rat, or mouse. The terms “subject” and “patient” are used interchangeably herein in reference, for example, to a mammalian subject, such as a human subject, in one embodiment, a human.

The term “host” refers to a unicellular or multicellular organism in which a virus can replicate, including, but not limited to, a cell, cell line, and animal, such as a human.

The terms “treat,” “treating,” and “treatment” are meant to include alleviating or abrogating a disorder, disease, or condition, or one or more of the symptoms associated with the disorder, disease, or condition; or alleviating or eradicating the cause(s) of the disorder, disease, or condition itself.

The terms “prevent,” “preventing,” and “prevention” are meant to include a method of delaying and/or precluding the onset of a disorder, disease, or condition, and/or its attendant symptoms; barring a subject from acquiring a disorder, disease, or condition; or reducing a subject's risk of acquiring a disorder, disease, or condition.

The term “therapeutically effective amount” are meant to include the amount of a compound that, when administered, is sufficient to prevent development of, or alleviate to some extent, one or more of the symptoms of the disorder, disease, or condition being treated. The term “therapeutically effective amount” also refers to the amount of a compound that is sufficient to elicit the biological or medical response of a biological molecule (e.g., a protein, enzyme, RNA, or DNA), cell, tissue, system, animal, or human, which is being sought by a researcher, veterinarian, medical doctor, or clinician.

The term “IC₅₀” or “EC₅₀” refers an amount, concentration, or dosage of a compound that is required for 50% inhibition of a maximal response in an assay that measures such a response.

The term “CC₅₀” refers an amount, concentration, or dosage of a compound that results in 50% reduction of the viability of a host. In certain embodiments, the CC₅₀ of a compound is the amount, concentration, or dosage of the compound that is required to reduce the viability of cells treated with the compound by 50%, in comparison with cells untreated with the compound.

The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 50%, 20%, 15%, 10%, 9%, 8%, 7%, 6%, 5%, 4%, 3%, 2%, 1%, 0.5%, or 0.05% of a given value or range.

The terms “active ingredient” and “active substance” refer to a compound, which is administered, alone or in combination with one or more pharmaceutically acceptable excipients, to a subject for treating, preventing, or ameliorating one or more symptoms of a condition, disorder, or disease. As used herein, “active ingredient” and “active substance” may be an optically active isomer or an isotopic variant of a compound described herein.

The terms “drug,” “therapeutic agent,” and “chemotherapeutic agent” refer to a compound, or a pharmaceutical composition thereof, which is administered to a subject for treating, preventing, or ameliorating one or more symptoms of a condition, disorder, or disease.

The term “hepatitis C virus” or “HCV” refers to a viral species or a variant thereof, a pathogenic strain of which causes hepatitis C. Examples of HCV include, but are not limited to, HCV genotypes 1, 2, 3, 4, 5, 6, 7, 8, 9, 10, 11, and subtype 1a, 1b, 1c, 2a, 2b, 2c, 3a, 3b, 4a, 4b, 4c, 4d, 4e, 5a, 6a, 7a, 7b, 8a, 8b, 9a, 10a, and 11a. In certain embodiments, an HCV variant is an HCV species that contains a protein substantially homologous to a native HCV protein, i.e., a protein having one or more naturally or non-naturally occurring amino acid deletions, insertions or substitutions (e.g., derivatives, homologs, and fragments), as compared to the amino acid sequence of the native protein. The amino acid sequence of a protein of an HCV variant is at least about 80% identical, at least about 90% identical, or at least about 95% identical to a native HCV protein. In certain embodiments, the HCV variant contains an NS5A protein variant.

The term “NS5A” refers to nonstructural protein 5A of an HCV, or a variant thereof. NS5A variants include proteins substantially homologous to a native NS5A, i.e., proteins having one or more naturally or non-naturally occurring amino acid deletions, insertions or substitutions (e.g., NS5A derivatives, homologs, and fragments), as compared to the amino acid sequence of a native NS5A. The amino acid sequence of an NS5A variant is at least about 80% identical, at least about 90% identical, or at least about 95% identical to a native NS5A.

The term “alkyl” refers to a linear or branched saturated monovalent hydrocarbon radical, wherein the alkyl may optionally be substituted with one or more substituents Q as described herein. For example, C₁₋₆ alkyl refers to a linear saturated monovalent hydrocarbon radical of 1 to 6 carbon atoms or a branched saturated monovalent hydrocarbon radical of 3 to 6 carbon atoms. In certain embodiments, the alkyl is a linear saturated monovalent hydrocarbon radical that has 1 to 20 (C₁₋₂₀), 1 to 15 (C₁₋₅), 1 to 10 (C₁₋₁₀), or 1 to 6 (C₁₋₆) carbon atoms, or branched saturated monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkyl groups are also referred as “lower alkyl.” Examples of alkyl groups include, but are not limited to, methyl, ethyl, propyl (including all isomeric forms), n-propyl, isopropyl, butyl (including all isomeric forms), n-butyl, isobutyl, sec-butyl, t-butyl, pentyl (including all isomeric forms), and hexyl (including all isomeric forms).

The term “alkylene” refers to a linear or branched saturated divalent hydrocarbon radical, wherein the alkylene may optionally be substituted with one or more substituents Q as described herein. For example, C₁₋₆ alkylene refers to a linear saturated divalent hydrocarbon radical of 1 to 6 carbon atoms or a branched saturated divalent hydrocarbon radical of 3 to 6 carbon atoms. In certain embodiments, the alkylene is a linear saturated divalent hydrocarbon radical that has 1 to 20 (C₁₋₂₀), 1 to 15 (C₁₋₅), 1 to 10 (C₁₋₁₀), or 1 to 6 (C₁₋₆) carbon atoms, or branched saturated divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ alkylene groups are also referred as “lower alkylene.” Examples of alkylene groups include, but are not limited to, methylene, ethylene, propylene (including all isomeric forms), n-propylene, isopropylene, butylene (including all isomeric forms), n-butylene, isobutylene, t-butylene, pentylene (including all isomeric forms), and hexylene (including all isomeric forms).

The term “heteroalkylene” refers to a linear or branched saturated divalent hydrocarbon radical that contains one or more heteroatoms in the hydrocarbon chain, each of which is independently selected from O, S, and N. For example, C₁₋₆ heteroalkylene refers to a linear saturated divalent hydrocarbon radical of 1 to 6 carbon atoms or a branched saturated divalent hydrocarbon radical of 3 to 6 carbon atoms. In certain embodiments, the heteroalkylene is a linear saturated divalent hydrocarbon radical that has 1 to 20 (C₁₋₂₀), 1 to 15 (C₁₋₅), 1 to 10 (C₁₋₁₀), or 1 to 6 (C₁₋₆) carbon atoms, or branched saturated divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. As used herein, linear C₁₋₆ and branched C₃₋₆ heteroalkylene groups are also referred as “lower heteroalkylene.” Examples of heteroalkylene groups include, but are not limited to, —CH₂O—, —CH₂OCH₂—, —CH₂CH₂O—, —CH₂NH—, —CH₂NHCH₂—, —CH₂CH₂NH—, —CH₂S—, —CH₂SCH₂—, and —CH₂CH₂S—. In certain embodiments, heteroalkylene may also be optionally substituted with one or more substituents Q as described herein.

The term “alkenyl” refers to a linear or branched monovalent hydrocarbon radical, which contains one or more, in one embodiment, one, two, three, four, or five, in another embodiment, one or two, carbon-carbon double bond(s). The alkenyl may be optionally substituted with one or more substituents Q as described herein. The term “alkenyl” embraces radicals having a “cis” or “trans” configuration or a mixture thereof, or alternatively, a “Z” or “E” configuration or a mixture thereof, as appreciated by those of ordinary skill in the art. For example, C₂₋₆ alkenyl refers to a linear unsaturated monovalent hydrocarbon radical of 2 to 6 carbon atoms or a branched unsaturated monovalent hydrocarbon radical of 3 to 6 carbon atoms. In certain embodiments, the alkenyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenyl groups include, but are not limited to, ethenyl, propen-1-yl, propen-2-yl, allyl, butenyl, and 4-methylbutenyl.

The term “alkenylene” refers to a linear or branched divalent hydrocarbon radical, which contains one or more, in one embodiment, one, two, three, four, or five, in another embodiment, one or two, carbon-carbon double bond(s). The alkenylene may be optionally substituted with one or more substituents Q as described herein. The term “alkenylene” embraces radicals having a “cis” or “trans” configuration or a mixture thereof, or alternatively, a “Z” or “E” configuration or a mixture thereof, as appreciated by those of ordinary skill in the art. For example, C₂₋₆ alkenylene refers to a linear unsaturated divalent hydrocarbon radical of 2 to 6 carbon atoms or a branched unsaturated divalent hydrocarbon radical of 3 to 6 carbon atoms. In certain embodiments, the alkenylene is a linear divalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkenylene groups include, but are not limited to, ethenylene, allylene, propenylene, butenylene, and 4-methylbutenylene.

The term “heteroalkenylene” refers to a linear or branched divalent hydrocarbon radical, which contains one or more, in one embodiment, one, two, three, four, or five, in another embodiment, one or two, carbon-carbon double bond(s), and which contains one or more heteroatoms in the hydrocarbon chain, each of which is independently selected from O, S, and N. The heteroalkenylene may be optionally substituted with one or more substituents Q as described herein. The term “heteroalkenylene” embraces radicals having a “cis” or “trans” configuration or a mixture thereof, or alternatively, a “Z” or “E” configuration or a mixture thereof, as appreciated by those of ordinary skill in the art. For example, C₂₋₆ heteroalkenylene refers to a linear unsaturated divalent hydrocarbon radical of 2 to 6 carbon atoms or a branched unsaturated divalent hydrocarbon radical of 3 to 6 carbon atoms. In certain embodiments, the heteroalkenylene is a linear divalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of heteroalkenylene groups include, but are not limited to, —CH═CHO—, —CH═CHOCH₂—, —CH═CHCH₂O—, —CH═CHS—, —CH═CHSCH₂—, —CH═CHCH₂S—, or —CH═CHCH₂NH—.

The term “alkynyl” refers to a linear or branched monovalent hydrocarbon radical, which contains one or more, in one embodiment, one, two, three, four, or five, in another embodiment, one or two, carbon-carbon triple bond(s). The alkynyl may be optionally substituted with one or more substituents Q as described herein. For example, C₂₋₆ alkynyl refers to a linear unsaturated monovalent hydrocarbon radical of 2 to 6 carbon atoms or a branched unsaturated monovalent hydrocarbon radical of 3 to 6 carbon atoms. In certain embodiments, the alkynyl is a linear monovalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched monovalent hydrocarbon radical of 3 to 20 (C₃₋₂₀), 3 to 15 (C₃₋₁₅), 3 to 10 (C₃₋₁₀), or 3 to 6 (C₃₋₆) carbon atoms. Examples of alkynyl groups include, but are not limited to, ethynyl (—C≡CH), propynyl (including all isomeric forms, e.g., 1-propynyl (—C≡CCH₃) and propargyl (—CH₂C≡CH)), butynyl (including all isomeric forms, e.g., 1-butyn-1-yl and 2-butyn-1-yl), pentynyl (including all isomeric forms, e.g., 1-pentyn-1-yl and 1-methyl-2-butyn-1-yl), and hexynyl (including all isomeric forms, e.g., 1-hexyn-1-yl).

The term “alkynylene” refers to a linear or branched divalent hydrocarbon radical, which contains one or more, in one embodiment, one, two, three, four, or five, in another embodiment, one or two, carbon-carbon triple bond(s). The alkynylene may be optionally substituted with one or more substituents Q as described herein. For example, C₂₋₆ alkynylene refers to a linear unsaturated divalent hydrocarbon radical of 2 to 6 carbon atoms or a branched unsaturated divalent hydrocarbon radical of 3 to 6 carbon atoms. In certain embodiments, the alkynylene is a linear divalent hydrocarbon radical of 2 to 20 (C₂₋₂₀), 2 to 15 (C₂₋₁₅), 2 to 10 (C₂₋₁₀), or 2 to 6 (C₂₋₆) carbon atoms, or a branched divalent hydrocarbon radical of 3 to 20 (C₃₋₂₀), 3 to 15 (C₃₋₁₅), 3 to 10 (C₃₋₁₀), or 3 to 6 (C₃₋₆) carbon atoms.

Examples of alkynylene groups include, but are not limited to, ethynylene, propynylene (including all isomeric forms, e.g., 1-propynylene and propargylene), butynylene (including all isomeric forms, e.g., 1-butyn-1-ylene and 2-butyn-1-ylene), pentynylene (including all isomeric forms, e.g., 1-pentyn-1-ylene and 1-methyl-2-butyn-1-ylene), and hexynylene (including all isomeric forms, e.g., 1-hexyn-1-ylene).

The term “cycloalkyl” refers to a cyclic monovalent hydrocarbon radical, which may be optionally substituted with one or more substituents Q as described herein. In one embodiment, cycloalkyl groups may be saturated or unsaturated but non-aromatic, and/or bridged, and/or non-bridged, and/or fused bicyclic groups. In certain embodiments, the cycloalkyl has from 3 to 20 (C₃₋₂₀), from 3 to 15 (C₃₋₁₅), from 3 to 10 (C₃₋₁₀), or from 3 to 7 (C₃₋₇) carbon atoms. Examples of cycloalkyl groups include, but are not limited to, cyclopropyl, cyclobutyl, cyclopentyl, cyclopentenyl, cyclohexyl, cyclohexenyl, cyclohexadienyl, cycloheptyl, cycloheptenyl, bicyclo[2.1.1]hexyl, bicyclo[2.2.1]heptyl, decalinyl, and adamantyl.

The term “cycloalkylene” refers to a cyclic divalent hydrocarbon radical, which may be optionally substituted with one or more substituents Q as described herein. In one embodiment, cycloalkyl groups may be saturated or unsaturated but non-aromatic, and/or bridged, and/or non-bridged, and/or fused bicyclic groups. In certain embodiments, the cycloalkylene has from 3 to 20 (C₃₋₂₀), from 3 to 15 (C₃₋₁₅), from 3 to 10 (C₃₋₁₀), or from 3 to 7 (C₃₋₇) carbon atoms. Examples of cycloalkylene groups include, but are not limited to, cyclopropylene (e.g., 1,1-cyclopropylene and 1,2-cyclopropylene), cyclobutylene (e.g., 1,1-cyclobutylene, 1,2-cyclobutylene, or 1,3-cyclobutylene), cyclopentylene (e.g., 1,1-cyclopentylene, 1,2-cyclopentylene, or 1,3-cyclopentylene), cyclohexylene (e.g., 1,1-cyclohexylene, 1,2-cyclohexylene, 1,3-cyclohexylene, or 1,4-cyclohexylene), cycloheptylene (e.g., 1,1-cycloheptylene, 1,2-cycloheptylene, 1,3-cycloheptylene, or 1,4-cycloheptylene), decalinylene, and adamantylene.

The term “aryl” refers to a monovalent monocyclic aromatic group or monovalent polycyclic aromatic group that contains at least one aromatic carbon ring. In certain embodiments, the aryl has from 6 to 20 (C₆₋₂₀), from 6 to 15 (C₆₋₁₅), or from 6 to 10 (C₆₋₁₀) ring atoms. Examples of aryl groups include, but are not limited to, phenyl, naphthyl, fluorenyl, azulenyl, anthryl, phenanthryl, pyrenyl, biphenyl, and terphenyl. Aryl also refers to bicyclic or tricyclic carbon rings, where one of the rings is aromatic and the others of which may be saturated, partially unsaturated, or aromatic, for example, dihydronaphthyl, indenyl, indanyl, or tetrahydronaphthyl (tetralinyl). In certain embodiments, aryl may be optionally substituted with one or more substituents Q as described herein.

The term “arylene” refers to a divalent monocyclic aromatic group or divalent polycyclic aromatic group that contains at least one aromatic carbon ring. In certain embodiments, the arylene has from 6 to 20 (C₆₋₂₀), from 6 to 15 (C₆₋₁₅), or from 6 to 10 (C₆₋₁₀) ring atoms. Examples of arylene groups include, but are not limited to, phenylene, naphthylene, fluorenylene, azulenylene, anthrylene, phenanthrylene, pyrenylene, biphenylene, and terphenylene. Arylene also refers to bicyclic or tricyclic carbon rings, where one of the rings is aromatic and the others of which may be saturated, partially unsaturated, or aromatic, for example, dihydronaphthylene, indenylene, indanylene, or tetrahydronaphthylene (tetralinylene). In certain embodiments, arylene may be optionally substituted with one or more substituents Q as described herein.

The term “aralkyl” or “arylalkyl” refers to a monovalent alkyl group substituted with one or more aryl groups. In certain embodiments, the aralkyl has from 7 to 30 (C₇₋₃₀), from 7 to 20 (C₇₋₂₀), or from 7 to 16 (C₇₋₁₆) carbon atoms. Examples of aralkyl groups include, but are not limited to, benzyl, 2-phenylethyl, and 3-phenylpropyl. In certain embodiments, aralkyl are optionally substituted with one or more substituents Q as described herein.

The term “heteroaryl” refers to a monovalent monocyclic aromatic group or monovalent polycyclic aromatic group that contains at least one aromatic ring, wherein at least one aromatic ring contains one or more heteroatoms in the ring, each of which is independently selected from O, S, and N. Heteroaryl groups are bonded to the rest of a molecule through the aromatic ring. Each ring of a heteroaryl group can contain one or two O atoms, one or two S atoms, and/or one to four N atoms, provided that the total number of heteroatoms in each ring is four or less and each ring contains at least one carbon atom. In certain embodiments, the heteroaryl has from 5 to 20, from 5 to 15, or from 5 to 10 ring atoms. Examples of monocyclic heteroaryl groups include, but are not limited to, furanyl, imidazolyl, isothiazolyl, isoxazolyl, oxadiazolyl, oxadiazolyl, oxazolyl, pyrazinyl, pyrazolyl, pyridazinyl, pyridyl, pyrimidinyl, pyrrolyl, thiadiazolyl, thiazolyl, thienyl, tetrazolyl, triazinyl, and triazolyl. Examples of bicyclic heteroaryl groups include, but are not limited to, benzofuranyl, benzimidazolyl, benzoisoxazolyl, benzopyranyl, benzothiadiazolyl, benzothiazolyl, benzothienyl, benzotriazolyl, benzoxazolyl, furopyridyl, imidazopyridinyl, imidazothiazolyl, indolizinyl, indolyl, indazolyl, isobenzofuranyl, isobenzothienyl, isoindolyl, isoquinolinyl, isothiazolyl, naphthyridinyl, oxazolopyridinyl, phthalazinyl, pteridinyl, purinyl, pyridopyridyl, pyrrolopyridyl, quinolinyl, quinoxalinyl, quinazolinyl, thiadiazolopyrimidyl, and thienopyridyl. Examples of tricyclic heteroaryl groups include, but are not limited to, acridinyl, benzindolyl, carbazolyl, dibenzofuranyl, perimidinyl, phenanthrolinyl, phenanthridinyl, phenarsazinyl, phenazinyl, phenothiazinyl, phenoxazinyl, and xanthenyl. In certain embodiments, heteroaryl may also be optionally substituted with one or more substituents Q as described herein.

The term “heteroarylene” refers to a divalent monocyclic aromatic group or divalent polycyclic aromatic group that contains at least one aromatic ring, wherein at least one aromatic ring contains one or more heteroatoms in the ring, each of which is independently selected from O, S, and N. Each ring of a heteroarylene group can contain one or two O atoms, one or two S atoms, and/or one to four N atoms, provided that the total number of heteroatoms in each ring is four or less and each ring contains at least one carbon atom. In certain embodiments, the heteroarylene has from 5 to 20, from 5 to 15, or from 5 to 10 ring atoms. Examples of monocyclic heteroarylene groups include, but are not limited to, furanylene, imidazolylene, isothiazolylene, isoxazolylene, oxadiazolylene, oxadiazolylene, oxazolylene, pyrazinylene, pyrazolylene, pyridazinylene, pyridylene, pyrimidinylene, pyrrolylene, thiadiazolylene, thiazolylene, thienylene, tetrazolylene, triazinylene, and triazolylene. Examples of bicyclic heteroarylene groups include, but are not limited to, benzofuranylene, benzimidazolylene, benzoisoxazolylene, benzopyranylene, benzothiadiazolylene, benzothiazolylene, benzothienylene, benzotriazolylene, benzoxazolylene, furopyridylene, imidazopyridinylene, imidazothiazolylene, indolizinylene, indolylene, indazolylene, isobenzofuranylene, isobenzothienylene, isoindolylene, isoquinolinylene, isothiazolylene, naphthyridinylene, oxazolopyridinylene, phthalazinylene, pteridinylene, purinylene, pyridopyridylene, pyrrolopyridylene, quinolinylene, quinoxalinylene, quinazolinylene, thiadiazolopyrimidylene, and thienopyridylene. Examples of tricyclic heteroarylene groups include, but are not limited to, acridinylene, benzindolylene, carbazolylene, dibenzofuranylene, perimidinylene, phenanthrolinylene, phenanthridinylene, phenarsazinylene, phenazinylene, phenothiazinylene, phenoxazinylene, and xanthenylene. In certain embodiments, heteroarylene may also be optionally substituted with one or more substituents Q as described herein.

The term “heterocyclyl” or “heterocyclic” refers to a monovalent monocyclic non-aromatic ring system or monovalent polycyclic ring system that contains at least one non-aromatic ring, wherein one or more of the non-aromatic ring atoms are heteroatoms independently selected from O, S, and N; and the remaining ring atoms are carbon atoms. In certain embodiments, the heterocyclyl or heterocyclic group has from 3 to 20, from 3 to 15, from 3 to 10, from 3 to 8, from 4 to 7, or from 5 to 6 ring atoms. Heterocyclyl groups are bonded to the rest of a molecule through the non-aromatic ring. In certain embodiments, the heterocyclyl is a monocyclic, bicyclic, tricyclic, or tetracyclic ring system, which may be fused or bridged, and in which nitrogen or sulfur atoms may be optionally oxidized, nitrogen atoms may be optionally quaternized, and some rings may be partially or fully saturated, or aromatic. The heterocyclyl may be attached to the main structure at any heteroatom or carbon atom which results in the creation of a stable compound. Examples of such heterocyclic groups include, but are not limited to, azepinyl, benzodioxanyl, benzodioxolyl, benzofuranonyl, benzopyranonyl, benzopyranyl, benzotetrahydrofuranyl, benzotetrahydrothienyl, benzothiopyranyl, benzoxazinyl, 3-carbolinyl, chromanyl, chromonyl, cinnolinyl, coumarinyl, decahydroisoquinolinyl, dihydrobenzisothiazinyl, dihydrobenzisoxazinyl, dihydrofuryl, dihydroisoindolyl, dihydropyranyl, dihydropyrazolyl, dihydropyrazinyl, dihydropyridinyl, dihydropyrimidinyl, dihydropyrrolyl, dioxolanyl, 1,4-dithianyl, furanonyl, imidazolidinyl, imidazolinyl, indolinyl, isobenzotetrahydrofuranyl, isobenzotetrahydrothienyl, isochromanyl, isocoumarinyl, isoindolinyl, isothiazolidinyl, isoxazolidinyl, morpholinyl, octahydroindolyl, octahydroisoindolyl, oxazolidinonyl, oxazolidinyl, oxiranyl, piperazinyl, piperidinyl, 4-piperidonyl, pyrazolidinyl, pyrazolinyl, pyrrolidinyl, pyrrolinyl, quinuclidinyl, tetrahydrofuryl, tetrahydroisoquinolinyl, tetrahydropyranyl, tetrahydrothienyl, thiamorpholinyl, thiazolidinyl, tetrahydroquinolinyl, and 1,3,5-trithianyl. In certain embodiments, heterocyclic may also be optionally substituted with one or more substituents Q as described herein.

The term “heterocyclylene” refers to a divalent monocyclic non-aromatic ring system or divalent polycyclic ring system that contains at least one non-aromatic ring, wherein one or more of the non-aromatic ring atoms are heteroatoms independently selected from O, S, and N; and the remaining ring atoms are carbon atoms. Heterocyclylene groups are bonded to the rest of a molecule through the non-aromatic ring. In certain embodiments, the heterocyclylene group has from 3 to 20, from 3 to 15, from 3 to 10, from 3 to 8, from 4 to 7, or from 5 to 6 ring atoms. In certain embodiments, the heterocyclylene is a monocyclic, bicyclic, tricyclic, or tetracyclic ring system, which may be fused or bridged, and in which nitrogen or sulfur atoms may be optionally oxidized, nitrogen atoms may be optionally quaternized, and some rings may be partially or fully saturated, or aromatic. The heterocyclylene may be attached to the main structure at any heteroatom or carbon atom which results in the creation of a stable compound. Examples of such heterocyclylene groups include, but are not limited to, azepinylene, benzodioxanylene, benzodioxolylene, benzofuranonylene, benzopyranonylene, benzopyranylene, benzotetrahydrofuranylene, benzotetrahydrothienylene, benzothiopyranylene, benzoxazinylene, β-carbolinylene, chromanylene, chromonylene, cinnolinylene, coumarinylene, decahydroisoquinolinylene, dihydrobenzisothiazinylene, dihydrobenzisoxazinylene, dihydrofurylene, dihydroisoindolylene, dihydropyranylene, dihydropyrazolylene, dihydropyrazinylene, dihydropyridinylene, dihydropyrimidinylene, dihydropyrrolylene, dioxolanylene, 1,4-dithianylene, furanonylene, imidazolidinylene, imidazolinylene, indolinylene, isobenzotetrahydrofuranylene, isobenzotetrahydrothienylene, isochromanylene, isocoumarinylene, isoindolinylene, isothiazolidinylene, isoxazolidinylene, morpholinylene, octahydroindolylene, octahydroisoindolylene, oxazolidinonylene, oxazolidinylene, oxiranylene, piperazinylene, piperidinylene, 4-piperidonylene, pyrazolidinylene, pyrazolinylene, pyrrolidinylene, pyrrolinylene, quinuclidinylene, tetrahydrofurylene, tetrahydroisoquinolinylene, tetrahydropyranylene, tetrahydrothienylene, thiamorpholinylene, thiazolidinylene, tetrahydroquinolinylene, and 1,3,5-trithianylene. In certain embodiments, heterocyclic may also be optionally substituted with one or more substituents Q as described herein.

The term “halogen”, “halide” or “halo” refers to fluorine, chlorine, bromine, and/or iodine.

The term “amino acid” refers to naturally occurring and synthetic ac, J3, y, or 6 amino acids, and includes but is not limited to, amino acids found in proteins, i.e. glycine, alanine, valine, leucine, isoleucine, methionine, phenylalanine, tryptophan, proline, serine, threonine, cysteine, tyrosine, asparagine, glutamine, aspartate, glutamate, lysine, arginine and histidine. In certain embodiments, the amino acid is in the L-configuration. In certain embodiments, the amino acid is in the D-configuration. Alternatively, the amino acid can be a derivative of alanyl, valinyl, leucinyl, isoleuccinyl, prolinyl, phenylalaninyl, tryptophanyl, methioninyl, glycinyl, serinyl, threoninyl, cysteinyl, tyrosinyl, asparaginyl, glutaminyl, aspartoyl, glutaroyl, lysinyl, argininyl, histidinyl, β-alanyl, β-valinyl, β-leucinyl, β-isoleuccinyl, β-prolinyl, β-phenylalaninyl, β-tryptophanyl, β-methioninyl, β-glycinyl, β-serinyl, β-threoninyl, β-cysteinyl, β-tyrosinyl, β-asparaginyl, β-glutaminyl, β-aspartoyl, β-glutaroyl, β-lysinyl, β-argininyl or β-histidinyl.

The term “amino acid derivative” refers to a group derivable from a naturally or non-naturally occurring amino acid, as described and exemplified herein. Amino acid derivatives are apparent to those of skill in the art and include, but are not limited to, ester, amino alcohol, amino aldehyde, amino lactone, and N-methyl derivatives of naturally and non-naturally occurring amino acids. In an embodiment, an amino acid derivative is provided as a substituent of a compound described herein, wherein the substituent is -G-C(O)-Q, wherein Q is sulfanyl, amino or alkoxyl and G is C₁-C₂ alkyl. In an embodiment, an amino acid derivative is provided as a substituent of a compound described herein, wherein the substituent is —NH-G(S_{C})—C(O)-Q¹, wherein Q¹ is —SR, —NRR or alkoxyl, R is hydrogen or alkyl, S_{C} is a side chain of a naturally occurring or non-naturally occurring amino acid and G is C₁-C₂ alkyl. In an embodiment, an amino acid derivative is provided as a substituent of a compound described herein, wherein the substituent is —O—C(O)-G(S_{C})—NH-Q², wherein Q² is hydrogen or alkoxyl, S_{C} is a side chain of a naturally occurring or non-naturally occurring amino acid and G is C₁-C₂ alkyl. In certain embodiments, G is C₁ alkyl and S_{C} is selected from the group consisting of hydrogen, alkyl, heteroalkyl, arylalkyl and heteroarylalkyl. In an embodiment, an amino acid derivative is provided as a substituent of a compound described herein, wherein the amino acid derivative is in the D-configuration. In an embodiment, an amino acid derivative is provided as a substituent of a compound described herein, wherein the amino acid derivative is in the L-configuration.

The term “optionally substituted” is intended to mean that a group or substituent, such as an alkyl, alkylene, heteroalkylene, alkenyl, alkenylene, heteroalkenylene, alkynyl, alkynylene, cycloalkyl, cycloalkylene, aryl, arylene, aralkyl, heteroaryl, heteroarylene, heterocyclyl, or heterocyclylene group, may be substituted with one or more, in one embodiment, one, two, three, or four, substituents Q, each of which is independently selected from, e.g., (a) oxo (═O), cyano (—CN), halo, or nitro (—NO₂); (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OP(O)(OR^{e})₂, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —P(O)(OR^{a})R^{d}, —CH₂P(O)(OR^{a})R^{d}, —CH₂OP(O)(OR^{e})₂, —CH₂OC(O)C(R^{a})₂NR^{b}R^{c}, —SR^{a}, —S(O)R^{a}, —S(O)₂R^{a}, —S(O)NR^{b}R^{c}, or —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a}; and each R^{e} is independently (i) hydrogen; (ii) a monovalent cation, in one embodiment, Na⁺ or K⁺; (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iv) two R^{e} together are a divalent cation, in one embodiment, Mg²⁺ or Ca²⁺. As used herein, all groups that can be substituted are “optionally substituted,” unless otherwise specified.

In one embodiment, each Q^{a} is independently selected from of (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; and (c) —C(O)R^{f}, —C(O)OR^{f}, —C(O)NR^{g}R^{h}, —C(NR^{f})NR^{g}R^{h}, —OR^{f}, —OC(O)R^{f}, —OC(O)OR^{f}, —OC(O)NR^{g}R^{h}, —OC(═NR^{f})NR^{g}R^{h}, —OP(O)(ORⁿ)₂, —OS(O)R^{f}, —OS(O)₂R^{f}, —OS(O)NR^{g}R^{h}, —OS(O)₂NR^{g}R^{h}, —NR^{g}R^{h}, —NR^{f}C(O)R^{k}, —NR^{f}C(O)OR^{k}, —NR^{f}C(O)NR^{g}R^{h}, —NR^{f}C(═NR^{k})NR^{g}R^{h}, —NR^{f}S(O)R^{k}, —NR^{f}S(O)₂R^{k}, —NR^{f}S(O)NR^{g}R^{h}, —NR^{f}S(O)₂NR^{g}R^{h}, —P(O)(OR^{f})R^{k}, —CH₂P(O)(OR^{f})R^{k}, —SR^{f}, —S(O)R^{f}, —S(O)₂R^{f}, —S(O)NR^{g}R^{h}, or —S(O)₂NR^{g}R^{h}; wherein each R^{f}, R^{g}, R^{h}, and R^{k} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{g} and R^{h} together with the N atom to which they are attached form heterocyclyl; and each Rⁿ is independently (i) hydrogen; (ii) a monovalent cation, in one embodiment, Na⁺ or K⁺; (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iv) two Rⁿ together are a divalent cation, in one embodiment, Mg²⁺ or Ca²⁺.

The terms “optically active” and “enantiomerically active” refer to a collection of molecules, which has an enantiomeric excess of no less than about 50%, no less than about 70%, no less than about 80%, no less than about 90%, no less than about 91%, no less than about 92%, no less than about 93%, no less than about 94%, no less than about 95%, no less than about 96%, no less than about 97%, no less than about 98%, no less than about 99%, no less than about 99.5%, or no less than about 99.8%. In certain embodiments, the compound comprises about 95% or more of one enantiomer and about 5% or less of the other enantiomer based on the total weight of the racemate in question.

In describing an optically active compound, the prefixes R and S are used to denote the absolute configuration of the molecule about its chiral center(s). The (+) and (−) are used to denote the optical rotation of the compound, that is, the direction in which a plane of polarized light is rotated by the optically active compound. The (−) prefix indicates that the compound is levorotatory, that is, the compound rotates the plane of polarized light to the left or counterclockwise. The (+) prefix indicates that the compound is dextrorotatory, that is, the compound rotates the plane of polarized light to the right or clockwise. However, the sign of optical rotation, (+) and (−), is not related to the absolute configuration of the molecule, R and S.

The term “isotopic variant” refers to a compound that contains an unnatural proportion of an isotope at one or more of the atoms that constitute such a compound. In certain embodiments, an “isotopic variant” of a compound contains unnatural proportions of one or more isotopes, including, but not limited to, hydrogen (H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine-123 (¹²³I), iodine-125 (¹²⁵I), iodine-127 (¹²⁷I), iodine-129 (¹²⁹I), and iodine-131 (¹³¹I). In certain embodiments, an “isotopic variant” of a compound is in a stable form, that is, non-radioactive. In certain embodiments, an “isotopic variant” of a compound contains unnatural proportions of one or more isotopes, including, but not limited to, hydrogen (¹H), deuterium (²H), carbon-12 (¹²C), carbon-13 (¹³C), nitrogen-14 (¹⁴N), nitrogen-15 (¹⁵N), oxygen-16 (¹⁶O), oxygen-17 (¹⁷O), oxygen-18 (¹⁸O), fluorine-17 (¹⁷F), phosphorus-31 (³¹P), sulfur-32 (³²S), sulfur-33 (³³S), sulfur-34 (³⁴S), sulfur-36 (³⁶S), chlorine-35 (³⁵Cl), chlorine-37 (³⁷Cl), bromine-79 (⁷⁹Br), bromine-81 (⁸¹Br), and iodine-127 (¹²⁷I). In certain embodiments, an “isotopic variant” of a compound is in an unstable form, that is, radioactive. In certain embodiments, an “isotopic variant” of a compound contains unnatural proportions of one or more isotopes, including, but not limited to, tritium (³H), carbon-11 (¹¹C), carbon-14 (¹⁴C), nitrogen-13 (¹³N), oxygen-14 (¹⁴O), oxygen-15 (¹⁵O), fluorine-18 (¹⁸F), phosphorus-32 (³²P), phosphorus-33 (³³P), sulfur-35 (³⁵S), chlorine-36 (³⁶Cl), iodine-123 (¹²³I), iodine-125 (¹²⁵I), iodine-129 (¹²⁹I), and iodine-131 (¹³¹I). It will be understood that, in a compound as provided herein, any hydrogen can be ²H, for example, or any carbon can be ¹³C, as example, or any nitrogen can be ¹⁵N, as example, and any oxygen can be ¹⁸O, where feasible according to the judgment of one of skill. In certain embodiments, an “isotopic variant” of a compound contains unnatural proportions of deuterium.

The term “solvate” refers to a complex or aggregate formed by one or more molecules of a solute, e.g., a compound provided herein, and one or more molecules of a solvent, which present in stoichiometric or non-stoichiometric amount. Suitable solvents include, but are not limited to, water, methanol, ethanol, n-propanol, isopropanol, and acetic acid. In certain embodiments, the solvent is pharmaceutically acceptable. In one embodiment, the complex or aggregate is in a crystalline form. In another embodiment, the complex or aggregate is in a noncrystalline form. Where the solvent is water, the solvate is a hydrate. Examples of hydrates include, but are not limited to, a hemihydrate, monohydrate, dihydrate, trihydrate, tetrahydrate, and pentahydrate.

The phrase “a single enantiomer, a racemic mixture, a diastereomer, a mixture of diastereomers, or an isotopic variant thereof; or a pharmaceutically acceptable salt or solvate thereof” has the same meaning as the phrase “a single enantiomer, a racemic mixture, a diastereomer, a mixture of diastereomers, or an isotopic variant of the compound referenced therein; or a pharmaceutically acceptable salt or solvate of the compound referenced therein, or a single enantiomer, a racemic mixture, a diastereomer, a mixture of diastereomers, or an isotopic variant of the compound referenced therein.”

### Compounds

HCV has a single positive-stranded RNA genome having about 9.6 kb in length that encodes a large polyprotein having about 3010 amino acids. This precursor polyprotein is then processed into a range of structural proteins, including core protein, C, and envelope glycoproteins, E1 and E2; and non-structural proteins, including NS2, NS3, NS4A, NS4B, NS5A, and NS5B, by host signal peptidases and two viral proteases, NS2-3 and NS3. The nonstructural protein 5A (NS5A) is a multifunctional protein essential for HCV replication. Because of its vital role in viral replication, HCV NS5A protein has been actively pursued as a drug target for developing anti-HCV therapy.

In one embodiment, provided herein is a compound of Formula I:

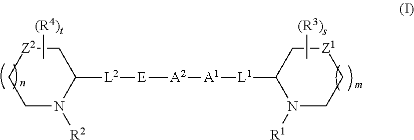

or a single enantiomer, a racemic mixture, a diastereomer, a mixture of diastereomers, or an isotopic variant thereof; or a pharmaceutically acceptable salt or solvate thereof;

A¹, A², and E are each independently (a) a bond; or (b) C₁₋₆ alkylene, C₂₋₆ alkenylene, C₂₋₆ alkynylene, C₂₋₂₀ cycloalkylene, C₆₋₂₀ arylene, heteroarylene, or heterocyclylene;

L¹ and L² are each independently (a) a bond; (b) C₁₋₆ alkylene, C₂₋₆ alkenylene, C₂₋₆ alkynylene, C₃₋₇cycloalkylene, C₆₋₁₄ arylene, heteroarylene, or heterocyclylene; or (c) —C(O)—, —C(O)O—, —C(O)NR^{1a}—, —C(═NR^{1a})NR^{1c}—, —O—, —OC(O)O—, —OC(O)NR^{1a}—, —OC(═NR^{1a})NR^{1c}—, —OP(O)(OR^{1a})—, —OP(O)(OR^{1a})O—, —NR^{1a}—, —NR^{1a}C(O)NR^{1c}—, —NR^{1a}C(═NR^{1b})NR^{1c}—, —NR^{1a}S(O)NR^{1c}—, —NR^{1a}S(O)₂NR^{1c}—, —S—, —S(O)—, —S(O)₂—, —S(O)NR^{1a}—, or —S(O)₂NR^{1a}—; wherein at least one of L¹ and L² is heteroarylene or heterocyclylene, which is substituted with —C₁₋₆alkylene-OP(O)(OR^{P1})₂ (in one embodiment, —CH₂—OP(O)(OR^{P1})₂), or —C₁₋₆ alkylene-O-linked amino acid or a derivative thereof (in one embodiment, —CH₂—OC(O)C(R^{a})₂NR^{1b}R^{1c});

Z¹ and Z² are each independently a bond, —O—, —S—, —S(O)—, —S(O₂)—, or —N(R^{N})—;

R¹ and R² are each independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (c) —C(O)R^{1a}, —C(O)CH(N(R^{1c})C(O)OR^{1b})R^{1a}, —C(O)OR^{1a}, —C(O)NR^{1b}R^{1c}, —C(NR^{a})NR^{1b}R^{1c}, —P(O)(OR^{1a})R^{1d}, —CH₂P(O)(OR^{a})R^{1d}, —S(O)R^{1a}, —S(O)₂R^{1a}, —S(O)NR^{1b}R^{1c}, or —S(O)₂NR^{1b}R^{1c}; (d) —C₁₋₆ alkylene-OP(O)(OR^{P1})₂, in one embodiment, —CH₂—OP(O)(OR^{P1})₂; or (e) —C₁₋₆ alkylene-O-linked amino acid or a derivative thereof, in one embodiment, —CH₂—OC(O)C(R^{aa})₂NR^{1b}R^{1c};

each R³ and R⁴ is independently (a) cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{a}, —OC(O)NR^{1b}R^{1a}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{a})R^{1d}, —CH₂P(O)(OR^{a})R^{1d}, —SR^{1a}, —S(O)R^{1a}, —S(O)₂R^{a}, —S(O)NR^{1b}R^{1c}, or —S(O)₂NR^{1b}R^{1c}; or

two R³ or two R⁴ that are attached to the same ring are linked together to form a bond, —O—, —NR^{N}—, —S—, C₁₋₆ alkylene, C₁₋₆ heteroalkylene, C₂₋₆ alkenylene, or C₂₋₆ heteroalkenylene;

each R^{N} is independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (c) —C(O)OR^{1a}, —C(O)NR^{1b}R^{1c}, —C(NR^{1a})NR^{1b}R^{1c}, —OR^{1a}, —OC(O)R^{a}, —OC(O)OR^{1a}, —OC(O)NR^{1b}R^{a}, —OC(═NR^{1a})NR^{1b}R^{1c}, —OS(O)R^{1a}, —OS(O)₂R^{1a}, —OS(O)NR^{1b}R^{f}C, —OS(O)₂NR^{1b}R^{1c}, —NR^{1b}R^{1c}, —NR^{1a}C(O)R^{d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —S(O)R^{1a}, —S(O)₂R^{1a}, —S(O)NR^{1b}R^{1c}, or —S(O)₂NR^{1b}R^{1c}; (d) —C₁₋₆ alkylene-OP(O)(OR^{P})₂, in one embodiment, —CH₂—OP(O)(OR^{P1})₂; or (e) —C₁₋₆ alkylene-O-linked amino acid or a derivative thereof, in one embodiment, —CH₂—OC(O)C(R^{aa})₂NR^{1b}R^{1c};

each R^{P1} is independently (a) hydrogen; (b) a monovalent cation, in one embodiment, Na⁺ or K⁺, in another embodiment, Li⁺, Rb⁺, or Cs+; (c) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (d) two R^{P1} together are a divalent cation, in one embodiment, Mg²⁺ or Ca²⁺;

each R^{1a}, R^{1b}, R^{1c}, and R^{1d} is independently hydrogen, C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or R^{1a} and R^{c} together with the C and N atoms to which they are attached form heterocyclyl; or R^{1b} and R^{1c} together with the N atom to which they are attached form heterocyclyl;

each R^{aa} independently is a side chain of a naturally occurring or non-naturally occurring amino acid; in one embodiment, each R^{aa} independently is hydrogen, C₁₋₆ alkyl, heteroalkyl, C₆₋₁₄ aryl-C₁₋₆alkyl and heteroaryl-C₁₋₆alkyl;

each m and n is independently an integer of 1, 2, 3, or 4; and

each s and t is independently an integer of 0, 1, 2, 3, 4, 5, 6, or 7;

wherein each alkyl, alkylene, heteroalkylene, alkenyl, alkenylene, heteroalkenylene, alkynyl, alkynylene, cycloalkyl, cycloalkylene, aryl, arylene, aralkyl, heteroaryl, heteroarylene, heterocyclyl, and heterocyclylene is optionally substituted with one or more, in one embodiment, one, two, three, or four, substituents Q, where each Q is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OP(O)(OR^{e})₂, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —P(O)(OR^{a})R^{d}, —CH₂P(O)(OR^{a})R^{d}, —CH₂OP(O)(OR^{e})₂, —CH₂OC(O)C(R^{a})₂NR^{b}R^{c}, —SR^{a}, —S(O)R^{a}, —S(O)₂R^{a}, —S(O)NR^{b}R^{c}, or —S(O)₂NR^{b}R^{c}, wherein each R^{a}, R^{b}, Re, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a}; and each R^{e} is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iv) two R^{e} together are a divalent cation (e.g., Mg²⁺ or Ca²⁺);

wherein each Q^{a} is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{f}, —C(O)OR^{f}, —C(O)NR^{g}R^{h}, —C(NR^{f})NR^{g}R^{h}, —OR^{f}, —OC(O)R^{f}, —OC(O)OR^{f}, —OC(O)NR^{g}R^{h}, —OC(═NR^{f})NR^{g}R^{h}, —OP(O)(ORⁿ)₂, —OS(O)R^{f}, —OS(O)₂R^{f}, —OS(O)NR^{g}R^{h}, —OS(O)₂NR^{g}R^{h}, —NR^{g}R^{h}, —NR^{f}C(O)R^{k}, —NR^{f}C(O)OR^{k}, —NR^{f}C(O)NR^{g}R^{h}, —NR^{f}C(═NR^{k})NR^{g}R^{h}, —NR^{f}S(O)R^{k}, —NR^{f}S(O)₂R^{k}, —NR^{f}S(O)NR^{g}R^{h}, —NR^{f}S(O)₂NR^{g}R^{h}, —P(O)(OR^{f})R^{k}, —CH₂P(O)(OR^{f})R^{k}, —SR^{f}, —S(O)R^{f}, —S(O)₂R, —S(O)NR^{g}R^{h}, or —S(O)₂NR^{g}R^{h}; wherein each R^{f}, R^{g}, R^{h}, and R^{k} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{g} and R^{h} together with the N atom to which they are attached form heterocyclyl; and each Rⁿ is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iv) two Rⁿ together are a divalent cation (e.g., Mg²⁺ or Ca²⁺).

In certain embodiments of the compound of Formula I, or single enantiomer, racemic mixture, diastereomer, mixture of diastereomers, or isotopic variant thereof; or pharmaceutically acceptable salt or solvate thereof;

A¹, A², and E are each independently (a) a bond; or (b) C₁₋₆ alkylene, C₂₋₆ alkenylene, C₂₋₆ alkynylene, C₂₋₂₀ cycloalkylene, C₆₋₂₀ arylene, heteroarylene, or heterocyclylene;

L¹ and L² are each independently (a) a bond; (b) C₁₋₆ alkylene, C₂₋₆ alkenylene, C₂₋₆ alkynylene, C₃₋₇ cycloalkylene, C₆₋₁₄ arylene, heteroarylene, or heterocyclylene; or (c) —C(O)—, —C(O)O—, —C(O)NR^{a}—, —C(═NR^{1a})NR^{1c}—, —O—, —OC(O)O—, —OC(O)NR^{1c}—, —OC(═NR^{1a})NR^{1c}—, —OP(O)(OR^{1a})—, —OP(O)(OR^{1a})O—, —NR^{1c}—, —NR^{1a}C(O)NR^{1c}—, —NR^{1a}C(═NR^{1b})NR^{1c}—, —NR^{1a}S(O)NR^{1c}—, —NR^{1a}S(O)₂NR^{1b}—, —S—, —S(O)—, —S(O)₂—, —S(O)NR^{1a}, or —S(O)₂NR^{1a}—; wherein at least one of L¹ and L² is heteroarylene or heterocyclylene, which is substituted with —C₁₋₆ alkylene-OP(O)(OR^{P1})₂, in one embodiment, —CH₂—OP(O)(OR^{P1})₂;

Z¹ and Z² are each independently a bond, —O—, —S—, —S(O)—, —S(O₂)—, or —N(R^{N})—;

R¹ and R² are each independently (a) hydrogen; (b) C₁₋₆ alkyl, C₂₋₆ alkenyl, C₂₋₆ alkynyl, C₃₋₇cycloalkyl, C₆₋₁₄ aryl, C₇₋₁₅ aralkyl, heteroaryl, or heterocyclyl; (c) —C(O)R^{1a}, —C(O)CH(N(R^{1c})C(O)OR^{1b})R^{1a}, —C(O)OR^{1a}, —C(O)NR^{1b}R^{1c}, —C(NR^{1a})NR^{1b}R^{1c}, —P(O)(OR^{1a})R^{1d}, —CH₂P(O)(OR^{a})R^{1d}, —S(O)R^{1a}, —S(O)₂R^{1a}, —S(O)NR^{1b}R^{1c}, or —S(O)₂NR^{1b}R^{1c}; or (d) —C₁₋₆ alkylene-OP(O)(OR^{P1})₂, in one embodiment, —CH₂—OP(O)(OR^{P1})₂;

each R³ and R⁴ is independently (a) cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —SR^{1a}, —S(O)R^{1a}, —S(O)₂R^{1a}, —S(O)NR^{1b}R^{1c}, or —S(O)₂NR^{1b}R^{1c}; or

two R³ or two R⁴ that are attached to the same ring are linked together to form a bond, —O—, —NR^{N}—, —S—, C₁₋₆ alkylene, C₁₋₆ heteroalkylene, C₂₋₆ alkenylene, or C₂₋₆ heteroalkenylene;

each R^{N} is independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (c) —C(O)OR^{a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{d})NR^{1b}R^{1c}, —NR^{1a}S(O)R^{1d}, —NR^{1a}S(O)₂R^{1d}, —NR^{1a}S(O)NR^{b}R^{c}, —NR^{1a}S(O)₂NR^{1b}R^{1c}, —P(O)(OR^{a})R^{1d}, —CH₂P(O)(OR^{1a})R^{1d}, —S(O)R^{1a}, —S(O)₂R^{1a}, —S(O)NR^{1b}R^{1c}, or —S(O)₂NR^{1b}R^{1c}; or (d) —C₁₋₆ alkylene-OP(O)(OR^{P1})₂, in one embodiment, —CH₂—OP(O)(OR^{P1})₂;

each R^{P1} is independently (a) hydrogen; (b) a monovalent cation, in one embodiment, Na⁺ or K⁺; or (c) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or two R^{P1} together are a divalent cation, in one embodiment, Mg²⁺ or Ca²⁺;

each R^{1a}, R^{1b}, R^{1c}, and R^{1d} is independently hydrogen, C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or R^{1a} and R^{1e} together with the C and N atoms to which they are attached form heterocyclyl; or R^{1b} and R^{1c} together with the N atom to which they are attached form heterocyclyl;

each m and n is independently an integer of 1, 2, 3, or 4; and

each s and t is independently an integer of 0, 1, 2, 3, 4, 5, 6, or 7;

wherein each alkyl, alkylene, heteroalkylene, alkenyl, alkenylene, heteroalkenylene, alkynyl, alkynylene, cycloalkyl, cycloalkylene, aryl, arylene, aralkyl, heteroaryl, heteroarylene, heterocyclyl, and heterocyclylene is optionally substituted with one or more, in one embodiment, one, two, three, or four, substituents Q, where each Q is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OP(O)(OR^{e})₂, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —P(O)(OR^{a})R^{d}, —CH₂P(O)(OR^{a})R^{d}, —SR^{a}, —S(O)R^{a}, —S(O)₂R^{a}, —S(O)NR^{b}R^{c}, or —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a}; and each R^{e} is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iv) two R^{e} together are a divalent cation (e.g., Mg²⁺ or Ca²⁺);

wherein each Q^{a} is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{f}, —C(O)OR^{f}, —C(O)NR^{g}R^{h}, —C(NR^{f})NR^{g}R^{h}, —OR^{f}, —OC(O)R^{f}, —OC(O)OR^{f}, —OC(O)NR^{g}R^{h}, —OC(═NR^{f})NR^{g}R^{h}, —OP(O)(ORⁿ)₂, —OS(O)R^{f}, —OS(O)₂R^{f}, —OS(O)NR^{g}R^{h}, —OS(O)₂NR^{g}R^{h}, —NR^{g}R^{h}, —NR^{f}C(O)R^{k}, —NR^{f}C(O)OR^{k}, —NR^{f}C(O)NR^{g}R^{h}, —NR^{f}C(═NR^{k})NR^{g}R^{h}, —NR^{f}S(O)R^{k}, —NR^{f}S(O)₂R^{k}, —NR^{f}S(O)NR^{g}R^{h}, —NR^{f}S(O)₂NR^{g}R^{h}, —P(O)(OR^{f})R^{k}, —CH₂P(O)(OR^{f})R^{k}, —SR^{f}, —S(O)R^{f}, —S(O)₂R, —S(O)NR^{g}R^{h}, or —S(O)₂NR^{g}R^{h}; wherein each R^{f}, R^{g}, R^{h}, and R^{k} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{g} and R^{h} together with the N atom to which they are attached form heterocyclyl; and each Rⁿ is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iv) two Rⁿ together are a divalent cation (e.g., Mg²⁺ or Ca²⁺).

In another embodiment, provided herein is a compound of Formula II:

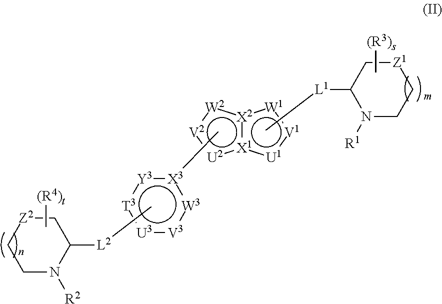

or a single enantiomer, a racemic mixture, a diastereomer, a mixture of diastereomers, or an isotopic variant thereof or a pharmaceutically acceptable salt or solvate thereof;
wherein:

T³ is a bond, C, N, O, S, CR⁷, or NR⁷;

U¹, U², U³, V¹, V², V³, W¹, W², W³, and Y³ are each independently C, N, O, S, CR⁷, or NR⁷;

X¹, X², and X³ are each independently C or N;

each R⁷ is independently (a) hydrogen, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q; (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{d}, —SR^{1a}, —S(O)R^{a}, —S(O)₂R^{1a}, —S(O)NR^{1b}R^{1c}, or —S(O)₂NR^{1b}R^{1c}; (d) —C₁₋₆ alkylene-OP(O)(OR^{P1})₂, in one embodiment, —CH₂—OP(O)(OR^{P1})₂; or (e) —C₁₋₆ alkylene-O-linked amino acid or a derivative thereof, in one embodiment, —CH₂—OC(O)C(R^{aa})₂NR^{1b}R^{1c}; and

R¹, R², R³, R⁴, R^{1a}, R^{1b}, R^{1c}, R^{1d}, R^{aa}, R^{P1}, L¹, L², Q, Z¹, Z², m, n, S, and t are each as defined herein.

In certain embodiments of the compound of Formula II, or single enantiomer, racemic mixture, diastereomer, mixture of diastereomers, or isotopic variant thereof; or pharmaceutically acceptable salt or solvate thereof;

T³ is a bond, C, N, O, S, CR⁷, or NR⁷;

U¹, U², U³, V¹, V², V³, W¹, W², W³, and Y³ are each independently C, N, O, S, CR⁷, or NR⁷;

X¹, X², and X³ are each independently C or N;

each R⁷ is independently (a) hydrogen, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q; (c) —C(O)R^{1a}, —C(O)OR^{1a}, —C(O)NR^{1b}R^{1a}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —SR^{1a}, —S(O)R^{1a}, —S(O)₂R^{1a}, —S(O)NR^{1b}R^{1c}, or —S(O)₂NR^{1b}R^{1c}; or (d) —C₁₋₆ alkylene-OP(O)(OR^{P1})₂, in one embodiment, —CH₂—OP(O)(OR^{P1})₂; and

R¹, R², R³, R⁴, R^{1a}, R^{1b}, R^{1c}, R^{1d}, R^{P1}, L¹, L², Q, Z¹, Z², m, n, s, and t are each as defined herein.

In yet another embodiment, provided herein is a compound of Formula III:

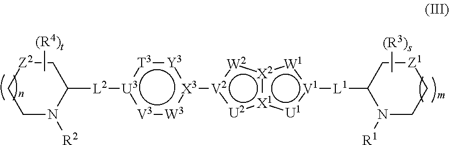

or a single enantiomer, a racemic mixture, a diastereomer, a mixture of diastereomers, or an isotopic variant thereof; or a pharmaceutically acceptable salt or solvate thereof; wherein R¹, R², R³, R⁴, L¹, L², T³, U¹, U², U³, V¹, V², V³, W¹, W², W³, X¹, X², X³, Y³, Z¹, Z², m, n, s, and t are each as defined herein.

In one embodiment, provided herein is a compound of Formula IIIa:

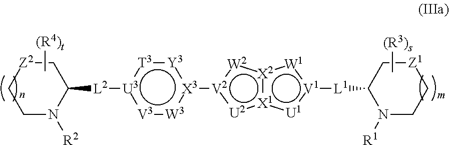

or a single enantiomer, a diastereomer, a mixture of diastereomers, or an isotopic variant thereof; or a pharmaceutically acceptable salt or solvate thereof; wherein R¹, R², R³, R⁴, L¹, L², T³, U¹, U², U³, V¹, V², V³, W¹, W², W³, X¹, X², X³, Y³, Z¹, Z², m, n, s, and t are each as defined herein.

In another embodiment, provided herein is a compound of Formula IIIb:

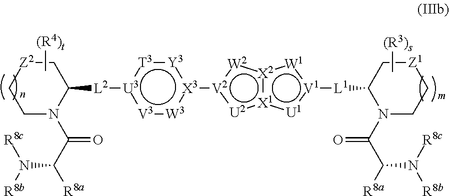

or a single enantiomer, a diastereomer, a mixture of diastereomers, or an isotopic variant thereof; or a pharmaceutically acceptable salt or solvate thereof; wherein:

each R^{8a} is independently (a) hydrogen; or (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q;

each R^{8b} and R^{8c} is independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q; (c) —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —S(O)R^{1a}, —S(O)₂R^{1a}, —S(O)NR^{1b}R^{1c}, or —S(O)₂NR^{1b}R^{1c}; (d) —C₁₋₆ alkylene-OP(O)(OR^{P1})₂, in one embodiment, —CH₂—OP(O)(OR^{P1})₂; or (e) —C₁₋₆ alkylene-O-linked amino acid or a derivative thereof, in one embodiment, —CH₂—OC(O)C(R^{aa})₂NR^{1b}R^{1c}; and

R³, R⁴, R^{1a}, R^{1b}, R^{1c}, R^{1d}, R^{aa}, R^{P1} L¹, L², Q, T³, U¹, U², U³, V¹, V², V³, W¹, W², W³, X¹, X², X³, Y¹, Z¹, Z², m, n, s, and t are each as defined herein.

In certain embodiments of the compound of Formula IIIb, or single enantiomer, racemic mixture, diastereomer, mixture of diastereomers, or isotopic variant thereof; or pharmaceutically acceptable salt or solvate thereof;

each R^{8a} is independently (a) hydrogen; or (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q; each R^{8b} and R^{8c} is independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q; (c) —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1a}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —S(O)R^{1a}, —S(O)₂R^{1a}, —S(O)NR^{1b}R^{1c}, or —S(O)₂NR^{1b}R^{1c}; or (d) —C₁₋₆ alkylene-OP(O)(OR^{P1})₂, in one embodiment, —CH₂—OP(O)(OR^{P1})₂; and
R³, R⁴, R^{1a}, R^{1b}, R^{1c}, R^{1d}, R^{P1}, L¹, L², Q, T³, U¹, U², U³, V¹, V², V³, W¹, W², W³, X¹, X², X³, Y³, Z¹, Z², m, n, s, and t are each as defined herein.

In yet another embodiment, provided herein is a compound of Formula IIIc:

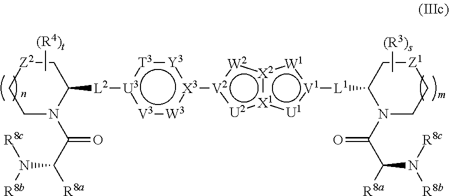

or a single enantiomer, a diastereomer, a mixture of diastereomers, or an isotopic variant thereof; or a pharmaceutically acceptable salt or solvate thereof; wherein R³, R⁴, R^{8a}, R^{8b}, R^{8c}, L¹, L², T³, U¹, U², U³, V¹, V², V³, W¹, W², W³, X¹, X², X³, Y³, Z¹, Z², m, n, s, and t are each as defined herein.

In yet another embodiment, provided herein is a compound of Formula IIId:

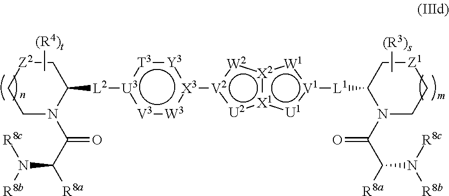

or a single enantiomer, a diastereomer, a mixture of diastereomers, or an isotopic variant thereof; or a pharmaceutically acceptable salt or solvate thereof; wherein R³, R⁴, R^{8a}, R^{8b}, R^{8c}, L¹, L², T³, U¹, U², U³, V¹, V², V³, W¹, W², W³, X¹, X², X³, Y³, Z¹, Z², m, n, s, and t are each as defined herein.

In still another embodiment, provided herein is a compound of Formula IIIe:

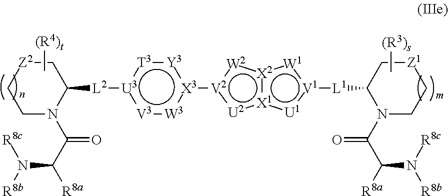

or a single enantiomer, a diastereomer, a mixture of diastereomers, or an isotopic variant thereof; or a pharmaceutically acceptable salt or solvate thereof; wherein R³, R⁴, R^{8a}, R^{8b}, R^{8c}, L¹, L², T³, U¹, U², U³, V¹, V², V³, W¹, W², W³, X¹, X², X³, Y³, Z¹, Z², m, n, s, and t are each as defined herein.

In yet another embodiment, provided herein is a compound of Formula IV:

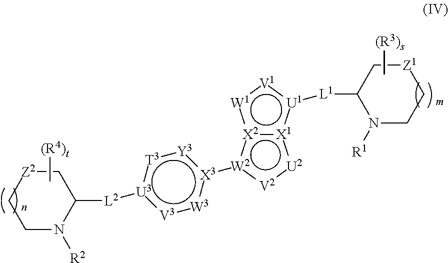

or a single enantiomer, a racemic mixture, a diastereomer, a mixture of diastereomers, or an isotopic variant thereof; or a pharmaceutically acceptable salt or solvate thereof; wherein R¹, R², R³, R⁴, L¹, L², T³, U¹, U², U³, V¹, V², V³, W¹, W², W³, X¹, X², X³, Y³, Z¹, Z², m, n, s, and t are each as defined herein.

In one embodiment, provided herein is a compound of Formula IVa:

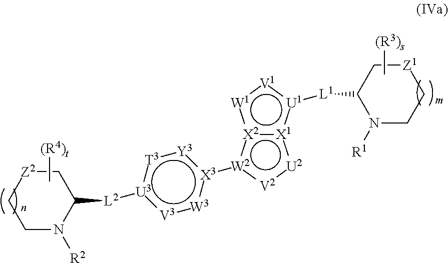

or a single enantiomer, a diastereomer, a mixture of diastereomers, or an isotopic variant thereof; or a pharmaceutically acceptable salt or solvate thereof; wherein R¹, R², R³, R⁴, L¹, L², T³, U¹, U², U³, V¹, V², V³, W¹, W², W³, X¹, X², X³, Y³, Z¹, Z², m, n, s, and t are each as defined herein.

In another embodiment, provided herein is a compound of Formula IVb:

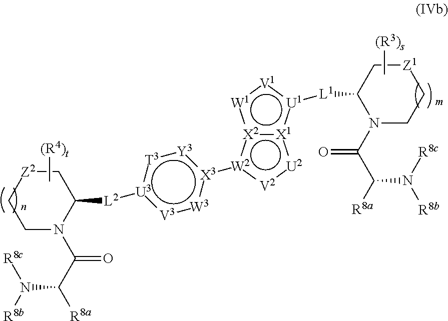

or a single enantiomer, a diastereomer, a mixture of diastereomers, or an isotopic variant thereof; or a pharmaceutically acceptable salt or solvate thereof; wherein R³, R⁴, R^{8a}, R^{8b}, R^{8c} L¹, L², T³, U¹, U², U³, V¹, V², V³, W¹, W², W³, X¹, X², X³, Y³, Z¹, Z², m, n, s, and t are each as defined herein.

In yet another embodiment, provided herein is a compound of Formula IVc:

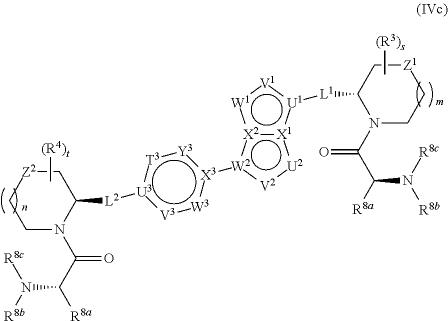

or a single enantiomer, a diastereomer, a mixture of diastereomers, or an isotopic variant thereof; or a pharmaceutically acceptable salt or solvate thereof; wherein R³, R⁴, R^{8a}, R^{8b}, R^{8c}, L¹, L², T³, U¹, U², U³, V¹, V², V³, W¹, W², W³, X¹, X², X³, Y³, Z¹, Z², m, n, s, and t are each as defined herein.

In yet another embodiment, provided herein is a compound of Formula IVd:

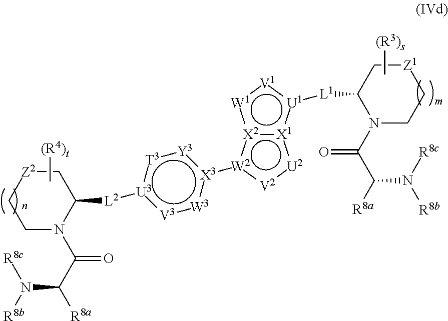

or a single enantiomer, a diastereomer, a mixture of diastereomers, or an isotopic variant thereof; or a pharmaceutically acceptable salt or solvate thereof; wherein R³, R⁴, R^{8a}, R^{8b}, R^{8c}, L¹, L², T³, U¹, U², U³, V¹, V², V³, W¹, W², W³, X¹, X², X³, Y³, Z¹, Z², m, n, s, and t are each as defined herein.

In still another embodiment, provided herein is a compound of Formula IVe:

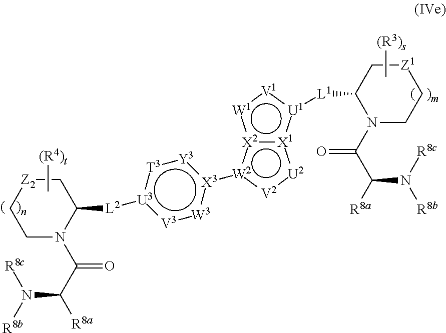

or a single enantiomer, a diastereomer, a mixture of diastereomers, or an isotopic variant thereof; or a pharmaceutically acceptable salt or solvate thereof; wherein R³, R⁴, R^{8a}, R^{8b}, R^{8c}, L¹, L², T³, U¹, U², U³, V¹, V², V³, W¹, W², W³, X¹, X², X³, Y³, Z¹, Z², m, n, s, and t are each as defined herein.

In yet another embodiment, provided herein is a compound of Formula V:

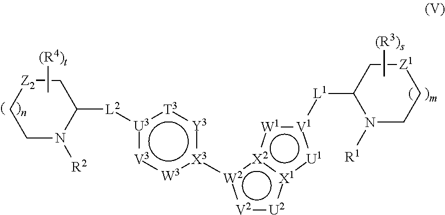

or a single enantiomer, a racemic mixture, a diastereomer, a mixture of diastereomers, or an isotopic variant thereof; or a pharmaceutically acceptable salt or solvate thereof; wherein R¹, R², R³, R⁴, L¹, L², T³, U¹, U², U³, V¹, V², V³, W¹, W², W³, X¹, X², X³, Y³, Z¹, Z², m, n, s, and t are each as defined herein.

In one embodiment, provided herein is a compound of Formula Va:

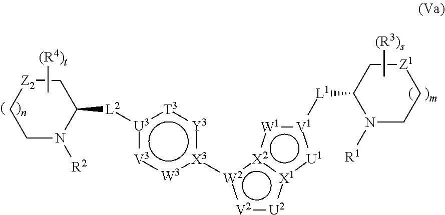

or a single enantiomer, a diastereomer, a mixture of diastereomers, or an isotopic variant thereof; or a pharmaceutically acceptable salt or solvate thereof; wherein R¹, R², R³, R⁴, L¹, L², T³, U¹, U², U³, V¹, V², V³, W¹, W², W³, X¹, X², X³, Y³, Z¹, Z², m, n, s, and t are each as defined herein.

In another embodiment, provided herein is a compound of Formula Vb:

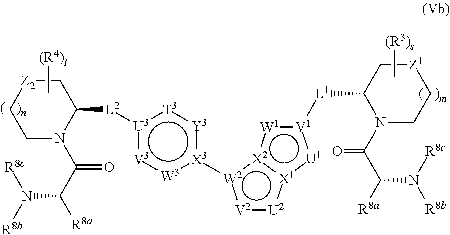

or a single enantiomer, a diastereomer, a mixture of diastereomers, or an isotopic variant thereof; or a pharmaceutically acceptable salt or solvate thereof; wherein R³, R⁴, R^{8a}, R^{8b}, R^{8c}, L¹, L², T³, U¹, U², U³, V¹, V², V³, W¹, W², W³, X¹, X², X³, Y³, Z¹, Z², m, n, s, and t are each as defined herein.

In yet another embodiment, provided herein is a compound of Formula Vc:

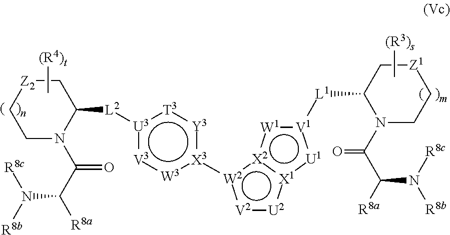

or a single enantiomer, a diastereomer, a mixture of diastereomers, or an isotopic variant thereof; or a pharmaceutically acceptable salt or solvate thereof; wherein R³, R⁴, R^{8a}, R^{8b}, R^{8c}, L¹, L², T³, U¹, U², U³, V¹, V², V³, W¹, W², W³, X¹, X², X³, Y³, Z¹, Z², m, n, s, and t are each as defined herein.

In yet another embodiment, provided herein is a compound of Formula Vd:

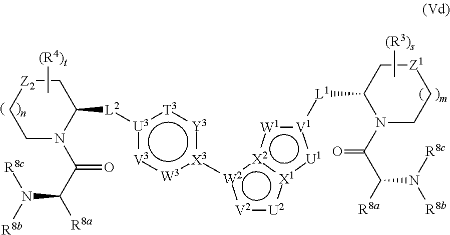

or a single enantiomer, a diastereomer, a mixture of diastereomers, or an isotopic variant thereof; or a pharmaceutically acceptable salt or solvate thereof; wherein R³, R⁴, R^{8a}, R^{8b}, R^{8c}, L¹, L², T³, U¹, U², U³, V¹, V², V³, W¹, W², W³, X¹, X², X³, Y³, Z¹, Z², m, n, s, and t are each as defined herein.

In still another embodiment, provided herein is a compound of Formula Ve:

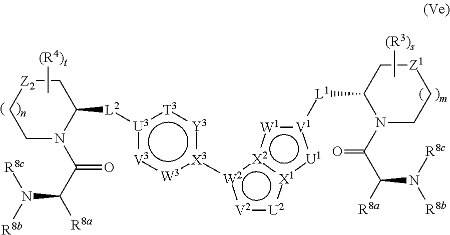

or a single enantiomer, a diastereomer, a mixture of diastereomers, or an isotopic variant thereof; or a pharmaceutically acceptable salt or solvate thereof; wherein R³, R⁴, R^{8a}, R^{8b}, R^{8c}, L¹, L², T³, U¹, U², U³, V¹, V², V³, W¹, W², W³, X¹, X², X³, Y³, Z¹, Z², m, n, s, and t are each as defined herein.

In yet another embodiment, provided herein is a compound of Formula VI:

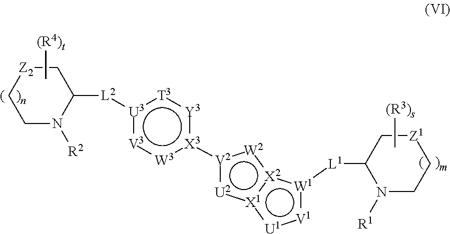

or a single enantiomer, a racemic mixture, a diastereomer, a mixture of diastereomers, or an isotopic variant thereof; or a pharmaceutically acceptable salt or solvate thereof; wherein R¹, R², R³, R⁴, L¹, L², T³, U¹, U², U³, V¹, V², V³, W¹, W², W³, X¹, X², X³, Y³, Z¹, Z², m, n, s, and t are each as defined herein.

In one embodiment, provided herein is a compound of Formula VIa:

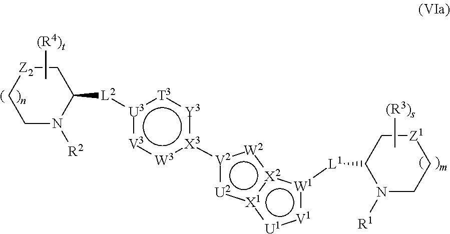

or a single enantiomer, a diastereomer, a mixture of diastereomers, or an isotopic variant thereof; or a pharmaceutically acceptable salt or solvate thereof; wherein R¹, R², R³, R⁴, L¹, L², T³, U¹, U², U³, V¹, V², V³, W¹, W², W³, X¹, X², X³, Y³, Z¹, Z², m, n, s, and t are each as defined herein.

In another embodiment, provided herein is a compound of Formula VIb:

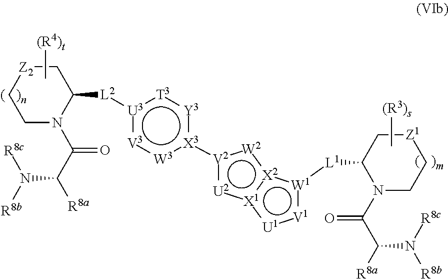

or a single enantiomer, a diastereomer, a mixture of diastereomers, or an isotopic variant thereof; or a pharmaceutically acceptable salt or solvate thereof; wherein R³, R⁴, R^{8a}, R^{8b}, R^{8c}, L¹, L², T³, U¹, U², U³, V¹, V², V³, W¹, W², W³, X¹, X², X³, Y³, Z¹, Z², m, n, s, and t are each as defined herein.

In yet another embodiment, provided herein is a compound of Formula VIc:

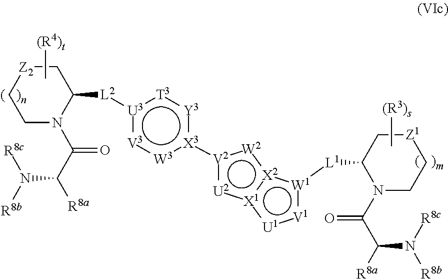

or a single enantiomer, a diastereomer, a mixture of diastereomers, or an isotopic variant thereof; or a pharmaceutically acceptable salt or solvate thereof; wherein R³, R⁴, R^{8a}, R^{8b}, R^{8c}, L¹, L², T³, U¹, U², U³, V¹, V², V³, W¹, W², W³, X¹, X², X³, Y³, Z¹, Z², m, n, s, and t are each as defined herein.

In yet another embodiment, provided herein is a compound of Formula VId:

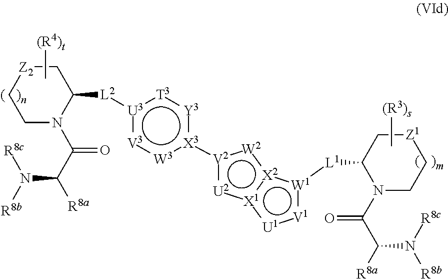

or a single enantiomer, a diastereomer, a mixture of diastereomers, or an isotopic variant thereof; or a pharmaceutically acceptable salt or solvate thereof; wherein R³, R⁴, R^{8a}, R^{8b}, R^{8c}, L¹, L², T³, U¹, U², U³, V¹, V², V³, W¹, W², W³, X¹, X², X³, Y³, Z¹, Z², m, n, s, and t are each as defined herein.

In still another embodiment, provided herein is a compound of Formula VIe:

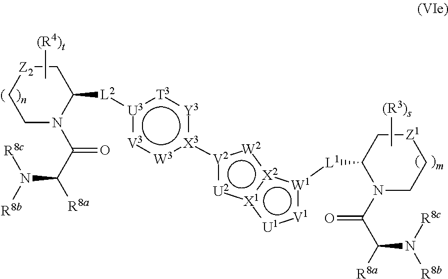

or a single enantiomer, a diastereomer, a mixture of diastereomers, or an isotopic variant thereof; or a pharmaceutically acceptable salt or solvate thereof; wherein R³, R⁴, R^{8a}, R^{8b}, R^{8c}, L¹, L², T³, U¹, U², U³, V¹, V², V³, W¹, W², W³, X¹, X², X³, Y³, Z¹, Z², m, n, s, and t are each as defined herein.

In yet another embodiment, provided herein is a compound of Formula VII:

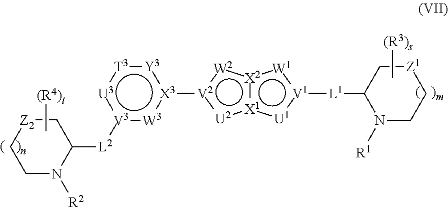

or a single enantiomer, a racemic mixture, a diastereomer, a mixture of diastereomers, or an isotopic variant thereof; or a pharmaceutically acceptable salt or solvate thereof; wherein R¹, R², R³, R⁴, L¹, L², T³, U¹, U², U³, V¹, V², V³, W¹, W², W³, X¹, X², X³, Y³, Z¹, Z², m, n, s, and t are each as defined herein.

In one embodiment, provided herein is a compound of Formula VIIa:

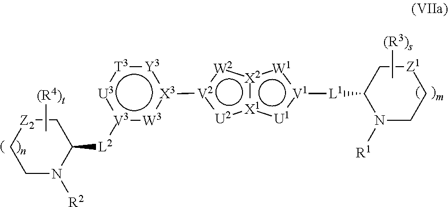

or a single enantiomer, a diastereomer, a mixture of diastereomers, or an isotopic variant thereof; or a pharmaceutically acceptable salt or solvate thereof; wherein R¹, R², R³, R⁴, L¹, L², T³, U¹, U², U³, V¹, V², V³, W¹, W², W³, X¹, X², X³, Y³, Z¹, Z², m, n, s, and t are each as defined herein.

In another embodiment, provided herein is a compound of Formula VIIb:

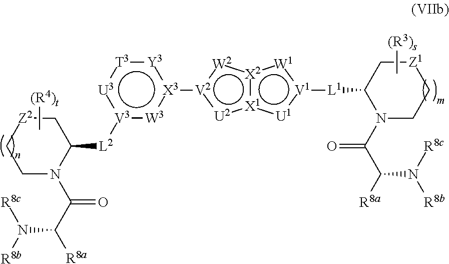

or a single enantiomer, a diastereomer, a mixture of diastereomers, or an isotopic variant thereof; or a pharmaceutically acceptable salt or solvate thereof; wherein R³, R⁴, R^{8a}, R^{8b}, R^{8c}, L¹, L², T³, U¹, U², U³, V¹, V², V³, W¹, W², W³, X¹, X², X³, Y³, Z¹, Z², m, n, s, and t are each as defined herein.

In yet another embodiment, provided herein is a compound of Formula VIIc:

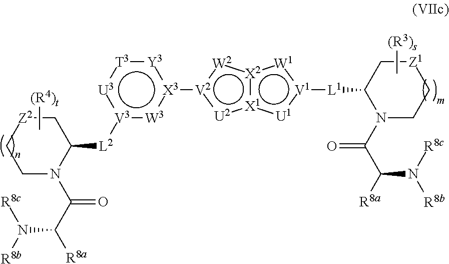

or a single enantiomer, a diastereomer, a mixture of diastereomers, or an isotopic variant thereof; or a pharmaceutically acceptable salt or solvate thereof; wherein R³, R⁴, R^{8a}, R^{8b}, R^{8c}, L¹, L², T³, U¹, U², U³, V¹, V², V³, W¹, W², W³, X¹, X², X³, Y³, Z¹, Z², m, n, s, and t are each as defined herein.

In yet another embodiment, provided herein is a compound of Formula VIId:

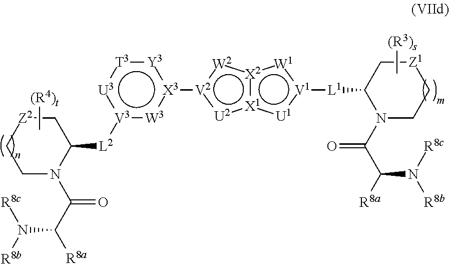

or a single enantiomer, a diastereomer, a mixture of diastereomers, or an isotopic variant thereof; or a pharmaceutically acceptable salt or solvate thereof; wherein R³, R⁴, R^{8a}, R^{8b}, R^{8c}, L¹, L², T³, U¹, U², U³, V¹, V², V³, W¹, W², W³, X¹, X², X³, Y³, Z¹, Z², m, n, s, and t are each as defined herein.

In still another embodiment, provided herein is a compound of Formula VIIe:

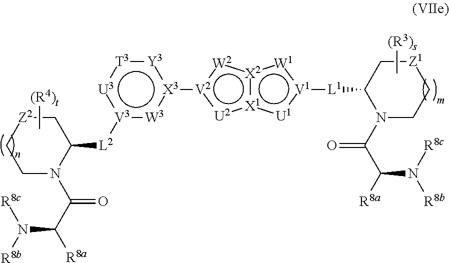

or a single enantiomer, a diastereomer, a mixture of diastereomers, or an isotopic variant thereof; or a pharmaceutically acceptable salt or solvate thereof; wherein R³, R⁴, R^{8a}, R^{8b}, R^{8c}, L¹, L², T³, U¹, U², U³, V¹, V², V³, W¹, W², W³, X¹, X², X³, Y³, Z¹, Z², m, n, s, and t are each as defined herein.

In yet another embodiment, provided herein is a compound of Formula VIII:

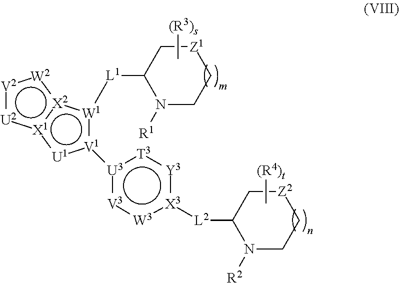

or a single enantiomer, a racemic mixture, a diastereomer, a mixture of diastereomers, or an isotopic variant thereof; or a pharmaceutically acceptable salt or solvate thereof; wherein R¹, R², R³, R⁴, L¹, L², T³, U¹, U², U³, V¹, V², V³, W¹, W², W³, X¹, X², X³, Y³, Z¹, Z², m, n, s, and t are each as defined herein.

In one embodiment, provided herein is a compound of Formula VIIIa:

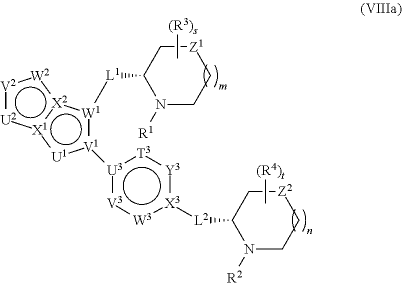

or a single enantiomer, a diastereomer, a mixture of diastereomers, or an isotopic variant thereof; or a pharmaceutically acceptable salt or solvate thereof; wherein R¹, R², R³, R⁴, L¹, L², T³, U¹, U², U³, V¹, V², V³, W¹, W², W³, X¹, X², X³, Y³, Z¹, Z², m, n, s, and t are each as defined herein.

In another embodiment, provided herein is a compound of Formula VIIIb:

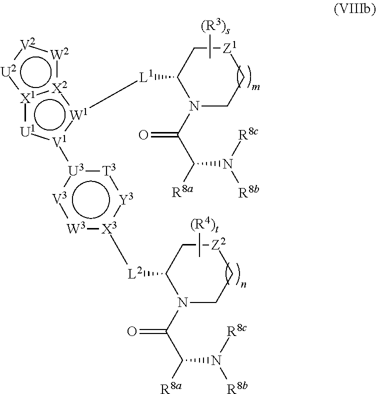

or a single enantiomer, a diastereomer, a mixture of diastereomers, or an isotopic variant thereof; or a pharmaceutically acceptable salt or solvate thereof; wherein R³, R⁴, R^{8a}, R^{8b}, R^{8c}, L¹, L², T³, U¹, U², U³, V¹, V², V³, W¹, W², W³, X¹, X², X³, Y³, Z¹, Z², m, n, s, and t are each as defined herein.

In yet another embodiment, provided herein is a compound of Formula VIIIc:

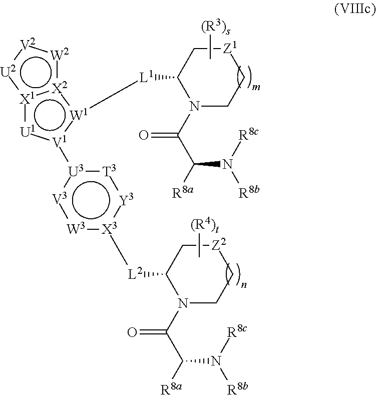

or a single enantiomer, a diastereomer, a mixture of diastereomers, or an isotopic variant thereof; or a pharmaceutically acceptable salt or solvate thereof; wherein R³, R⁴, R^{8a}, R^{8b}, R^{8c}, L¹, L², T³, U¹, U², U³, V¹, V², V³, W¹, W², W³, X¹, X², X³, Y³, Z¹, Z², m, n, s, and t are each as defined herein.

In yet another embodiment, provided herein is a compound of Formula VIIId:

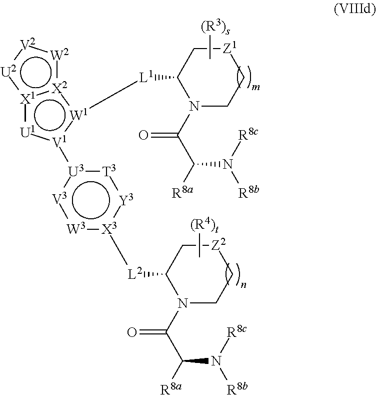

or a single enantiomer, a diastereomer, a mixture of diastereomers, or an isotopic variant thereof; or a pharmaceutically acceptable salt or solvate thereof; wherein R³, R⁴, R^{8a}, R^{8b}, R^{8c}, L¹, L², T³, U¹, U², U³, V¹, V², V³, W¹, W², W³, X¹, X², X³, Y³, Z¹, Z², m, n, s, and t are each as defined herein.

In yet another embodiment, provided herein is a compound of Formula VIIIe:

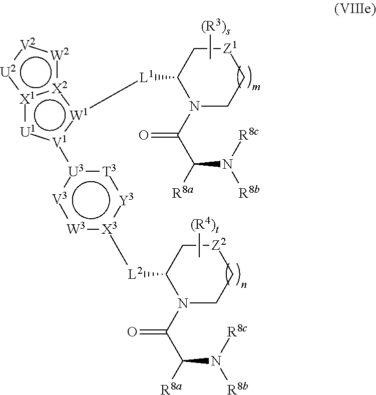

or a single enantiomer, a diastereomer, a mixture of diastereomers, or an isotopic variant thereof; or a pharmaceutically acceptable salt or solvate thereof; wherein R³, R⁴, R^{8a}, R^{8b}, R^{8c}, L¹, L², T³, U¹, U², U³, V¹, V², V³, W¹, W², W³, X¹, X², X³, Y³, Z¹, Z², m, n, s, and t are each as defined herein.

In one embodiment, in Formulae II to VIII, IIIa to VIIIa, IIIb to VIIIb, IIIc to VIIIc, IIId to VIIId, and IIIe to VIIIe, the divalent moiety

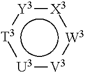

is phenylene, optionally substituted with one, two, three, or four R^{7a}, where each R^{7a} is independently (a) cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; each of which is optionally substituted with one or more, in one embodiment, one, two, three, or four, substituents Q; (c) —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —S(O)R^{1a}, —S(O)₂R^{1a}, —S(O)NR^{1b}R^{1c}, or —S(O)₂NR^{1b}R^{1c}; or (d) —C₁₋₆ alkylene-OP(O)(OR^{P1})₂, in one embodiment, —CH₂—OP(O)(OR^{P1})₂; and R^{1a}, R^{1b}, R^{1c}, R^{1d}, R^{P1}, and Q are each as defined herein.

In another embodiment, in Formulae II to VIII, IIIa to VIIIa, IIIb to VIIIb, IIIc to VIIIc, IIId to VIIId, and IIIe to VIIIe, the divalent moiety

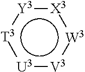

is phenylene, optionally substituted with one, two, three, or four R^{7a}, where each R^{7a} is independently (a) cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; each of which is optionally substituted with one or more, in one embodiment, one, two, three, or four, substituents Q; (c) —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —S(O)R^{1a}, —S(O)₂R^{1a}, —S(O)NR^{1b}R^{1c}, or —S(O)₂NR^{1b}R^{1c}; (d) —C₁₋₆ alkylene-OP(O)(OR^{P1})₂, in one embodiment, —CH₂—OP(O)(OR^{P1})₂; or (e) —C₁₋₆ alkylene-O-linked amino acid or a derivative thereof, in one embodiment, —CH₂—OC(O)C(R^{aa})₂NR^{1b}R^{1c}; and R^{1a}, R^{1b}, R^{1c}, R^{1d}, R^{P1}, and Q are each as defined herein.

In another embodiment, in Formulae II to VIII, IIIa to VIIIa, IIIb to VIIIb, IIIc to VIIIc, IIId to VIIId, and IIIe to VIIIe, the divalent moiety

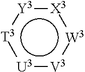

is phenylene, optionally substituted with one, two, three, or four R^{7a}, where each R^{7a} is independently (a) cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; each of which is optionally substituted with one or more, in one embodiment, one, two, three, or four, substituents Q; or (c) —C(O)OR¹, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{b}R^{1a}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —S(O)R^{1a}, —S(O)₂R^{1a}, —S(O)NR^{1b}R^{1c}, or —S(O)₂NR^{1b}R^{1c}; or (d) —C₁₋₆ alkylene-OP(O)(OR^{P1})₂, in one embodiment, —CH₂—OP(O)(OR^{P1})₂; and R^{1a}, R^{1b}, R^{1c}, R^{1d}, R^{P1}, and Q are each as defined herein.

In another embodiment, in Formulae II to VIII, IIIa to VIIIa, IIIb to VIIIb, IIIc to VIIIc, IIId to VIIId, and IIIe to VIIIe, the divalent moiety

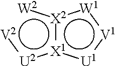

is divalent thieno[3,2-b]thienylene, optionally substituted with one or two R^{7a}, where R^{7a} is as defined herein.

In yet another embodiment, in Formulae II to VIII, IIIa to VIIIa, IIIb to VIIIb, IIIc to VIIIc, IIId to VIIId, and IIIe to VIIIe, the divalent moiety

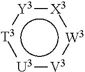

is phenylene, optionally substituted with one, two, three, or four R^{7a}; and the divalent moiety

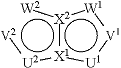

is divalent thieno[3,2-b]thienylene, optionally substituted with one or two R^{7a}; where R^{7a} is as defined herein.

In yet another embodiment, provided herein is a compound of Formula IX:

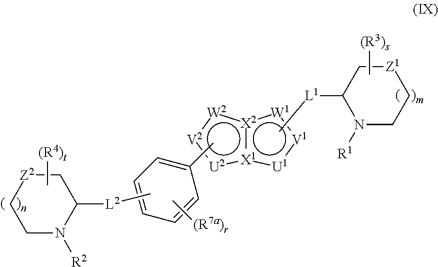

or a single enantiomer, a racemic mixture, a diastereomer, a mixture of diastereomers, or an isotopic variant thereof; or a pharmaceutically acceptable salt or solvate thereof; wherein:

each R^{7a} is independently (a) cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; each of which is optionally substituted with one or more, in one embodiment, one, two, three, or four, substituents Q; (c) —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —S(O)R^{a}, —S(O)₂R^{1a}, —S(O)NR^{1b}R^{1c}, or —S(O)₂NR^{1b}R^{1c}; (d) —C₁₋₆ alkylene-OP(O)(OR^{P1})₂, in one embodiment, —CH₂—OP(O)(OR^{P1})₂; or (e) —C₁₋₆ alkylene-O-linked amino acid or a derivative thereof, in one embodiment, —CH₂—OC(O)C(R^{aa})₂NR^{1b}R^{1c};

r is an integer of 0, 1, 2, 3, or 4; and

R¹, R², R³, R⁴, R^{1a}, R^{1b}, R^{1c}, R^{1d}, R^{P1}, L¹, L², Q, U¹, U², V¹, V², W¹, W², X¹, X², Z¹, Z², m, n, s, and t are each as defined herein.

In certain embodiments of the compound of Formula IX, or single enantiomer, racemic mixture, diastereomer, mixture of diastereomers, or isotopic variant thereof; or pharmaceutically acceptable salt or solvate thereof;

each R^{7a} is independently (a) cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; each of which is optionally substituted with one or more, in one embodiment, one, two, three, or four, substituents Q; or (c) —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —S(O)R^{1a}, —S(O)₂R^{1a}, —S(O)NR^{1b}R^{1c}, or —S(O)₂NR^{1b}R^{1c}; or (d) —C₁₋₆ alkylene-OP(O)(OR^{P})₂, in one embodiment, —CH₂—OP(O)(OR^{P1})₂;

r is an integer of 0, 1, 2, 3, or 4; and

R¹, R², R³, R⁴, R^{1a}, R^{1b}, R^{1c}, R^{1d}, R^{P1}, L¹, L², Q, U¹, U², V¹, V², W¹, W², X¹, X², Z¹, Z² m, n, s, and t are each as defined herein.

In yet another embodiment, provided herein is a compound of Formula X:

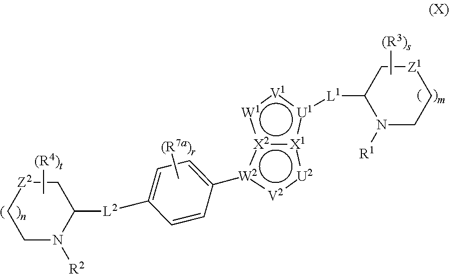

or a single enantiomer, a racemic mixture, a diastereomer, a mixture of diastereomers, or an isotopic variant thereof; or a pharmaceutically acceptable salt or solvate thereof; wherein R¹, R², R³, R⁴, R^{7a}, L¹, L², T³, U¹, U², V¹, V², W¹, W², X¹, X², Z¹, Z², m, n, r, s, and t are each as defined herein.

In one embodiment, provided herein is a compound of Formula Xa:

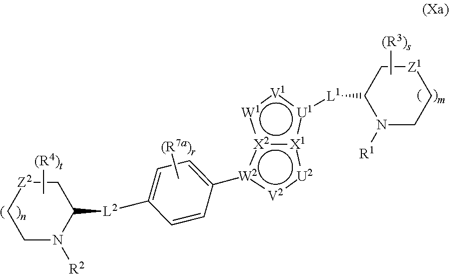

or a single enantiomer, a diastereomer, a mixture of diastereomers, or an isotopic variant thereof; or a pharmaceutically acceptable salt or solvate thereof; wherein R¹, R², R³, R⁴, R^{7a}, L¹, L², T³, U¹, U², V¹, V², W¹, W², X¹, X², Z¹, Z², m, n, r, s, and t are each as defined herein.

In another embodiment, provided herein is a compound of Formula Xb:

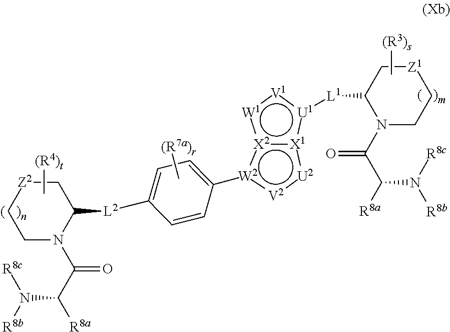

or a single enantiomer, a diastereomer, a mixture of diastereomers, or an isotopic variant thereof; or a pharmaceutically acceptable salt or solvate thereof; wherein R³, R⁴, R^{7a}, R^{8a}, R^{8b}, R^{8c}, L¹, L², U¹, U², V¹, V², W¹, W², X¹, X², Z¹, Z², m, n, r, s, and t are each as defined herein.

In yet another embodiment, provided herein is a compound of Formula Xc:

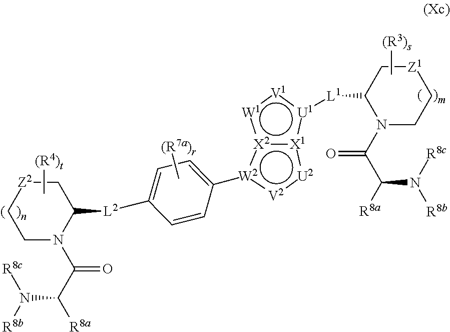

or a single enantiomer, a diastereomer, a mixture of diastereomers, or an isotopic variant thereof; or a pharmaceutically acceptable salt or solvate thereof; wherein R³, R⁴, R^{7a}, R^{8a}, R^{8b}, R^{8c}, L¹, L², U¹, U², V¹, V², W¹, W², X¹, X², Z¹, Z², m, n, r, s, and t are each as defined herein.

In yet another embodiment, provided herein is a compound of Formula Xd:

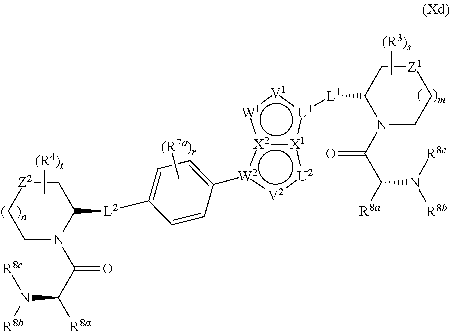

or a single enantiomer, a diastereomer, a mixture of diastereomers, or an isotopic variant thereof; or a pharmaceutically acceptable salt or solvate thereof; wherein R³, R⁴, R^{7a}, R^{8a}, R^{8b}, R^{8c}, L¹, L², U¹, U², V¹, V², W¹, W², X¹, X², Z¹, Z², m, n, r, s, and t are each as defined herein.

In still another embodiment, provided herein is a compound of Formula Xe:

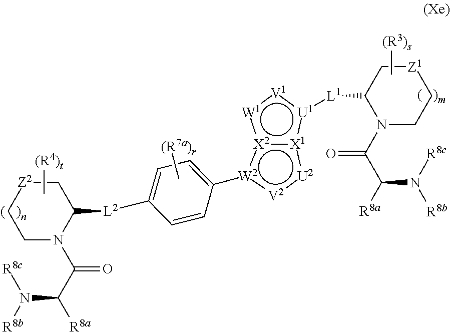

or a single enantiomer, a diastereomer, a mixture of diastereomers, or an isotopic variant thereof; or a pharmaceutically acceptable salt or solvate thereof; wherein R³, R⁴, R^{7a}, R^{8a}, R^{8b}, R^{8c}, L¹, L², U¹, U², V¹, V², W¹, W², X¹, X², Z¹, Z², m, n, r, s, and t are each as defined herein.

In yet another embodiment, provided herein is a compound of Formula XI:

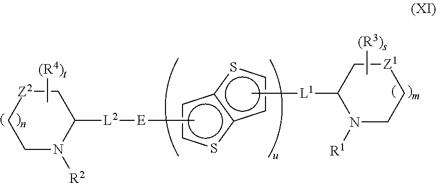

or a single enantiomer, a racemic mixture, a diastereomer, a mixture of diastereomers, or an isotopic variant thereof; or a pharmaceutically acceptable salt or solvate thereof; wherein:

u is an integer of 1 or 2;

each thieno[3,2-b]thienylene is independently and optionally substituted with one or two R^{7a}; and

R¹, R², R³, R⁴, R^{7a}, E, L¹, L², Z¹, Z², m, n, s, and t are each as defined herein.

In yet another embodiment, provided herein is a compound of Formula XII:

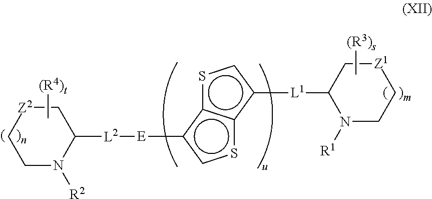

or a single enantiomer, a racemic mixture, a diastereomer, a mixture of diastereomers, or an isotopic variant thereof; or a pharmaceutically acceptable salt or solvate thereof; wherein each thieno[3,2-b]thienylene is independently and optionally substituted with one or two R^{7a}; and R¹, R², R³, R⁴, R^{7a}, E, L¹, L², Z¹, Z², m, n, S, t, and u are each as defined herein.

In one embodiment, provided herein is a compound of Formula XIIa:

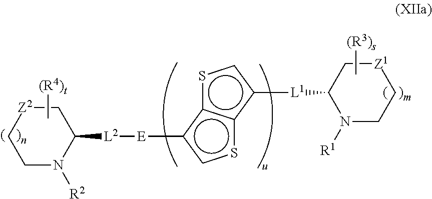

or a single enantiomer, a diastereomer, a mixture of diastereomers, or an isotopic variant thereof; or a pharmaceutically acceptable salt or solvate thereof; wherein each thieno[3,2-b]thienylene is independently and optionally substituted with one or two R^{7a}; and R¹, R², R³, R⁴, R^{7a}, E, L¹, L², Z¹, Z², m, n, s, t, and u are each as defined herein.

In yet another embodiment, provided herein is a compound of Formula XIIb:

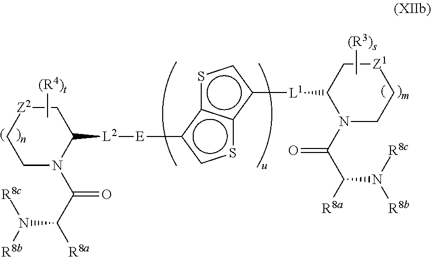

or a single enantiomer, a diastereomer, a mixture of diastereomers, or an isotopic variant thereof; or a pharmaceutically acceptable salt or solvate thereof; wherein each thieno[3,2-b]thienylene is independently and optionally substituted with one or two R^{7a}; and R³, R⁴, R^{7a}, R^{8a}, R^{8b}, R^{8c}, E, L¹, L², Z¹, Z², m, n, s, t, and u are each as defined herein.

In yet another embodiment, provided herein is a compound of Formula XIIc:

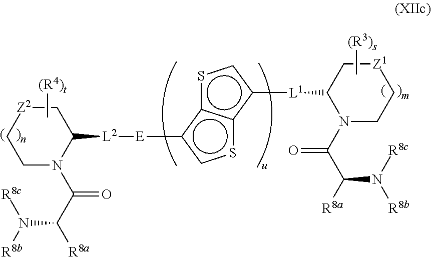

or a single enantiomer, a diastereomer, a mixture of diastereomers, or an isotopic variant thereof; or a pharmaceutically acceptable salt or solvate thereof; wherein each thieno[3,2-b]thienylene is independently and optionally substituted with one or two R^{7a}; and R³, R⁴, R^{7a}, R^{8a}, R^{8b}, R^{8c}, E, L¹, L², Z¹, Z², m, n, s, t, and u are each as defined herein.

In yet another embodiment, provided herein is a compound of Formula XIId:

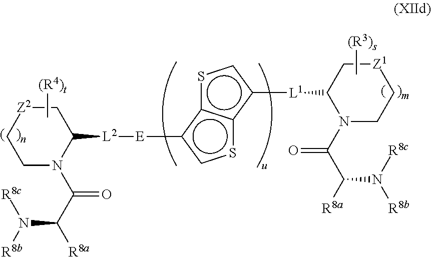

or a single enantiomer, a diastereomer, a mixture of diastereomers, or an isotopic variant thereof; or a pharmaceutically acceptable salt or solvate thereof; wherein each thieno[3,2-b]thienylene is independently and optionally substituted with one or two R^{7a}; and R³, R⁴, R^{7a}, R^{8a}, R^{8b}, R^{8c}, E, L¹, L², Z¹, Z², m, n, s, t, and u are each as defined herein.

In yet another embodiment, provided herein is a compound of Formula XIIe:

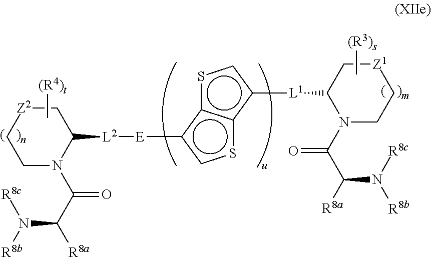

or a single enantiomer, a diastereomer, a mixture of diastereomers, or an isotopic variant thereof; or a pharmaceutically acceptable salt or solvate thereof; wherein each thieno[3,2-b]thienylene is independently and optionally substituted with one or two R^{7a}; and R³, R⁴, R^{7a}, R^{8a}, R^{8b}, R^{8c}, E, L¹, L², Z¹, Z², m, n, s, t, and u are each as defined herein.

In yet another embodiment, provided herein is a compound of Formula XIII:

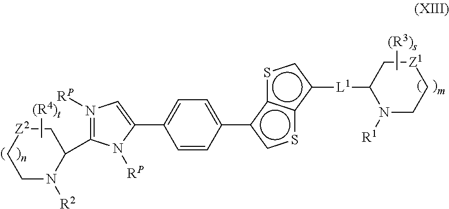

or a single enantiomer, a racemic mixture, a diastereomer, a mixture of diastereomers, or an isotopic variant thereof; or a pharmaceutically acceptable salt or solvate thereof; wherein:

each R^{P} is independently absent, hydrogen, —C₁₋₆ alkylene-OP(O)(OR^{P1})₂ (in one embodiment, —CH₂—OP(O)(OR^{P1})₂), or —C₁₋₆ alkylene-O-linked amino acid or a derivative thereof (in one embodiment, —CH₂—OC(O)C(R^{aa})₂NR^{1b}R^{1c}); wherein, when the two R^{P} groups attached to the imidazolylene are neither absent nor hydrogen, the imidazolyene group carries a positive charge; and wherein at least one of the R^{P} groups is neither absent nor hydrogen;

the phenylene and thieno[3,2-b]thienylene are each independently and optionally substituted with one to more, in one embodiment, one, two, three, or four, R^{7a};

the imidazolylene is independently and optionally substituted with a substituent Q; and

R¹, R², R³, R⁴, R^{1b}, R^{1c}, R^{7a}, R^{aa}, R^{P1}, Q, Z¹, Z², m, n, S, and t are each as defined herein.

In certain embodiments of the compound of Formula XIII, or single enantiomer, racemic mixture, diastereomer, mixture of diastereomers, or isotopic variant thereof; or pharmaceutically acceptable salt or solvate thereof;

each R^{P} is independently absent, hydrogen, or —C₁₋₆ alkylene-OP(O)(OR^{P1})₂, in one embodiment, —CH₂—OP(O)(OR^{P1})₂; wherein, when the two R^{P} groups attached to the imidazolylene are neither absent nor hydrogen, the imidazolyene group carries a positive charge; and wherein at least one of the R^{P} groups is neither absent nor hydrogen;

the phenylene and thieno[3,2-b]thienylene are each independently and optionally substituted with one to more, in one embodiment, one, two, three, or four, R^{7a};

the imidazolylene is independently and optionally substituted with a substituent Q; and

R¹, R², R³, R⁴, R^{7a}, R^{P1}, L, Q, Z¹, Z², m, n, s, and t are each as defined herein.

In one embodiment, provided herein is a compound of Formula XIIIa:

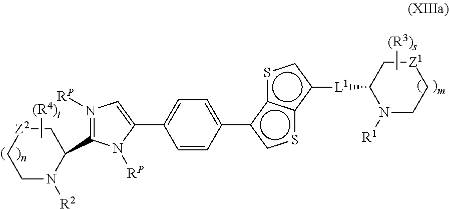

or a single enantiomer, a diastereomer, a mixture of diastereomers, or an isotopic variant thereof; or a pharmaceutically acceptable salt or solvate thereof; wherein the phenylene and thieno[3,2-b]thienylene are each independently and optionally substituted with one to more, in one embodiment, one, two, three, or four, R^{7a}; the imidazolylene is independently and optionally substituted with a substituent Q; and R¹, R², R³, R⁴, R^{7a}, R^{P}, L¹, Q, Z¹, Z², m, n, s, and t are each as defined herein.

In another embodiment, provided herein is a compound of Formula XIIIb:

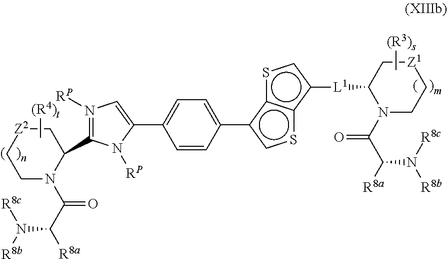

or a single enantiomer, a diastereomer, a mixture of diastereomers, or an isotopic variant thereof; or a pharmaceutically acceptable salt or solvate thereof; wherein the phenylene and thieno[3,2-b]thienylene are each independently and optionally substituted with one to more, in one embodiment, one, two, three, or four, R^{7a}; the imidazolylene is independently and optionally substituted with a substituent Q; and R³, R⁴, R^{7a}, R^{8a}, R^{8b}, R^{8c}, R^{P}, L¹, Q, Z¹, Z², m, n, s, and t are each as defined herein.

In yet another embodiment, provided herein is a compound of Formula XIIIc:

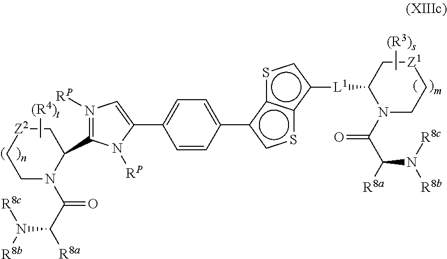

or a single enantiomer, a diastereomer, a mixture of diastereomers, or an isotopic variant thereof; or a pharmaceutically acceptable salt or solvate thereof; wherein the phenylene and thieno[3,2-b]thienylene are each independently and optionally substituted with one to more, in one embodiment, one, two, three, or four, R^{7a}; the imidazolylene is independently and optionally substituted with a substituent Q; and R³, R⁴, R^{7a}, R^{8a}, R^{8b}, R^{8c}, R^{P}, L¹, Q, Z¹, Z², m, n, s, and t are each as defined herein.

In yet another embodiment, provided herein is a compound of Formula XIIId:

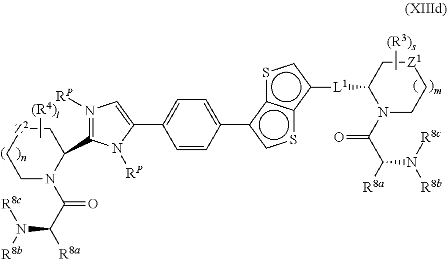

or a single enantiomer, a diastereomer, a mixture of diastereomers, or an isotopic variant thereof; or a pharmaceutically acceptable salt or solvate thereof; wherein the phenylene and thieno[3,2-b]thienylene are each independently and optionally substituted with one to more, in one embodiment, one, two, three, or four, R^{7a}; the imidazolylene is independently and optionally substituted with a substituent Q; and R³, R⁴, R^{7a}, R^{8a}, R^{8b}, R^{8c}, R^{P}, L¹, Q, Z¹, Z², m, n, s, and t are each as defined herein.

In still another embodiment, provided herein is a compound of Formula XIIIe:

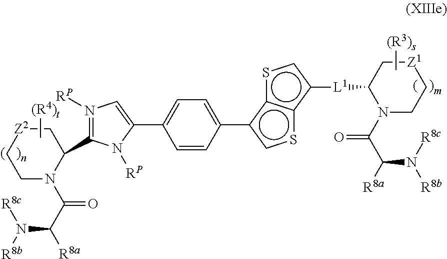

or a single enantiomer, a diastereomer, a mixture of diastereomers, or an isotopic variant thereof; or a pharmaceutically acceptable salt or solvate thereof; wherein the phenylene and thieno[3,2-b]thienylene are each independently and optionally substituted with one to more, in one embodiment, one, two, three, or four, R^{7a}; the imidazolylene is independently and optionally substituted with a substituent Q; and R³, R⁴, R^{7a}, R^{8a}, R^{8b}, R^{8c}, R^{P}, L¹, Q, Z¹, Z², m, n, s, and t are each as defined herein.

In still another embodiment, provided herein is a compound of Formula XIV:

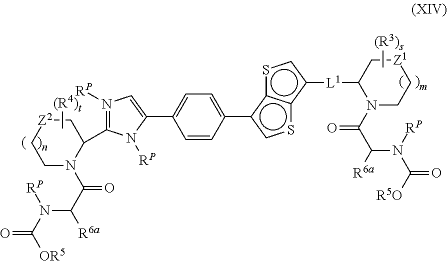

or a single enantiomer, a racemic mixture, a diastereomer, a mixture of diastereomers, or an isotopic variant thereof; or a pharmaceutically acceptable salt or solvate thereof; wherein the phenylene and thieno[3,2-b]thienylene are each independently and optionally substituted with one to more, in one embodiment, one, two, three, or four, R^{7a}; the imidazolylene is independently and optionally substituted with a substituent Q; with the proviso that at lease on of the R^{P} groups is neither absent or hydrogen; and R³, R⁴, R⁵, R^{6a}, R^{7a}, R^{P}, L¹, Q, Z¹, Z², m, n, s, and t are each as defined herein.

In one embodiment, provided herein is a compound of Formula XIVa:

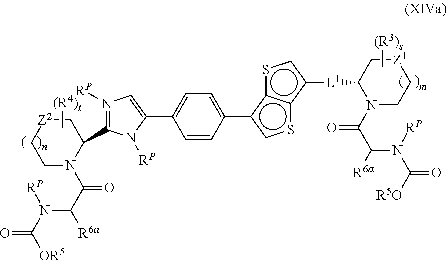

or a single enantiomer, a diastereomer, a mixture of diastereomers, or an isotopic variant thereof; or a pharmaceutically acceptable salt or solvate thereof; wherein the phenylene and thieno[3,2-b]thienylene are each independently and optionally substituted with one to more, in one embodiment, one, two, three, or four, R^{7a}; the imidazolylene is independently and optionally substituted with a substituent Q; and R³, R⁴, R⁵, R^{6a}, R^{7a}, R^{P}, L¹, Q, Z¹, Z², m, n, s, and t are each as defined herein.

In another embodiment, provided herein is a compound of Formula XIVb:

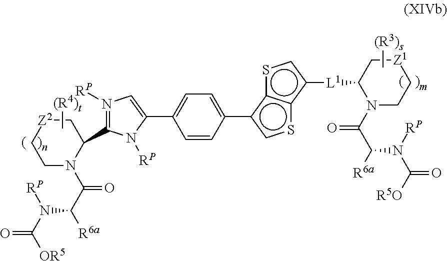

or a single enantiomer, a diastereomer, a mixture of diastereomers, or an isotopic variant thereof; or a pharmaceutically acceptable salt or solvate thereof; wherein the phenylene and thieno[3,2-b]thienylene are each independently and optionally substituted with one to more, in one embodiment, one, two, three, or four, R^{7a}; the imidazolylene is independently and optionally substituted with a substituent Q; and R³, R⁴, R⁵, R^{6a}, R^{7a}, R^{P}, L¹, Q, Z¹, Z², m, n, s, and t are each as defined herein.

In yet another embodiment, provided herein is a compound of Formula XIVc:

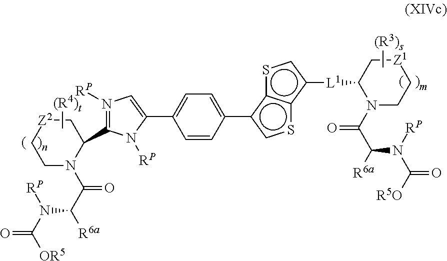

or a single enantiomer, a diastereomer, a mixture of diastereomers, or an isotopic variant thereof; or a pharmaceutically acceptable salt or solvate thereof; wherein the phenylene and thieno[3,2-b]thienylene are each independently and optionally substituted with one to more, in one embodiment, one, two, three, or four, R^{7a}; the imidazolylene is independently and optionally substituted with a substituent Q; and R³, R⁴, R⁵, R^{6a}, R^{7a}, R^{P}, L¹, Q, Z¹, Z², m, n, s, and t are each as defined herein.

In yet another embodiment, provided herein is a compound of Formula XIVd:

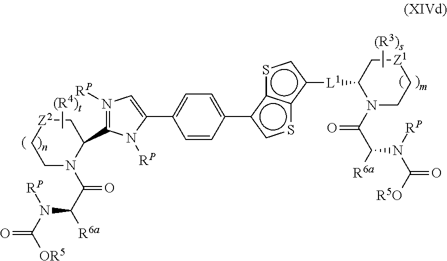

or a single enantiomer, a diastereomer, a mixture of diastereomers, or an isotopic variant thereof; or a pharmaceutically acceptable salt or solvate thereof; wherein the phenylene and thieno[3,2-b]thienylene are each independently and optionally substituted with one to more, in one embodiment, one, two, three, or four, R^{7a}; the imidazolylene is independently and optionally substituted with a substituent Q; and R³, R⁴, R⁵, R^{6a}, R^{7a}, R^{P}, L¹, Q, Z¹, Z², m, n, s, and t are each as defined herein.

In still another embodiment, provided herein is a compound of Formula XIVe:

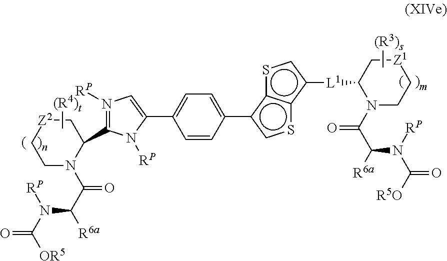

or a single enantiomer, a diastereomer, a mixture of diastereomers, or an isotopic variant thereof; or a pharmaceutically acceptable salt or solvate thereof; wherein the phenylene and thieno[3,2-b]thienylene are each independently and optionally substituted with one to more, in one embodiment, one, two, three, or four, R^{7a}; the imidazolylene is independently and optionally substituted with a substituent Q; and R³, R⁴, R⁵, R^{6a}, R^{7a}, R^{P}, L¹, Q, Z¹, Z², m, n, s, and t are each as defined herein.

In yet another embodiment, provided herein is a compound of Formula IA:

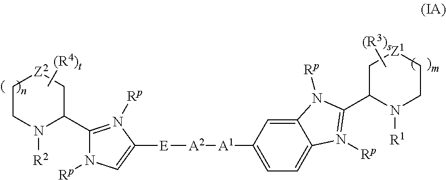

or a single enantiomer, a racemic mixture, a diastereomer, a mixture of diastereomers, or an isotopic variant thereof; or a pharmaceutically acceptable salt or solvate thereof;

A¹, A², and E are each independently (a) a bond; or (b) C₁₋₆ alkylene, C₂₋₆ alkenylene, C₂₋₆ alkynylene, C₂₋₂₀ cycloalkylene, C₆₋₂₀ arylene, heteroarylene; or heterocyclylene;

Z¹ and Z² are each independently a bond, —O—, —S—, —S(O)—, —S(O₂)—, or —N(R^{N})—;

R¹ and R² are each independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (c) —C(O)R^{1a}, —C(O)CH(N(R^{1c})C(O)OR^{1b})R^{1a}, —C(O)OR^{1a}, —C(O)NR^{1b}R^{1c}, —C(NR^{1a})NR^{1b}R^{1c}, —P(O)(OR^{1a})R^{1d}, —CH₂P(O)(OR^{1a})R^{1d}, —S(O)R^{1a}, —S(O)₂R^{1a}, —S(O)NR^{1b}R^{1c}, or —S(O)₂NR^{1b}R^{1c}; (d) —C₁₋₆ alkylene-OP(O)(OR^{P1})₂, in one embodiment, —CH₂—OP(O)(OR^{P1})₂; or (e) —C₁₋₆ alkylene-O-linked amino acid or a derivative thereof, in one embodiment, —CH₂—OC(O)C(R^{aa})₂NR^{1b}R^{1c};

each R³ and R⁴ is independently (a) cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —SR^{1a}, —S(O)R^{1a}, —S(O)₂R^{1a}, —S(O)NR^{1b}R^{1c}, or —S(O)₂NR^{1b}R^{1c}; or

two R³ or two R⁴ that are attached to the same ring are linked together to form a bond, —O—, —NR^{N}—, —S—, C₁₋₆ alkylene, C₁₋₆ heteroalkylene, C₂₋₆ alkenylene, or C₂₋₆ heteroalkenylene;

each R^{N} is independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (c) —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R¹, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —S(O)R^{1a}, —S(O)₂R^{1a}, —S(O)NR^{1b}R^{1c}, or —S(O)₂NR^{1b}R^{1c}; (d) —C₁₋₆ alkylene-OP(O)(OR^{P1})₂, in one embodiment, —CH₂—OP(O)(OR^{P1})₂; or (e) —C₁₋₆ alkylene-O-linked amino acid or a derivative thereof, in one embodiment, —CH₂—OC(O)C(R^{aa})₂NR^{1b}R^{1c};

each R^{P} is independently absent, hydrogen, or —C₁₋₆ alkylene-OP(O)(OR^{P1})₂ (in one embodiment, —CH₂—OP(O)(OR^{P1})₂), or —C₁₋₆ alkylene-O-linked amino acid or a derivative thereof (in one embodiment, —CH₂—OC(O)C(R^{aa})₂NR^{1b}R^{1c}); wherein, when the two R^{P} groups attached to the imidazolylene are neither absent nor hydrogen, the imidazolyene group carries a positive charge; and when the two R^{P} groups attached to the benzimidazolylene are neither absent nor hydrogen, the benzimidazolylene group carries a positive charge; and wherein at least one of the R^{P} groups is neither absent nor hydrogen;

each R^{P1} is independently (a) hydrogen; (b) a monovalent cation, in one embodiment, Na⁺ or K⁺, in another embodiment, Li⁺, Rb⁺, or Cs⁺; (c) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (d) or two R^{P1} together are a divalent cation, in one embodiment, Mg²⁺ or Ca²⁺;

each R^{1a}, R^{1b}, R^{1c}, and R^{1d} is independently hydrogen, C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or R^{1a} and R^{1c} together with the C and N atoms to which they are attached form heterocyclyl; or R^{1b} and R^{1c} together with the N atom to which they are attached form heterocyclyl;

each R^{aa} independently is a side chain of a naturally occurring or non-naturally occurring amino acid; in one embodiment, each R^{aa} independently is hydrogen, C₁₋₆ alkyl, heteroalkyl, C₆₋₁₄ aryl-C₁₋₆ alkyl and heteroaryl-C₁₋₆ alkyl;

each m and n is independently an integer of 1, 2, 3, or 4; and

each s and t is independently an integer of 0, 1, 2, 3, 4, 5, 6, or 7;

wherein each alkyl, alkylene, heteroalkylene, alkenyl, alkenylene, heteroalkenylene, alkynyl, alkynylene, cycloalkyl, cycloalkylene, aryl, arylene, aralkyl, heteroaryl, heteroarylene, imidazolylene, benzimidazolyl, heterocyclyl, and heterocyclylene is optionally substituted with one or more, in one embodiment, one, two, three, or four, substituents Q, where each Q is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is further optionally substituted with one or more, in one embodiment, one, two, three, or four, substituents Q^{a}; or (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OP(O)(OR^{e})₂, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —P(O)(OR^{a})R^{d}, —CH₂P(O)(OR^{a})R^{d}, —CH₂OP(O)(OR^{e})₂, —CH₂OC(O)C(R^{a})₂NR^{b}R^{c}, —SR^{a}, —S(O)R^{a}, —S(O)₂R^{a}, —S(O)NR^{b}R^{c}, or —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a}; and each R^{e} is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iv) two R^{e} together are a divalent cation (e.g., Mg²⁺ or Ca²⁺);

wherein each Q^{a} is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{f}, —C(O)OR^{f}, —C(O)NR^{g}R^{h}, —C(NR^{f})NR^{g}R^{h}, —OR^{f}, —OC(O)R^{f}, —OC(O)OR^{f}, —OC(O)NR^{g}R^{h}, —OC(═NR^{f})NR^{g}R^{h}, —OP(O)(ORⁿ)₂, —OS(O)R^{f}, —OS(O)₂R^{f}, —OS(O)NR^{g}R^{h}, —OS(O)₂NR^{g}R^{h}, —NR^{g}R^{h}, —NR^{f}C(O)R^{k}, —NR^{f}C(O)OR^{k}, —NR^{f}C(O)NR^{g}R^{h}, —NR^{f}C(═NR^{k})NR^{g}R^{h}, —NR^{f}S(O)R^{k}, —NR^{f}S(O)₂R^{k}, —NR^{f}S(O)NR^{g}R^{h}, —NR^{f}S(O)₂NR^{g}R^{h}, —P(O)(OR^{f})R^{k}, —CH₂P(O)(OR^{f})R^{k}, —SR^{f}, —S(O)R^{f}, —S(O)₂R, —S(O)NR^{g}R^{h}, or —S(O)₂NR^{g}R^{h}; wherein each R^{f}, R^{g}, R^{h}, and R^{k} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{g} and R^{h} together with the N atom to which they are attached form heterocyclyl; and each Rⁿ is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iv) two Rⁿ together are a divalent cation (e.g., Mg²⁺ or Ca²⁺).

In certain embodiments of the compound of Formula IA, or single enantiomer, racemic mixture, diastereomer, mixture of diastereomers, or isotopic variant thereof; or pharmaceutically acceptable salt or solvate thereof;

A¹, A², and E are each independently (a) a bond; or (b) C₁₋₆ alkylene, C₂₋₆ alkenylene, C₂₋₆ alkynylene, C₂₋₂₀ cycloalkylene, C₆₋₂₀ arylene, heteroarylene; or heterocyclylene;

Z¹ and Z² are each independently a bond, —O—, —S—, —S(O)—, —S(O₂)—, or —N(R^{N})—;

R¹ and R² are each independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (c) —C(O)R^{1a}, —C(O)CH(N(R^{1c})C(O)OR^{1b})R^{1a}, —C(O)OR^{1a}, —C(O)NR^{1b}R^{1c}, —C(NR^{1a})NR^{1b}R^{1c}, —P(O)(OR^{1a})R^{1d}, —CH₂P(O)(OR^{1a})R^{1d}, —S(O)R^{1a}, —S(O)₂R^{1a}, —S(O)NR^{1b}R^{1c}, or —S(O)₂NR^{1b}R^{1c}; or (d) —C₁₋₆ alkylene-OP(O)(OR^{P1})₂, in one embodiment, —CH₂—OP(O)(OR^{P1})₂;

each R³ and R⁴ is independently (a) cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{a})R^{d}, —SR^{1a}, —S(O)R^{1a}, —S(O)₂R^{a}, —S(O)NR^{1b}R^{1c}, or —S(O)₂NR^{1b}R^{1c}; or

two R³ or two R⁴ that are attached to the same ring are linked together to form a bond, —O—, —NR^{N}—, —S—, C₁₋₆ alkylene, C₁₋₆ heteroalkylene, C₂₋₆ alkenylene, or C₂₋₆ heteroalkenylene;

each R^{N} is independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (c) —C(O)OR¹, —C(O)NR^{1b}R^{1c}, —C(NR^{a})NR^{1b}R^{1c}, —OR^{1a}, —OC(O)R^{1a}, —OC(O)OR^{1a}, —OC(O)NR^{b}R^{c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{a}C(═NR^{d})NR^{1b}R^{1c}, —NR^{1a}S(O)R^{1d}, —NR^{1a}S(O)₂R^{d}, —NR^{1a}S(O)NR^{1b}R^{1c}, —NR^{1a}S(O)₂NR^{1b}R^{1c}, —P(O)(OR^{1a})R^{d}, —CH₂P(O)(OR^{1a})R^{1d}, —S(O)R^{1a}, —S(O)₂R^{1a}, —S(O)NR^{1b}R^{1c}, or —S(O)₂NR^{1b}R^{1c}; or (d) —C₁₋₆ alkylene-OP(O)(OR^{P1})₂, in one embodiment, —CH₂—OP(O)(OR^{P1})₂;

each R^{P} is independently absent, hydrogen, or —C₁₋₆ alkylene-OP(O)(OR^{P1})₂, in one embodiment, —CH₂—OP(O)(OR^{P1})₂; wherein, when the two R^{P} groups attached to the imidazolylene are neither absent nor hydrogen, the imidazolyene group carries a positive charge; and when the two R^{P} groups attached to the benzimidazolylene are neither absent nor hydrogen, the benzimidazolylene group carries a positive charge; and wherein at least one of the R^{P} groups is neither absent nor hydrogen;

each R^{P1} is independently (a) hydrogen; (b) a monovalent cation, in one embodiment, Na⁺ or K⁺; or (c) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or two R^{P1} together are a divalent cation, in one embodiment, Mg²⁺ or Ca²⁺;

each R^{1a}, R^{1b}, R^{1c}, and R^{1d} is independently hydrogen, C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or R^{1a} and R^{1c} together with the C and N atoms to which they are attached form heterocyclyl; or R^{1b} and R^{1c} together with the N atom to which they are attached form heterocyclyl;

each m and n is independently an integer of 1, 2, 3, or 4; and

each s and t is independently an integer of 0, 1, 2, 3, 4, 5, 6, or 7;

wherein each alkyl, alkylene, heteroalkylene, alkenyl, alkenylene, heteroalkenylene, alkynyl, alkynylene, cycloalkyl, cycloalkylene, aryl, arylene, aralkyl, heteroaryl, heteroarylene, imidazolylene, benzimidazolyl, heterocyclyl, and heterocyclylene is optionally substituted with one or more, in one embodiment, one, two, three, or four, substituents Q, where each Q is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is further optionally substituted with one or more, in one embodiment, one, two, three, or four, substituents Q^{a}; and (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OP(O)(OR^{e})₂, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —P(O)(OR^{a})Rd, —CH₂P(O)(OR^{a})R^{d}, —SR^{a}, —S(O)R^{a}, —S(O)₂R^{a}, —S(O)NR^{b}R^{1c}, or —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a}; and each R^{e} is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iv) two R^{e} together are a divalent cation (e.g., Mg²⁺ or Ca²⁺);

wherein each Q^{a} is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; and (c) —C(O)R^{f}, —C(O)OR^{f}, —C(O)NR^{g}R^{h}, —C(NR^{f})NR^{g}R^{h}, —OR^{f}, —OC(O)R^{f}, —OC(O)OR^{f}, —OC(O)NR^{g}R^{h}, —OC(═NR^{f})NR^{g}R^{h}, —OP(O)(ORⁿ)₂, —OS(O)R^{f}, —OS(O)₂R^{f}, —OS(O)NR^{g}R^{h}, —OS(O)₂NR^{g}R^{h}, —NR^{g}R^{h}, —NR^{f}C(O)R^{k}, —NR^{f}C(O)OR^{k}, —NR^{f}C(O)NR^{g}R^{h}, —NR^{f}C(═NR^{k})NR^{g}R^{h}, —NR^{f}S(O)R^{k}, —NR^{f}S(O)₂R^{k}, —NR^{f}S(O)NR^{g}R^{h}, —NR^{f}S(O)₂NR^{g}R^{h}, —P(O)(OR^{f})R^{k}, —CH₂P(O)(OR^{f})R^{k}, —SR^{f}, —S(O)R^{f}, —S(O)₂R, —S(O)NR^{g}R^{h}, or —S(O)₂NR^{g}R^{h}; wherein each R^{f}, R^{g}, R^{h}, and R^{k} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{g} and R^{h} together with the N atom to which they are attached form heterocyclyl; and each Rⁿ is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iv) two Rⁿ together are a divalent cation (e.g., Mg²⁺ or Ca²⁺).

In one embodiment, provided herein is a compound of Formula IAa:

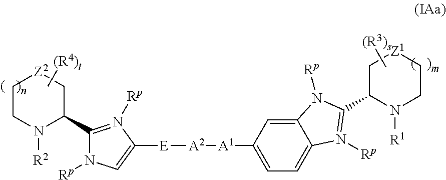

or a single enantiomer, a diastereomer, a mixture of diastereomers, or an isotopic variant thereof; or a pharmaceutically acceptable salt or solvate thereof; wherein the imidazolylene and benzimidazolylene are each independently and optionally substituted with one, two, or three substituents Q; and R¹, R², R³, R⁴, R^{P}, A¹, A², E, Q, Z¹, Z², m, n, s, and t are each as defined herein.

In another embodiment, provided herein is a compound of Formula IAb:

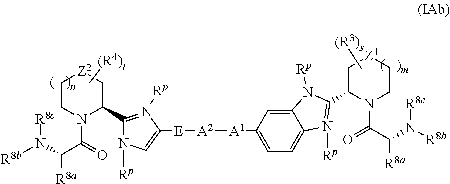

or a single enantiomer, a diastereomer, a mixture of diastereomers, or an isotopic variant thereof; or a pharmaceutically acceptable salt or solvate thereof; wherein the imidazolylene and benzimidazolylene are each independently and optionally substituted with one, two, or three substituents Q; and R³, R⁴, R^{8a}, R^{8b}, R^{8c}, R^{P}, A¹, A², E, Q, Z¹, Z², m, n, S, and t are each as defined herein.

In yet another embodiment, provided herein is a compound of Formula IAc:

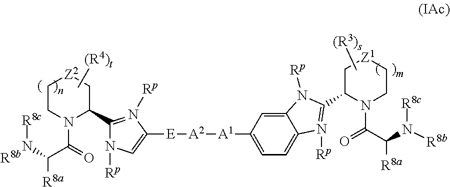

or a single enantiomer, a diastereomer, a mixture of diastereomers, or an isotopic variant thereof; or a pharmaceutically acceptable salt or solvate thereof; wherein the imidazolylene and benzimidazolylene are each independently and optionally substituted with one, two, or three substituents Q; and R³, R⁴, R^{8a}, R^{8b}, R^{8c}, R^{P}, A¹, A², E, Q, Z¹, Z², m, n, S, and t are each as defined herein.

In yet another embodiment, provided herein is a compound of Formula IAd:

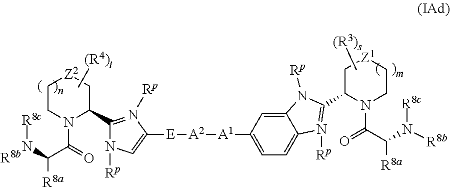

or a single enantiomer, a diastereomer, a mixture of diastereomers, or an isotopic variant thereof; or a pharmaceutically acceptable salt or solvate thereof; wherein the imidazolylene and benzimidazolylene are each independently and optionally substituted with one, two, or three substituents Q; and R³, R⁴, R^{8a}, R^{8b}, R^{8c}, R^{P}, A¹, A², E, Q, Z¹, Z², m, n, S, and t are each as defined herein.

In still another embodiment, provided herein is a compound of Formula IAe:

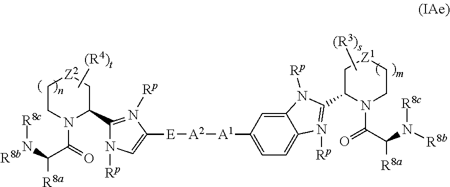

or a single enantiomer, a diastereomer, a mixture of diastereomers, or an isotopic variant thereof; or a pharmaceutically acceptable salt or solvate thereof; wherein the imidazolylene and benzimidazolylene are each independently and optionally substituted with one, two, or three substituents Q; and R³, R⁴, R^{8a}, R^{8b}, R^{8c}, R^{P}, A¹, A², E, Q, Z¹, Z², m, n, S, and t are each as defined herein.

In yet another embodiment, provided herein is a compound of Formula IIA:

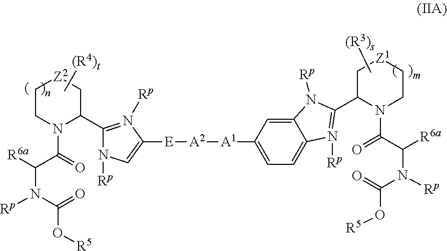

or a single enantiomer, a racemic mixture, a diastereomer, a mixture of diastereomers, or an isotopic variant thereof; or a pharmaceutically acceptable salt or solvate thereof; wherein: each R⁵ is independently C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl;

each R^{6a} is independently hydrogen, C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl;

the imidazolylene and benzimidazolylene are each independently and optionally substituted with one, two, or three substituents Q;

with the proviso that at least one of the R^{P} groups is neither absent or hydrogen; and

R³, R⁴, R^{P}, R^{P1}, A¹, A², E, Q, Z¹, Z², m, n, s, and t are each as defined herein.

In one embodiment, provided herein is a compound of Formula IIAa:

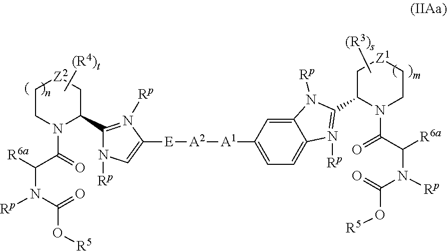

or a single enantiomer, a diastereomer, a mixture of diastereomers, or an isotopic variant thereof; or a pharmaceutically acceptable salt or solvate thereof; wherein the imidazolylene and benzimidazolylene are each independently and optionally substituted with one, two, or three substituents Q; with the proviso that at least one of the R^{P} groups is —C₁₋₆ alkylene-OP(O)(OR^{P1})₂ (in one embodiment, —CH₂—OP(O)(OR^{P1})₂) or —C₁₋₆ alkylene-O-linked amino acid or a derivative thereof (in one embodiment, —CH₂—OC(O)C(R^{aa})₂NR^{1b}R^{1c}); and R³, R⁴, R⁵, R^{1b}, R^{1c}, R^{6a}, R^{6a}, R^{P}, R^{P1}, A¹, A², E, Q, Z¹, Z², m, n, S, and t are each as defined herein.

In certain embodiments of the compound of Formula IIAa, or single enantiomer, diastereomer, mixture of diastereomers, or isotopic variant thereof; or pharmaceutically acceptable salt or solvate thereof;

the imidazolylene and benzimidazolylene are each independently and optionally substituted with one, two, or three substituents Q; with the proviso that at least one of the R^{P} groups is —C₁₋₆ alkylene-OP(O)(OR^{P1})₂, in one embodiment, —CH₂—OP(O)(OR^{P1})₂; and R³, R⁴, R⁵, R^{6a}, R^{P}, R^{P1}, A¹, A², E, Q, Z¹, Z², m, n, s, and t are each as defined herein.

In yet another embodiment, provided herein is a compound of Formula IIAb:

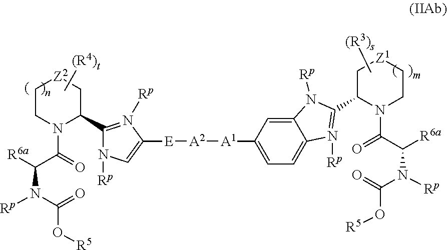

or a single enantiomer, a diastereomer, a mixture of diastereomers, or an isotopic variant thereof; or a pharmaceutically acceptable salt or solvate thereof; wherein the imidazolylene and benzimidazolylene are each independently and optionally substituted with one, two, or three substituents Q; with the proviso that at least one of the R^{P} groups is —C₁₋₆ alkylene-OP(O)(OR^{P1})₂ (in one embodiment, —CH₂—OP(O)(OR^{P1})₂) or —C₁₋₆ alkylene-O-linked amino acid or a derivative thereof (in one embodiment, —CH₂—OC(O)C(R^{aa})₂NR^{1b}R^{1c}); and R³, R⁴, R⁵, R^{1b}, R^{1c}, R^{6a}, R^{aa}, R^{P}, R^{P1}, A¹, A², E, Q, Z¹, Z², m, n, S, and t are each as defined herein.

In certain embodiments of the compound of Formula IIAb, or single enantiomer, diastereomer, mixture of diastereomers, or isotopic variant thereof; or pharmaceutically acceptable salt or solvate thereof;

the imidazolylene and benzimidazolylene are each independently and optionally substituted with one, two, or three substituents Q; with the proviso that at least one of the R^{P} groups is —C₁₋₆ alkylene-OP(O)(OR^{P1})₂, in one embodiment, —CH₂—OP(O)(OR^{P1})₂; and R³, R⁴, R⁵, R^{6a}, R^{P}, R^{P1}, A¹, A², E, Q, Z¹, Z², m, n, s, and t are each as defined herein.

In yet another embodiment, provided herein is a compound of Formula IIAc:

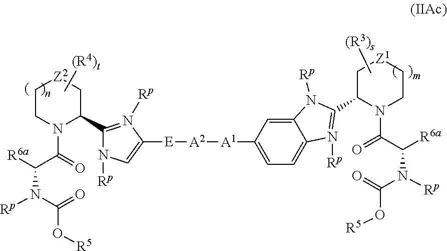

or a single enantiomer, a diastereomer, a mixture of diastereomers, or an isotopic variant thereof, or a pharmaceutically acceptable salt or solvate thereof, wherein the imidazolylene and benzimidazolylene are each independently and optionally substituted with one, two, or three substituents Q; with the proviso that at least one of the R^{P} groups is —C₁₋₆ alkylene-OP(O)(OR^{P1})₂ (in one embodiment, —CH₂—OP(O)(OR^{P1})₂) or —C₁₋₆alkylene-O-linked amino acid or a derivative thereof (in one embodiment, —CH₂—OC(O)C(R^{a})₂NR^{1b}R^{1c}); and R³, R⁴, R⁵, R^{1b}, R^{6a}, R^{aa}, R^{P}, R^{P1}, A¹, A², E, Q, Z¹, Z², m, n, s, and t are each as defined herein.

In certain embodiments of the compound of Formula IIAc, or single enantiomer, diastereomer, mixture of diastereomers, or isotopic variant thereof or pharmaceutically acceptable salt or solvate thereof, the imidazolylene and benzimidazolylene are each independently and optionally substituted with one, two, or three substituents Q; with the proviso that at least one of the R^{P} groups is —C₁₋₆ alkylene-OP(O)(OR^{P1})₂, in one embodiment, —CH₂—OP(O)(OR^{P1})₂; and R³, R⁴, R⁵, R^{6a}, R^{P}, R^{P1}, A¹, A², E, Q, Z¹, Z², m, n, s, and t are each as defined herein.

In yet another embodiment, provided herein is a compound of Formula IIAd:

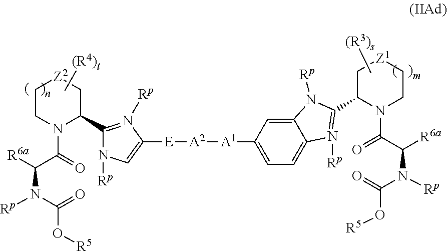

or a single enantiomer, a diastereomer, a mixture of diastereomers, or an isotopic variant thereof; or a pharmaceutically acceptable salt or solvate thereof; wherein the imidazolylene and benzimidazolylene are each independently and optionally substituted with one, two, or three substituents Q; with the proviso that at least one of the R^{P} groups is —C₁₋₆ alkylene-OP(O)(OR^{P1})₂ (in one embodiment, —CH₂—OP(O)(OR^{P1})₂) or —C₁₋₆ alkylene-O-linked amino acid or a derivative thereof (in one embodiment, —CH₂—OC(O)C(R^{aa})₂NR^{1b}R^{1c}); and R³, R⁴, R⁵, R^{1b}, R^{1c}, R^{6a}, R^{aa}, R^{P}, R^{P1}, A¹, A², E, Q, Z¹, Z², m, n, S, and t are each as defined herein.

In certain embodiments of the compound of Formula IIAd, or single enantiomer, diastereomer, mixture of diastereomers, or isotopic variant thereof; or pharmaceutically acceptable salt or solvate thereof;

the imidazolylene and benzimidazolylene are each independently and optionally substituted with one, two, or three substituents Q; with the proviso that at least one of the R^{P} groups is —C₁₋₆ alkylene-OP(O)(OR^{P1})₂, in one embodiment, —CH₂—OP(O)(OR^{P1})₂; and R³, R⁴, R⁵, R^{6a}, R^{P}, R^{P1}, A¹, A², E, Q, Z¹, Z², m, n, s, and t are each as defined herein.

In still another embodiment, provided herein is a compound of Formula IIAe:

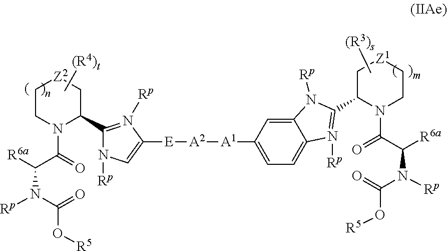

or a single enantiomer, a diastereomer, a mixture of diastereomers, or an isotopic variant thereof; or a pharmaceutically acceptable salt or solvate thereof; wherein the imidazolylene and benzimidazolylene are each independently and optionally substituted with one, two, or three substituents Q; with the proviso that at least one of the RP groups is —C₁₋₆ alkylene-OP(O)(OR^{P1})₂ (in one embodiment, —CH₂—OP(O)(OR^{P1})₂) or —C₁₋₆ alkylene-O-linked amino acid or a derivative thereof (in one embodiment, —CH₂—OC(O)C(R^{aa})₂NR^{1b}R^{1c}); and R³, R⁴, R⁵, R^{1b}, R^{1c}, R^{6a}, R^{aa}, R^{P}, R^{P1}, A¹, A², E, Q, Z¹, Z², m, n, S, and t are each as defined herein.

In certain embodiments of the compound of Formula IIAe, or single enantiomer, diastereomer, mixture of diastereomers, or isotopic variant thereof; or pharmaceutically acceptable salt or solvate thereof;

the imidazolylene and benzimidazolylene are each independently and optionally substituted with one, two, or three substituents Q; with the proviso that at least one of the R^{P} groups is —C₁₋₆ alkylene-OP(O)(OR^{P1})₂, in one embodiment, —CH₂—OP(O)(OR^{P1})₂; and R³, R⁴, R⁵, R^{6a}, R^{P}, R^{P1}, A¹, A², E, Q, Z¹, Z², m, n, s, and t are each as defined herein.

In one embodiment, the divalent moiety -E-A²-A¹- in Formula IA, IAa, IAb, IAc, IAd, IAe, IIA, IIAa, IIAb, IIAc, IIAd, or IIAe is one as defined in any of Formulae II to XIII.

In still another embodiment, provided herein is a compound of Formula IB:

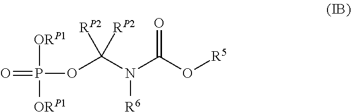

or a single enantiomer, a racemic mixture, a diastereomer, a mixture of diastereomers, or an isotopic variant thereof; or a pharmaceutically acceptable salt or solvate thereof;

R⁵ is C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl;

R⁶ is (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —CHR^{6a}C(O)R^{6b};

R^{6a} is hydrogen, C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl;

R^{6b} is:

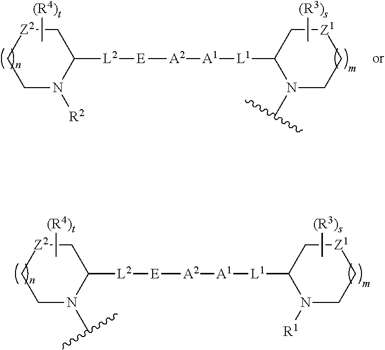

wherein:
A¹, A², and E are each independently (a) a bond; or (b) C₁₋₆ alkylene, C₂₋₆ alkenylene, C₂₋₆ alkynylene, C₂₋₂₀ cycloalkylene, C₆₋₂₀ arylene, heteroarylene; or heterocyclylene;
L¹ and L² are each independently (a) a bond; (b) C₁₋₆ alkylene, C₂₋₆ alkenylene, C₂₋₆ alkynylene, C₃₋₇ cycloalkylene, C₆₋₁₄ arylene, heteroarylene, or heterocyclylene; or (c) —C(O)—, —C(O)O—, —C(O)NR^{1a}—, —C(═NR^{1a})NR^{1c}—, —O—, —OC(O)O—, —OC(O)NR^{1a}—, —OC(═NR¹¹)NR^{1c}—, —OP(O)(OR^{1a})—, —OP(O)(OR^{1a})O—, —NR^{a}—, —NR^{1a}C(O)NR^{1c}—, —NR^{1a}C(═NR^{1b})NR^{1a}—, —NR^{1a}S(O)NR^{1c}—, —NR^{1a}S(O)₂NR^{1a}—, —S—, —S(O)—, —S(O)₂—, —S(O)NR^{1a}—, or —S(O)₂NR^{a}—;
Z¹ and Z² are each independently a bond, —O—, —S—, —S(O)—, —S(O₂)—, or —N(R^{N})—;
R¹ and R² are each independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (c) —C(O)R^{1a}, —C(O)CH(N(R^{1c})C(O)OR^{1b})R^{1a}, —C(O)OR^{1a}, —C(O)NR^{1b}R^{1c}, —C(NR^{1a})NR^{1b}R^{1c}, —P(O)(OR^{1a})R^{1d}, —CH₂P(O)(OR^{1a})R^{1d}, —S(O)R^{a}, —S(O)₂R^{1a}, —S(O)NR^{1b}R^{1c}, or —S(O)₂NR^{1b}R^{1c}; (d) —C₁₋₆ alkylene-OP(O)(OR^{P1})₂, in one embodiment, —CH₂—OP(O)(OR^{P1})₂; or (e) —C₁₋₆ alkylene-O-linked amino acid or a derivative thereof, in one embodiment, —CH₂—OC(O)C(R^{aa})₂NR^{1b}R^{1c};
each R³ and R⁴ is independently (a) cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —SR^{1a}, —S(O)R^{1a}, —S(O)₂R^{1a}, —S(O)NR^{1b}R^{1c}, or —S(O)₂NR^{1b}R^{1c}; or
two R³ or two R⁴ that are attached to the same ring are linked together to form a bond, —O—, —NR^{N}—, —S—, C₁₋₆ alkylene, C₁₋₆ heteroalkylene, C₂₋₆ alkenylene, or C₂₋₆ heteroalkenylene;
each R^{N} is independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (c) —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1d}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —S(O)R^{1a}, —S(O)₂R^{1a}, —S(O)NR^{1b}R^{1c}, or —S(O)₂NR^{1b}R^{1c}; (d) —C₁₋₆ alkylene-OP(O)(OR^{P1})₂, in one embodiment, —CH₂—OP(O)(OR^{P1})₂; or (e) —C₁₋₆ alkylene-O-linked amino acid or a derivative thereof, in one embodiment, —CH₂—OC(O)C(R^{aa})₂NR^{1b}R^{1c};
each m and n is independently an integer of 1, 2, 3, or 4; and
each s and t is independently an integer of 0, 1, 2, 3, 4, 5, 6, or 7;

each R^{P1} is independently (a) hydrogen; (b) a monovalent cation, in one embodiment, Na⁺ or K⁺, in another embodiment, Li⁺, Rb⁺, or Cs⁺; (c) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (d) two R^{P1} together are a divalent cation, in one embodiment, Mg²⁺ or Ca²⁺; and

each R^{P2} is independently (a) hydrogen, cyano, halo, or nitro; or (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl;

each R^{1a}, R^{1b}, R^{1c}, and R^{1d} is independently hydrogen, C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or R^{1a} and R^{1c} together with the C and N atoms to which they are attached form heterocyclyl; or R^{1b} and R^{1c} together with the N atom to which they are attached form heterocyclyl;

each R^{aa} independently is a side chain of a naturally occurring or non-naturally occurring amino acid; in one embodiment, each R^{aa} independently is hydrogen, C₁₋₆ alkyl, heteroalkyl, C₆₋₁₄ aryl-C₁₋₆ alkyl and heteroaryl-C₁₋₆ alkyl;

wherein each alkyl, alkylene, heteroalkylene, alkenyl, alkenylene, heteroalkenylene, alkynyl, alkynylene, cycloalkyl, cycloalkylene, aryl, arylene, aralkyl, heteroaryl, heteroarylene, heterocyclyl, and heterocyclylene is optionally substituted with one or more, in one embodiment, one, two, three, or four, substituents Q, where each Q is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is further optionally substituted with one or more, in one embodiment, one, two, three, or four, substituents Q^{a}; or (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OP(O)(OR^{e})₂, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —P(O)(OR^{a})R^{d}, —CH₂P(O)(OR^{a})R^{d}, —CH₂OP(O)(OR^{e})₂, —CH₂OC(O)C(R^{a})₂NR^{b}R^{c}, —SR^{a}, —S(O)R^{a}, —S(O)₂R^{a}, —S(O)NR^{b}R^{1c}, or —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a}; and each R^{e} is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iv) two R^{e} together are a divalent cation (e.g., Mg²⁺ or Ca²⁺);

wherein each Q^{a} is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{f}, —C(O)OR^{f}, —C(O)NR^{g}R^{h},

—C(NR^{f})NR^{g}R^{h}, —OR^{f}, —OC(O)R^{f}, —OC(O)OR^{f}, —OC(O)NR^{g}R^{h}, —OC(═NR^{f})NR^{g}R^{h}, —OP(O)(ORⁿ)₂, —OS(O)R^{f}, —OS(O)₂R^{f}, —OS(O)NR^{g}R^{h}, —OS(O)₂NR^{g}R^{h}, —NR^{g}R^{h}, —NR^{f}C(O)R^{k}, —NR^{f}C(O)OR^{k}, —NR^{f}C(O)NR^{g}R^{h}, —NR^{f}C(═NR^{k})NR^{g}R^{h}, —NR^{f}S(O)R^{k}, —NR^{f}S(O)₂R^{k}, —NR^{f}S(O)NR^{g}R^{h}, —NR^{f}S(O)₂NR^{g}R^{h}, —P(O)(OR^{f})R^{k}, —CH₂P(O)(OR^{f})R^{k}, —SR^{f}, —S(O)R^{f}, —S(O)₂R, —S(O)NR^{g}R^{h}, or —S(O)₂NR^{g}R^{h}; wherein each R^{f}, R^{g}, R^{h}, and R^{k} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{g} and R^{h} together with the N atom to which they are attached form heterocyclyl; and each Rⁿ is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iv) two Rⁿ together are a divalent cation (e.g., Mg²⁺ or Ca²⁺).

In certain embodiments of the compound of Formula IB, or single enantiomer, diastereomer, mixture of diastereomers, or isotopic variant thereof; or pharmaceutically acceptable salt or solvate thereof;

R⁵ is C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl;

R⁶ is (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —CHR^{6a}C(O)R^{6b};

R^{6a} is hydrogen, C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl;

R^{6b} is:

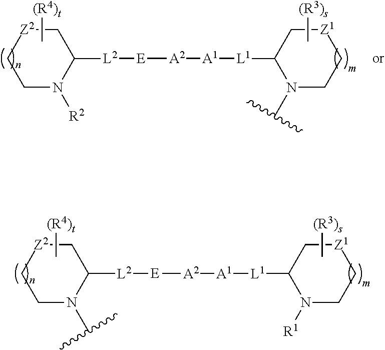

wherein:
A¹, A², and E are each independently (a) a bond; or (b) C₁₋₆ alkylene, C₂₋₆ alkenylene, C₂₋₆ alkynylene, C₂₋₂₀ cycloalkylene, C₆₋₂₀ arylene, heteroarylene; or heterocyclylene;
L¹ and L² are each independently (a) a bond; (b) C₁₋₆ alkylene, C₂₋₆ alkenylene, C₂₋₆ alkynylene, C₃₋₇ cycloalkylene, C₆₋₁₄ arylene, heteroarylene, or heterocyclylene; or (c) —C(O)—, —C(O)O—, —C(O)NR^{1a}—, —C(═NR^{1a})NR^{1c}, —O—, —OC(O)O—, —OC(O)NR^{1a}—, —OC(═NR^{1a})NR^{1c}—, —OP(O)(OR^{1a})—, —OP(O)(OR^{1a})O—, —NR^{1a}—, —NR^{1a}C(O)NR^{1c}—, —NR^{1a}C(═NR^{1b})NR^{1c}—, —NR^{1a}S(O)NR^{1c}—, —NR^{1a}S(O)₂NR^{1c}—, —S—, —S(O)—, —S(O)₂—, —S(O)NR^{1a}—, or —S(O)₂NR^{1a}—;
Z¹ and Z² are each independently a bond, —O—, —S—, —S(O)—, —S(O₂)—, or —N(R^{N})—;
R¹ and R² are each independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (c) —C(O)R^{1a}, —C(O)CH(N(R^{1c})C(O)OR^{1b})R^{1a}, —C(O)OR^{1a}, —C(O)NR^{1b}R^{1c}, —C(NR^{1a})NR^{1b}R^{1c}, —P(O)(OR^{1a})R^{1d}, —CH₂P(O)(OR^{1a})R^{1d}, —S(O)R^{1a}, —S(O)₂R^{1a}, —S(O)NR^{1b}R^{1c}, or —S(O)₂NR^{1b}R^{1c}; or (d) —C₁₋₆ alkylene-OP(O)(OR^{P1})₂, in one embodiment, —CH₂—OP(O)(OR^{P1})₂;
each R³ and R⁴ is independently (a) cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —SR^{1a}, —S(O)R^{1a}, —S(O)₂R^{1a}, —S(O)NR^{1b}R^{1c}, or —S(O)₂NR^{1b}R^{1c}; or
two R³ or two R⁴ that are attached to the same ring are linked together to form a bond, —O—, —NR^{N}—, —S—, C₁₋₆ alkylene, C₁₋₆ heteroalkylene, C₂₋₆ alkenylene, or C₂₋₆ heteroalkenylene;
each R^{N} is independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (c) —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{d}, —NR^{1a}S(O)₂R^{d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —S(O)R^{1a}, —S(O)₂R^{1a}, —S(O)NR^{1b}R^{1c}, or —S(O)₂NR^{1b}R^{1c}; or (d) —C₁₋₆ alkylene-OP(O)(OR^{P1})₂, in one embodiment, —CH₂—OP(O)(OR^{P1})₂;
each m and n is independently an integer of 1, 2, 3, or 4; and
each s and t is independently an integer of 0, 1, 2, 3, 4, 5, 6, or 7;

each R^{P1} is independently (a) hydrogen; (b) a monovalent cation, in one embodiment, Na⁺ or K⁺; (c) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (d) two R^{P1} together are a divalent cation, in one embodiment, Mg²⁺ or Ca²⁺; and

each R^{P2} is independently (a) hydrogen, cyano, halo, or nitro; or (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl;

each R^{1a}, R^{1b}, R^{1c}, and R^{1d} is independently hydrogen, C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or R^{1a} and R^{1c} together with the C and N atoms to which they are attached form heterocyclyl; or R^{1b} and R^{1c} together with the N atom to which they are attached form heterocyclyl;

wherein each alkyl, alkylene, heteroalkylene, alkenyl, alkenylene, heteroalkenylene, alkynyl, alkynylene, cycloalkyl, cycloalkylene, aryl, arylene, aralkyl, heteroaryl, heteroarylene, heterocyclyl, and heterocyclylene is optionally substituted with one or more, in one embodiment, one, two, three, or four, substituents Q, where each Q is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is further optionally substituted with one or more, in one embodiment, one, two, three, or four, substituents Q^{a}; or (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OP(O)(OR^{e})₂, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —P(O)(OR^{a})R^{d}, —CH₂P(O)(OR^{a})R^{d}, —SR^{a}, —S(O)R^{a}, —S(O)₂R^{a}, —S(O)NR^{b}R^{c}, or —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a}; and each R^{e} is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iv) two R^{e} together are a divalent cation (e.g., Mg²⁺ or Ca²⁺);

wherein each Q^{a} is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, ord heterocyclyl; or (c) —C(O)R^{f}, —C(O)OR^{f}, —C(O)NR^{g}R^{h},

—C(NR^{f})NR^{g}R^{h}, —OR^{f}, —OC(O)R^{f}, —OC(O)OR^{f}, —OC(O)NR^{g}R^{h}, —OC(═NR^{f})NR^{g}R^{h}, —OP(O)(ORⁿ)₂, —OS(O)R^{f}, —OS(O)₂R^{f}, —OS(O)NR^{g}R^{h}, —OS(O)₂NR^{g}R^{h}, —NR^{g}R^{h}, —NR^{f}C(O)R^{k}, —NR^{f}C(O)OR^{k}, —NR^{f}C(O)NR^{g}R^{h}, —NR^{f}C(═NR^{k})NR^{g}R^{h}, —NR^{f}S(O)R^{k}, —NR^{f}S(O)₂R^{k}, —NR^{f}S(O)NR^{g}R^{h}, —NR^{f}S(O)₂NR^{g}R^{h}, —P(O)(OR^{f})R^{k}, —CH₂P(O)(OR^{f})R^{k}, —SR^{f}, —S(O)R^{f}, —S(O)₂R^{f}, —S(O)NR^{g}R^{h}, or —S(O)₂NR^{g}R^{h}; wherein each R^{f}, R^{g}, R^{h}, and R^{k} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{g} and R^{h} together with the N atom to which they are attached form heterocyclyl; and each Rⁿ is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iv) two Rⁿ together are a divalent cation (e.g., Mg²⁺ or Ca²⁺).

In one embodiment, provided herein is a compound of Formula IIB:

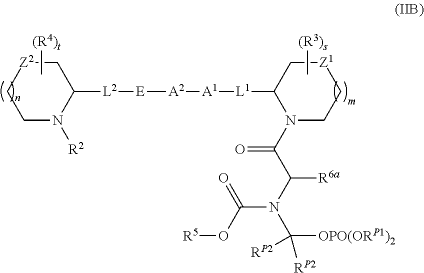

or a single enantiomer, a racemic mixture, a diastereomer, a mixture of diastereomers, or an isotopic variant thereof; or a pharmaceutically acceptable salt or solvate thereof; wherein R², R³, R⁴, R⁵, R^{6a}, R^{P1}, R^{P2}, A¹, A², E, L¹, L², Z, Z², m, n, s, and t are each as defined herein.

In one embodiment, provided herein is a compound of Formula IIBa:

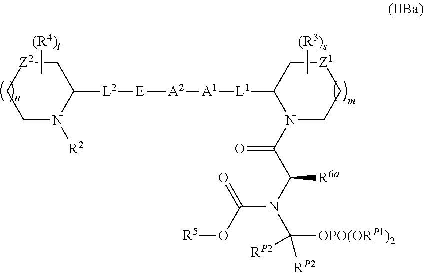

or a single enantiomer, a diastereomer, a mixture of diastereomers, or an isotopic variant thereof; or a pharmaceutically acceptable salt or solvate thereof; wherein R², R³, R⁴, R⁵, R^{6a}, R^{P1}, R^{P2}, A¹, A², E, L¹, L², Z¹, Z², m, n, s, and t are each as defined herein.

In another embodiment, provided herein is a compound of Formula IIBb:

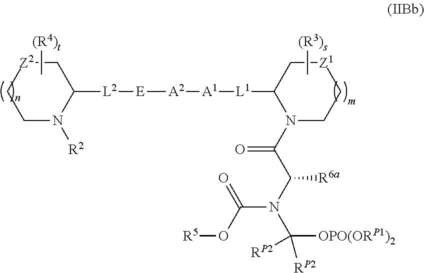

or a single enantiomer, a diastereomer, a mixture of diastereomers, or an isotopic variant thereof; or a pharmaceutically acceptable salt or solvate thereof; wherein R², R³, R⁴, R⁵, R^{6a}, R^{P1}, R^{P2}, A¹, A², E, L¹, L², Z¹, Z², m, n, s, and t are each as defined herein.

In yet another embodiment, provided herein is a compound of Formula IIBc:

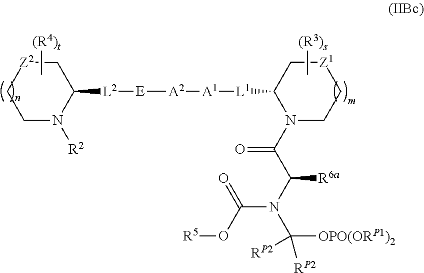

or a single enantiomer, a diastereomer, a mixture of diastereomers, or an isotopic variant thereof; or a pharmaceutically acceptable salt or solvate thereof; wherein R², R³, R⁴, R⁵, R^{6a}, R^{P1}, R^{P2}, A¹, A², E, L¹, L², Z¹, Z², m, n, s, and t are each as defined herein.

In still another embodiment, provided herein is a compound of Formula IIBd:

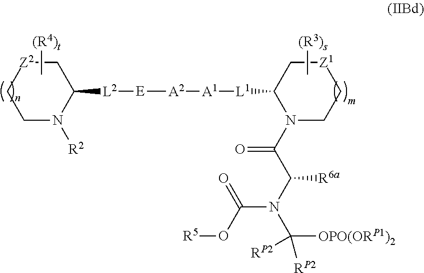

or a single enantiomer, a diastereomer, a mixture of diastereomers, or an isotopic variant thereof; or a pharmaceutically acceptable salt or solvate thereof; wherein R², R³, R⁴, R⁵, R^{6a}, R^{P1}, R^{P2}, A¹, A², E, L¹, L², Z¹, Z², m, n, s, and t are each as defined herein.

In one embodiment, the monovalent moiety

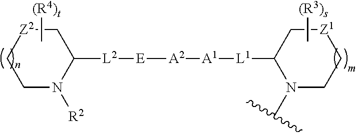

in Formula IB, IIB, IIBa, IIBb, IIBc, or IIBd is one as defined in any of Formulae II to XIV, IIa to XIVa, IIb to XIVb, IIc to XIVc, IId to XIVd, IIe to XIVe, IA to IAe, and IIA to IIAe.

In still another embodiment, provided herein is a compound of Formula IIIB:

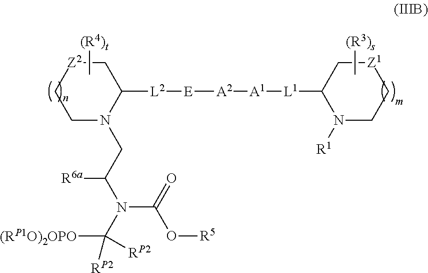

or a single enantiomer, a racemic mixture, a diastereomer, a mixture of diastereomers, or an isotopic variant thereof; or a pharmaceutically acceptable salt or solvate thereof; wherein R¹, R³, R⁴, R⁵, R^{6a}, R^{P1}, R^{P2}, A¹, A², E, L¹, L², Z, Z², m, n, s, and t are each as defined herein.

In one embodiment, provided herein is a compound of Formula IIIBa:

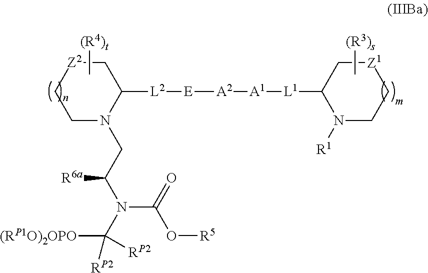

or a single enantiomer, a diastereomer, a mixture of diastereomers, or an isotopic variant thereof; or a pharmaceutically acceptable salt or solvate thereof; wherein R¹, R³, R⁴, R⁵, R^{6a}, R^{P1}, R^{P2}, A¹, A², E, L¹, L², Z¹, Z², m, n, s, and t are each as defined herein.

In another embodiment, provided herein is a compound of Formula IIIBb:

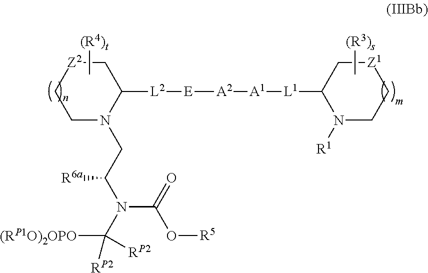

or a single enantiomer, a diastereomer, a mixture of diastereomers, or an isotopic variant thereof; or a pharmaceutically acceptable salt or solvate thereof; wherein R², R³, R⁴, R⁵, R^{6a}, R^{P1}, R^{P2}, A¹, A², E, L¹, L², Z¹, Z², m, n, s, and t are each as defined herein.

In yet another embodiment, provided herein is a compound of Formula IIIBc:

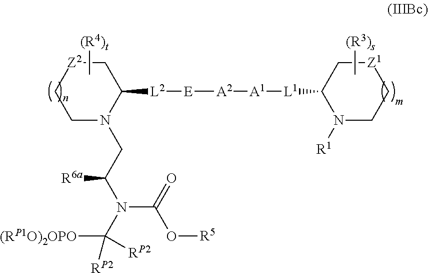

or a single enantiomer, a diastereomer, a mixture of diastereomers, or an isotopic variant thereof; or a pharmaceutically acceptable salt or solvate thereof; wherein R¹, R³, R⁴, R⁵, R^{6a}, R^{P1}, R^{P2}, A¹, A², E, L¹, L², Z¹, Z², m, n, s, and t are each as defined herein.

In still another embodiment, provided herein is a compound of Formula IIIBd:

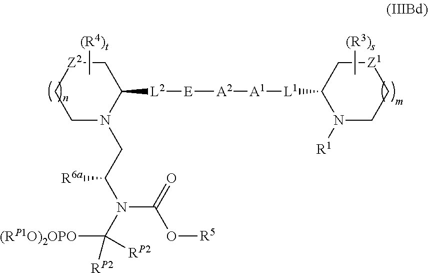

or a single enantiomer, a diastereomer, a mixture of diastereomers, or an isotopic variant thereof; or a pharmaceutically acceptable salt or solvate thereof; wherein R¹, R³, R⁴, R⁵, R^{6a}, R^{P1}, R^{P2}, A¹, A², E, L¹, L², Z¹, Z², m, n, s, and t are each as defined herein.

In one embodiment, the monovalent moiety

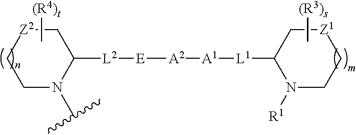

in Formula IB, IIIB, IIIBa, IIIBb, IIIBc, or IIIBd is one as defined in any of Formulae II to XIV, IIa to XIVa, IIb to XIVb, IIc to XIVc, IId to XIVd, lie to XIVe, IA to IAe, and IIA to IIAe.

In one embodiment, provided herein is a compound of Formula IC:

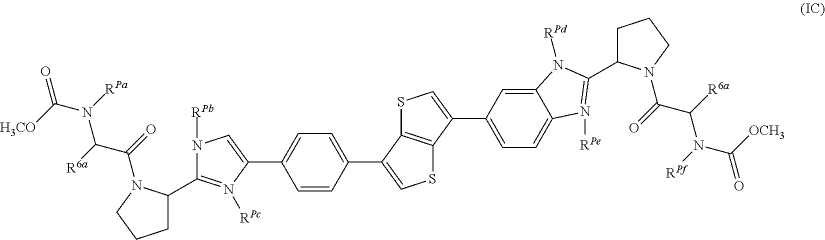

or a single enantiomer, a racemic mixture, a diastereomer, a mixture of diastereomers, or an isotopic variant thereof; or a pharmaceutically acceptable salt or solvate thereof; wherein R^{Pa}, R^{Pb}, R^{Pc}, R^{Pd}, R^{Pe}, and R^{Pf} are each independently absent, hydrogen or —C₁₋₆ alkylene-OP(O)(OR^{P1})₂, in one embodiment, —CH₂—OP(O)(OR^{P1})₂; or alternatively, wherein R^{Pa}, R^{Pb}, R^{Pc}, R^{Pd}, R^{Pe}, and R^{Pf} are each independently absent, hydrogen, —C₁₋₆ alkylene-OP(O)(OR^{P1})₂ (in one embodiment, —CH₂—OP(O)(OR^{P1})₂), or —C₁₋₆ alkylene-O-linked amino acid or a derivative thereof (in one embodiment, —CH₂—OC(O)C(R^{a})₂NR^{1b}R^{1c}); wherein, when R^{Pb} and R^{Pc} are neither absent nor hydrogen, the imidazolyene group carries a positive charge; and when R^{Pd} and R^{Pe} are neither absent nor hydrogen, the benzimidazolylene group carries a positive charge; wherein at least one of R^{Pa}, R^{Pb}, R^{Pc}, R^{Pd}, R^{Pe}, and R^{Pf} is neither absent nor hydrogen; and wherein the alkylene is optionally substituted with one or more, in one embodiment, one, two, three, or four, substituents Q; and R^{1b}, R^{1c}, R^{6a}, R^{aa}, R^{P1}, and Q are each as defined herein.

In certain embodiments of the compound of Formula IC, or single enantiomer, racemic mixture, diastereomer, mixture of diastereomers, or isotopic variant thereof; or pharmaceutically acceptable salt or solvate thereof;

R^{Pa}, R^{Pb}, R^{Pc}, R^{Pd}, R^{Pe}, and R^{Pf} are each independently absent, hydrogen, or —C₁₋₆ alkylene-OP(O)(OR^{P1})₂, in one embodiment, —CH₂—OP(O)(OR^{P1})₂; wherein, when R^{Pb} and R^{Pc} are neither absent nor hydrogen, the imidazolyene group carries a positive charge; and when R^{Pd} and R^{Pe} are neither absent nor hydrogen, the benzimidazolylene group carries a positive charge; wherein at least one of R^{Pa}, R^{Pb}, R^{Pc}, R^{Pd}, R^{Pe}, and R^{Pf} is neither absent nor hydrogen; and wherein the alkylene is optionally substituted with one or more, in one embodiment, one, two, three, or four, substituents Q; and R^{6a}, R^{P1}, and Q are each as defined herein.

In one embodiment, provided herein is a compound of Formula ICa:

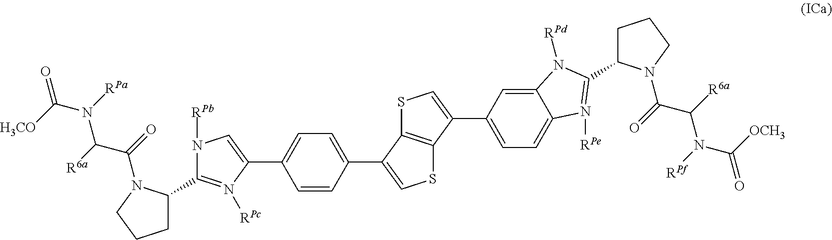

or a single enantiomer, a diastereomer, a mixture of diastereomers, or an isotopic variant thereof; or a pharmaceutically acceptable salt or solvate thereof; wherein R^{6a}, R^{Pa}, R^{Pb}, R^{Pc}, R^{Pd}, R^{Pe}, and R^{Pf} are each as defined herein.

In another embodiment, provided herein is a compound of Formula ICb:

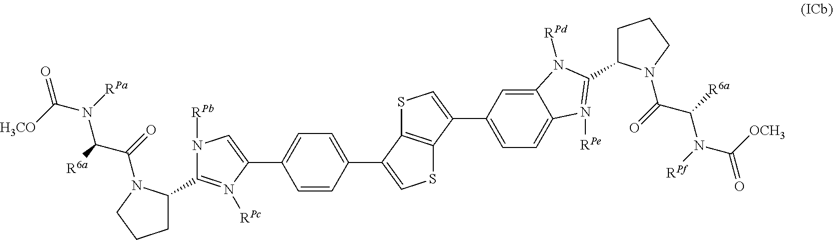

or a single enantiomer or an isotopic variant thereof or a pharmaceutically acceptable salt or solvate thereof wherein R^{6a}, R^{Pa}, R^{Pb}, R^{Pc}, R^{Pd}, R^{Pe}, and R^{Pf} are each as defined herein.

In yet another embodiment, provided herein is a compound of Formula ICc:

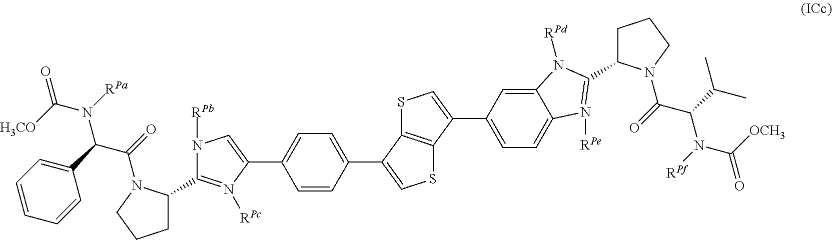

or a single enantiomer or an isotopic variant thereof; or a pharmaceutically acceptable salt or solvate thereof; wherein R^{Pa}, R^{Pb}, R^{Pc}, R^{Pd}, R^{Pe}, and R^{Pf} are each as defined herein.

In another embodiment, provided herein is a compound of Formula ID:

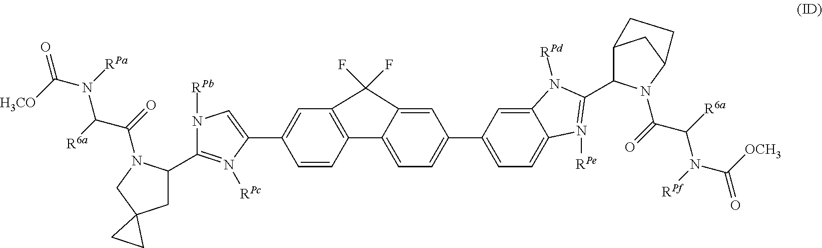

or a single enantiomer, a racemic mixture, a diastereomer, a mixture of diastereomers, or an isotopic variant thereof; or a pharmaceutically acceptable salt or solvate thereof; wherein R^{Pa}, R^{Pb}, R^{Pc}, R^{Pd}, R^{Pe}, and R^{Pf} are each independently absent, hydrogen, or —C₁₋₆ alkylene-OP(O)(OR^{P1})₂, in one embodiment, —CH₂—OP(O)(OR^{P1})₂; or alternatively, wherein R^{Pa}, R^{Pb}, R^{Pc}, R^{Pd}, R^{Pe}, and R^{Pf} are each independently absent, hydrogen, —C₁₋₆alkylene-OP(O)(OR^{P1})₂ (in one embodiment, —CH₂—OP(O)(OR^{P1})₂), or —C₁₋₆ alkylene-O-linked amino acid or a derivative thereof (in one embodiment, —CH₂—OC(O)C(R^{aa})₂NR^{1b}R^{1c}); wherein, when R^{Pb} and R^{Pc} are neither absent nor hydrogen, the imidazolyene group carries a positive charge; and when R^{Pd} and R^{Pe} are neither absent nor hydrogen, the benzimidazolylene group carries a positive charge; and wherein at least one of R^{Pa}, R^{Pb}, R^{Pc}, R^{Pd}, R^{Pe}, and R^{Pf} is neither absent nor hydrogen; and wherein the alkylene is optionally substituted with one or more, in one embodiment, one, two, three, or four, substituents Q; and R^{1b}, R^{1c}, R^{6a}, R^{aa}, R^{P1}, and Q are each as defined herein. In certain embodiments of the compound of Formula ID, or single enantiomer, racemic mixture, diastereomer, mixture of diastereomers, or isotopic variant thereof; or pharmaceutically acceptable salt or solvate thereof;
R^{Pa}, R^{Pb}, R^{Pc}, R^{Pd}, R^{Pe}, and R^{Pf} are each independently absent, hydrogen, or —C₁₋₆ alkylene-OP(O)(OR^{P1})₂, in one embodiment, —CH₂—OP(O)(OR^{P1})₂; wherein, when R^{Pb} and R^{Pc} are neither absent nor hydrogen, the imidazolyene group carries a positive charge; and when R^{Pd} and R^{Pe} are neither absent nor hydrogen, the benzimidazolylene group carries a positive charge; wherein at least one of R^{Pa}, R^{Pb}, R^{Pc}, R^{Pd}, R^{Pe}, and R^{Pf} is neither absent nor hydrogen; and wherein the alkylene is optionally substituted with one or more, in one embodiment, one, two, three, or four, substituents Q; and R^{6a}, R^{P1}, and Q are each as defined herein.

In one embodiment, provided herein is a compound of Formula IDa:

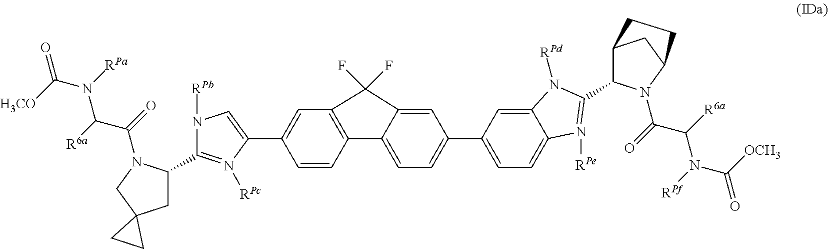

or a single enantiomer, a diastereomer, a mixture of diastereomers, or an isotopic variant thereof; or a pharmaceutically acceptable salt or solvate thereof; wherein R^{6a}, R^{P1}, R^{Pa}, R^{Pb}, R^{Pc}, R^{Pd}, R^{Pe}, and R^{Pf} are each as defined herein.

In another embodiment, provided herein is a compound of Formula IDb:

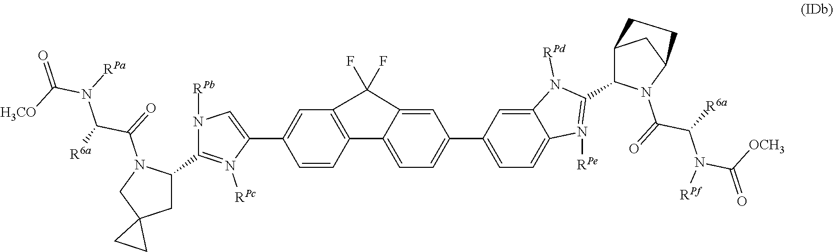

or a single enantiomer or an isotopic variant thereof; or a pharmaceutically acceptable salt or solvate thereof; wherein R^{6a}, R^{P1}, R^{Pa}, R^{Pb}, R^{Pc}, R^{Pd}, R^{Pe}, and R^{Pf} are each as defined herein.

In yet another embodiment, provided herein is a compound of Formula IDc:

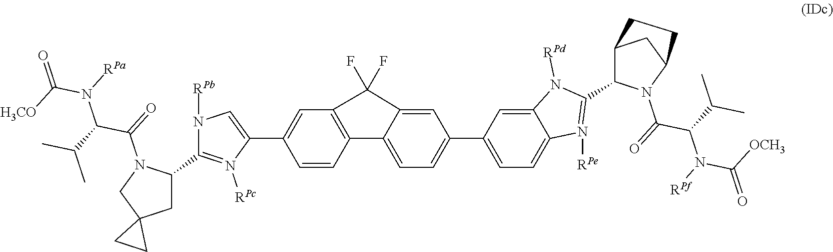

or a single enantiomer or an isotopic variant thereof; or a pharmaceutically acceptable salt or solvate thereof; wherein R^{Pa}, R^{Pb}, R^{Pc}, R^{Pd}, R^{Pe}, and R^{Pf} are each as defined herein.

In still another embodiment, provided herein is a compound of Formula IDd:

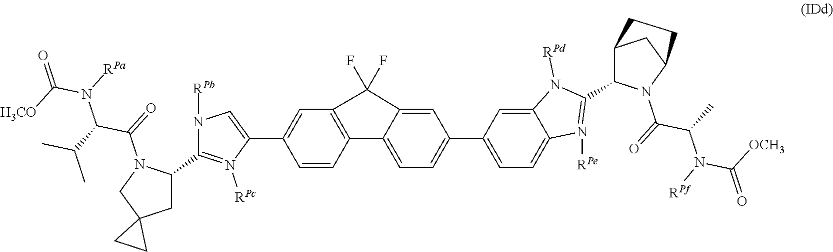

or a single enantiomer or an isotopic variant thereof; or a pharmaceutically acceptable salt or solvate thereof; wherein R^{Pa}, R^{Pb}, R^{Pc}, R^{Pd}, R^{Pe}, and R^{Pf} are each as defined herein.

In still another embodiment, provided herein is a compound of Formula IE:

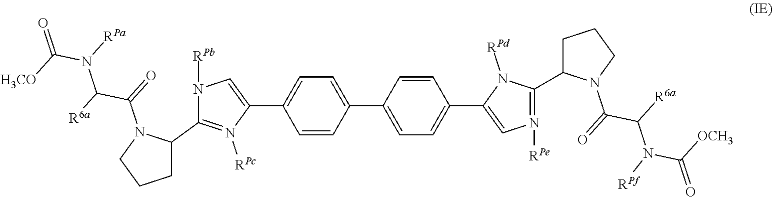

or a single enantiomer, a racemic mixture, a diastereomer, a mixture of diastereomers, or an isotopic variant thereof; or a pharmaceutically acceptable salt or solvate thereof; wherein R^{Pa}, R^{Pb}, R^{Pc}, R^{Pd}, R^{Pe}, and R^{Pf} are each independently absent, hydrogen, or —C₁₋₆ alkylene-OP(O)(OR^{P1})₂, in one embodiment, —CH₂—OP(O)(OR^{P1})₂; or alternatively, wherein R^{Pa}, R^{Pb}, R^{Pc}, R^{Pd}, R^{Pe}, and R^{Pf} are each independently absent, hydrogen, —C₁₋₆alkylene-OP(O)(OR^{P1})₂ (in one embodiment, —CH₂—OP(O)(OR^{P1})₂), or —C₁₋₆ alkylene-O-linked amino acid or a derivative thereof (in one embodiment, —CH₂—OC(O)C(R)₂NR^{1b}R^{1c}); wherein, when R^{Pb} and R^{Pc} are neither absent nor hydrogen, the imidazolyene group carries a positive charge; and when R^{Pd} and R^{Pe} are neither absent nor hydrogen, the benzimidazolylene group carries a positive charge; and wherein at least one of R^{Pa}, R^{Pb}, R^{Pc}, R^{Pd}, R^{Pe}, and R^{Pf} is neither absent nor hydrogen; and wherein the alkylene is optionally substituted with one or more, in one embodiment, one, two, three, or four, substituents Q; and R^{1b}, R^{1c}, R^{6a}, R^{aa}, R^{P1}, and Q are each as defined herein.

In certain embodiments of the compound of Formula IE, or single enantiomer, racemic mixture, diastereomer, mixture of diastereomers, or isotopic variant thereof; or pharmaceutically acceptable salt or solvate thereof;

R^{Pa}, R^{Pb}, R^{Pc}, R^{Pd}, R^{Pe}, and R^{Pf} are each independently absent, hydrogen, or —C₁₋₆ alkylene-OP(O)(OR^{P1})₂, in one embodiment, —CH₂—OP(O)(OR^{P1})₂; wherein, when R^{Pb} and R^{Pc} are neither absent nor hydrogen, the imidazolyene group carries a positive charge; and when R^{Pd} and R^{Pe} are neither absent nor hydrogen, the benzimidazolylene group carries a positive charge; and wherein at least one of R^{Pa}, R^{Pb}, R^{Pc}, R^{Pd}, R^{Pe}, and R^{Pf} is neither absent nor hydrogen; and wherein the alkylene is optionally substituted with one or more, in one embodiment, one, two, three, or four, substituents Q; and R^{6a}, R^{P1}, and Q are each as defined herein.

In one embodiment, provided herein is a compound of Formula IEa:

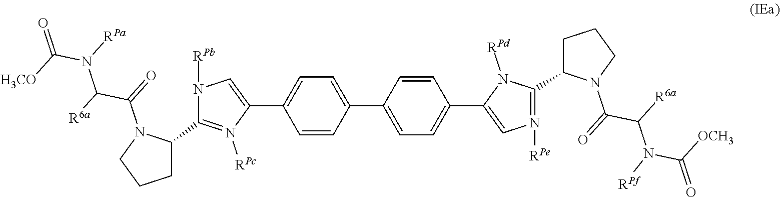

or a single enantiomer, a diastereomer, a mixture of diastereomers, or an isotopic variant thereof; or a pharmaceutically acceptable salt or solvate thereof; wherein R^{6a}, R^{Pa}, R^{Pb}, R^{Pc}, R^{Pd}, R^{Pe}, and R^{Pf} are each as defined herein.

In another embodiment, provided herein is a compound of Formula IEb:

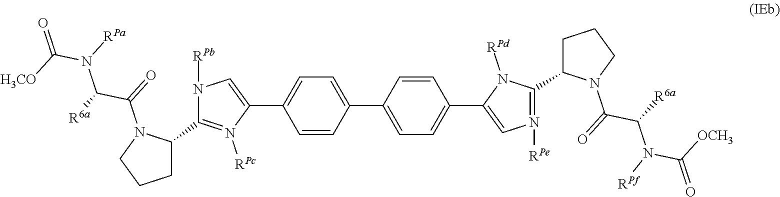

or a single enantiomer or an isotopic variant thereof; or a pharmaceutically acceptable salt or solvate thereof; wherein R^{6a}, R^{Pa}, R^{Pb}, R^{Pc}, R^{Pd}, R^{Pe}, and R^{Pf} are each as defined herein.

In another embodiment, provided herein is a compound of Formula IEc:

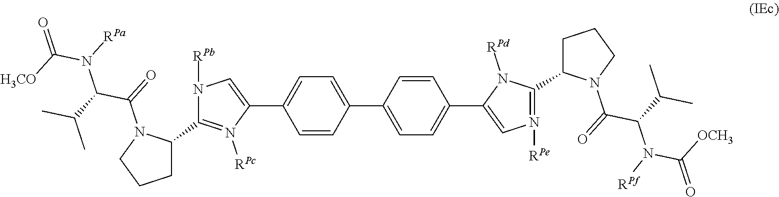

or a single enantiomer or an isotopic variant thereof; or a pharmaceutically acceptable salt or solvate thereof; wherein R^{Pa}, R^{Pb}, R^{Pc}, R^{Pd}, R^{Pe}, and R^{Pf} are each as defined herein.

The groups, R¹, R², R³, R⁴, R⁵, R⁶, R⁷, R^{6a}, R^{6b}, R^{7a}, R^{8a}, R^{8b}, R^{8c}, R^{P}, R^{P1}, R^{P2}, A¹, A², E, T³, U¹, U², U³, V¹, V², V³, W¹, W², W³, X¹, X², X³, Y³, Z Z², m, n, r, s, t, and u in formulae described herein, including Formulae I to XIV, IIIa to XIVa, IIIb to XIVb, IIIc to XIVc, IIId to XIVd, IIIe to XIVe, IA to IAe, IIA to IIAe, IB, IIB to IIBd, IIIB to IIIBd, IC to ICc, ID to IDd, and IE to IEc are further defined herein. All combinations of the embodiments provided herein for such groups are within the scope of this disclosure.

In certain embodiments, R¹ is hydrogen. In certain embodiments, R¹ is C₁₋₆ alkyl, optionally substituted with one or more substituents Q. In certain embodiments, R¹ is C₂₋₆ alkenyl, optionally substituted with one or more substituents Q. In certain embodiments, R¹ is C₂₋₆ alkynyl, optionally substituted with one or more substituents Q. In certain embodiments, R¹ is C₃₋₇cycloalkyl, optionally substituted with one or more substituents Q. In certain embodiments, R¹ is C₆₋₁₄ aryl, optionally substituted with one or more substituents Q. In certain embodiments, R¹ is C₇₋₁₅ aralkyl, optionally substituted with one or more substituents Q. In certain embodiments, R¹ is heteroaryl, optionally substituted with one or more substituents Q. In certain embodiments, R¹ is heterocyclyl, optionally substituted with one or more substituents Q.

In certain embodiments, R¹ is —C(O)R^{1a}, wherein R^{1a} is as defined herein. In certain embodiments, R¹ is —C(O)CH(N(R^{c})C(O)OR^{1b})R^{1a}, wherein R^{1a}, R^{1b}, and R^{1c} are each as defined herein. In certain embodiments, R¹ is —C(O)OR^{1a}, wherein R^{1a} is as defined herein. In certain embodiments, R¹ is —C(O)NR^{1b}R^{1c}, wherein R^{1b} and R^{1c} are each as defined herein. In certain embodiments, R¹ is —C(NR^{1a})NR^{1b}R^{1a}, wherein R^{1a}, R^{1b}, and R^{1c} are each as defined herein. In certain embodiments, R¹ is —P(O)(OR^{1a})R^{1d}, wherein R^{1a} and R^{1d} are each as defined herein. In certain embodiments, R¹ is —CH₂P(O)(OR^{1a})R^{1d}, wherein R^{1a} and R^{1d} are each as defined herein. In certain embodiments, R¹ is —S(O)R^{1a}, wherein R^{1a} is as defined herein. In certain embodiments, R¹ is —S(O)₂R^{1a}, wherein R^{1a} is as defined herein. In certain embodiments, R¹ is —S(O)NR^{1b}R^{1c}, wherein R^{1b} and R^{1c} are each as defined herein. In certain embodiments, R¹ is —S(O)₂NR^{1b}R^{1c}, wherein R^{1b} and R^{1c} are each as defined herein. In certain embodiments, R¹ is —C₁₋₆ alkylene-OP(O)(OR^{P1})₂, wherein R^{P1} is as defined herein. In certain embodiments, R¹ is —C(R^{P2})₂—OP(O)(OR^{P})₂, wherein R^{P1} and R^{P2} are each as defined herein. In certain embodiments, R¹ is —CH₂—OP(O)(OR^{P1})₂, wherein R^{P1} is as defined herein. In certain embodiments, R¹ is —CH₂—OP(O)(OH)₂. In certain embodiments, R¹ is —CH₂—OP(O)(ONa)₂. In certain embodiments, R¹ is —C₁₋₆ alkylene-O-linked amino acid or a derivative thereof. In certain embodiments, R¹ is —CH₂—OC(O)C(R^{aa})₂NR^{1b}R^{1c}, where R^{aa}, R^{1b}, and R^{1c} are defined as herein. In particular embodiments, R¹ is —CH₂—OC(O)CH(i-propyl)NH₂.

In certain embodiments, R² is hydrogen. In certain embodiments, R² is C₁₋₆ alkyl, optionally substituted with one or more substituents Q. In certain embodiments, R² is C₂₋₆ alkenyl, optionally substituted with one or more substituents Q. In certain embodiments, R² is C₂₋₆ alkynyl, optionally substituted with one or more substituents Q. In certain embodiments, R² is C₃₋₇ cycloalkyl, optionally substituted with one or more substituents Q. In certain embodiments, R² is C₆₋₁₄ aryl, optionally substituted with one or more substituents Q. In certain embodiments, R² is C₇₋₁₅ aralkyl, optionally substituted with one or more substituents Q. In certain embodiments, R² is heteroaryl, optionally substituted with one or more substituents Q. In certain embodiments, R² is heterocyclyl, optionally substituted with one or more substituents Q.

In certain embodiments, R² is —C(O)R^{1a} wherein R^{1a} is as defined herein. In certain embodiments, R² is —C(O)CH(N(R^{1c})C(O)OR^{1b})R^{1a}, wherein R^{1a}, R^{1b}, and R^{1c} are each as defined herein. In certain embodiments, R² is —C(O)OR^{1a}, wherein R^{1a} is as defined herein. In certain embodiments, R² is —C(O)NR^{1b}R^{1c}, wherein R^{1b} and R^{1c} are each as defined herein. In certain embodiments, R² is —C(NR^{1a})NR^{1b}R^{1c}, wherein R^{1a}, R^{1b}, and R^{1c} are each as defined herein. In certain embodiments, R² is —P(O)(OR^{1a})R^{1d}, wherein R^{1a} and R^{1d} are each as defined herein. In certain embodiments, R² is —CH₂P(O)(OR^{1a})R^{1d}, wherein R^{1a} and R^{1d} are each as defined herein. In certain embodiments, R² is —S(O)R^{1a}, wherein R^{1a} is as defined herein. In certain embodiments, R² is —S(O)₂R^{1a}, wherein R^{1a} is as defined herein. In certain embodiments, R² is —S(O)NR^{1b}R^{1c}, wherein R^{1b} and R^{1c} are each as defined herein. In certain embodiments, R² is —S(O)₂NR^{1b}R^{1c}, wherein R^{1b} and R^{1c} are each as defined herein. In certain embodiments, R² is —C₁₋₆ alkylene-OP(O)(OR^{P1})₂, wherein R^{P1} is as defined herein. In certain embodiments, R² is —C(R^{P2})₂—OP(O)(OR^{P1})₂, wherein R^{P1} and R^{P2} are each as defined herein. In certain embodiments, R² is —CH₂—OP(O)(OR^{P1})₂, wherein R^{P1} is as defined herein. In certain embodiments, R² is —CH₂—OP(O)(OH)₂. In certain embodiments, R² is —CH₂—OP(O)(ONa)₂. In certain embodiments, R² is —C₁₋₆ alkylene-O-linked amino acid or a derivative thereof. In certain embodiments, R² is —CH₂—OC(O)C(R^{aa})₂NR^{1b}R^{1c}, where R^{aa}, R^{1b}, and R^{1c} are defined as herein. In particular embodiments, R² is —CH₂—OC(O)CH(i-propyl)NH₂.

In certain embodiments, R¹ and R² are each independently selected from 2(R)-(dimethylamino)propionyl, 2-(methoxycarbonylamino)propionyl, 2(R)-(methoxy-carbonylamino)propionyl, 2-(ethoxycarbonylamino)propionyl, 2(R)-(methoxycarbonyl-amino)-3-methoxy-propionyl, 2(R)-(methoxycarbonylamino)-3-aminocarbonyl-propionyl, 2-(methoxycarbonylamino)-2-methylpropionyl, 2(R)-(methoxycarbonylamino)-3(R)-hydroxy-butanoyl, 2(R)-(methoxycarbonylamino)-3(S)-hydroxybutanoyl, 2(R)-(methoxycarbonyl-amino)-3-methylbutanoyl, 2(S)-(methoxycarbonylamino)-3-methylbutanoyl, 2(R)-(ethoxycarbonyl-amino)-3-methylbutanoyl, 2(S)-(ethoxycarbonylamino)-3-methylbutanoyl, 2(R)-(isoproxycarbonyl-amino)-3-methylbutanoyl, 2(S)-(isopropoxycarbonylamino)-3-methylbutanoyl, 2(R)-(tert-butoxycarbonylamino)-3-methylbutanoyl, 2(S)-(tert-butoxycarbonylamino)-3-methylbutanoyl, 2(R)-(methoxycarbonylamino)-3-hydroxy-3-methylbutanoyl, 2-(methoxycarbonylamino)-2-cyclopropyl-acetyl, 2-(methoxycarbonylamino)pentanoyl, 2-(methoxycarbonylamino)pent-4-enoyl, 1-(methoxycarbonylamino)cyclopropylcarbonyl, 1-(methoxycarbonylamino)-cyclobutylcarbonyl, 1-(methoxycarbonylamino)-cyclopentyl-carbonyl, 2(R)-(methoxycarbonylamino)-2-phenylacetyl, 2(R)-(ethoxycarbonylamino)-2-phenylacetyl, 2(R)-(isopropoxycarbonylamino)-2-phenylacetyl, 2(R)-(tert-butoxycarbonylamino)-2-phenylacetyl, 2(S)-(tert-butoxycarbonylamino)-2-phenylacetyl, 2(R)-(methoxycarbonyl-amino)-2-(2-chlorophenyl)acetyl, 2(R)-(dimethylamino)-2-phenylacetyl, 2-(dimethylamino)-2-(4-nitrophenyl)acetyl, 2-(dimethylamino)-2-(2-fluorophenyl)acetyl, 2(R)-(dimethylamino)-2-(2-fluorophenyl)acetyl, 2(S)-(dimethylamino)-2-(2-fluorophenyl)acetyl, 2-(dimethyl-amino)-2-(3-fluorophenyl)acetyl, 2-(dimethylamino)-2-(2-chlorophenyl)acetyl, 2(R)-(dimethylamino)-2-(2-chlorophenyl)acetyl, 2-(dimethylamino)-2-(3-chlorophenyl)acetyl, 2-(dimethylamino)-2-(4-chlorophenyl)acetyl, 2-(dimethylamino)-2-(2-trifluoromethyl-phenyl)acetyl, 2-(dimethyl-amino)-2-(3-trifluoromethylphenyl)acetyl, 2-(dimethylamino)-2-(thien-2-yl)acetyl, 2-(dimethylamino)-2-(thien-3-yl)acetyl, 2-(dimethylamino)-2-(2-methylthiazol-4-yl)acetyl, 2-(dimethylamino)-2-(benzothien-3-yl)acetyl, 2-(dimethylamino)-2-(2-methyl-benzothiazol-5-yl)acetyl, 2-(dimethylamino)-2-(benzoisoxazol-3-yl)acetyl, 2-(dimethylamino)-2-(quinolin-3-yl)acetyl, 2(R)-(diethylamino)-2-phenylacetyl, 2(R)-(methylethylamino)-2-phenylacetyl, 2-(dimethylamino)-2-naphth-1-ylacetyl, 2(R)-(pyrrolidin-1-yl)-2-phenylacetyl, 2-(3(S)-fluoropyrrolidin-1-yl)-2-phenylacetyl, 2(R)-(morpholin-4-yl)-2-phenylacetyl, 2(R)-(piperidin-1-yl)-2-phenylacetyl, 2(R)-(piperidin-1-yl)-2-(2-fluorophenyl)acetyl, 2-(4-hydroxy-piperidin-1-yl)-2-phenylacetyl, 2-(4-phenylpiperidin-1-yl)-2-phenylacetyl, 2(R)-(4-hydroxy-4-methylpiperidin-1-yl)-2-phenylacetyl, 2(R)-(4-hydroxy-4-phenylpiperidin-1-yl)-2-phenylacetyl, 2-(3-oxopiperazin-1-yl)-2-phenylacetyl, 2-(4-methylpiperazin-1-yl)-2-phenylacetyl, 2-(dimethylamino)-2-(pyridin-2-yl)acetyl, 2-(dimethylamino)-2-(pyridin-3-yl)acetyl, 2-(dimethylamino)-2-(pyridin-4-yl)acetyl, 2-(dimethylamino)-2-(6-chloropyridin-3-yl)acetyl, 2-(2-dimethylaminomethyl)phenylacetyl, 2-(2-pyrrolin-1-ylmethyl)phenylacetyl, 2-(2-piperidin-1-ylmethyl)phenylacetyl, 2-(2-morpholin-4-ylmethyl)phenylacetyl, 2-(2-(4-methylpiperazin-1-ylmethyl)phenylacetyl, 1-methylpyrrolidine-2(R)-carbonyl, 1-methyl-4(R)-fluoro-pyrrolidine-2(R)-carbonyl, 2-(R)-(methylaminoarbonylamino)-2-phenylacetyl, 2-(R)-(ethylaminoarbonylamino)-2-phenylacetyl, 2(R)-(cyclopentylaminoarbonylamino)-2-phenylacetyl, 2(R)-(dimethylaminoarbonylamino)-2-phenylacetyl, (N,N-benzylmethyl-amino)acetyl, or 2-(N,N-benzylmethylamino)-3-methylbutanoyl. Further examples of R¹ and R² can be found, e.g., in U.S. Pat. Appl. Publ. Nos. 2009/0202478 and 2009/0202483; U.S. Pat. No. 8,362,068; and International Pat. Appl. Nos. WO 2008/144380 and WO 2009/102694, the disclosure of each of which is incorporated herein by reference in its entirety.

In certain embodiments, R³ is cyano. In certain embodiments, R³ is halo. In certain embodiments, R³ is nitro. In certain embodiments, R³ is C₁₋₆ alkyl, optionally substituted with one or more substituents Q. In certain embodiments, R³ is C₂₋₆ alkenyl, optionally substituted with one or more substituents Q. In certain embodiments, R³ is C₂₋₆ alkynyl, optionally substituted with one or more substituents Q. In certain embodiments, R³ is C₃₋₇ cycloalkyl, optionally substituted with one or more substituents Q. In certain embodiments, R³ is cyclohexyl, optionally substituted with one or more substituents Q. In certain embodiments, R³ is cyclohexyl. In certain embodiments, R³ is C₆₋₁₄ aryl, optionally substituted with one or more substituents Q. In certain embodiments, R³ is C₇₋₁₅ aralkyl, optionally substituted with one or more substituents Q. In certain embodiments, R³ is heteroaryl, optionally substituted with one or more substituents Q. In certain embodiments, R³ is heterocyclyl, optionally substituted with one or more substituents Q.

In certain embodiments, R³ is —C(O)R^{1a}, where R^{1a} is as defined herein. In certain embodiments, R³ is —C(O)OR^{1a}, where R^{1a} is as defined herein. In certain embodiments, R³ is —C(O)NR^{1b}R^{1c}, where R^{1b} and R^{1c} are each as defined herein. In certain embodiments, R³ is —C(NR^{1a})NR^{1b}R^{1c}, where R^{1a}, R^{1b}, and R^{1c} are each as defined herein. In certain embodiments, R³ is —OR^{1a}, where R^{1a} is as defined herein. In certain embodiments, R³ is —OH. In certain embodiments, R³ is —OC(O)R^{1a}, where R^{1a} is as defined herein. In certain embodiments, R³ is —OC(O)OR^{1a}, where R^{1a} is as defined herein. In certain embodiments, R³ is —OC(O)NR^{1b}R¹, where R^{1b} and R^{1c} are each as defined herein. In certain embodiments, R³ is —OC(═NR^{a})NR^{1b}R^{1c}, where R^{1a}, R^{1b}, and R^{1c} are each as defined herein. In certain embodiments, R³ is —OS(O)R^{1a}, where R^{1a} is as defined herein. In certain embodiments, R³ is —OS(O)₂R^{1a}, where R^{1a} is as defined herein. In certain embodiments, R³ is —OS(O)NR^{1b}R^{1c}, where R^{1b} and R^{1c} are each as defined herein. In certain embodiments, R³ is —OS(O)₂NR^{1b}R^{1c}, where R^{1b} and R^{1c} are each as defined herein. In certain embodiments, R³ is —NR^{1b}R^{1c}, where R^{1b} and R^{1c} are each as defined herein. In certain embodiments, R³ is —NR^{1a}C(O)R^{1d}, where R^{1a} and R^{1d} are each as defined herein. In certain embodiments, R³ is —NR^{1a}C(O)OR^{1d}, where R^{1a} and R^{1d} are each as defined herein. In certain embodiments, R³ is —NR^{a}C(O)NR^{1b}R^{1c}, where R^{1a}, R^{1b}, and R^{1c} are each as defined herein. In certain embodiments, R³ is —NR^{1a}C(═NR^{d})NR^{1b}R^{1c}, where R^{1a}, R^{1b}, R^{1c}, and R^{1d} are each as defined herein. In certain embodiments, R³ is —NR^{1a}S(O)R^{1d}, where R^{1a} and R^{1d} are each as defined herein. In certain embodiments, R³ is —NR^{1a}S(O)₂R^{1d}, where R^{1a} and R^{1d} are each defined herein. In certain embodiments, R³ is —NR^{1a}S(O)NR^{1b}R^{1c}, where R^{1a}, R^{1b}, and R^{1c} are each as defined herein. In certain embodiments, R³ is —NR^{1a}S(O)₂NR^{1b}R^{1c}, where R^{1a}, R^{1b}, and R^{1c} are each as defined herein. In certain embodiments, R³ is —P(O)(OR^{1a})R^{1d}, where R^{1a} and R^{1d} are each defined herein. In certain embodiments, R³ is —CH₂P(O)(OR^{1a})R^{1d}, where R^{1a} and R^{1d} are each defined herein. In certain embodiments, R³ is —SR^{1a}, where R^{1a} is as defined herein. In certain embodiments, R³ is —S(O)R^{1a}, where R^{1a} is as defined herein. In certain embodiments, R³ is —S(O)₂R^{1a}, where R^{1a} is as defined herein. In certain embodiments, R³ is —S(O)NR^{1b}R^{1c}, where R^{1b} and R^{1c} are each as defined herein. In certain embodiments, R³ is —S(O)₂NR^{1b}R^{1c}, where R^{1b} and R^{1c} are each as defined herein. In certain embodiments, R³ is chloro, fluoro, nitro, amino, methyl, trifluoromethyl, phenyl, or methoxy.

In certain embodiments, two R³ are linked together to form a bond. In certain embodiments, two R³ are linked together to form —O—. In certain embodiments, two R³ are linked together to form —NR^{N}—, where R^{N} is as defined herein. In certain embodiments, two R³ are linked together to form —S—. In certain embodiments, two R³ are linked together to form C₁₋₆ alkylene, optionally substituted with one or more substituents Q. In certain embodiments, two R³ are linked together to form methylene, ethylene, or propylene, each optionally substituted with one or more substituents Q. In certain embodiments, two R³ are linked together to form C₁₋₆ heteroalkylene, optionally substituted with one or more substituents Q. In certain embodiments, two R³ are linked together to form C₂₋₆ alkenylene, optionally substituted with one or more substituents Q. In certain embodiments, two R³ are linked together to form C₂₋₆ heteroalkenylene, optionally substituted with one or more substituents Q. In certain embodiments, two R³ are linked together to form a fused ring. In certain embodiments, two R³ are linked together to form a bridged ring. In certain embodiments, two R³ are linked together to form a spiro ring.

In certain embodiments, R⁴ is cyano. In certain embodiments, R⁴ is halo. In certain embodiments, R⁴ is nitro. In certain embodiments, R⁴ is C₁₋₆ alkyl, optionally substituted with one or more substituents Q. In certain embodiments, R⁴ is C₂₋₆ alkenyl, optionally substituted with one or more substituents Q. In certain embodiments, R⁴ is C₂₋₆ alkynyl, optionally substituted with one or more substituents Q. In certain embodiments, R⁴ is C₃₋₇ cycloalkyl, optionally substituted with one or more substituents Q. In certain embodiments, R⁴ is cyclohexyl, optionally substituted with one or more substituents Q. In certain embodiments, R⁴ is cyclohexyl. In certain embodiments, R⁴ is C₆₋₁₄ aryl, optionally substituted with one or more substituents Q. In certain embodiments, R⁴ is C₇₋₁₅ aralkyl, optionally substituted with one or more substituents Q. In certain embodiments, R⁴ is heteroaryl, optionally substituted with one or more substituents Q. In certain embodiments, R⁴ is heterocyclyl, optionally substituted with one or more substituents Q.

In certain embodiments, R⁴ is —C(O)R^{a}, where R^{1a} is as defined herein. In certain embodiments, R⁴ is —C(O)OR^{a}, where R^{1a} is as defined herein. In certain embodiments, R⁴ is —C(O)NR^{1b}R^{1c}, where R^{1b} and R^{1c} are each as defined herein. In certain embodiments, R⁴ is —C(NR^{1a})NR^{1b}R^{1c}, where R^{1a}, R^{1b}, and R^{1c} are each as defined herein. In certain embodiments, R⁴ is —OR^{1a}, where R^{1a} is as defined herein. In certain embodiments, R⁴ is —OH. In certain embodiments, R⁴ is —OC(O)R^{1a}, where R^{1a} is as defined herein. In certain embodiments, R⁴ is —OC(O)OR^{1a}, where R^{1a} is as defined herein. In certain embodiments, R⁴ is —OC(O)NR^{1b}R^{1c}, where R^{1b} and R^{1c} are each as defined herein. In certain embodiments, R⁴ is —OC(═NR^{1a})NR^{1b}R^{1c}, where R^{1a}, R^{1b}, and R^{1c} are each as defined herein. In certain embodiments, R⁴ is —OS(O)R^{1a}, where R^{1a} is as defined herein. In certain embodiments, R⁴ is —OS(O)₂R^{1a}, where R^{1a} is as defined herein. In certain embodiments, R⁴ is —OS(O)NR^{1b}R^{1c}, where R^{1b} and R^{1c} are each as defined herein. In certain embodiments, R⁴ is —OS(O)₂NR^{1b}R^{1c}, where R^{1b} and R^{1c} are each as defined herein. In certain embodiments, R⁴ is —NR^{1b}R^{1c}, where R^{1b} and R^{1c} are each as defined herein. In certain embodiments, R⁴ is —NR^{1a}C(O)R^{1d}, where R^{1a} and R^{1d} are each as defined herein. In certain embodiments, R⁴ is —NR^{1a}C(O)OR^{1d}, where R^{1a} and R^{d} are each as defined herein. In certain embodiments, R⁴ is —NR^{1a}C(O)NR^{1b}R^{1c}, where R^{1a}, R^{1b}, and R^{1c} are each as defined herein. In certain embodiments, R⁴ is —NR^{1a}C(═NR^{1d})NR^{1b}R^{1c}, where R^{1a}, Rb, R^{1c}, and R^{d} are each as defined herein. In certain embodiments, R⁴ is —NR^{1a}S(O)R^{1d}, where R^{1a} and R^{d} are each as defined herein. In certain embodiments, R⁴ is —NR^{a}S(O)₂R^{1d}, where R^{1a} and R^{1d} are each defined herein. In certain embodiments, R⁴ is —NR^{1a}S(O)NR^{1b}R^{1c}, where R^{1a}, R^{1b}, and R^{1c} are each as defined herein. In certain embodiments, R⁴ is —NR^{1a}S(O)₂NR^{1b}R^{1c}, where R^{1a}, R^{1b}, and R^{1c} are each as defined herein. In certain embodiments, R⁴ is —P(O)(OR^{1a})R^{1d}, where R^{1a} and R^{d} are each defined herein. In certain embodiments, R⁴ is —CH₂P(O)(OR^{1a})R^{1d}, where R^{1a} and R^{1d} are each defined herein. In certain embodiments, R⁴ is —SR^{1a}, where R^{1a} is as defined herein. In certain embodiments, R⁴ is —S(O)R^{1a}, where R^{1a} is as defined herein. In certain embodiments, R⁴ is —S(O)_{2R}^{1a}, where R^{1a} is as defined herein. In certain embodiments, R⁴ is —S(O)NR^{1b}R^{1c}, where R^{1b} and R^{1c} are each as defined herein. In certain embodiments, R⁴ is —S(O)₂NR^{1b}R^{1c}, where R^{1b} and R^{1c} are each as defined herein. In certain embodiments, R⁴ is chloro, fluoro, nitro, amino, methyl, trifluoromethyl, phenyl, or methoxy.

In certain embodiments, two R⁴ are linked together to form a bond. In certain embodiments, two R⁴ are linked together to form —O—. In certain embodiments, two R⁴ are linked together to form —NR^{N}—, where R^{N} is as defined herein. In certain embodiments, two R⁴ are linked together to form —S—. In certain embodiments, two R⁴ are linked together to form C₁₋₆ alkylene, optionally substituted with one or more substituents Q. In certain embodiments, two R⁴ are linked together to form methylene, ethylene, or propylene, each optionally substituted with one or more substituents Q. In certain embodiments, two R⁴ are linked together to form C₁₋₆ heteroalkylene, optionally substituted with one or more substituents Q. In certain embodiments, two R⁴ are linked together to form C₂₋₆ alkenylene, optionally substituted with one or more substituents Q. In certain embodiments, two R⁴ are linked together to form C₂₋₆ heteroalkenylene, optionally substituted with one or more substituents Q. In certain embodiments, two R⁴ are linked together to form a fused ring. In certain embodiments, two R⁴ are linked together to form a bridged ring. In certain embodiments, two R⁴ are linked together to form a spiro ring.

In certain embodiments, R⁵ is C₁₋₆ alkyl, optionally substituted with one or more substituents Q. In certain embodiments, R⁵ is methyl. In certain embodiments, R⁵ is C₂₋₆ alkenyl, optionally substituted with one or more substituents Q. In certain embodiments, R⁵ is C₂₋₆ alkynyl, optionally substituted with one or more substituents Q. In certain embodiments, R⁵ is C₃₋₇ cycloalkyl, optionally substituted with one or more substituents Q. In certain embodiments, R⁵ is C₆₋₁₄ aryl, optionally substituted with one or more substituents Q. In certain embodiments, R⁵ is C₇₋₁₅ aralkyl, optionally substituted with one or more substituents Q. In certain embodiments, R⁵ is heteroaryl, optionally substituted with one or more substituents Q. In certain embodiments, R⁵ is heterocyclyl, optionally substituted with one or more substituents Q.

In certain embodiments, R⁶ is hydrogen. In certain embodiments, R⁶ is C₁₋₆ alkyl, optionally substituted with one or more substituents Q. In certain embodiments, R⁶ is methyl. In certain embodiments, R⁶ is C₂₋₆ alkenyl, optionally substituted with one or more substituents Q. In certain embodiments, R⁶ is C₂₋₆ alkynyl, optionally substituted with one or more substituents Q. In certain embodiments, R⁶ is C₃₋₇ cycloalkyl, optionally substituted with one or more substituents Q. In certain embodiments, R⁶ is C₆₋₁₄ aryl, optionally substituted with one or more substituents Q. In certain embodiments, R⁶ is C₇₋₁₅ aralkyl, optionally substituted with one or more substituents Q. In certain embodiments, R⁶ is heteroaryl, optionally substituted with one or more substituents Q. In certain embodiments, R⁶ is heterocyclyl, optionally substituted with one or more substituents Q. In certain embodiments, R⁶ is heterocyclyl, optionally substituted with one or more substituents Q.

In certain embodiments, R⁶ is —CHR^{6a}C(O)R^{6b}, where R^{6a} and R^{6b} are each as defined herein. In certain embodiments, R^{6a} is hydrogen. In certain embodiments, R^{6a} is C₁₋₆ alkyl, optionally substituted with one or more substituents Q. In certain embodiments, R^{6a} is methyl. In certain embodiments, R^{6a} is C₂₋₆ alkenyl, optionally substituted with one or more substituents Q. In certain embodiments, R^{6a} is C₂₋₆ alkynyl, optionally substituted with one or more substituents Q. In certain embodiments, R^{6a} is C₃₋₇ cycloalkyl, optionally substituted with one or more substituents Q. In certain embodiments, R^{6a} is C₆₋₁₄ aryl, optionally substituted with one or more substituents Q. In certain embodiments, R^{6a} is C₇₋₁₅ aralkyl, optionally substituted with one or more substituents Q. In certain embodiments, R^{6a} is heteroaryl, optionally substituted with one or more substituents Q. In certain embodiments, R^{6a} is heterocyclyl, optionally substituted with one or more substituents Q. In certain embodiments, R^{6a} is heterocyclyl, optionally substituted with one or more substituents Q. In certain embodiments, R^{6b} is

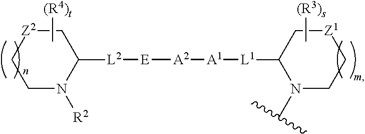

where R², R³, R⁴, A¹, A², E, L¹, L², Z¹, Z², m, n, s, and t are each as defined herein. In certain embodiments, R^{6b} is

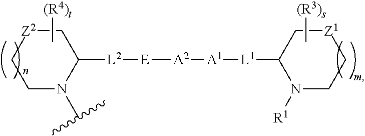

where R¹, R³, R⁴, A¹, A², E, L¹, L², Z¹, Z², m, n, s, and t are each as defined herein.

In certain embodiments, R⁷ is hydrogen. In certain embodiments, R⁷ is cyano. In certain embodiments, R⁷ is halo. In certain embodiments, R⁷ is nitro. In certain embodiments, R⁷ is C₁₋₆ alkyl, optionally substituted with one or more substituents Q. In certain embodiments, R⁷ is C₂₋₆ alkenyl, optionally substituted with one or more substituents Q. In certain embodiments, R⁷ is C₂₋₆ alkynyl, optionally substituted with one or more substituents Q. In certain embodiments, R⁷ is C₃₋₇ cycloalkyl, optionally substituted with one or more substituents Q. In certain embodiments, R⁷ is cyclohexyl, optionally substituted with one or more substituents Q. In certain embodiments, R⁷ is cyclohexyl. In certain embodiments, R⁷ is C₆₋₁₄ aryl, optionally substituted with one or more substituents Q. In certain embodiments, R⁷ is C₇₋₁₅ aralkyl, optionally substituted with one or more substituents Q. In certain embodiments, R⁷ is heteroaryl, optionally substituted with one or more substituents Q. In certain embodiments, R⁷ is heterocyclyl, optionally substituted with one or more substituents Q.

In certain embodiments, R⁷ is —C(O)R^{1a}, where R^{1a} is as defined herein. In certain embodiments, R⁷ is —C(O)OR^{1a}, where R^{1a} is as defined herein. In certain embodiments, R¹ is —C(O)NR^{1b}R^{1c}, where R^{1b} and R^{1c} are each as defined herein. In certain embodiments, R⁷ is —C(NR^{1a})NR^{1b}R^{1c}, where R^{1a}, R^{1b}, and R^{1c} are each as defined herein. In certain embodiments, R⁷ is —OR^{1a}, where R^{1a} is as defined herein. In certain embodiments, R⁷ is —OH. In certain embodiments, R⁷ is —OC(O)R^{1a}, where R^{1a} is as defined herein. In certain embodiments, R⁷ is —OC(O)OR^{1a}, where R^{1a} is as defined herein. In certain embodiments, R⁷ is —OC(O)NR^{1b}R^{1c}, where R^{1b} and R^{1c} are each as defined herein. In certain embodiments, R⁷ is —OC(═NR^{a})NR^{1b}R^{1c}, where R^{1a}, R^{1b}, and R^{1c} are each as defined herein. In certain embodiments, R⁷ is —OS(O)R^{1a}, where R^{1a} is as defined herein. In certain embodiments, R⁷ is —OS(O)₂R^{1a}, where R^{1a} is as defined herein. In certain embodiments, R⁷ is —OS(O)NR^{1b}R^{1c}, where R^{1b} and R^{1c} are each as defined herein. In certain embodiments, R⁷ is —OS(O)₂NR^{1b}R^{1c}, where R^{1b} and R^{1c} are each as defined herein. In certain embodiments, R⁷ is —NR^{1b}R^{1c}, where R^{1b} and R^{1c} are each as defined herein. In certain embodiments, R⁷ is —NR^{1a}C(O)R^{1d}, where R^{1a} and R^{1d} are each as defined herein. In certain embodiments, R⁷ is —NR^{1a}C(O)OR^{1d}, where R^{1a} and R^{1d} are each as defined herein. In certain embodiments, R⁷ is —NR^{1a}C(O)NR^{1b}R^{1c}, where R^{1a}, R^{1b}, and R^{1c} are each as defined herein. In certain embodiments, R⁷ is —NR^{1a}C(═NR^{d})NR^{1b}R^{1c}, where R^{1a}, R^{1b}, R^{1c}, and R^{1d} are each as defined herein. In certain embodiments, R⁷ is —NR^{1a}S(O)R^{1d}, where R^{1a} and R^{1d} are each as defined herein. In certain embodiments, R⁷ is —NR^{a}S(O)₂R^{1d}, where R^{1a} and R^{1d} are each defined herein. In certain embodiments, R⁷ is —NR^{1a}S(O)NR^{1b}R^{1c}, where R^{1a}, R^{1b}, and R^{1c} are each as defined herein. In certain embodiments, R⁷ is —NR^{a}S(O)₂NR^{1b}R^{1c}, where R^{1a}, R^{1b}, and R^{1c} are each as defined herein. In certain embodiments, R⁷ is —P(O)(OR^{1a})R^{1d}, where R^{1a} and R^{1d} are each defined herein. In certain embodiments, R⁷ is —CH₂P(O)(OR^{1a})R^{1d}, where R^{1a} and R^{1d} are each defined herein. In certain embodiments, R⁷ is —SR^{1a}, where R^{1a} is as defined herein. In certain embodiments, R⁷ is —S(O)R^{1a}, where R^{1a} is as defined herein. In certain embodiments, R⁷ is —S(O)₂R^{1a}, where R^{1a} is as defined herein. In certain embodiments, R⁷ is —S(O)NR^{1b}R^{1c}, where R^{1b} and R^{1c} are each as defined herein. In certain embodiments, R⁷ is —S(O)₂NR^{1b}R^{1c}, where R^{1b} and R^{1c} are each as defined herein. In certain embodiments, R⁷ is —C₁₋₆ alkylene-OP(O)(OR^{P1})₂, wherein R^{P1} is as defined herein. In certain embodiments, R⁷ is —C(R^{P2})₂—OP(O)(OR^{P1})₂, wherein R^{P1} and R^{P2} are each as defined herein. In certain embodiments, R⁷ is —CH₂—OP(O)(OR^{P1})₂, wherein R^{P1} is as defined herein. In certain embodiments, R⁷ is —CH₂—OP(O)(OH)₂. In certain embodiments, R⁷ is —CH₂—OP(O)(ONa)₂. In certain embodiments, R⁷ is —C₁₋₆ alkylene-O-linked amino acid or a derivative thereof. In certain embodiments, R⁷ is —CH₂—OC(O)C(R^{aa})₂NR^{1b}R^{1c}, where R^{aa}, R^{1b}, and R^{1c} are defined as herein. In particular embodiments, R⁷ is —CH₂—OC(O)CH(i-propyl)NH₂.

In certain embodiments, R^{7a} is cyano. In certain embodiments, R^{7a} is halo. In certain embodiments, R^{7a} is nitro. In certain embodiments, R^{7a} is C₁₋₆ alkyl, optionally substituted with one or more substituents Q. In certain embodiments, R^{7a} is C₂₋₆ alkenyl, optionally substituted with one or more substituents Q. In certain embodiments, R^{7a} is C₂₋₆ alkynyl, optionally substituted with one or more substituents Q. In certain embodiments, R^{7a} is C₃₋₇ cycloalkyl, optionally substituted with one or more substituents Q. In certain embodiments, R^{7a} is cyclohexyl, optionally substituted with one or more substituents Q. In certain embodiments, R^{7a} is cyclohexyl. In certain embodiments, R^{7a} is C₆₋₁₄ aryl, optionally substituted with one or more substituents Q. In certain embodiments, R^{7a} is C₇₋₁₅ aralkyl, optionally substituted with one or more substituents Q. In certain embodiments, R^{7a} is heteroaryl, optionally substituted with one or more substituents Q. In certain embodiments, R^{7a} is heterocyclyl, optionally substituted with one or more substituents Q.

In certain embodiments, R^{7a} is —C(O)R^{1a}, where R^{1a} is as defined herein. In certain embodiments, R^{7a} is —C(O)OR^{1a}, where R^{1a} is as defined herein. In certain embodiments, R^{7a} is —C(O)NR^{1b}R^{1c}, where R^{1b} and R¹ are each as defined herein. In certain embodiments, R^{7a} is —C(NR^{1a})NR^{1b}R^{1c}, where R, R^{1b}, and R^{1c} are each as defined herein. In certain embodiments, R^{7a} is —OR^{1a}, where R^{1a} is as defined herein. In certain embodiments, R^{7a} is —OH. In certain embodiments, R^{7a} is —OC(O)R^{1a}, where R^{1a} is as defined herein. In certain embodiments, R^{7a} is —OC(O)OR^{a}, where R^{1a} is as defined herein. In certain embodiments, R^{7a} is —OC(O)NR^{1b}R^{1c}, where R^{1b} and R^{1c} are each as defined herein. In certain embodiments, R^{7a} is —OC(═NR^{a})NR^{1b}R^{1c}, where R^{1a}, R^{1b}, and R^{1c} are each as defined herein. In certain embodiments, R^{7a} is —OS(O)R^{1a}, where R^{1a} is as defined herein. In certain embodiments, R^{7a} is —OS(O)₂R^{1a}, where R^{aa} is as defined herein. In certain embodiments, R^{7a} is —OS(O)NR^{1b}R^{1c}, where R^{1b} and R^{1c} are each as defined herein. In certain embodiments, R^{7a} is —OS(O)₂NR^{1b}R^{1c}, where R^{1b} and R^{1c} are each as defined herein. In certain embodiments, R^{7a} is —NR^{1b}R^{1c}, where R^{1b} and R^{1c} are each as defined herein. In certain embodiments, R^{7a} is —NR^{1a}C(O)R^{1d}, where R^{a} and R^{d} are each as defined herein. In certain embodiments, R^{7a} is —NR^{1a}C(O)OR^{1d}, where R^{1a} and R^{d} are each as defined herein. In certain embodiments, R^{7a} is —NR^{1a}C(O)NR^{1b}R^{1c}, where R^{1a}, R^{1b}, and R^{1c} are each as defined herein. In certain embodiments, R^{7a} is —NR^{1a}C(═NR^{d})NR^{1b}R^{1c}, where R^{1a}, R^{1b}, R^{1c}, and R^{1d} are each as defined herein. In certain embodiments, R^{7a} is —NR^{1a}S(O)R^{1d}, where R^{1a} and R^{1d} are each as defined herein. In certain embodiments, R^{7a} is —NR^{a}S(O)₂R^{1d}, where R^{1a} and R^{1d} are each defined herein. In certain embodiments, R^{7a} is —NR^{1a}S(O)NR^{1b}R^{1c}, where R^{1a}, R^{1b}, and R^{1c} are each as defined herein. In certain embodiments, R^{7a} is —NR^{1a}S(O)₂NR^{b}R^{c}, where R^{1a}, R^{1b}, and R^{1c} are each as defined herein. In certain embodiments, R^{7a} is —P(O)(OR^{1a})R^{1d}, where R^{1a} and R^{1d} are each defined herein. In certain embodiments, R^{7a} is —CH₂P(O)(OR^{1a})R^{1d}, where R^{1a} and R^{1d} are each defined herein. In certain embodiments, R^{7a} is —SR)^{a}, where R^{1a} is as defined herein. In certain embodiments, R^{7′} is —S(O)R^{1a}, where R^{1a} is as defined herein. In certain embodiments, R^{7a} is —S(O)₂R^{a}, where R^{1a} is as defined herein. In certain embodiments, R^{7a} is —S(O)NR^{1b}R^{1c}, where R^{1b} and R^{1c} are each as defined herein. In certain embodiments, R^{7a} is —S(O)₂NR^{1b}R^{1c}, where R^{1b} and R^{1c} are each as defined herein. In certain embodiments, R^{7a} is —C₁₋₆ alkylene-OP(O)(OR^{P1})₂, wherein R^{P1} is as defined herein. In certain embodiments, R^{7a} is —C(R^{P2})₂—OP(O)(OR^{P1})₂, wherein R^{P1} and R^{P2} are each as defined herein. In certain embodiments, R^{7a} is —CH₂—OP(O)(OR^{P1})₂, wherein R^{P1} is as defined herein. In certain embodiments, R^{7a} is —CH₂—OP(O)(OH)₂. In certain embodiments, R^{7a} is —CH₂—OP(O)(ONa)₂. In certain embodiments, R^{7a} is —C₁₋₆ alkylene-O-linked amino acid or a derivative thereof. In certain embodiments, R^{7a} is —CH₂—OC(O)C(R^{aa})₂NR^{1b}R^{1c}, where R^{aa}, R^{1b}, and R^{1c} are defined as herein. In particular embodiments, R^{7a} is —CH₂—OC(O)CH(i-propyl)NH₂.

In certain embodiments, R^{8a} is hydrogen. In certain embodiments, R^{8a} is C₁₋₆ alkyl, optionally substituted with one or more substituents Q. In certain embodiments, R^{8a} is isopropyl, optionally substituted with one or more substituents Q. In certain embodiments, R^{8a} is C₂₋₆ alkenyl, optionally substituted with one or more substituents Q. In certain embodiments, R^{8a} is C₂₋₆ alkynyl, optionally substituted with one or more substituents Q. In certain embodiments, R^{8a} is C₃₋₇ cycloalkyl, optionally substituted with one or more substituents Q. In certain embodiments, R^{8a} is cyclohexyl, optionally substituted with one or more substituents Q. In certain embodiments, R^{8a} is cyclohexyl. In certain embodiments, R^{8a} is C₆₋₁₄ aryl, optionally substituted with one or more substituents Q. In certain embodiments, R^{8a} is phenyl, optionally substituted with one or more substituents Q. In certain embodiments, R^{8a} is C₇₋₁₅ aralkyl, optionally substituted with one or more substituents Q. In certain embodiments, R^{8a} is heteroaryl, optionally substituted with one or more substituents Q. In certain embodiments, R^{8a} is heterocyclyl, optionally substituted with one or more substituents Q.

In certain embodiments, R^{8b} is hydrogen. In certain embodiments, R^{8b} is cyano. In certain embodiments, R^{8b} is halo. In certain embodiments, R^{8b} is nitro. In certain embodiments, R^{8b} is C₁₋₆ alkyl, optionally substituted with one or more substituents Q. In certain embodiments, R^{8b} is C₂₋₆ alkenyl, optionally substituted with one or more substituents Q. In certain embodiments, R^{8b} is C₂₋₆ alkynyl, optionally substituted with one or more substituents Q. In certain embodiments, R^{8b} is C₃₋₇ cycloalkyl, optionally substituted with one or more substituents Q. In certain embodiments, R^{8b} is cyclohexyl, optionally substituted with one or more substituents Q. In certain embodiments, R^{8b} is cyclohexyl. In certain embodiments, R^{8b} is C₆₋₁₄ aryl, optionally substituted with one or more substituents Q. In certain embodiments, R^{8b} is C₇₋₁₅ aralkyl, optionally substituted with one or more substituents Q. In certain embodiments, R^{8b} is heteroaryl, optionally substituted with one or more substituents Q. In certain embodiments, R^{8b} is heterocyclyl, optionally substituted with one or more substituents Q.

In certain embodiments, R^{8b} is —C(O)R^{1a}, where R^{a} is as defined herein. In certain embodiments, R^{8b} is —C(O)OR^{1a}, where R^{1a} is as defined herein. In certain embodiments, R^{8b} is —C(O)OCH₃. In certain embodiments, R^{8b} is —C(O)NR^{1b}R^{1c}, where R^{1b} and R^{1c} are each as defined herein. In certain embodiments, R^{8b} is —C(NR^{1a})NR^{1b}R^{1c}, where R^{1a}, R^{1b}, and R^{1c} are each as defined herein. In certain embodiments, R^{8b} is —OR^{1a}, where R^{1a} is as defined herein. In certain embodiments, R^{8b} is —OH. In certain embodiments, R^{8b} is —OC(O)R^{1a}, where R^{1a} is as defined herein. In certain embodiments, R^{8b} is —OC(O)OR^{1a}, where R^{1a} is as defined herein. In certain embodiments, R^{8b} is —OC(O)NR^{1b}R^{1c}, where R^{1b} and R^{1c} are each as defined herein. In certain embodiments, R^{8b} is —OC(═NR^{1a})NR^{1b}R^{1c}, where R^{1a}, R^{1b}, and R^{1c} are each as defined herein. In certain embodiments, R^{8b} is —OS(O)R^{1a}, where R¹ is as defined herein. In certain embodiments, R^{8b} is —OS(O)₂R^{1a}, where R^{1a} is as defined herein. In certain embodiments, R^{8b} is —OS(O)NR^{1b}R^{1c}, where R^{1b} and R^{1c} are each as defined herein. In certain embodiments, R^{8b} is —OS(O)₂NR^{1b}R^{1c}, where R^{1b} and R^{1c} are each as defined herein. In certain embodiments, R^{8b} is —NR^{1b}R^{1c}, where R^{1b} and R^{1c} are each as defined herein. In certain embodiments, R^{8b} is —NR^{1a}C(O)R^{1d}, where R^{1a} and R^{1d} are each as defined herein. In certain embodiments, R^{8b} is —NR^{1a}C(O)OR^{1d}, where R^{1a} and R^{d} are each as defined herein. In certain embodiments, R^{8b} is —NR^{1a}C(O)NR^{1b}R^{1c}, where R^{1a}, R^{1b}, and R^{1c} are each as defined herein. In certain embodiments, R^{8b} is —NR^{1a}C(═NR^{1d})NR^{1b}R^{1c}, where R^{1a}, R^{1b}, R^{1c}, and R^{1d} are each as defined herein. In certain embodiments, R^{8b} is —NR^{1a}S(O)R^{1d}, where R^{1a} and R^{1d} are each as defined herein. In certain embodiments, R^{8b} is —NR^{1a}S(O)₂R^{1d}, where R^{1a} and R^{1d} are each defined herein. In certain embodiments, R^{8b} is —NR^{1a}S(O)NR^{1b}R^{1c}, where R^{1a}, R^{1b}, and R^{1c} are each as defined herein. In certain embodiments, R^{8b} is —NR^{1a}S(O)₂NR^{1b}R^{1c}, where R^{1a}, R^{1b}, and R^{1c} are each as defined herein. In certain embodiments, R^{8b} is —P(O)(OR^{1a})R^{1d}, where R^{1a} and R^{1d} are each defined herein. In certain embodiments, R^{8b} is —CH₂P(O)(OR^{1a})R^{1d}, where R^{1a} and R^{1d} are each defined herein. In certain embodiments, R^{8b} is —SR^{1a}, where R^{1a} is as defined herein. In certain embodiments, R^{8b} is —S(O)R^{1a}, where R^{1a} is as defined herein. In certain embodiments, R^{8b} is —S(O)₂R^{a}, where R^{1a} is as defined herein. In certain embodiments, R^{8b} is —S(O)NR^{1b}R¹, where R^{1b} and R^{1c} are each as defined herein. In certain embodiments, R^{8b} is —S(O)₂NR^{1b}R^{1c}, where R^{1b} and R^{1c} are each as defined herein. In certain embodiments, R^{8b} is —C₁₋₆ alkylene-OP(O)(OR^{P1})₂, wherein R^{P1} is as defined herein. In certain embodiments, R^{8b} is —C(R^{P2})₂—OP(O)(OR^{P1})₂, wherein R^{P1} and R^{P2} are each as defined herein. In certain embodiments, R^{8b} is —CH₂—OP(O)(OR^{P1})₂, wherein R^{P1} is as defined herein. In certain embodiments, R^{8b} is —CH₂—OP(O)(OH)₂. In certain embodiments, R^{8b} is —CH₂—OP(O)(ONa)₂. In certain embodiments, R^{8b} is —C₁₋₆ alkylene-O-linked amino acid or a derivative thereof. In certain embodiments, R^{8b} is —CH₂—OC(O)C(R^{aa})₂NR^{1b}R^{1c}, where R^{aa}, R^{1b}, and R^{1c} are defined as herein. In particular embodiments, R^{8b} is —CH₂—OC(O)CH(i-propyl)NH₂.

In certain embodiments, R^{8c} is hydrogen. In certain embodiments, R^{8c} is cyano. In certain embodiments, R^{8c} is halo. In certain embodiments, R^{8c} is nitro. In certain embodiments, R^{8c} is C₁₋₆ alkyl, optionally substituted with one or more substituents Q. In certain embodiments, R^{8c} is C₂₋₆ alkenyl, optionally substituted with one or more substituents Q. In certain embodiments, R^{8c} is C₂₋₆ alkynyl, optionally substituted with one or more substituents Q. In certain embodiments, R^{8c} is C₃₋₇ cycloalkyl, optionally substituted with one or more substituents Q. In certain embodiments, R^{8c} is cyclohexyl, optionally substituted with one or more substituents Q. In certain embodiments, R^{8c} is cyclohexyl. In certain embodiments, R^{8c} is C₆₋₁₄ aryl, optionally substituted with one or more substituents Q. In certain embodiments, R^{8c} is C₇₋₁₅ aralkyl, optionally substituted with one or more substituents Q. In certain embodiments, R^{8c} is heteroaryl, optionally substituted with one or more substituents Q. In certain embodiments, R^{8c} is heterocyclyl, optionally substituted with one or more substituents Q.

In certain embodiments, R^{8c} is —C(O)R^{1a}, where R^{1a} is as defined herein. In certain embodiments, R^{8c} is —C(O)OR^{1a}, where R^{1a} is as defined herein. In certain embodiments, R^{8c} is —C(O)OCH₃. In certain embodiments, R^{8c} is —C(O)NR^{1b}R^{1c}, where R^{1b} and R^{1c} are each as defined herein. In certain embodiments, R^{8c} is —C(NR^{1a})NR^{1b}R^{1c}, where R^{1a}, R^{1b}, and R^{1c} are each as defined herein. In certain embodiments, R^{8c} is —OR^{1a}, where R^{1a} is as defined herein. In certain embodiments, R^{8c} is —OH. In certain embodiments, R^{8c} is —OC(O)R^{1a}, where R^{1a} is as defined herein. In certain embodiments, R^{8c} is —OC(O)OR^{1a}, where R^{1a} is as defined herein. In certain embodiments, R^{8c} is —OC(O)NR^{1b}R^{1c}, where R^{1b} and R^{1c} are each as defined herein. In certain embodiments, R^{8c} is —OC(═NR^{1a})NR^{1b}R^{1c}, where R^{1a}, R^{1b}, and R^{1c} are each as defined herein. In certain embodiments, R^{8c} is —OS(O)R^{1a}, where R^{1a} is as defined herein. In certain embodiments, R^{8c} is —OS(O)₂R^{1a}, where R^{1a} is as defined herein. In certain embodiments, R^{8c} is —OS(O)NR^{1b}R^{1c}, where R^{1b} and R^{1c} are each as defined herein. In certain embodiments, R^{8c} is —OS(O)₂NR^{1b}R^{1c}, where R^{1b} and R^{1c} are each as defined herein. In certain embodiments, R^{8c} is —NR^{1b}R^{1c}, where R^{1b} and R^{1c} are each as defined herein. In certain embodiments, R^{8c} is —NR^{1a}C(O)R^{1d}, where R^{1a} and R^{1d} are each as defined herein. In certain embodiments, R^{8c} is —NR^{1a}C(O)OR^{1d}, where R^{1a} and R^{1d} are each as defined herein. In certain embodiments, R^{8c} is —NR^{1a}C(O)NR^{1b}R^{1a}, where R^{1a}, R^{1b}, and R^{1c} are each as defined herein. In certain embodiments, R^{8c} is —NR^{1a}C(═NR^{1d})NR^{1b}R^{1a}, where R^{1a}, R^{1b}, R^{1c}, and R^{1d} are each as defined herein. In certain embodiments, R^{8c} is —NR^{1a}S(O)R^{1d}, where R^{1a} and R^{1d} are each as defined herein. In certain embodiments, R^{8c} is —NR^{1a}S(O)₂R^{1d}, where R^{1a} and R^{1d} are each defined herein. In certain embodiments, R^{8c} is —NR^{1a}S(O)NR^{1b}R^{1a}, where R^{1a}, R^{1b}, and R^{1c} are each as defined herein. In certain embodiments, R^{8c} is —NR^{1a}S(O)₂NR^{1b}R^{1a}, where R^{1a}, R^{1b}, and R^{1c} are each as defined herein. In certain embodiments, R^{8c} is —P(O)(OR^{1a})R^{1d}, where R^{1a} and R^{1d} are each defined herein. In certain embodiments, R^{8c} is —CH₂P(O)(OR^{1a})R^{1d}, where R^{1a} and R^{1d} are each defined herein. In certain embodiments, R^{8c} is —SR^{1a}, where R^{1a} is as defined herein. In certain embodiments, R^{8c} is —S(O)R^{1a}, where R^{1a} is as defined herein. In certain embodiments, R^{8c} is —S(O)₂R^{1a}, where R^{1a} is as defined herein. In certain embodiments, R^{8c} is —S(O)NR^{1b}R^{1c}, where R^{1b} and R^{1c} are each as defined herein. In certain embodiments, R^{8c} is —S(O)₂NR^{b}R^{1c}, where R^{1b} and R^{1c} are each as defined herein. In certain embodiments, R^{8c} is —C₁₋₆ alkylene-OP(O)(OR^{P1})₂, wherein R^{P1} is as defined herein. In certain embodiments, R^{8c} is —C(R^{P2})₂—OP(O)(OR^{P1})₂, wherein R^{P1} and R^{P2} are each as defined herein. In certain embodiments, R^{8c} is —CH₂—OP(O)(OR^{P1})₂, wherein R^{P1} is as defined herein. In certain embodiments, R^{8c} is —CH₂—OP(O)(OH)₂. In certain embodiments, R^{8c} is —CH₂—OP(O)(ONa)₂. In certain embodiments, R^{8c} is —C₁₋₆ alkylene-O-linked amino acid or a derivative thereof. In certain embodiments, R^{8c} is —CH₂—OC(O)C(R^{aa})₂NR^{1b}R^{1c}, where R^{1a}, R^{1b}, and R^{1c} are defined as herein. In particular embodiments, R^{8c} is —CH₂—OC(O)CH(i-propyl)NH₂.

In certain embodiments, R^{P} is —C₁₋₆ alkylene-OP(O)(OR^{P1})₂, wherein R^{P1} is as defined herein. In certain embodiments, R^{P} is —C(R^{P2})₂—OP(O)(OR^{P1})₂, wherein R^{P1} and R^{P2} are each as defined herein. In certain embodiments, R^{P} is —CH₂—OP(O)(OR^{P1})₂, wherein R^{P1} is as defined herein. In certain embodiments, R^{P} is —CH₂—OP(O)(OH)₂. In certain embodiments, R^{P} is —CH₂—OP(O)(ONa)₂. In certain embodiments, R^{P} is —C₁₋₆ alkylene-O-linked amino acid or a derivative thereof. In certain embodiments, R^{P} is —CH₂—OC(O)C(R^{aa})₂NR^{1b}R^{1c}, where R^{aa}, R^{1b}, and R^{1c} are defined as herein. In particular embodiments, R^{P} is —CH₂—OC(O)CH(i-propyl)NH₂.

In certain embodiments, R^{P1} is hydrogen. In certain embodiments, R^{P1} is a monovalent cation. In certain embodiments, R^{P1} is an alkali metal ion. In certain embodiments, R^{P1} is Li⁺, Na⁺, K⁺, Rb⁺, or Cs⁺. In certain embodiments, R^{P1} is Li⁺. In certain embodiments, R^{P1} is Na⁺. In certain embodiments, R^{P1} is K⁺. In certain embodiments, R^{P1} is Rb⁺. In certain embodiments, R^{P1} is Cs⁺. In certain embodiments, R^{P1} is C₁₋₆ alkyl, optionally substituted with one or more substituents Q. In certain embodiments, R^{P1} is methyl, ethyl, or t-butyl. In certain embodiments, R^{P1} is C₂₋₆ alkenyl, optionally substituted with one or more substituents Q. In certain embodiments, R^{P1} is C₂₋₆ alkynyl, optionally substituted with one or more substituents Q. In certain embodiments, R^{P1} is C₃₋₇ cycloalkyl, optionally substituted with one or more substituents Q. In certain embodiments, R^{P1} is C₆₋₁₄ aryl, optionally substituted with one or more substituents Q. In certain embodiments, R^{P1} is C₇₋₁₅ aralkyl, optionally substituted with one or more substituents Q. In certain embodiments, R^{P1} is benzyl, optionally substituted with one or more substituents Q. In certain embodiments, R^{P1} is heteroaryl, optionally substituted with one or more substituents Q. In certain embodiments, R^{P1} is heterocyclyl, optionally substituted with one or more substituents Q. In certain embodiments, two R^{P1} together are a divalent cation. In certain embodiments, two R^{P1} together are analkaline earth metal ion. In certain embodiments, two R^{P1} together are Mg²⁺. In certain embodiments, two R^{P1} together are Ca²⁺.

In certain embodiments, R^{P2} is hydrogen. In certain embodiments, R^{P2} is cyano. In certain embodiments, R^{P2} is halo. In certain embodiments, R^{P2} is nitro. In certain embodiments, R^{P2} is C₁₋₆ alkyl, optionally substituted with one or more substituents Q. In certain embodiments, R^{P2} is C₂₋₆ alkenyl, optionally substituted with one or more substituents Q. In certain embodiments, R^{P2} is C₂₋₆ alkynyl, optionally substituted with one or more substituents Q. In certain embodiments, R^{P2} is C₃₋₇ cycloalkyl, optionally substituted with one or more substituents Q. In certain embodiments, R^{P2} is C₆₋₁₄ aryl, optionally substituted with one or more substituents Q. In certain embodiments, R^{P2} is C₇₋₁₅ aralkyl, optionally substituted with one or more substituents Q. In certain embodiments, R^{P2} is heteroaryl, optionally substituted with one or more substituents Q. In certain embodiments, R^{P2} is heterocyclyl, optionally substituted with one or more substituents Q.

In certain embodiments, A¹ is a bond. In certain embodiments, A¹ is C₁₋₆ alkylene, optionally substituted with one or more substituents Q. In certain embodiments, A¹ is C₂₋₆ alkenylene, optionally substituted with one or more substituents Q. In certain embodiments, A¹ is ethenylene, optionally substituted with one or more substituents Q. In certain embodiments, A¹ is C₂₋₆ alkynylene, optionally substituted with one or more substituents Q. In certain embodiments, A¹ is ethynylene. In certain embodiments, A¹ is C₂₋₂₀ cycloalkylene, optionally substituted with one or more substituents Q. In certain embodiments, A¹ is C₆₋₂₀ arylene, optionally substituted with one or more substituents Q. In certain embodiments, A¹ is 6-membered arylene, optionally substituted with one or more substituents Q. In certain embodiments, A¹ is phenylene, optionally substituted with one or more substituents Q. In certain embodiments, A¹ is 1,4-phenylene, optionally substituted with one or more substituents Q. In certain embodiments, A¹ is bicyclic arylene, optionally substituted with one or more substituents Q. In certain embodiments, A¹ is naphthylene, optionally substituted with one or more substituents Q. In certain embodiments, A¹ is tricyclic arylene, optionally substituted with one or more substituents Q. In certain embodiments, A¹ is tricyclic arylene, optionally substituted with one or more substituents Q. In certain embodiments, A¹ is fluorenylene, optionally substituted with one or more substituents Q. In certain embodiments, A¹ is 9,9-difluoro-9H-fluorenylene, optionally substituted with one or more substituents Q. In certain embodiments, A¹ is 9,9-difluoro-9H-fluorenylene, optionally substituted with one or more substituents Q. In certain embodiments, A¹ is 9,9-difluoro-9H-fluorenyl-2,7-ene, optionally substituted with one or more substituents Q. In certain embodiments, A¹ is tetracyclic arylene, optionally substituted with one or more substituents Q. In certain embodiments, A¹ is heteroarylene; optionally substituted with one or more substituents Q. In certain embodiments, A¹ is monocyclic heteroarylene; optionally substituted with one or more substituents Q. In certain embodiments, A¹ is bicyclic heteroarylene; optionally substituted with one or more substituents Q. In certain embodiments, A¹ is 5,5-fused heteroarylene; optionally substituted with one or more substituents Q. In certain embodiments, A¹ is 5,6-fused heteroarylene; optionally substituted with one or more substituents Q. In certain embodiments, A¹ is 6,6-fused heteroarylene; optionally substituted with one or more substituents Q. In certain embodiments, A¹ is tricyclic heteroarylene; optionally substituted with one or more substituents Q. In certain embodiments, A¹ is tetracyclic heteroarylene; optionally substituted with one or more substituents Q. In certain embodiments, A¹ is heterocyclylene, optionally substituted with one or more substituents Q. In certain embodiments, A¹ is monocyclic heterocyclylene, optionally substituted with one or more substituents Q. In certain embodiments, A¹ is bicyclic heterocyclylene, optionally substituted with one or more substituents Q. In certain embodiments, A¹ is tricyclic heterocyclylene, optionally substituted with one or more substituents Q. In certain embodiments, A¹ is tetracyclic heterocyclylene, optionally substituted with one or more substituents Q.

In certain embodiments, A¹ is thieno[3,2-b]thienylene, pyrrolo[3,4-c]pyrrolylene, 4H-thieno[3,2-b]pyrrolylene, 6H-thieno[2,3-b]pyrrolylene, imidazo[2,1-b]oxazolylene, imidazo[2,1-b]thiazolylene, or 4H-pyrrolo[3,2-d]thiazolylene, each of which is independently and optionally substituted with one or more substituents Q. In certain embodiments, A¹ is thieno[3,2-b]thien-2,6-ylene, thieno[3,2-b]thien-3,6-ylene, pyrrolo[3,4-c]pyrrol-1,4-ylene, 4H-thieno[3,2-b]pyrrol-2,5-ylene, 6H-thieno[2,3-b]pyrrol-3,6-ylene, imidazo[2,1-b]oxazol-2,6-ylene, imidazo[2,1-b]thiazol-2,6-ylene, or 4H-pyrrolo[3,2-d]thiazol-2,5-ylene, each of which is independently and optionally substituted with one or more substituents Q. In certain embodiments, A¹ is 3H-pyrrolizinylene, 4H-furo[3,2-b]pyrrolylene, furo[3,2-b]furanylene, 1,4-dihydropyrrolo[3,2-b]pyrrolylene, 5H-pyrrolo[1,2-c]imidazolylene, 4H-furo[3,2-b]pyrrolylene, 6H-pyrrolo[1,2-b]pyrazolylene, 5H-pyrrolo[1,2-a]imidazolylene, thieno[3,2-b]furanylene, 1H-furo[3,2-c]pyrazolylene, 1H-thieno[3,2-c]pyrazolylene, 1,4-dihydropyrrolo[3,2-c]pyrazolylene, 1H-imidazo[1,2-a]imidazolylene, pyrazolo[5,1-b]oxazolylene, pyrazolo[5,1-b]thiazolylene, 5H-imidazo[1,2-b]pyrazolylene, imidazo[1,2-b]isoxazolylene, imidazo[1,2-b]isothiazolylene, imidazo[1,5-b]isoxazolylene, imidazo[1,5-b]isothiazolylene, imidazo[5,1-b]oxazolylene, imidazo[5,1-b]thiazolylene, 1H-imidazo[1,5-a]imidazolylene, 6H-pyrrolo[3,2-d]isoxazolylene, 6H-pyrrolo[3,2-d]isothiazolylene, pyrrolo[2,1-b][1,3,4]oxadiazolylene, pyrrolo[2,1-b][1,3,4]thiadiazolylene, 1H-pyrrolo[1,2-b][1,2,4]triazolylene, 3H-furo[2,3-d]imidazolylene, 3H-thieno[2,3-d]imidazolylene, 3,4-dihydropyrrolo[2,3-d]imidazolylene, furo[3,2-d]thiazolylene, thieno[3,2-d]thiazolylene, 4H-pyrrolo[3,2-d]thiazolylene, 4H-pyrazolo[3,4-d]isoxazolylene, 4H-pyrazolo[3,4-d]isothiazolylene, 1,4-dihydropyrazolo[4,3-c]pyrazolylene, isoxazolo[5,4-d]isoxazolylene, isothiazolo[5,4-d]isothiazolylene, imidazo[2,1-b][1,3,4]thiadiazolylene, 1H-imidazo[1,5-a]imidazolylene, imidazo[2,1-b]oxazolylene, imidazo[2,1-b]thiazolylene, 1H-imidazo[1,2-a]imidazolylene, 1H-imidazo[1,2-a]imidazolylene, thieno[3,2-b]furanylene, or thiazolo[5,4-d]thiazolylene, each of which is independently and optionally substituted with one or more substituents Q.

In certain embodiments, A¹ is imidazo[2,1-b]thiazol-5,6-ylene, 3H-pyrrolizin-1,5-ylene, 3H-pyrrolizin-2,6-ylene, 4H-furo[3,2-b]pyrrol-2,5-ylene, 4H-furo[3,2-b]pyrrol-3,6-ylene, furo[3,2-b]furan-2,5-ylene, furo[3,2-b]furan-3,6-ylene, 1,4-dihydropyrrolo[3,2-b]pyrrol-2,5-ylene, 1,4-dihydropyrrolo[3,2-b]pyrrol-3,6-ylene, 5H-pyrrolo[1,2-c]imidazol-3,7-ylene, 4H-furo[3,2-b]pyrrol-2,4-ylene, 4H-furo[3,2-b]pyrrol-2,5-ylene, 4H-furo[3,2-b]pyrrol-3,4-ylene, 4H-furo[3,2-b]pyrrol-3,6-ylene, 6H-pyrrolo[1,2-b]pyrazol-2,5-ylene, 5H-pyrrolo[1,2-a]imidazol-2,6-ylene, 5H-pyrrolo[1,2-a]imidazol-3,7-ylene, thieno[3,2-b]furan-2,5-ylene, thieno[3,2-b]furan-3,6-ylene, 1H-furo[3,2-c]pyrazol-3,6-ylene, 1H-thieno[3,2-c]pyrazol-3,6-ylene, 1,4-dihydropyrrolo[3,2-c]pyrazol-3,6-ylene, 1H-imidazo[1,2-a]imidazol-2,6-ylene, pyrazolo[5,1-b]oxazol-2,6-ylene, pyrazolo[5,1-b]oxazol-3,7-ylene, pyrazolo[5,1-b]thiazol-2,6-ylene, pyrazolo[5,1-b]thiazol-3,7-ylene, 5H-imidazo[1,2-b]pyrazol-2,6-ylene, 5H-imidazo[1,2-b]pyrazol-3,7-ylene, imidazo[1,2-b]isoxazol-2,6-ylene, imidazo[1,2-b]isoxazol-3,7-ylene, imidazo[1,2-b]isothiazol-2,6-ylene, imidazo[1,2-b]isothiazol-3,7-ylene, imidazo[1,5-b]isoxazol-3,7-ylene, imidazo[1,5-b]isothiazol-3,6-ylene, imidazo[5,1-b]oxazol-3,7-ylene, imidazo[5,1-b]thiazol-3,7-ylene, 1H-imidazo[1,5-a]imidazol-3,7-ylene, 6H-pyrrolo[3,2-d]isoxazol-3,6-ylene, 6H-pyrrolo[3,2-d]isothiazol-3,6-ylene, pyrrolo[2,1-b][1,3,4]oxadiazol-2,6-ylene, pyrrolo[2,1-b][1,3,4]thiadiazol-2,6-ylene, 1H-pyrrolo[1,2-b][1,2,4]triazol-1,5-ylene, 1H-pyrrolo[1,2-b][1,2,4]triazol-2,6-ylene, 3H-furo[2,3-d]imidazol-2,5-ylene, 3H-furo[2,3-d]imidazol-3,6-ylene, 3H-thieno[2,3-d]imidazol-2,5-ylene, 3H-thieno[2,3-d]imidazol-3,6-ylene, 3,4-dihydropyrrolo[2,3-d]imidazol-2,5-ylene, 3,4-dihydropyrrolo[2,3-d]imidazol-3,6-ylene, furo[3,2-d]thiazol-2,5-ylene, thieno[3,2-d]thiazol-2,5-ylene, 4H-pyrrolo[3,2-d]thiazol-2,5-ylene, 4H-pyrazolo[3,4-d]isoxazol-3,6-ylene, 4H-pyrazolo[3,4-d]isothiazol-3,6-ylene, 1,4-dihydropyrazolo[4,3-c]pyrazol-1,4-ylene, 1,4-dihydropyrazolo[4,3-c]pyrazol-3,6-ylene, isoxazolo[5,4-d]isoxazol-3,6-ylene, isothiazolo[5,4-d]isothiazol-3,6-ylene, imidazo[2,1-b][1,3,4]thiadiazol-2,5-ylene, imidazo[2,1-b][1,3,4]thiadiazol-2,6-ylene, 6H-pyrrolo[3,2-d]isoxazol-3,6-ylene, 1H-imidazo[1,5-a]imidazol-1,5-ylene, imidazo[2,1-b]oxazol-2,5-ylene, imidazo[2,1-b]thiazol-2,5-ylene, 1H-imidazo[1,2-a]imidazol-2,5-ylene, 1H-imidazo[1,2-a]imidazol-1,5-ylene, thieno[3,2-b]furan-3,6-ylene, or thiazolo[5,4-d]thiazol-2,5-ylene, each of which is independently and optionally substituted with one or more substituents Q.

In certain embodiments, A¹ is selected from:

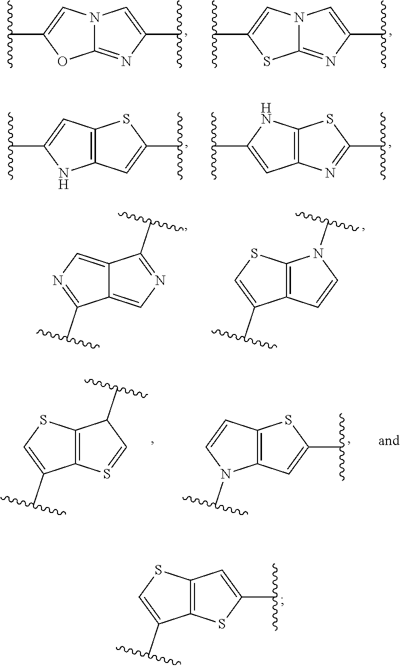

wherein each divalent moiety is optionally substituted with one, two, three, or four, in one embodiment, one or two, substituents Q.

In certain embodiments, A² is a bond. In certain embodiments, A² is C₁₋₆ alkylene, optionally substituted with one or more substituents Q. In certain embodiments, A² is C₂₋₆ alkenylene, optionally substituted with one or more substituents Q. In certain embodiments, A² is ethenylene, optionally substituted with one or more substituents Q. In certain embodiments, A² is C₂₋₆ alkynylene, optionally substituted with one or more substituents Q. In certain embodiments, A² is ethynylene. In certain embodiments, A² is C₂₋₂₀ cycloalkylene, optionally substituted with one or more substituents Q. In certain embodiments, A² is C₆₋₂₀ arylene, optionally substituted with one or more substituents Q. In certain embodiments, A² is 6-membered arylene, optionally substituted with one or more substituents Q. In certain embodiments, A² is phenylene, optionally substituted with one or more substituents Q. In certain embodiments, A² is 1,4-phenylene, optionally substituted with one or more substituents Q. In certain embodiments, A² is bicyclic arylene, optionally substituted with one or more substituents Q. In certain embodiments, A² is naphthylene, optionally substituted with one or more substituents Q. In certain embodiments, A² is tricyclic arylene, optionally substituted with one or more substituents Q. In certain embodiments, A² is tricyclic arylene, optionally substituted with one or more substituents Q. In certain embodiments, A² is fluorenylene, optionally substituted with one or more substituents Q. In certain embodiments, A² is 9,9-difluoro-9H-fluorenylene, optionally substituted with one or more substituents Q. In certain embodiments, A² is 9,9-difluoro-9H-fluorenylene, optionally substituted with one or more substituents Q. In certain embodiments, A² is 9,9-difluoro-9H-fluorenyl-2,7-ene, optionally substituted with one or more substituents Q. In certain embodiments, A² is tetracyclic arylene, optionally substituted with one or more substituents Q. In certain embodiments, A² is heteroarylene; optionally substituted with one or more substituents Q. In certain embodiments, A² is monocyclic heteroarylene; optionally substituted with one or more substituents Q. In certain embodiments, A² is bicyclic heteroarylene; optionally substituted with one or more substituents Q. In certain embodiments, A² is 5,5-fused heteroarylene; optionally substituted with one or more substituents Q. In certain embodiments, A² is 5,6-fused heteroarylene; optionally substituted with one or more substituents Q. In certain embodiments, A² is 6,6-fused heteroarylene; optionally substituted with one or more substituents Q. In certain embodiments, A² is tricyclic heteroarylene; optionally substituted with one or more substituents Q. In certain embodiments, A² is tetracyclic heteroarylene; optionally substituted with one or more substituents Q. In certain embodiments, A² is heterocyclylene, optionally substituted with one or more substituents Q. In certain embodiments, A² is monocyclic heterocyclylene, optionally substituted with one or more substituents Q. In certain embodiments, A² is bicyclic heterocyclylene, optionally substituted with one or more substituents Q. In certain embodiments, A² is tricyclic heterocyclylene, optionally substituted with one or more substituents Q. In certain embodiments, A² is tetracyclic heterocyclylene, optionally substituted with one or more substituents Q.

In certain embodiments, A² is thieno[3,2-b]thienylene, pyrrolo[3,4-c]pyrrolylene, 4H-thieno[3,2-b]pyrrolylene, 6H-thieno[2,3-b]pyrrolylene, imidazo[2,1-b]oxazolylene, imidazo[2,1-b]thiazolylene, or 4H-pyrrolo[3,2-d]thiazolylene, each of which is independently and optionally substituted with one or more substituents Q. In certain embodiments, A² is thieno[3,2-b]thien-2,6-ylene, thieno[3,2-b]thien-3,6-ylene, pyrrolo[3,4-c]pyrrol-1,4-ylene, 4H-thieno[3,2-b]pyrrol-2,5-ylene, 6H-thieno[2,3-b]pyrrol-3,6-ylene, imidazo[2,1-b]oxazol-2,6-ylene, imidazo[2,1-b]thiazol-2,6-ylene, or 4H-pyrrolo[3,2-d]thiazol-2,5-ylene, each of which is independently and optionally substituted with one or more substituents Q. In certain embodiments, A² is 3H-pyrrolizinylene, 4H-furo[3,2-b]pyrrolylene, furo[3,2-b]furanylene, 1,4-dihydropyrrolo[3,2-b]pyrrolylene, 5H-pyrrolo[1,2-c]imidazolylene, 4H-furo[3,2-b]pyrrolylene, 6H-pyrrolo[1,2-b]pyrazolylene, 5H-pyrrolo[1,2-a]imidazolylene, thieno[3,2-b]furanylene, 1H-furo[3,2-c]pyrazolylene, 1H-thieno[3,2-c]pyrazolylene, 1,4-dihydropyrrolo[3,2-c]pyrazolylene, 1H-imidazo[1,2-a]imidazolylene, pyrazolo[5,1-b]oxazolylene, pyrazolo[5,1-b]thiazolylene, 5H-imidazo[1,2-b]pyrazolylene, imidazo[1,2-b]isoxazolylene, imidazo[1,2-b]isothiazolylene, imidazo[1,5-b]isoxazolylene, imidazo[1,5-b]isothiazolylene, imidazo[5,1-b]oxazolylene, imidazo[5,1-b]thiazolylene, 1H-imidazo[1,5-a]imidazolylene, 6H-pyrrolo[3,2-d]isoxazolylene, 6H-pyrrolo[3,2-d]isothiazolylene, pyrrolo[2,1-b][1,3,4]oxadiazolylene, pyrrolo[2,1-b][1,3,4]thiadiazolylene, 1H-pyrrolo[1,2-b][1,2,4]triazolylene, 3H-furo[2,3-d]imidazolylene, 3H-thieno[2,3-d]imidazolylene, 3,4-dihydropyrrolo[2,3-d]imidazolylene, furo[3,2-d]thiazolylene, thieno[3,2-d]thiazolylene, 4H-pyrrolo[3,2-d]thiazolylene, 4H-pyrazolo[3,4-d]isoxazolylene, 4H-pyrazolo[3,4-d]isothiazolylene, 1,4-dihydropyrazolo[4,3-c]pyrazolylene, isoxazolo[5,4-d]isoxazolylene, isothiazolo[5,4-d]isothiazolylene, imidazo[2,1-b][1,3,4]thiadiazolylene, 1H-imidazo[1,5-a]imidazolylene, imidazo[2,1-b]oxazolylene, imidazo[2,1-b]thiazolylene, 1H-imidazo[1,2-a]imidazolylene, 1H-imidazo[1,2-a]imidazolylene, thieno[3,2-b]furanylene, or thiazolo[5,4-d]thiazolylene, each of which is independently and optionally substituted with one or more substituents Q.

In certain embodiments, A² is imidazo[2,1-b]thiazol-5,6-ylene, 3H-pyrrolizin-1,5-ylene, 3H-pyrrolizin-2,6-ylene, 4H-furo[3,2-b]pyrrol-2,5-ylene, 4H-furo[3,2-b]pyrrol-3,6-ylene, furo[3,2-b]furan-2,5-ylene, furo[3,2-b]furan-3,6-ylene, 1,4-dihydropyrrolo[3,2-b]pyrrol-2,5-ylene, 1,4-dihydropyrrolo[3,2-b]pyrrol-3,6-ylene, 5H-pyrrolo[1,2-c]imidazol-3,7-ylene, 4H-furo[3,2-b]pyrrol-2,4-ylene, 4H-furo[3,2-b]pyrrol-2,5-ylene, 4H-furo[3,2-b]pyrrol-3,4-ylene, 4H-furo[3,2-b]pyrrol-3,6-ylene, 6H-pyrrolo[1,2-b]pyrazol-2,5-ylene, 5H-pyrrolo[1,2-a]imidazol-2,6-ylene, 5H-pyrrolo[1,2-a]imidazol-3,7-ylene, thieno[3,2-b]furan-2,5-ylene, thieno[3,2-b]furan-3,6-ylene, 1H-furo[3,2-c]pyrazol-3,6-ylene, 1H-thieno[3,2-c]pyrazol-3,6-ylene, 1,4-dihydropyrrolo[3,2-c]pyrazol-3,6-ylene, 1H-imidazo[1,2-a]imidazol-2,6-ylene, pyrazolo[5,1-b]oxazol-2,6-ylene, pyrazolo[5,1-b]oxazol-3,7-ylene, pyrazolo[5,1-b]thiazol-2,6-ylene, pyrazolo[5,1-b]thiazol-3,7-ylene, 5H-imidazo[1,2-b]pyrazol-2,6-ylene, 5H-imidazo[1,2-b]pyrazol-3,7-ylene, imidazo[1,2-b]isoxazol-2,6-ylene, imidazo[1,2-b]isoxazol-3,7-ylene, imidazo[1,2-b]isothiazol-2,6-ylene, imidazo[1,2-b]isothiazol-3,7-ylene, imidazo[1,5-b]isoxazol-3,7-ylene, imidazo[1,5-b]isothiazol-3,6-ylene, imidazo[5,1-b]oxazol-3,7-ylene, imidazo[5,1-b]thiazol-3,7-ylene, 1H-imidazo[1,5-a]imidazol-3,7-ylene, 6H-pyrrolo[3,2-d]isoxazol-3,6-ylene, 6H-pyrrolo[3,2-d]isothiazol-3,6-ylene, pyrrolo[2,1-b][1,3,4]oxadiazol-2,6-ylene, pyrrolo[2,1-b][1,3,4]thiadiazol-2,6-ylene, 1H-pyrrolo[1,2-b][1,2,4]triazol-1,5-ylene, 1H-pyrrolo[1,2-b][1,2,4]triazol-2,6-ylene, 3H-furo[2,3-d]imidazol-2,5-ylene, 3H-furo[2,3-d]imidazol-3,6-ylene, 3H-thieno[2,3-d]imidazol-2,5-ylene, 3H-thieno[2,3-d]imidazol-3,6-ylene, 3,4-dihydropyrrolo[2,3-d]imidazol-2,5-ylene, 3,4-dihydropyrrolo[2,3-d]imidazol-3,6-ylene, furo[3,2-d]thiazol-2,5-ylene, thieno[3,2-d]thiazol-2,5-ylene, 4H-pyrrolo[3,2-d]thiazol-2,5-ylene, 4H-pyrazolo[3,4-d]isoxazol-3,6-ylene, 4H-pyrazolo[3,4-d]isothiazol-3,6-ylene, 1,4-dihydropyrazolo[4,3-c]pyrazol-1,4-ylene, 1,4-dihydropyrazolo[4,3-c]pyrazol-3,6-ylene, isoxazolo[5,4-d]isoxazol-3,6-ylene, isothiazolo[5,4-d]isothiazol-3,6-ylene, imidazo[2,1-b][1,3,4]thiadiazol-2,5-ylene, imidazo[2,1-b][1,3,4]thiadiazol-2,6-ylene, 6H-pyrrolo[3,2-d]isoxazol-3,6-ylene, 1H-imidazo[1,5-a]imidazol-1,5-ylene, imidazo[2,1-b]oxazol-2,5-ylene, imidazo[2,1-b]thiazol-2,5-ylene, 1H-imidazo[1,2-a]imidazol-2,5-ylene, 1H-imidazo[1,2-a]imidazol-1,5-ylene, thieno[3,2-b]furan-3,6-ylene, or thiazolo[5,4-d]thiazol-2,5-ylene, each of which is independently and optionally substituted with one or more substituents Q.

In certain embodiments, E is a bond. In certain embodiments, E is C₁₋₆ alkylene, optionally substituted with one or more substituents Q. In certain embodiments, E is C₂₋₆ alkenylene, optionally substituted with one or more substituents Q. In certain embodiments, E is ethenylene, optionally substituted with one or more substituents Q. In certain embodiments, E is C₂₋₆ alkynylene, optionally substituted with one or more substituents Q. In certain embodiments, E is ethynylene. In certain embodiments, E is C₂₋₂₀ cycloalkylene, optionally substituted with one or more substituents Q. In certain embodiments, E is C₆₋₂₀ arylene, optionally substituted with one or more substituents Q. In certain embodiments, E is 6-membered arylene, optionally substituted with one or more substituents Q. In certain embodiments, E is phenylene, optionally substituted with one or more substituents Q. In certain embodiments, E is 1,4-phenylene, optionally substituted with one or more substituents Q. In certain embodiments, E is bicyclic arylene, optionally substituted with one or more substituents Q. In certain embodiments, E is naphthylene, optionally substituted with one or more substituents Q. In certain embodiments, E is tricyclic arylene, optionally substituted with one or more substituents Q. In certain embodiments, E is tricyclic arylene, optionally substituted with one or more substituents Q. In certain embodiments, E is fluorenylene, optionally substituted with one or more substituents Q. In certain embodiments, E is 9,9-difluoro-9H-fluorenylene, optionally substituted with one or more substituents Q. In certain embodiments, E is 9,9-difluoro-9H-fluorenylene, optionally substituted with one or more substituents Q. In certain embodiments, E is 9,9-difluoro-9H-fluorenyl-2,7-ene, optionally substituted with one or more substituents Q. In certain embodiments, E is tetracyclic arylene, optionally substituted with one or more substituents Q. In certain embodiments, E is heteroarylene; optionally substituted with one or more substituents Q. In certain embodiments, E is monocyclic heteroarylene; optionally substituted with one or more substituents Q. In certain embodiments, E is bicyclic heteroarylene; optionally substituted with one or more substituents Q. In certain embodiments, E is 5,5-fused heteroarylene; optionally substituted with one or more substituents Q. In certain embodiments, E is 5,6-fused heteroarylene; optionally substituted with one or more substituents Q. In certain embodiments, E is 6,6-fused heteroarylene; optionally substituted with one or more substituents Q. In certain embodiments, E is tricyclic heteroarylene; optionally substituted with one or more substituents Q. In certain embodiments, E is tetracyclic heteroarylene; optionally substituted with one or more substituents Q. In certain embodiments, E is heterocyclylene, optionally substituted with one or more substituents Q. In certain embodiments, E is monocyclic heterocyclylene, optionally substituted with one or more substituents Q. In certain embodiments, E is bicyclic heterocyclylene, optionally substituted with one or more substituents Q. In certain embodiments, E is tricyclic heterocyclylene, optionally substituted with one or more substituents Q. In certain embodiments, E is tetracyclic heterocyclylene, optionally substituted with one or more substituents Q.

In certain embodiments, E is selected from:

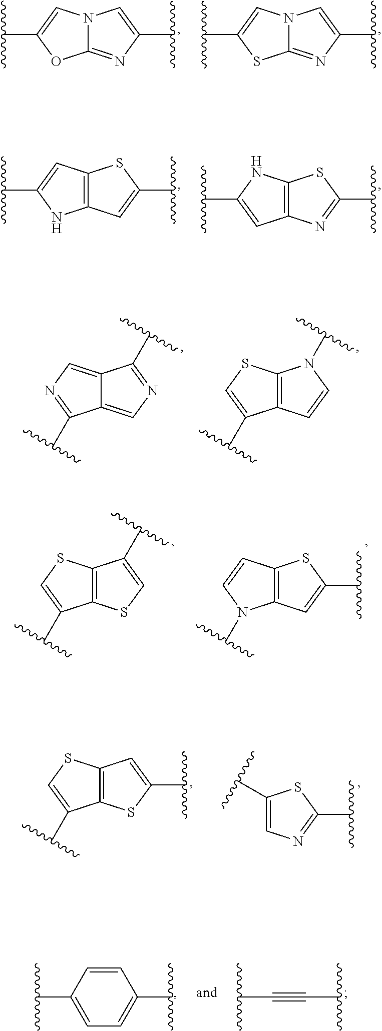

wherein each divalent moiety is optionally substituted with one, two, three, or four, in one embodiment, one or two, R^{7a} groups, where R^{7a} is as defined herein. In certain embodiments, each R^{7a} is independently chloro, fluoro, nitro, amino, methyl, trifluoromethyl, phenyl, or methoxy.

In certain embodiments, E is selected from:

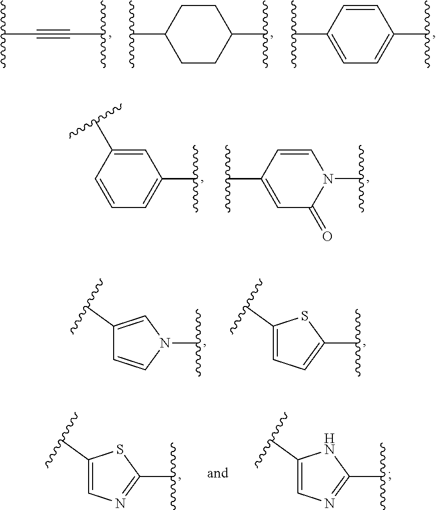

wherein each divalent moiety is optionally substituted with one, two, three, or four, in one embodiment, one or two, R^{7a} groups, where R^{7a} is as defined herein. In certain embodiments, each R^{7a} is independently oxo, chloro, fluoro, nitro, hydroxy, amino, methyl, trifluoromethyl, cyclohexyl, phenyl, methoxy, or methoxycarbonyl.

In certain embodiments, A¹, A², or the divalent moiety

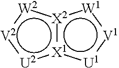

is selected from:

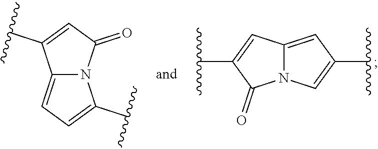 [SMILES: *C1=CC(=O)n2c(*)ccc21 ; *C1=Cc2cc(*)cn2C1=O]

wherein each divalent moiety is optionally substituted with one, two, three, or four, in one embodiment, one or two, R^{7a} groups, where R^{7a} is as defined herein. In certain embodiments, each R^{7a} is independently oxo, chloro, fluoro, nitro, amino, methyl, trifluoromethyl, cyclohexyl, phenyl, or methoxy.

In certain embodiments, A¹, A², or the divalent moiety

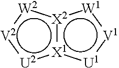

is selected from:

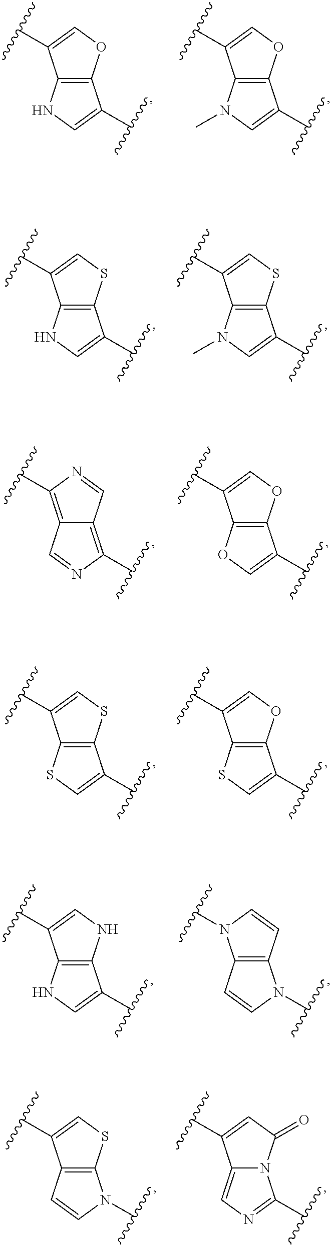

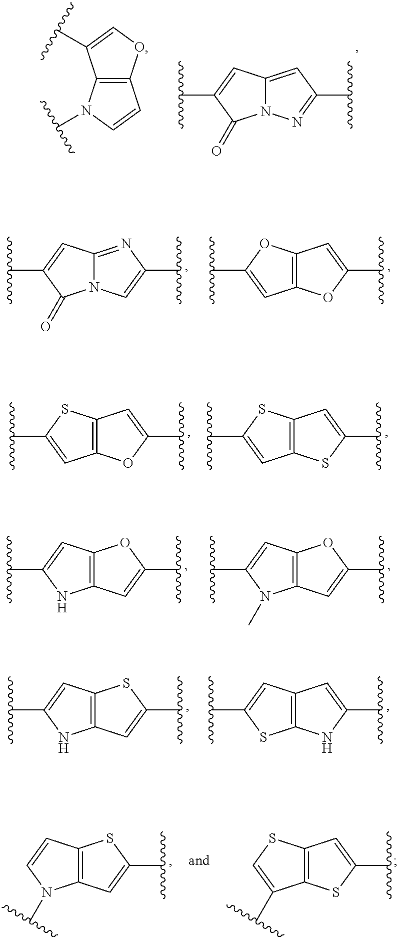

wherein each divalent moiety is optionally substituted with one, two, three, or four, in one embodiment, one or two, R^{7a} groups, where R^{7a} is as defined herein. In certain embodiments, each R^{7a} is independently oxo, chloro, fluoro, nitro, amino, methyl, trifluoromethyl, cyclohexyl, phenyl, or methoxy.

In certain embodiments, A¹, A², or the divalent moiety

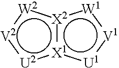

is selected from:

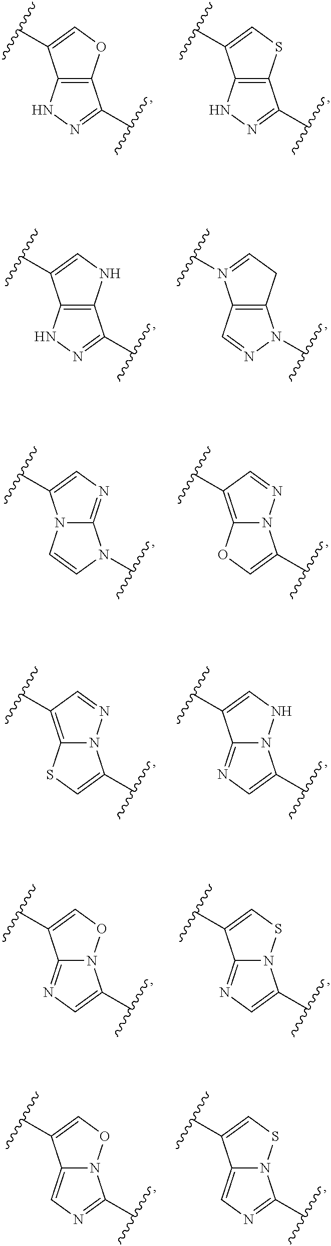

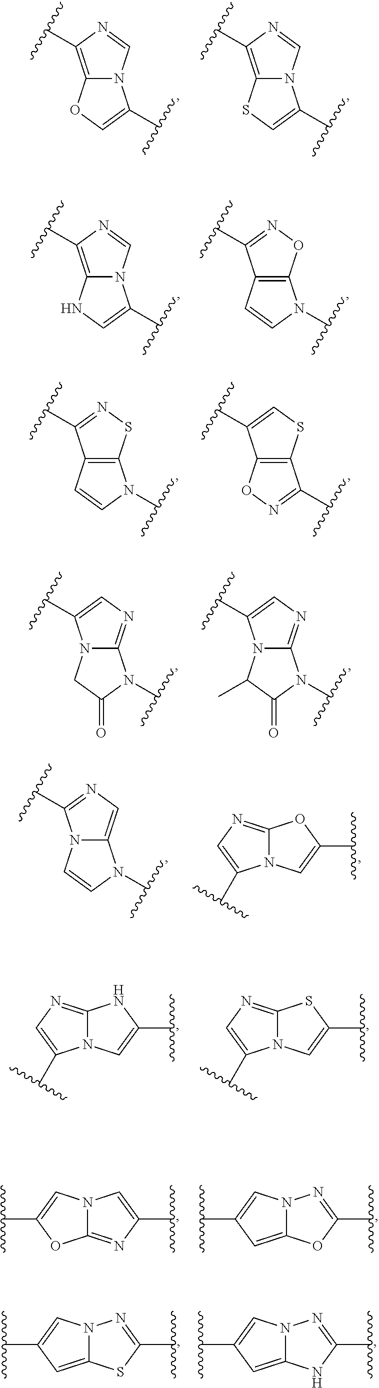

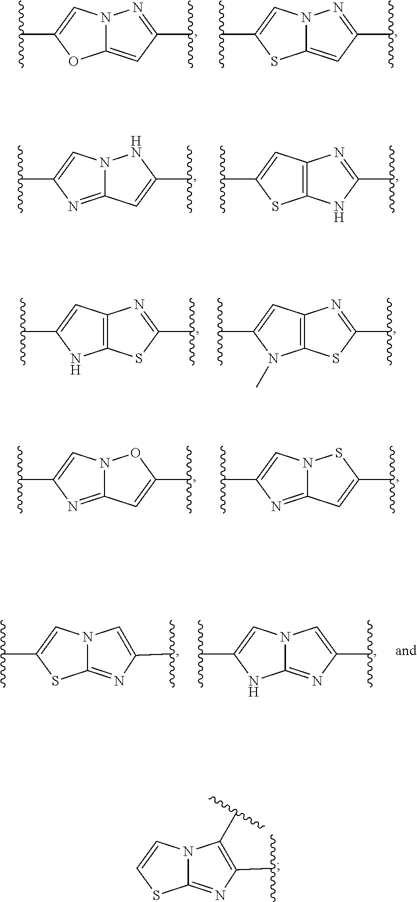

wherein each divalent moiety is optionally substituted with one, two, three, or four, in one embodiment, one or two, R^{7a} groups, where R^{7a} is as defined herein. In certain embodiments, each R^{7a} is independently oxo, chloro, fluoro, nitro, amino, methyl, trifluoromethyl, cyclohexyl, phenyl, or methoxy.

In certain embodiments, A¹, A², or the divalent moiety

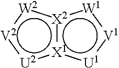

is selected from:

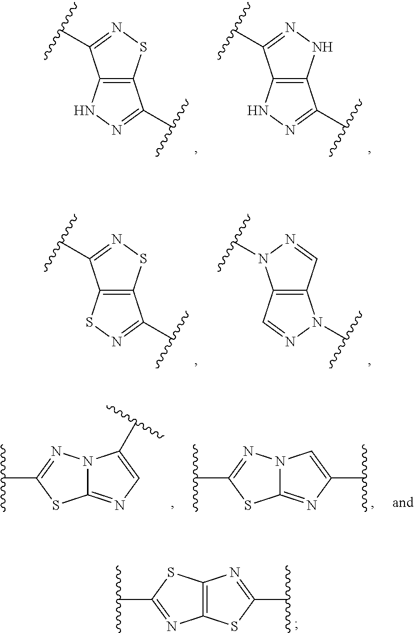

wherein each divalent moiety is optionally substituted with one, two, three, or four, in one embodiment, one or two, R^{7a} groups, where R^{7a} is as defined herein. In certain embodiments, each R^{7a} is independently oxo, chloro, fluoro, nitro, amino, methyl, trifluoromethyl, cyclohexyl, phenyl, or methoxy.

In certain embodiments, L¹ is a bond. In certain embodiments, L¹ is not a bond. In certain embodiments, L¹ is C₁₋₆ alkylene, optionally substituted with one or more substituents Q. In certain embodiments, L¹ is C₂₋₆ alkenylene, optionally substituted with one or more substituents Q. In certain embodiments, L¹ is C₂₋₆ alkynylene, optionally substituted with one or more substituents Q. In certain embodiments, L¹ is ethynylene. In certain embodiments, L¹ is C₃₋₇ cycloalkylene, optionally substituted with one or more substituents Q. In certain embodiments, L¹ is C₆₋₁₄ arylene, optionally substituted with one or more substituents Q. In certain embodiments, L¹ is heteroarylene, optionally substituted with one or more substituents Q. In certain embodiments, L¹ is five- or six-membered heteroarylene, each optionally substituted with one or more substituents Q. In certain embodiments, L¹ is pyrazolylene, imidazolylene, or triazolylene, each optionally substituted with one or more substituents Q. In certain embodiments, L¹ is not thiazolylene. In certain embodiments, L¹ is pyrazolylene, imidazolylene, oxazolylene, 1,3,4-oxadiazolylene, 1,2,3-triazolylene, or 1,2,4-triazolylene, each optionally substituted with one or more substituents Q. In certain embodiments, L¹ is pyrazol-3,5-ylene, oxazol-2,5-ylene, imidazol-2,4-ylene, 1,3,4-oxadiazol-2,5-ylene, 1,2,3-triazol-1,4-ylene, 1,2,3-triazol-2,4-ylene, or 1,2,4-triazol-3,5-ylene, each optionally substituted with one or more substituents Q. In certain embodiments, L¹ is 5,6-fused heteroarylene, each optionally substituted with one or more substituents Q. In certain embodiments, L¹ is benzimidazolylene, each optionally substituted with one or more substituents Q. In certain embodiments, L¹ is benzimidazolyl-2,5-ene, each optionally substituted with one or more substituents Q. In certain embodiments, L¹ is heterocyclylene; optionally substituted with one or more substituents Q.

In certain embodiments, L¹ is —C(O)—. In certain embodiments, L¹ is —C(O)O—. In certain embodiments, L¹ is —C(O)NR^{1a}—, where R^{1a} is as defined herein. In certain embodiments, L¹ is —C(O)NH—. In certain embodiments, L¹ is —C(═NR^{1a})NR^{1c}—, where R^{1a} and R^{1c} are each as defined herein. In certain embodiments, L¹ is —O—. In certain embodiments, L¹ is —OC(O)O—. In certain embodiments, L¹ is —OC(O)NR^{1a}—, where R^{1a} is as defined herein. In certain embodiments, L¹ is —OC(═NR^{1a})NR^{1c}—, where R^{1a} and R^{1c} are each as defined herein. In certain embodiments, L¹ is —OP(O)(OR^{1a})—, where R^{1a} is as defined herein. In certain embodiments, L¹ is —NR^{1a}—, where R^{1a} is as defined herein. In certain embodiments, L¹ is —NR^{1a}C(O)NR^{1c}—, where R^{1a} and R^{1c} are each as defined herein. In certain embodiments, L¹ is —NR^{a}C(═NR^{1b})NR^{1c}—, where R^{1a}, R^{1b}, and R^{1c} are each as defined herein. In certain embodiments, L¹ is —NR^{1a}S(O)NR^{1c}—, where R^{1a} and R^{1c} are each as defined herein. In certain embodiments, L¹ is —NR^{1a}S(O)₂NR^{1c}—, where R^{1a} and R^{1c} are each as defined herein. In certain embodiments, L¹ is —S—. In certain embodiments, L¹ is —S(O)—. In certain embodiments, L¹ is —S(O)₂—. In certain embodiments, L¹ is —S(O)NR^{1a}—, where R^{1a} is as defined herein. In certain embodiments, L¹ is —S(O)₂NR^{1a}—, where R^{1a} is as defined herein.

In certain embodiments, L² is a bond. In certain embodiments, L² is not a bond. In certain embodiments, L² is C₁₋₆ alkylene, optionally substituted with one or more substituents Q. In certain embodiments, L² is C₂₋₆ alkenylene, optionally substituted with one or more substituents Q. In certain embodiments, L² is C₂₋₆ alkynylene, optionally substituted with one or more substituents Q. In certain embodiments, L² is ethynylene. In certain embodiments, L² is C₃₋₇ cycloalkylene, optionally substituted with one or more substituents Q. In certain embodiments, L² is C₆₋₁₄ arylene, optionally substituted with one or more substituents Q. In certain embodiments, L² is heteroarylene, optionally substituted with one or more substituents Q. In certain embodiments, L² is five- or six-membered heteroarylene, each optionally substituted with one or more substituents Q. In certain embodiments, L² is pyrazolylene, imidazolylene, or triazolylene, each optionally substituted with one or more substituents Q. In certain embodiments, L² is not thiazolylene. In certain embodiments, L² is pyrazolylene, imidazolylene, oxazolylene, 1,3,4-oxadiazolylene, 1,2,3-triazolylene, or 1,2,4-triazolylene, each optionally substituted with one or more substituents Q. In certain embodiments, L² is pyrazol-3,5-ylene, oxazol-2,5-ylene, imidazol-2,4-ylene, 1,3,4-oxadiazol-2,5-ylene, 1,2,3-triazol-1,4-ylene, 1,2,3-triazol-2,4-ylene, or 1,2,4-triazol-3,5-ylene, each optionally substituted with one or more substituents Q. In certain embodiments, L² is 5,6-fused heteroarylene, each optionally substituted with one or more substituents Q. In certain embodiments, L² is benzimidazolylene, each optionally substituted with one or more substituents Q. In certain embodiments, L² is benzimidazolyl-2,5-ene, each optionally substituted with one or more substituents Q. In certain embodiments, L² is heterocyclylene; optionally substituted with one or more substituents Q.

In certain embodiments, L² is —C(O)—. In certain embodiments, L² is —C(O)O—. In certain embodiments, L² is —C(O)NR^{1a}—, where R^{1a} is as defined herein. In certain embodiments, L² is —C(O)NH—. In certain embodiments, L² is —C(═NR^{1a})NR^{1c}—, where R^{1a} and R^{1c} are each as defined herein. In certain embodiments, L² is —O—. In certain embodiments, L² is —OC(O)O—. In certain embodiments, L² is —OC(O)NR^{1a}—, where R^{1a} is as defined herein. In certain embodiments, L² is —OC(═NR^{1a})NR¹—, where R^{1a} and R^{1c} are each as defined herein. In certain embodiments, L² is —OP(O)(OR^{1a})—, where R^{1a} is as defined herein. In certain embodiments, L² is —NR^{1a}—, where R^{1a} is as defined herein. In certain embodiments, L² is —NR^{1a}C(O)NR^{1c}—, where R^{1a} and R^{1c} are each as defined herein. In certain embodiments, L² is —NR^{a}C(═NR^{1b})NR^{1c}—, where R^{1a}, R^{1b}, and R^{1c} are each as defined herein. In certain embodiments, L² is —NR^{1a}S(O)NR^{1c}—, where R^{1a} and R^{1c} are each as defined herein. In certain embodiments, L² is —NR^{1a}S(O)₂NR^{1c}—, where R^{1a} and R^{1c} are each as defined herein. In certain embodiments, L² is —S—. In certain embodiments, L² is —S(O)—. In certain embodiments, L² is —S(O)₂—. In certain embodiments, L² is —S(O)NR^{1a}—, where R^{1a} is as defined herein. In certain embodiments, L² is —S(O)₂NR^{1a}—, where R^{1a} is as defined herein.

In certain embodiments, T³ is a bond. In certain embodiments, T³ is C. In certain embodiments, T³ is N. In certain embodiments, T³ is O. In certain embodiments, T³ is S. In certain embodiments, T³ is CR^{7a}, wherein R^{7a} is as defined herein. In certain embodiments, T³ is CH. In certain embodiments, T³ is NR^{7a}, wherein R^{7a} is as defined herein. In certain embodiments, T³ is NH.

In certain embodiments, U¹ is C. In certain embodiments, U¹ is N. In certain embodiments, U¹ is O. In certain embodiments, U¹ is S. In certain embodiments, U¹ is CR^{7a}, wherein R^{7a} is as defined herein. In certain embodiments, U¹ is CH. In certain embodiments, U¹ is NR^{7a}, wherein R^{7a} is as defined herein. In certain embodiments, U¹ is NH.

In certain embodiments, U² is C. In certain embodiments, U² is N. In certain embodiments, U² is O. In certain embodiments, U² is S. In certain embodiments, U² is CR^{7a}, wherein R^{7a} is as defined herein. In certain embodiments, U² is CH. In certain embodiments, U² is NR^{7a}, wherein R^{7a} is as defined herein. In certain embodiments, U² is NH.

In certain embodiments, U³ is C. In certain embodiments, U³ is N. In certain embodiments, U³ is O. In certain embodiments, U³ is S. In certain embodiments, U³ is CR^{7a}, wherein R^{7a} is as defined herein. In certain embodiments, U³ is CH. In certain embodiments, U³ is NR^{7a}, wherein R^{7a} is as defined herein. In certain embodiments, U³ is NH.

In certain embodiments, V¹ is C. In certain embodiments, V¹ is N. In certain embodiments, V¹ is O. In certain embodiments, V¹ is S. In certain embodiments, V¹ is CR^{7a}, wherein R^{7a} is as defined herein. In certain embodiments, V¹ is CH. In certain embodiments, V¹ is NR^{7a}, wherein R^{7a} is as defined herein. In certain embodiments, V¹ is NH.

In certain embodiments, V² is C. In certain embodiments, V² is N. In certain embodiments, V² is O. In certain embodiments, V² is S. In certain embodiments, V² is CR^{7a}, wherein R^{7a} is as defined herein. In certain embodiments, V² is CH. In certain embodiments, V² is NR^{7a}, wherein R^{7a} is as defined herein. In certain embodiments, V² is NH.

In certain embodiments, V³ is C. In certain embodiments, V³ is N. In certain embodiments, V³ is O. In certain embodiments, V³ is S. In certain embodiments, V³ is CR^{7a}, wherein R^{7a} is as defined herein. In certain embodiments, V³ is CH. In certain embodiments, V³ is NR^{7a}, wherein R^{7a} is as defined herein. In certain embodiments, V³ is NH.

In certain embodiments, W¹ is C. In certain embodiments, W¹ is N. In certain embodiments, W¹ is O. In certain embodiments, W¹ is S. In certain embodiments, W¹ is CR^{7a}, wherein R^{7a} is as defined herein. In certain embodiments, W¹ is CH. In certain embodiments, W¹ is NR^{7a}, wherein R^{7a} is as defined herein. In certain embodiments, W¹ is NH.

In certain embodiments, W² is C. In certain embodiments, W² is N. In certain embodiments, W² is O. In certain embodiments, W² is S. In certain embodiments, W² is CR^{7a}, wherein R^{7a} is as defined herein. In certain embodiments, W² is CH. In certain embodiments, W² is NR^{7a}, wherein R^{7a} is as defined herein. In certain embodiments, W² is NH.

In certain embodiments, W³ is C. In certain embodiments, W³ is N. In certain embodiments, W³ is O. In certain embodiments, W³ is S. In certain embodiments, W³ is CR^{7a}, wherein R^{7a} is as defined herein. In certain embodiments, W³ is CH. In certain embodiments, W³ is NR^{7a}, wherein R^{7a} is as defined herein. In certain embodiments, W³ is NH.

In certain embodiments, X¹ is C. In certain embodiments, X¹ is N.

In certain embodiments, X² is C. In certain embodiments, X² is N.

In certain embodiments, X³ is C. In certain embodiments, X³ is N.

In certain embodiments, Y³ is C. In certain embodiments, Y³ is N. In certain embodiments, Y³ is O. In certain embodiments, Y³ is S. In certain embodiments, Y³ is CR^{7a}, wherein R^{7a} is as defined herein. In certain embodiments, Y³ is CH. In certain embodiments, Y³ is NR^{7a}, wherein R^{7a} is as defined herein. In certain embodiments, Y³ is NH.

In certain embodiments, Z¹ is a bond. In certain embodiments, Z¹ is —O—. In certain embodiments, Z¹ is —S—. In certain embodiments, Z¹ is —S(O)—. In certain embodiments, Z¹ is —S(O₂)—. In certain embodiments, Z¹ is —N(R^{N})—, where R^{N} is as defined herein. In certain embodiments, Z¹ is —NH—. In certain embodiments, Z¹ is —N(C(O)R^{a})—, where R^{1a} is as defined herein. In certain embodiments, Z¹ is —N(C(O)C₁₋₆alkyl)-. In certain embodiments, Z¹ is —N(C(O)CH₃)—.

In certain embodiments, Z² is a bond. In certain embodiments, Z² is —O—. In certain embodiments, Z² is —S—. In certain embodiments, Z² is —S(O)—. In certain embodiments, Z² is —S(O₂)—. In certain embodiments, Z² is —N(R^{N})—, where R^{N} is as defined herein. In certain embodiments, Z² is —NH—. In certain embodiments, Z² is —N(C(O)R^{1a})—, where R^{1a} is as defined herein. In certain embodiments, Z² is —N(C(O)C₁₋₆alkyl)-. In certain embodiments, Z² is —N(C(O)CH₃)—.

In certain embodiments, m is 1. In certain embodiments, m is 2. In certain embodiments, m is 3. In certain embodiments, m is 4.

In certain embodiments, n is 1. In certain embodiments, n is 2. In certain embodiments, n is 3. In certain embodiments, n is 4.

In certain embodiments, r is 0. In certain embodiments, r is 1. In certain embodiments, r is 2. In certain embodiments, r is 3. In certain embodiments, r is 4.

In certain embodiments, s is 0. In certain embodiments, s is 1. In certain embodiments, s is 2. In certain embodiments, s is 3. In certain embodiments, s is 4. In certain embodiments, s is 5. In certain embodiments, s is 6. In certain embodiments, s is 7.

In certain embodiments, t is 0. In certain embodiments, t is 1. In certain embodiments, t is 2. In certain embodiments, t is 3. In certain embodiments, t is 4. In certain embodiments, t is 5. In certain embodiments, t is 6. In certain embodiments, t is 7.

In certain embodiments, u is 1. In certain embodiments, u is 2.

In certain embodiments, the moiety

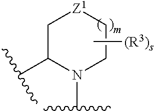

has the structure of:

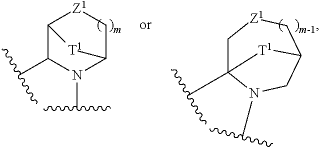

wherein Z¹ and m are each as defined herein; and each T¹ is independently a bond, —O—, —NR^{N}—, —S—, C₁₋₆ alkylene, C₁₋₆ heteroalkylene, C₂₋₆ alkenylene, or C₂₋₆ heteroalkenylene, where R^{N} is as defined herein.

In certain embodiments, the moiety

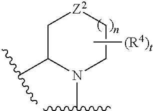

has the structure of:

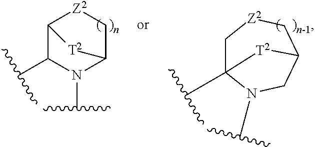

wherein Z² and n are each as defined herein; and each T² is independently a bond, —O—, —NR^{N}—, —S—, C₁₋₆ alkylene, C₁₋₆ heteroalkylene, C₂₋₆ alkenylene, or C₂₋₆ heteroalkenylene, where R^{N} is as defined herein.

In one embodiment, the moiety

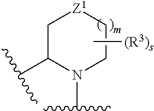

has the structure of

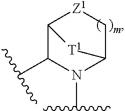

and the moiety

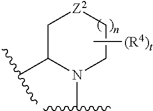

has the structure of

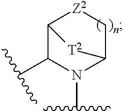

wherein T¹, T² Z¹, Z², m, and n are each as defined herein.

In another embodiment, the moiety

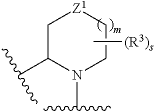

has the structure of

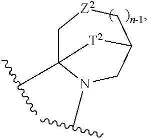

and the moiety

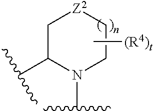

has the structure of

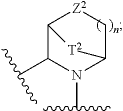

wherein T¹, T² Z¹, Z², m, and n are each as defined herein.

In yet another embodiment, the moiety

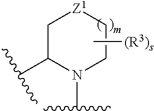

has the structure of

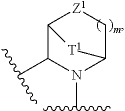

and the moiety

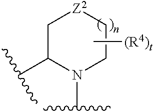

has the structure of

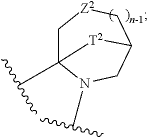

wherein T¹, T² Z¹, Z², m, and n are each as defined herein.

In still another embodiment, the moiety

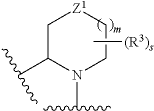

has the structure of

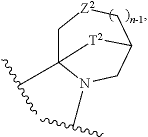

and the moiety

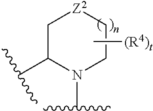

has the structure of

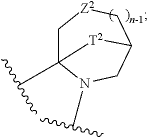

wherein T¹, T² Z¹, Z², m, and n are each as defined herein.

In certain embodiments, T is a bond. In certain embodiments, T is —O—. In certain embodiments, T is —NR^{N}—, where R^{N} is as defined herein. In certain embodiments, T is —S—. In certain embodiments, T is C₁₋₆ alkylene, optionally substituted with one or more substituents Q. In certain embodiments, T is methylene or ethylene. In certain embodiments, T is C₁₋₆ heteroalkylene, optionally substituted with one or more substituents Q. In certain embodiments, T is C₂₋₆ alkenylene, optionally substituted with one or more substituents Q. In certain embodiments, T is C₂₋₆ heteroalkenylene, optionally substituted with one or more substituents Q. In certain embodiments, each T is independently —O—, —NR^{N}—, —S—, C₁₋₆ alkylene, C₁₋₆ heteroalkylene, C₂₋₆ alkenylene, or C₂₋₆ heteroalkenylene, where R^{N} is as defined herein.

In certain embodiments, T² is a bond. In certain embodiments, T² is —O—. In certain embodiments, T² is —NR^{N}—, where R^{N} is as defined herein. In certain embodiments, T² is —S—. In certain embodiments, T² is C₁₋₆ alkylene, optionally substituted with one or more substituents Q. In certain embodiments, T² is methylene or ethylene. In certain embodiments, T² is C₁₋₆ heteroalkylene, optionally substituted with one or more substituents Q. In certain embodiments, T² is C₂₋₆ alkenylene, optionally substituted with one or more substituents Q. In certain embodiments, T² is C₂₋₆ heteroalkenylene, optionally substituted with one or more substituents Q. In certain embodiments, each T² is independently —O—, —NR^{N}—, —S—, C₁₋₆ alkylene, C₁₋₆ heteroalkylene, C₂₋₆ alkenylene, or C₂₋₆ heteroalkenylene, where R⁷ is as defined herein.

In certain embodiments, the moieties

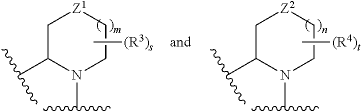

are each independently selected from:

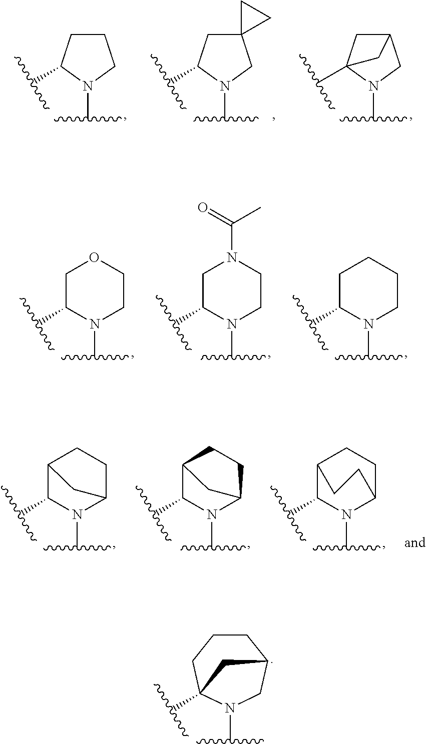

In certain embodiments, the moieties

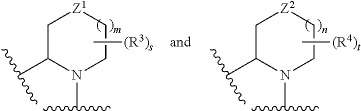

are each independently selected from:

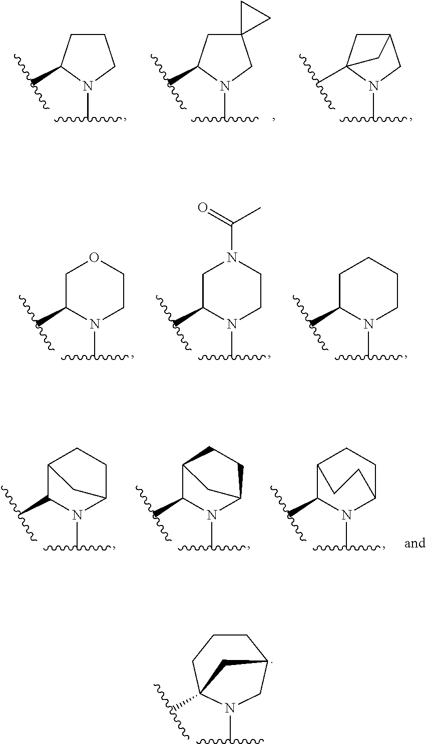

In one embodiment, provided herein is a compound selected from:

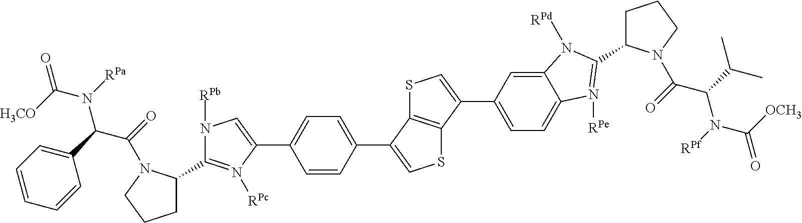

| Cmpd. | R^{Pa} | R^{Pb} | R^{Pc} | R^{Pd} | R^{Pe} | R^{Pf} |
|---|---|---|---|---|---|---|
| A1 | H | —CH₂O(O)P(ONa)₂ | Absent | H | Absent | H |
| A2 | H | H | Absent | —CH₂O(O)P(ONa)₂ | Absent | H |
| A3 | H | H | Absent | Absent | —CH₂O(O)P(ONa)₂ | H |
| A4 | H | Absent | —CH₂O(O)P(ONa)₂ | H | Absent | H |
| A5 | H | —CH₂O(O)P(ONa)₂ | Absent | Absent | —CH₂O(O)P(ONa)₂ | H |
| A6 | H | —CH₂O(O)P(ONa)₂ | Absent | —CH₂O(O)P(ONa)₂ | Absent | H |
| 7b | H | —CH₂O(O)P(OH)₂ | Absent | Absent | —CH₂O(O)P(OH)₂ | H |
| 6a | H | —CH₂O(O)P(OH)₂ | Absent | H | Absent | H |
| 6b | H | Absent | —CH₂O(O)P(OH)₂ | H | Absent | H |
| 6d | H | H | Absent | Absent | —CH₂O(O)P(OH)₂ | H |
| 6c | H | H | Absent | —CH₂O(O)P(OH)₂ | Absent | H |
| A12 | H | Absent | —CH₂O(O)P(ONa)₂ | Absent | —CH₂O(O)P(ONa)₂ | H |
| A13 | H | Absent | —CH₂O(O)P(ONa)₂ | —CH₂O(O)P(ONa)₂ | Absent | H |
| 7a | H | —CH₂O(O)P(OH)₂ | Absent | —CH₂O(O)P(OH)₂ | Absent | H |
| 7c | H | Absent | —CH₂O(O)P(OH)₂ | —CH₂O(O)P(OH)₂ | Absent | H |
| 7d | H | Absent | —CH₂O(O)P(OH)₂ | Absent | —CH₂O(O)P(OH)₂ | H |
| 10a | H | 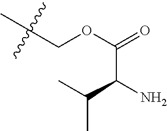 [SMILES: *COC(=O)[C@@H](N)C(C)C] | Absent | H | Absent | H |
| 10b | H | Absent | 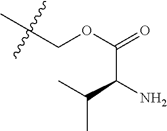 [SMILES: *COC(=O)[C@@H](N)C(C)C] | H | Absent | H |
| 10c | H | H | Absent | 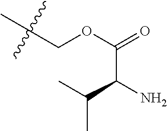 [SMILES: *COC(=O)[C@@H](N)C(C)C] | Absent | H |
| 10d | H | H | Absent | Absent | 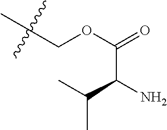 [SMILES: *COC(=O)[C@@H](N)C(C)C] | H |
| 11a | H | 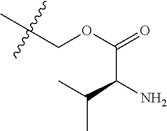 [SMILES: *COC(=O)[C@@H](N)C(C)C] | Absent | 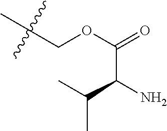 [SMILES: *COC(=O)[C@@H](N)C(C)C] | Absent | H |
| 11b | H | 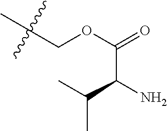 [SMILES: *COC(=O)[C@@H](N)C(C)C] | Absent | Absent | 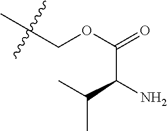 [SMILES: *COC(=O)[C@@H](N)C(C)C] | H |
| 11c | H | Absent | 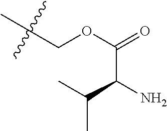 [SMILES: *COC(=O)[C@@H](N)C(C)C] | 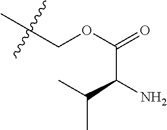 [SMILES: *COC(=O)[C@@H](N)C(C)C] | Absent | H |
| 11d | H | Absent | 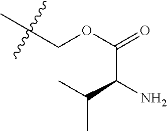 [SMILES: *COC(=O)[C@@H](N)C(C)C] | Absent | 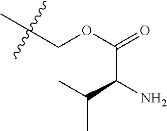 [SMILES: *COC(=O)[C@@H](N)C(C)C] | H |

and isotopic variants thereof; and pharmaceutically acceptable solvates thereof.

The compounds provided herein are intended to encompass all possible stereoisomers, unless a particular stereochemistry is specified. Where the compound provided herein contains an alkenyl or alkenylene group, the compound may exist as one or mixture of geometric cis/trans (or Z/E) isomers. Where structural isomers are interconvertible, the compound may exist as a single tautomer or a mixture of tautomers. This can take the form of proton tautomerism in the compound that contains, for example, an imino, keto, or oxime group; or so-called valence tautomerism in the compound that contain an aromatic moiety. It follows that a single compound may exhibit more than one type of isomerism.

For example, the heterocyclic moieties,

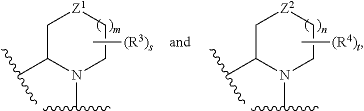

each contain at least one chiral center as indicated by star symbols. As result, the heterocyclic moiety may exist in at least two different stereoisomeric forms as shown below.

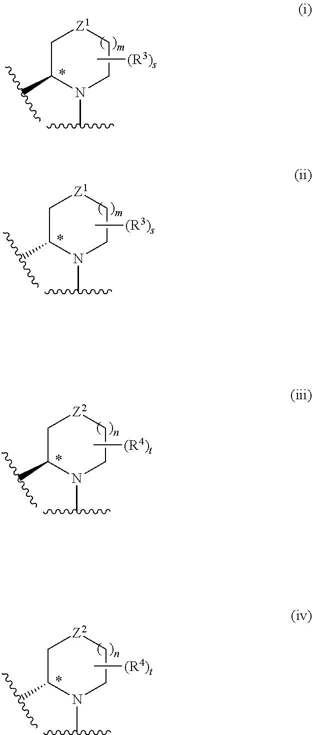

In certain embodiments, the heterocyclic moiety

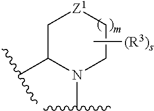

is in configuration (i) or (ii). In certain embodiments, the heterocyclic moiety

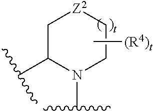

is in configuration (iii) or (iv).

The compounds provided herein may be enantiomerically pure, such as a single enantiomer or a single diastereomer, or be stereoisomeric mixtures, such as a mixture of enantiomers, e.g., a racemic mixture of two enantiomers; or a mixture of two or more diastereomers. As such, one of skill in the art will recognize that administration of a compound in its (R) form is equivalent, for compounds that undergo epimerization in vivo, to administration of the compound in its (S) form. Conventional techniques for the preparation/isolation of individual enantiomers include synthesis from a suitable optically pure precursor, asymmetric synthesis from achiral starting materials, or resolution of an enantiomeric mixture, for example, chiral chromatography, recrystallization, resolution, diastereomeric salt formation, or derivatization into diastereomeric adducts followed by separation.

When the compound provided herein contains an acidic or basic moiety, it may also be provided as a pharmaceutically acceptable salt. See, Berge et al., J. Pharm. Sci. 1977, 66, 1-19; and Handbook of Pharmaceutical Salts, Properties, and Use; Stahl and Wermuth, Ed.; Wiley-VCH and VHCA: Zurich, Switzerland, 2002.

Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, boric acid, (+)-camphoric acid, camphorsulfonic acid, (+)-(1S)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxoglutaric acid, glycolic acid, hippuric acid, hydrobromic acid, hydrochloric acid, hydroiodic acid, (+)-L-lactic acid, (+)-DL-lactic acid, lactobionic acid, lauric acid, maleic acid, (−)-L-malic acid, malonic acid, (+)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, undecylenic acid, and valeric acid.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, 1H-imidazole, L-lysine, morpholine, 4-(2-hydroxyethyl)-morpholine, methylamine, piperidine, piperazine, propylamine, pyrrolidine, 1-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, and tromethamine.

The compound provided herein may also be provided as a prodrug, which is a functional derivative of the compound, and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis. See, Harper, Progress in Drug Research 1962, 4, 221-294; Morozowich et al. in Design of Biopharmaceutical Properties through Prodrugs and Analogs; Roche Ed., APHA Acad. Pharm. Sci.: 1977; Gangwar et al., Des. Biopharm. Prop. Prodrugs Analogs, 1977, 409-421; Bundgaard, Arch. Pharm. Chem. 1979, 86, 1-39; Farquhar et al., J. Pharm. Sci. 1983, 72, 324-325; Wernuth in Drug Design: Fact or Fantasy; Jolles et al. Eds.; Academic Press: London, 1984; pp 47-72; Design of Prodrugs; Bundgaard et al. Eds.; Elsevier: 1985; Fleisher et al., Methods Enzymol. 1985, 112, 360-381; Stella et al., Drugs 1985, 29, 455-473; Bioreversible Carriers in Drug in Drug Design, Theory and Application; Roche Ed.; APHA Acad. Pharm. Sci.: 1987; Bundgaard, Controlled Drug Delivery 1987, 17, 179-96; Waller et al., Br. J. Clin. Pharmac. 1989, 28, 497-507; Balant et al., Eur. J. Drug Metab. Pharmacokinet. 1990, 15, 143-53; Freeman et al., J. Chem. Soc., Chem. Commun. 1991, 875-877; Bundgaard, Adv. Drug Delivery Rev. 1992, 8, 1-38; Nathwani and Wood, Drugs 1993, 45, 866-94; Friis and Bundgaard, Eur. J. Pharm. Sci. 1996, 4, 49-59; Fleisher et al., Adv. Drug Delivery Rev. 1996, 19, 115-130; Sinhababu and Thakker, Adv. Drug Delivery Rev. 1996, 19, 241-273; Taylor, Adv. Drug Delivery Rev. 1996, 19, 131-148; Gaignault et al., Pract. Med. Chem. 1996, 671-696; Browne, Clin. Neuropharmacol. 1997, 20, 1-12; Valentino and Borchardt, Drug Discovery Today 1997, 2, 148-155; Pauletti et al., Adv. Drug. Delivery Rev. 1997, 27, 235-256; Mizen et al., Pharm. Biotech. 1998, 11, 345-365; Wiebe and Knaus, Adv. Drug Delivery Rev. 1999, 39, 63-80; Tan et al., Adv. Drug Delivery Rev. 1999, 39, 117-151; Balimane and Sinko, Adv. Drug Delivery Rev. 1999, 39, 183-209; Wang et al., Curr. Pharm. Design 1999, 5, 265-287; Han et al., AAPS Pharmsci. 2000, 2, 1-11; Asghamejad in Transport Processes in Pharmaceutical Systems; Amidon et al., Eds.; Marcell Dekker: 2000; pp 185-218; Sinha et al., Pharm. Res. 2001, 18, 557-564; Anand et al., Expert Opin. Biol. Ther. 2002, 2, 607-620; Rao, Resonace 2003, 19-27; Sloan et al., Med. Res. Rev. 2003, 23, 763-793; Patterson et al., Curr. Pharm. Des. 2003, 9, 2131-2154; Hu, IDrugs 2004, 7, 736-742; Robinson et al., Proc. Natl. Acad. Sci. U.S.A. 2004, 101, 14527-14532; Erion et al., J. Pharmacol. Exp. Ther. 2005, 312, 554-560; Fang et al., Curr. Drug Discov. Technol. 2006, 3, 211-224; Stanczak et al., Pharmacol. Rep. 2006, 58, 599-613; Sloan et al., Pharm. Res. 2006, 23, 2729-2747; Stella et al., Adv. Drug Deliv. Rev. 2007, 59, 677-694; Gomes et al., Molecules 2007, 12, 2484-2506; Krafz et al., ChemMedChem 2008, 3, 20-53; Rautio et al., AAPS J. 2008, 10, 92-102; Rautio et al., Nat. Rev. Drug. Discov. 2008, 7, 255-270; Pavan et al., Molecules, 2008, 13, 1035-1065; Sandros et al., Molecules 2008, 13, 1156-1178; Singh et al., Curr. Med. Chem. 2008, 15, 1802-1826; Onishi et al., Molecules, 2008, 13, 2136-2155; Huttunen et al., Curr. Med. Chem. 2008, 15, 2346-2365; and Serafin et al., Mini Rev. Med. Chem. 2009, 9, 481-497.

### Methods of Synthesis

The compounds provided herein can be prepared, isolated, or obtained by any method known to one of skill in the art. For an example, a compound of Formula IA can be prepared as shown in Scheme I. The reaction of compound 1 with Cl—CH₂—OP(OtBu)₂ yields compound 2, a compound of Formula IA, wherein at least one of the four R^{P} groups is —CH₂—OP(OtBu)₂. Compound 2 is further treated with an acid, such as TFA, to yield compound 3, wherein at least one of the four R^{P} groups is —CH₂—OP(OH)₂.

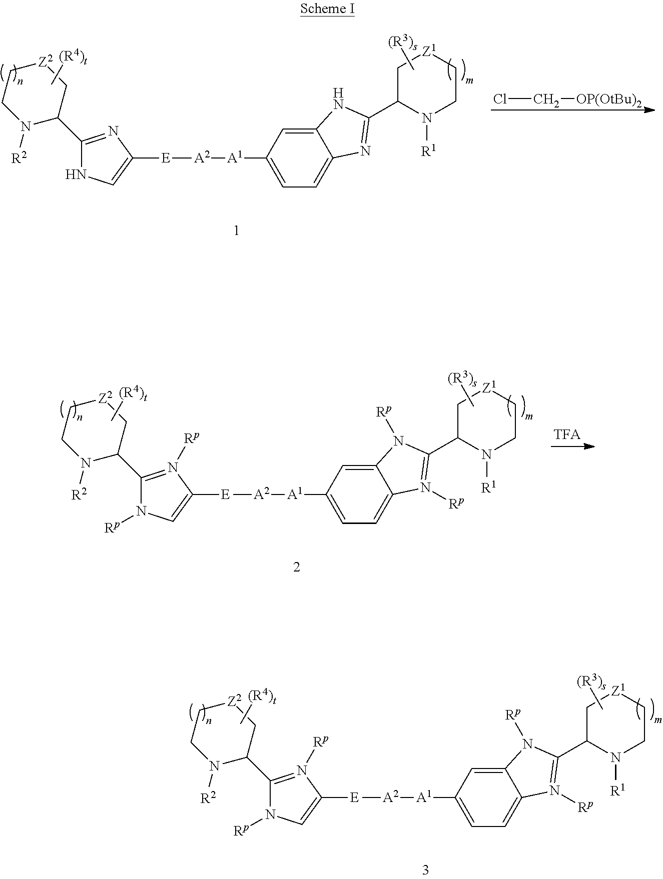

Alternatively, the compounds provided herein can be prepared by introducing a phosphonooxyalkyl group in the synthesis before coupling the imidazole and benzimidazole moieties as show in Schemes II and III. The phosphonooxyalkyled imidazole 5 or 6 is coupled with a suitable benzimidazole moiety via the Stille or Suzuki reaction to form a compound provided herein, as described in U.S. Pat. No. 8,273,341 or 8,362,068. Similarly, the phosphonooxyalkyled benzimidazole 8 or 9 is coupled with a suitable imidazole moiety via the Stille or Suzuki reaction to form a compound provided herein.

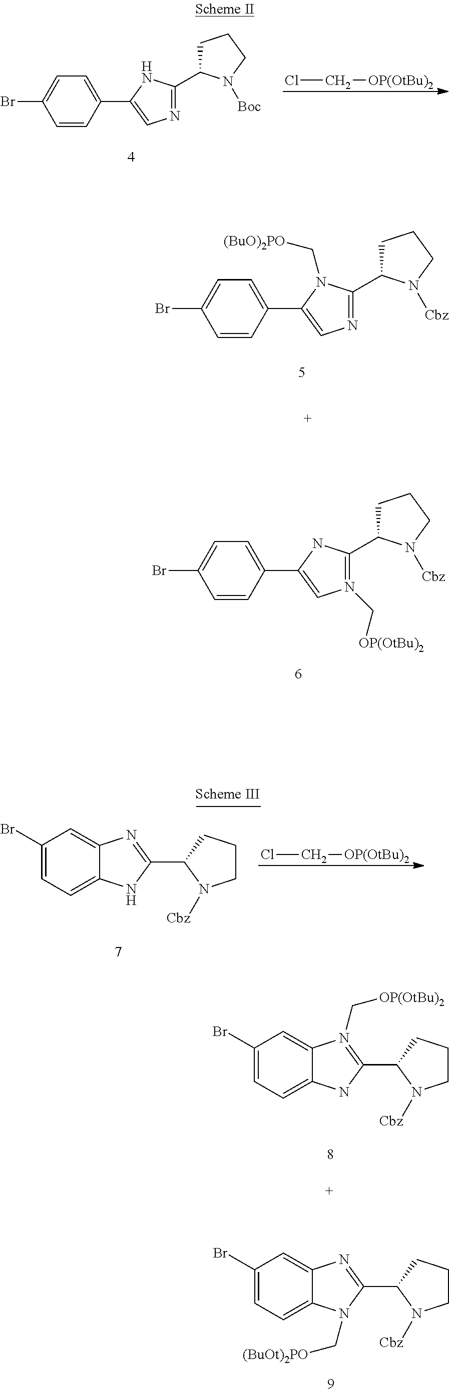

The starting materials, compound 1 and Cl—CH₂—OP(OtBu)₂ used in the synthesis of the compounds provided herein are either commercially available or can be prepared by a method known to one of skill in the art. For example, compound 1 can be prepared according to the methods described in U.S. Pat. No. 8,273,341 or 8,362,068, the disclosure of each of which is incorporated herein by reference in its entirety. The starting material Cl—CH₂—OP(OtBu)₂ can be prepared according to the methods described in Krise et al., J. Med. Chem. 1999, 42, 3094-3100; the disclosure of which is incorporated herein by reference in its entirety.

### Pharmaceutical Compositions

Provided herein are pharmaceutical compositions comprising a compound provided herein, e.g., a compound of any of Formulae disclosed herein, including Formulae I to XIV, IIIa to XIVa, IIIb to XIVb, IIIc to XIVc, IIId to XIVd, IIIe to XIVe, IA to IAe, IIA to IIAe, IB, IIB to IIBd, IIIB to IIIBd, IC to ICc, ID to IDd, or IE to IEc, as an active ingredient, including a single enantiomer, a racemic mixture, a diastereomer, a mixture of diastereomers, or an isotopic variant thereof; or a pharmaceutically acceptable salt or solvate thereof; in combination with a pharmaceutically acceptable vehicle, carrier, diluent, or excipient, or a mixture thereof.

Suitable excipients are well known to those skilled in the art, and non-limiting examples of suitable excipients are provided herein. Whether a particular excipient is suitable for incorporation into a pharmaceutical composition or dosage form depends on a variety of factors well known in the art, including, but not limited to, the method of administration. For example, oral dosage forms such as tablets may contain excipients not suited for use in parenteral dosage forms. The suitability of a particular excipient may also depend on the specific active ingredients in the dosage form. For example, the decomposition of some active ingredients may be accelerated by some excipients such as lactose, or when exposed to water. Active ingredients that comprise primary or secondary amines are particularly susceptible to such accelerated decomposition. Consequently, provided herein are pharmaceutical compositions and dosage forms that contain little, if any, lactose, or other mono- or di-saccharides. As used herein, the term “lactose-free” means that the amount of lactose present, if any, is insufficient to substantially increase the degradation rate of an active ingredient. In one embodiment, lactose-free compositions comprise an active ingredient provided herein, a binder/filler, and a lubricant. In another embodiment, lactose-free dosage forms comprise an active ingredient, microcrystalline cellulose, pre-gelatinized starch, and magnesium stearate.

The compound provided herein may be administered alone, or in combination with one or more other compounds provided herein. The pharmaceutical compositions that comprise a compound provided herein, e.g., a compound of any of Formulae disclosed herein, including Formulae I to XIV, IIIa to XIVa, IIIb to XIVb, IIIc to XIVc, IIId to XIVd, IIIe to XIVe, IA to IAe, IIA to IIAe, IB, IIB to IIBd, IIIB to IIIBd, IC to ICc, ID to IDd, and IE to IEc, including a single enantiomer, a racemic mixture, a diastereomer, a mixture of diastereomers, or an isotopic variant thereof; or a pharmaceutically acceptable salt or solvate thereof, can be formulated in various dosage forms for oral, parenteral, and topical administration. The pharmaceutical compositions can also be formulated as modified release dosage forms, including delayed-, extended-, prolonged-, sustained-, pulsatile-, controlled-, accelerated-, fast-, targeted-, programmed-release, and gastric retention dosage forms. These dosage forms can be prepared according to conventional methods and techniques known to those skilled in the art (see, Remington: The Science and Practice of Pharmacy, supra; Modified-Release Drug Delivery Technology, 2nd ed.; Rathbone et al., Eds.; Marcel Dekker, Inc.: New York, N.Y., 2008).

In one embodiment, the pharmaceutical compositions are provided in a dosage form for oral administration, which comprise a compound provided herein, e.g., a compound of any of Formulae disclosed herein, including Formulae I to XIV, IIIa to XIVa, IIIb to XIVb, IIIc to XIVc, IIId to XIVd, IIIe to XIVe, IA to IAe, IIA to IIAe, IB, IIB to IIBd, IIIB to IIIBd, IC to ICc, ID to IDd, and IE to IEc, including a single enantiomer, a racemic mixture, a diastereomer, a mixture of diastereomers, or an isotopic variant thereof; or a pharmaceutically acceptable salt or solvate thereof; and one or more pharmaceutically acceptable excipients or carriers.

In another embodiment, the pharmaceutical compositions are provided in a dosage form for parenteral administration, which comprise a compound provided herein, e.g., a compound of any of Formulae disclosed herein, including Formulae I to XIV, IIIa to XIVa, IIIb to XIVb, IIIc to XIVc, IIId to XIVd, IIIe to XIVe, IA to IAe, IIA to IIAe, IB, IIB to IIBd, IIIB to IIIBd, IC to ICc, ID to IDd, and IE to IEc, including a single enantiomer, a racemic mixture, a diastereomer, a mixture of diastereomers, or an isotopic variant thereof; or a pharmaceutically acceptable salt or solvate thereof; and one or more pharmaceutically acceptable excipients or carriers.

In yet another embodiment, the pharmaceutical compositions are provided in a dosage form for topical administration, which comprise a compound provided herein, e.g., a compound of any of Formulae disclosed herein, including Formulae I to XIV, IIIa to XIVa, IIIb to XIVb, IIIc to XIVc, IIId to XIVd, IIIe to XIVe, IA to IAe, IIA to IIAe, IB, IIB to IIBd, IIIB to IIIBd, IC to ICc, ID to IDd, and IE to IEc, including a single enantiomer, a racemic mixture, a diastereomer, a mixture of diastereomers, or an isotopic variant thereof; or a pharmaceutically acceptable salt or solvate thereof; and one or more pharmaceutically acceptable excipients or carriers.

The pharmaceutical compositions provided herein can be provided in a unit-dosage form or multiple-dosage form. A unit-dosage form, as used herein, refers to physically discrete a unit suitable for administration to a human and animal subject, and packaged individually as is known in the art. Each unit-dose contains a predetermined quantity of an active ingredient(s) sufficient to produce the desired therapeutic effect, in association with the required pharmaceutical carriers or excipients. Examples of a unit-dosage form include an ampoule, syringe, and individually packaged tablet and capsule. For example, a 100 mg unit dose contains about 100 mg of an active ingredient in a packaged tablet or capsule. A unit-dosage form may be administered in fractions or multiples thereof. A multiple-dosage form is a plurality of identical unit-dosage forms packaged in a single container to be administered in segregated unit-dosage form. Examples of a multiple-dosage form include a vial, bottle of tablets or capsules, or bottle of pints or gallons.

The pharmaceutical compositions provided herein can be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

### A. Oral Administration

The pharmaceutical compositions provided herein for oral administration can be provided in solid, semisolid, or liquid dosage forms for oral administration. As used herein, oral administration also includes buccal, lingual, and sublingual administration. Suitable oral dosage forms include, but are not limited to, tablets, fastmelts, chewable tablets, capsules, pills, strips, troches, lozenges, pastilles, cachets, pellets, medicated chewing gum, bulk powders, effervescent or non-effervescent powders or granules, oral mists, solutions, emulsions, suspensions, wafers, sprinkles, elixirs, and syrups. In addition to the active ingredient(s), the pharmaceutical compositions can contain one or more pharmaceutically acceptable carriers or excipients, including, but not limited to, binders, fillers, diluents, disintegrants, wetting agents, lubricants, glidants, coloring agents, dye-migration inhibitors, sweetening agents, flavoring agents, emulsifying agents, suspending and dispersing agents, preservatives, solvents, non-aqueous liquids, organic acids, and sources of carbon dioxide.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The amount of a binder or filler in the pharmaceutical compositions provided herein varies upon the type of formulation, and is readily discernible to those of ordinary skill in the art. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets. The amount of a diluent in the pharmaceutical compositions provided herein varies upon the type of formulation, and is readily discernible to those of ordinary skill in the art.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of a disintegrant in the pharmaceutical compositions provided herein varies upon the type of formulation, and is readily discernible to those of ordinary skill in the art. The amount of a disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. The pharmaceutical compositions provided herein may contain about 0.1 to about 5% by weight of a lubricant.

Suitable glidants include, but are not limited to, colloidal silicon dioxide, CAB-O-SIL® (Cabot Co. of Boston, Mass.), and asbestos-free talc. Suitable coloring agents include, but are not limited to, any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Suitable flavoring agents include, but are not limited to, natural flavors extracted from plants, such as fruits, and synthetic blends of compounds which produce a pleasant taste sensation, such as peppermint and methyl salicylate. Suitable sweetening agents include, but are not limited to, sucrose, lactose, mannitol, syrups, glycerin, and artificial sweeteners, such as saccharin and aspartame. Suitable emulsifying agents include, but are not limited to, gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suitable suspending and dispersing agents include, but are not limited to, sodium carboxymethylcellulose, pectin, tragacanth, Veegum, acacia, sodium carbomethylcellulose, hydroxypropyl methylcellulose, and polyvinylpyrrolidone. Suitable preservatives include, but are not limited to, glycerin, methyl and propylparaben, benzoic add, sodium benzoate and alcohol. Suitable wetting agents include, but are not limited to, propylene glycol monostearate, sorbitan monooleate, diethylene glycol monolaurate, and polyoxyethylene lauryl ether. Suitable solvents include, but are not limited to, glycerin, sorbitol, ethyl alcohol, and syrup. Suitable non-aqueous liquids utilized in emulsions include, but are not limited to, mineral oil and cottonseed oil. Suitable organic acids include, but are not limited to, citric and tartaric acid. Suitable sources of carbon dioxide include, but are not limited to, sodium bicarbonate and sodium carbonate.

It should be understood that many carriers and excipients may serve a plurality of functions, even within the same formulation.

The pharmaceutical compositions provided herein for oral administration can be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenyl salicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms can be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein for oral administration can be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

The pharmaceutical compositions provided herein for oral administration can be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquid or solvent, emulsifying agent, and preservative. Suspensions may include a pharmaceutically acceptable suspending agent and preservative. Aqueous alcoholic solutions may include a pharmaceutically acceptable acetal, such as a di(lower alkyl) acetal of a lower alkyl aldehyde, e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations can further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration can be also provided in the forms of liposomes, micelles, microspheres, or nanosystems. Micellar dosage forms can be prepared as described in U.S. Pat. No. 6,350,458.

The pharmaceutical compositions provided herein for oral administration can be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents.

Pharmaceutically acceptable carriers and excipients used in the effervescent granules or powders may include organic acids and a source of carbon dioxide.

Coloring and flavoring agents can be used in all of the above dosage forms.

The pharmaceutical compositions provided herein for oral administration can be formulated as immediate or modified release dosage forms, including delayed-, sustained, pulsed-, controlled, targeted-, and programmed-release forms.

### B. Parenteral Administration

The pharmaceutical compositions provided herein can be administered parenterally by injection, infusion, or implantation, for local or systemic administration. Parenteral administration, as used herein, include intravenous, intraarterial, intraperitoneal, intrathecal, intraventricular, intraurethral, intrasternal, intracranial, intramuscular, intrasynovial, intravesical, and subcutaneous administration.

The pharmaceutical compositions provided herein for parenteral administration can be formulated in any dosage forms that are suitable for parenteral administration, including solutions, suspensions, emulsions, micelles, liposomes, microspheres, nanosystems, and solid forms suitable for solutions or suspensions in liquid prior to injection. Such dosage forms can be prepared according to conventional methods known to those skilled in the art of pharmaceutical science (see, Remington: The Science and Practice of Pharmacy, supra).

The pharmaceutical compositions intended for parenteral administration can include one or more pharmaceutically acceptable carriers and excipients, including, but not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, cryoprotectants, lyoprotectants, thickening agents, pH adjusting agents, and inert gases.

Suitable aqueous vehicles include, but are not limited to, water, saline, physiological saline or phosphate buffered saline (PBS), sodium chloride injection, Ringers injection, isotonic dextrose injection, sterile water injection, dextrose and lactated Ringers injection. Suitable non-aqueous vehicles include, but are not limited to, fixed oils of vegetable origin, castor oil, corn oil, cottonseed oil, olive oil, peanut oil, peppermint oil, safflower oil, sesame oil, soybean oil, hydrogenated vegetable oils, hydrogenated soybean oil, and medium-chain triglycerides of coconut oil, and palm seed oil. Suitable water-miscible vehicles include, but are not limited to, ethanol, 1,3-butanediol, liquid polyethylene glycol (e.g., polyethylene glycol 300 and polyethylene glycol 400), propylene glycol, glycerin, N-methyl-2-pyrrolidone, N,N-dimethylacetamide, and dimethyl sulfoxide.

Suitable antimicrobial agents or preservatives include, but are not limited to, phenols, cresols, mercurials, benzyl alcohol, chlorobutanol, methyl and propyl p-hydroxybenzoates, thimerosal, benzalkonium chloride (e.g., benzethonium chloride), methyl- and propyl-parabens, and sorbic acid. Suitable isotonic agents include, but are not limited to, sodium chloride, glycerin, and dextrose. Suitable buffering agents include, but are not limited to, phosphate and citrate. Suitable antioxidants are those as described herein, including bisulfite and sodium metabisulfite. Suitable local anesthetics include, but are not limited to, procaine hydrochloride. Suitable suspending and dispersing agents are those as described herein, including sodium carboxymethylcelluose, hydroxypropyl methylcellulose, and polyvinylpyrrolidone. Suitable emulsifying agents are those described herein, including polyoxyethylene sorbitan monolaurate, polyoxyethylene sorbitan monooleate 80, and triethanolamine oleate. Suitable sequestering or chelating agents include, but are not limited to EDTA. Suitable pH adjusting agents include, but are not limited to, sodium hydroxide, hydrochloric acid, citric acid, and lactic acid. Suitable complexing agents include, but are not limited to, cyclodextrins, including α-cyclodextrin, β-cyclodextrin, hydroxypropyl-β-cyclodextrin, sulfobutylether-β-cyclodextrin, and sulfobutylether 7-β-cyclodextrin (CAPTISOL®, CyDex, Lenexa, Kans.).

When the pharmaceutical compositions provided herein are formulated for multiple dosage administration, the multiple dosage parenteral formulations must contain an antimicrobial agent at bacteriostatic or fungistatic concentrations. All parenteral formulations must be sterile, as known and practiced in the art.

In one embodiment, the pharmaceutical compositions for parenteral administration are provided as ready-to-use sterile solutions. In another embodiment, the pharmaceutical compositions are provided as sterile dry soluble products, including lyophilized powders and hypodermic tablets, to be reconstituted with a vehicle prior to use.

In yet another embodiment, the pharmaceutical compositions are provided as ready-to-use sterile suspensions. In yet another embodiment, the pharmaceutical compositions are provided as sterile dry insoluble products to be reconstituted with a vehicle prior to use. In still another embodiment, the pharmaceutical compositions are provided as ready-to-use sterile emulsions.

The pharmaceutical compositions provided herein for parenteral administration can be formulated as immediate or modified release dosage forms, including delayed-, sustained, pulsed-, controlled, targeted-, and programmed-release forms.

The pharmaceutical compositions provided herein for parenteral administration can be formulated as a suspension, solid, semi-solid, or thixotropic liquid, for administration as an implanted depot. In one embodiment, the pharmaceutical compositions provided herein are dispersed in a solid inner matrix, which is surrounded by an outer polymeric membrane that is insoluble in body fluids but allows the active ingredient in the pharmaceutical compositions diffuse through.

Suitable inner matrixes include, but are not limited to, polymethylmethacrylate, polybutyl-methacrylate, plasticized or unplasticized polyvinylchloride, plasticized nylon, plasticized polyethylene terephthalate, natural rubber, polyisoprene, polyisobutylene, polybutadiene, polyethylene, ethylene-vinyl acetate copolymers, silicone rubbers, polydimethylsiloxanes, silicone carbonate copolymers, hydrophilic polymers, such as hydrogels of esters of acrylic and methacrylic acid, collagen, cross-linked polyvinyl alcohol, and cross-linked partially hydrolyzed polyvinyl acetate.

Suitable outer polymeric membranes include but are not limited to, polyethylene, polypropylene, ethylene/propylene copolymers, ethylene/ethyl acrylate copolymers, ethylene/vinyl acetate copolymers, silicone rubbers, polydimethyl siloxanes, neoprene rubber, chlorinated polyethylene, polyvinylchloride, vinyl chloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer.

### C. Topical Administration

The pharmaceutical compositions provided herein can be administered topically to the skin, orifices, or mucosa. The topical administration, as used herein, includes (intra)dermal, conjunctival, intracomeal, intraocular, ophthalmic, auricular, transdermal, nasal, vaginal, urethral, respiratory, and rectal administration.

The pharmaceutical compositions provided herein can be formulated in any dosage forms that are suitable for topical administration for local or systemic effect, including emulsions, solutions, suspensions, creams, gels, hydrogels, ointments, dusting powders, dressings, elixirs, lotions, suspensions, tinctures, pastes, foams, films, aerosols, irrigations, sprays, suppositories, bandages, and dermal patches. The topical formulation of the pharmaceutical compositions provided herein can also comprise liposomes, micelles, microspheres, nanosystems, and mixtures thereof.

Pharmaceutically acceptable carriers and excipients suitable for use in the topical formulations provided herein include, but are not limited to, aqueous vehicles, water-miscible vehicles, non-aqueous vehicles, antimicrobial agents or preservatives against the growth of microorganisms, stabilizers, solubility enhancers, isotonic agents, buffering agents, antioxidants, local anesthetics, suspending and dispersing agents, wetting or emulsifying agents, complexing agents, sequestering or chelating agents, penetration enhancers, cryoprotectants, lyoprotectants, thickening agents, and inert gases.

The pharmaceutical compositions can also be administered topically by electroporation, iontophoresis, phonophoresis, sonophoresis, or microneedle or needle-free injection, such as POWDERJECT™ (Chiron Corp., Emeryville, Calif.), and BIOJECT™ (Bioject Medical Technologies Inc., Tualatin, Oreg.).

The pharmaceutical compositions provided herein can be provided in the forms of ointments, creams, and gels. Suitable ointment vehicles include oleaginous or hydrocarbon vehicles, including lard, benzoinated lard, olive oil, cottonseed oil, and other oils, white petrolatum; emulsifiable or absorption vehicles, such as hydrophilic petrolatum, hydroxystearin sulfate, and anhydrous lanolin; water-removable vehicles, such as hydrophilic ointment; water-soluble ointment vehicles, including polyethylene glycols of varying molecular weight; emulsion vehicles, either water-in-oil (W/O) emulsions or oil-in-water (O/W) emulsions, including cetyl alcohol, glyceryl monostearate, lanolin, and stearic acid (see, Remington: The Science and Practice of Pharmacy, supra). These vehicles are emollient but generally require addition of antioxidants and preservatives.

Suitable cream base can be oil-in-water or water-in-oil. Suitable cream vehicles may be water-washable, and contain an oil phase, an emulsifier, and an aqueous phase. The oil phase is also called the “internal” phase, which is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation may be a nonionic, anionic, cationic, or amphoteric surfactant.

Gels are semisolid, suspension-type systems. Single-phase gels contain organic macromolecules distributed substantially uniformly throughout the liquid carrier. Suitable gelling agents include, but are not limited to, crosslinked acrylic acid polymers, such as carbomers, carboxypolyalkylenes, and CARBOPOL®; hydrophilic polymers, such as polyethylene oxides, polyoxyethylene-polyoxypropylene copolymers, and polyvinylalcohol; cellulosic polymers, such as hydroxypropyl cellulose, hydroxyethyl cellulose, hydroxypropyl methylcellulose, hydroxypropyl methylcellulose phthalate, and methylcellulose; gums, such as tragacanth and xanthan gum; sodium alginate; and gelatin. In order to prepare a uniform gel, dispersing agents such as alcohol or glycerin can be added, or the gelling agent can be dispersed by trituration, mechanical mixing, and/or stirring.

The pharmaceutical compositions provided herein can be administered rectally, urethrally, vaginally, or perivaginally in the forms of suppositories, pessaries, bougies, poultices or cataplasm, pastes, powders, dressings, creams, plasters, contraceptives, ointments, solutions, emulsions, suspensions, tampons, gels, foams, sprays, or enemas.

These dosage forms can be manufactured using conventional processes as described in Remington: The Science and Practice of Pharmacy, supra.

Rectal, urethral, and vaginal suppositories are solid bodies for insertion into body orifices, which are solid at ordinary temperatures but melt or soften at body temperature to release the active ingredient(s) inside the orifices. Pharmaceutically acceptable carriers utilized in rectal and vaginal suppositories include bases or vehicles, such as stiffening agents, which produce a melting point in the proximity of body temperature, when formulated with the pharmaceutical compositions provided herein; and antioxidants as described herein, including bisulfite and sodium metabisulfite. Suitable vehicles include, but are not limited to, cocoa butter (theobroma oil), glycerin-gelatin, carbowax (polyoxyethylene glycol), spermaceti, paraffin, white and yellow wax, and appropriate mixtures of mono-, di- and triglycerides of fatty acids, and hydrogels, such as polyvinyl alcohol, hydroxyethyl methacrylate, and polyacrylic acid. Combinations of the various vehicles can also be used. Rectal and vaginal suppositories may be prepared by compressing or molding. The typical weight of a rectal and vaginal suppository is about 2 to about 3 g.

The pharmaceutical compositions provided herein can be administered ophthalmically in the forms of solutions, suspensions, ointments, emulsions, gel-forming solutions, powders for solutions, gels, ocular inserts, and implants.

The pharmaceutical compositions provided herein can be administered intranasally or by inhalation to the respiratory tract. The pharmaceutical compositions can be provided in the form of an aerosol or solution for delivery using a pressurized container, pump, spray, atomizer, such as an atomizer using electrohydrodynamics to produce a fine mist, or nebulizer, alone or in combination with a suitable propellant, such as 1,1,1,2-tetrafluoroethane or 1,1,1,2,3,3,3-heptafluoropropane. The pharmaceutical compositions can also be provided as a dry powder for insufflation, alone or in combination with an inert carrier such as lactose or phospholipids; and nasal drops. For intranasal use, the powder can comprise a bioadhesive agent, including chitosan or cyclodextrin.

Solutions or suspensions for use in a pressurized container, pump, spray, atomizer, or nebulizer can be formulated to contain ethanol, aqueous ethanol, or a suitable alternative agent for dispersing, solubilizing, or extending release of the active ingredient provided herein; a propellant as solvent; and/or a surfactant, such as sorbitan trioleate, oleic acid, or an oligolactic acid.

The pharmaceutical compositions provided herein can be micronized to a size suitable for delivery by inhalation, such as about 50 micrometers or less, or about 10 micrometers or less. Particles of such sizes can be prepared using a comminuting method known to those skilled in the art, such as spiral jet milling, fluid bed jet milling, supercritical fluid processing to form nanoparticles, high pressure homogenization, or spray drying.

Capsules, blisters, and cartridges for use in an inhaler or insufflator can be formulated to contain a powder mix of the pharmaceutical compositions provided herein; a suitable powder base, such as lactose or starch; and a performance modifier, such as 1-leucine, mannitol, or magnesium stearate. The lactose may be anhydrous or in the form of the monohydrate. Other suitable excipients or carriers include, but are not limited to, dextran, glucose, maltose, sorbitol, xylitol, fructose, sucrose, and trehalose. The pharmaceutical compositions provided herein for inhaled/intranasal administration can further comprise a suitable flavor, such as menthol and levomenthol; and/or sweeteners, such as saccharin and saccharin sodium.

The pharmaceutical compositions provided herein for topical administration can be formulated to be immediate release or modified release, including delayed-, sustained-, pulsed-, controlled-, targeted, and programmed release.

### D. Modified Release

The pharmaceutical compositions provided herein can be formulated as a modified release dosage form. As used herein, the term “modified release” refers to a dosage form in which the rate or place of release of the active ingredient(s) is different from that of an immediate dosage form when administered by the same route. Modified release dosage forms include, but are not limited to, delayed-, extended-, prolonged-, sustained-, pulsatile-, controlled-, accelerated- and fast-, targeted-, programmed-release, and gastric retention dosage forms. The pharmaceutical compositions in modified release dosage forms can be prepared using a variety of modified release devices and methods known to those skilled in the art, including, but not limited to, matrix controlled release devices, osmotic controlled release devices, multiparticulate controlled release devices, ion-exchange resins, enteric coatings, multilayered coatings, microspheres, liposomes, and combinations thereof. The release rate of the active ingredient(s) can also be modified by varying the particle sizes and polymorphorism of the active ingredient(s).

Examples of modified release include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,958,458; 5,972,891; 5,980,945; 5,993,855; 6,045,830; 6,087,324; 6,113,943; 6,197,350; 6,248,363; 6,264,970; 6,267,981; 6,270,798; 6,375,987; 6,376,461; 6,419,961; 6,589,548; 6,613,358; 6,623,756; 6,699,500; 6,793,936; 6,827,947; 6,902,742; 6,958,161; 7,255,876; 7,416,738; 7,427,414; 7,485,322; Bussemer et al., Crit. Rev. Ther. Drug Carrier Syst. 2001, 18, 433-458; Modified-Release Drug Delivery Technology, 2nd ed.; Rathbone et al., Eds.; Marcel Dekker AG: 2005; Maroni et al., Expert. Opin. Drug Deliv. 2005, 2, 855-871; Shi et al., Expert Opin. Drug Deliv. 2005, 2, 1039-1058; Polymers in Drug Delivery; Ijeoma et al., Eds.; CRC Press LLC: Boca Raton, Fla., 2006; Badawy et al., J. Pharm. Sci. 2007, 9, 948-959; Modified-Release Drug Delivery Technology, supra; Conway, Recent Pat. Drug Deliv. Formul. 2008, 2, 1-8; Gazzaniga et al., Eur. J. Pharm. Biopharm. 2008, 68, 11-18; Nagarwal et al., Curr. Drug Deliv. 2008, 5, 282-289; Gallardo et al., Pharm. Dev. Technol. 2008, 13, 413-423; Chrzanowski, AAPS PharmSciTech. 2008, 9, 635-638; Chrzanowski, AAPS PharmSciTech. 2008, 9, 639-645; Kalantzi et al., Recent Pat. Drug Deliv. Formul. 2009, 3, 49-63; Saigal et al., Recent Pat. Drug Deliv. Formul. 2009, 3, 64-70; and Roy et al., J. Control Release 2009, 134, 74-80.

### 1. Matrix Controlled Release Devices

The pharmaceutical compositions provided herein in a modified release dosage form can be fabricated using a matrix controlled release device known to those skilled in the art. See, Takada et al. in Encyclopedia of Controlled Drug Delivery; Mathiowitz Ed.; Wiley: 1999; Vol 2.

In certain embodiments, the pharmaceutical compositions provided herein in a modified release dosage form is formulated using an erodible matrix device, which is water-swellable, erodible, or soluble polymers, including, but not limited to, synthetic polymers, and naturally occurring polymers and derivatives, such as polysaccharides and proteins.

Materials useful in forming an erodible matrix include, but are not limited to, chitin, chitosan, dextran, and pullulan; gum agar, gum arabic, gum karaya, locust bean gum, gum tragacanth, carrageenans, gum ghatti, guar gum, xanthan gum, and scleroglucan; starches, such as dextrin and maltodextrin; hydrophilic colloids, such as pectin; phosphatides, such as lecithin; alginates; propylene glycol alginate; gelatin; collagen; cellulosics, such as ethyl cellulose (EC), methylethyl cellulose (MEC), carboxymethyl cellulose (CMC), CMEC, hydroxyethyl cellulose (HEC), hydroxypropyl cellulose (HPC), cellulose acetate (CA), cellulose propionate (CP), cellulose butyrate (CB), cellulose acetate butyrate (CAB), CAP, CAT, hydroxypropyl methyl cellulose (HPMC), HPMCP, HPMCAS, hydroxypropyl methyl cellulose acetate trimellitate (HPMCAT), and ethyl hydroxyethyl cellulose (EHEC); polyvinyl pyrrolidone; polyvinyl alcohol; polyvinyl acetate; glycerol fatty acid esters; polyacrylamide; polyacrylic acid; copolymers of ethacrylic acid or methacrylic acid (EUDRAGIT®, Rohm America, Inc., Piscataway, N.J.); poly(2-hydroxyethyl-methacrylate); polylactides; copolymers of L-glutamic acid and ethyl-L-glutamate; degradable lactic acid-glycolic acid copolymers; poly-D-(−)-3-hydroxybutyric acid; and other acrylic acid derivatives, such as homopolymers and copolymers of butylmethacrylate, methyl methacrylate, ethyl methacrylate, ethylacrylate, (2-dimethylaminoethyl)methacrylate, and (trimethylaminoethyl)methacrylate chloride.

In certain embodiments, the pharmaceutical compositions provided herein are formulated with a non-erodible matrix device. The active ingredient(s) is dissolved or dispersed in an inert matrix and is released primarily by diffusion through the inert matrix once administered. Materials suitable for use as a non-erodible matrix device include, but are not limited to, insoluble plastics, such as polyethylene, polypropylene, polyisoprene, polyisobutylene, polybutadiene, polymethylmethacrylate, polybutylmethacrylate, chlorinated polyethylene, polyvinylchloride, methyl acrylate-methyl methacrylate copolymers, ethylene-vinyl acetate copolymers, ethylene/propylene copolymers, ethylene/ethyl acrylate copolymers, vinyl chloride copolymers with vinyl acetate, vinylidene chloride, ethylene and propylene, ionomer polyethylene terephthalate, butyl rubbers, epichlorohydrin rubbers, ethylene/vinyl alcohol copolymer, ethylene/vinyl acetate/vinyl alcohol terpolymer, ethylene/vinyloxyethanol copolymer, polyvinyl chloride, plasticized nylon, plasticized polyethylene terephthalate, natural rubber, silicone rubbers, polydimethylsiloxanes, and silicone carbonate copolymers; hydrophilic polymers, such as ethyl cellulose, cellulose acetate, crospovidone, and cross-linked partially hydrolyzed polyvinyl acetate; and fatty compounds, such as carnauba wax, microcrystalline wax, and triglycerides.

In a matrix controlled release system, the desired release kinetics can be controlled, for example, via the polymer type employed, the polymer viscosity, the particle sizes of the polymer and/or the active ingredient(s), the ratio of the active ingredient(s) versus the polymer, and other excipients or carriers in the compositions.

The pharmaceutical compositions provided herein in a modified release dosage form can be prepared by methods known to those skilled in the art, including direct compression, dry or wet granulation followed by compression, and melt-granulation followed by compression.

### 2. Osmotic Controlled Release Devices

The pharmaceutical compositions provided herein in a modified release dosage form can be fabricated using an osmotic controlled release device, including, but not limited to, one-chamber system, two-chamber system, asymmetric membrane technology (AMT), and extruding core system (ECS). In general, such devices have at least two components: (a) a core which contains an active ingredient; and (b) a semipermeable membrane with at least one delivery port, which encapsulates the core. The semipermeable membrane controls the influx of water to the core from an aqueous environment of use so as to cause drug release by extrusion through the delivery port(s).

In addition to the active ingredient(s), the core of the osmotic device optionally includes an osmotic agent, which creates a driving force for transport of water from the environment of use into the core of the device. One class of osmotic agents is water-swellable hydrophilic polymers, which are also referred to as “osmopolymers” and “hydrogels.” Suitable water-swellable hydrophilic polymers as osmotic agents include, but are not limited to, hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP), crosslinked PVP, polyvinyl alcohol (PVA), PVA/PVP copolymers, PVA/PVP copolymers with hydrophobic monomers such as methyl methacrylate and vinyl acetate, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl, cellulose (CEC), sodium alginate, polycarbophil, gelatin, xanthan gum, and sodium starch glycolate.

The other class of osmotic agents is osmogens, which are capable of imbibing water to affect an osmotic pressure gradient across the barrier of the surrounding coating. Suitable osmogens include, but are not limited to, inorganic salts, such as magnesium sulfate, magnesium chloride, calcium chloride, sodium chloride, lithium chloride, potassium sulfate, potassium phosphates, sodium carbonate, sodium sulfite, lithium sulfate, potassium chloride, and sodium sulfate; sugars, such as dextrose, fructose, glucose, inositol, lactose, maltose, mannitol, raffinose, sorbitol, sucrose, trehalose, and xylitol; organic acids, such as ascorbic acid, benzoic acid, fumaric acid, citric acid, maleic acid, sebacic acid, sorbic acid, adipic acid, edetic acid, glutamic acid, p-toluenesulfonic acid, succinic acid, and tartaric acid; urea; and mixtures thereof.

Osmotic agents of different dissolution rates can be employed to influence how rapidly the active ingredient(s) is initially delivered from the dosage form. For example, amorphous sugars, such as MANNOGEM™ EZ (SPI Pharma, Lewes, Del.) can be used to provide faster delivery during the first couple of hours to promptly produce the desired therapeutic effect, and gradually and continually release of the remaining amount to maintain the desired level of therapeutic or prophylactic effect over an extended period of time. In this case, the active ingredient(s) is released at such a rate to replace the amount of the active ingredient metabolized and excreted.

The core can also include a wide variety of other excipients and carriers as described herein to enhance the performance of the dosage form or to promote stability or processing.

Materials useful in forming the semipermeable membrane include various grades of acrylics, vinyls, ethers, polyamides, polyesters, and cellulosic derivatives that are water-permeable and water-insoluble at physiologically relevant pHs, or are susceptible to being rendered water-insoluble by chemical alteration, such as crosslinking. Examples of suitable polymers useful in forming the coating, include plasticized, unplasticized, and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxylated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes, and synthetic waxes.

Semipermeable membrane can also be a hydrophobic microporous membrane, wherein the pores are substantially filled with a gas and are not wetted by the aqueous medium but are permeable to water vapor, as disclosed in U.S. Pat. No. 5,798,119. Such hydrophobic but water-vapor permeable membrane are typically composed of hydrophobic polymers such as polyalkenes, polyethylene, polypropylene, polytetrafluoroethylene, polyacrylic acid derivatives, polyethers, polysulfones, polyethersulfones, polystyrenes, polyvinyl halides, polyvinylidene fluoride, polyvinyl esters and ethers, natural waxes, and synthetic waxes.

The delivery port(s) on the semipermeable membrane can be formed post-coating by mechanical or laser drilling. Delivery port(s) can also be formed in situ by erosion of a plug of water-soluble material or by rupture of a thinner portion of the membrane over an indentation in the core. In addition, delivery ports can be formed during coating process, as in the case of asymmetric membrane coatings of the type disclosed in U.S. Pat. Nos. 5,612,059 and 5,698,220.

The total amount of the active ingredient(s) released and the release rate can substantially by modulated via the thickness and porosity of the semipermeable membrane, the composition of the core, and the number, size, and position of the delivery ports.

The pharmaceutical compositions in an osmotic controlled-release dosage form can further comprise additional conventional excipients or carriers as described herein to promote performance or processing of the formulation.

The osmotic controlled-release dosage forms can be prepared according to conventional methods and techniques known to those skilled in the art. See, Remington: The Science and Practice of Pharmacy, supra; Santus and Baker, J. Controlled Release 1995, 35, 1-21; Verma et al., Drug Development and Industrial Pharmacy 2000, 26, 695-708; and Verma et al., J. Controlled Release 2002, 79, 7-27.

In certain embodiments, the pharmaceutical compositions provided herein are formulated as AMT controlled-release dosage form, which comprises an asymmetric osmotic membrane that coats a core comprising the active ingredient(s) and other pharmaceutically acceptable excipients or carriers. See, U.S. Pat. No. 5,612,059 and International Pat. Appl. Publ. No. WO 2002/17918. The AMT controlled-release dosage forms can be prepared according to conventional methods and techniques known to those skilled in the art, including direct compression, dry granulation, wet granulation, and a dip-coating method.

In certain embodiments, the pharmaceutical compositions provided herein are formulated as ESC controlled-release dosage form, which comprises an osmotic membrane that coats a core comprising the active ingredient(s), a hydroxylethyl cellulose, and other pharmaceutically acceptable excipients or carriers.

### 3. Multiparticulate Controlled Release Devices

The pharmaceutical compositions provided herein in a modified release dosage form can be fabricated as a multiparticulate controlled release device, which comprises a multiplicity of particles, granules, or pellets, ranging from about 10 μm to about 3 mm, about 50 μm to about 2.5 mm, or from about 100 μm to about 1 mm in diameter. Such multiparticulates can be made by the processes known to those skilled in the art, including wet- and dry-granulation, extrusion/spheronization, roller-compaction, melt-congealing, and by spray-coating seed cores. See, for example, Multiparticulate Oral Drug Delivery; Ghebre-Sellassie Ed.; Marcel Dekker: 1994; and Pharmaceutical Pelletization Technology; Ghebre-Sellassie Ed.; Marcel Dekker: 1989.

Other excipients or carriers as described herein can be blended with the pharmaceutical compositions to aid in processing and forming the multiparticulates. The resulting particles can themselves constitute the multiparticulate device or can be coated by various film-forming materials, such as enteric polymers, water-swellable, and water-soluble polymers. The multiparticulates can be further processed as a capsule or a tablet.

### 4. Targeted Delivery

The pharmaceutical compositions provided herein can also be formulated to be targeted to a particular tissue, receptor, or other area of the body of the subject to be treated, including liposome-, resealed erythrocyte-, and antibody-based delivery systems. Examples include, but are not limited to, those disclosed in U.S. Pat. Nos. 5,709,874; 5,759,542; 5,840,674; 5,900,252; 5,972,366; 5,985,307; 6,004,534; 6,039,975; 6,048,736; 6,060,082; 6,071,495; 6,120,751; 6,131,570; 6,139,865; 6,253,872; 6,271,359; 6,274,552; 6,316,652; and 7,169,410.

### Methods of Use

In one embodiment, provided herein are methods for treating or preventing a hepatitis C viral infection in a subject, which comprises administering to the subject a therapeutically effective amount of a compound provided herein, e.g., a compound of any of Formulae disclosed herein, including Formulae I to XIV, IIIa to XIVa, IIIb to XIVb, IIIc to XIVc, IIId to XIVd, Ille to XIVe, IA to IAe, IIA to IIAe, IB, IIB to IIBd, IIIB to IIIBd, IC to ICc, ID to IDd, and IE to IEc, including a single enantiomer, a racemic mixture, a diastereomer, a mixture of diastereomers, or an isotopic variant thereof; or a pharmaceutically acceptable salt or solvate thereof.

In another embodiment, provided herein are methods for treating, preventing, or ameliorating one or more symptoms of a liver disease or disorder associated with an HCV infection, comprising administering to a subject a therapeutically effective amount of a compound disclosed herein, e.g., a compound of any of Formulae disclosed herein, including Formulae I to XIV, IIIa to XIVa, IIIb to XIVb, IIIc to XIVc, IIId to XIVd, IIIe to XIVe, IA to IAe, IIA to IIAe, IB, IIB to IIBd, IIIB to IIIBd, IC to ICc, ID to IDd, and IE to IEc, including a single enantiomer, a racemic mixture, a diastereomer, a mixture of diastereomers, or an isotopic variant thereof; or a pharmaceutically acceptable salt or solvate thereof. Non-limiting examples of diseases associated with HCV infection include chronic hepatitis, cirrhosis, hepatocarcinoma, or extra hepatic manifestation.

In yet another embodiment, provided herein are methods for treating or preventing a drug-resistant hepatitis C viral infection in a subject, which comprises administering to the subject a therapeutically effective amount of a compound provided herein, e.g., a compound of any of Formulae disclosed herein, including Formulae I to XIV, IIIa to XIVa, IIIb to XIVb, IIIc to XIVc, IIId to XIVd, IIIe to XIVe, IA to IAe, IIA to IIAe, IB, IIB to IIBd, IIIB to IIIBd, IC to ICc, ID to IDd, and IE to IEc, including a single enantiomer, a racemic mixture, a diastereomer, a mixture of diastereomers, or an isotopic variant thereof; or a pharmaceutically acceptable salt or solvate thereof.

In yet another embodiment, provided herein are methods for treating, preventing, or ameliorating one or more symptoms of a liver disease or disorder associated with a drug-resistant HCV infection, comprising administering to a subject a therapeutically effective amount of a compound disclosed herein, e.g., a compound of any of Formulae disclosed herein, including Formulae I to XIV, IIIa to XIVa, IIIb to XIVb, IIIc to XIVc, IIId to XIVd, IIIe to XIVe, IA to IAe, IIA to IIAe, IB, IIB to IIBd, IIIB to IIIBd, IC to ICc, ID to IDd, and IE to IEc, including a single enantiomer, a racemic mixture, a diastereomer, a mixture of diastereomers, or an isotopic variant thereof; or a pharmaceutically acceptable salt or solvate thereof. Non-limiting examples of diseases associated with drug-resistant HCV infection include chronic hepatitis, cirrhosis, hepatocarcinoma, or extra hepatic manifestation.

In certain embodiments, the HCV infection is caused by a hepatitis C virus or variant thereof as described herein.

In certain embodiments, the drug-resistant HCV is resistant to an anti-HCV agent. In certain embodiments, the anti-HCV agent is an interferon. In certain embodiments, the anti-HCV agent is ribaririn. In certain embodiments, the anti-HCV agent is amantadine. In certain embodiments, the anti-HCV agent is an interleukin. In certain embodiments, the anti-HCV agent is a phenanthrenequinone. In certain embodiments, the anti-HCV agent is a thiazolidine. In certain embodiments, the anti-HCV agent is a benzanilide. In certain embodiments, the anti-HCV agent is a helicase inhibitor. In certain embodiments, the anti-HCV agent is a nucleotide analogue. In certain embodiments, the anti-HCV agent is a gliotoxin. In certain embodiments, the anti-HCV agent is a cerulenin. In certain embodiments, the anti-HCV agent is an antisense phopshorothioate ologodexoynucleotide. In certain embodiments, the anti-HCV agent is an inhibitor of IRES-dependent translation. In certain embodiments, the anti-HCV agent is a ribozyme. In certain embodiments, the anti-HCV agent is a cyclophilin inhibitor. In certain embodiments, the anti-HCV agent is SYC-635.

In certain embodiments, the anti-HCV agent is a protease inhibitor. In certain embodiments, the anti-HCV agent is a cysteine protease inhibitor. In certain embodiments, the anti-HCV agent is a caspase inhibitor. In certain embodiments, the anti-HCV agent is GS 9450. In certain embodiments, the anti-HCV agent is a serine protease inhibitor. In certain embodiments, the anti-HCV agent is an NS3/4A serine protease inhibitor. In certain embodiments, the anti-HCV agent is a serine protease inhibitor selected from ABT-450, faldaprevir (BI-201335), asunaprevir (BMS-650032), boceprevir (SCH 503034), danoprevir (ITMN-191/R7227), GS-9256, GS-9451, IDX136, IDX316, IDX320, MK-5172, SCH₉₀₀₅₁₈, telaprevir (VX-950), TMC 435, vaniprevir (MK-7009), VX-985, and mixtures thereof.

In certain embodiments, the anti-HCV agent is a polymerase inhibitor. In certain embodiments, the anti-HCV agent is an NS5B polymerase inhibitor. In certain embodiments, the anti-HCV agent is a polymerase inhibitor selected from ABT-072, ABT-333, AG-02154, ANA598, ANA773, deleobuvir (BI 207127), GS-9190, GS-9669, HCV-796, IDX184, IDX375, JTK-109, MK-0608, MK-3281, NM283, PF-868554, PSI-879, PSI-938, PSI-6130, PSI-7851, sofosbuvir (PSI-7977), R1626, R7128, RG7128, VCH-759, VCH-916, VX-222 (VCH-222), and mixtures thereof. In certain embodiments, the NS5B polymerase inhibitor is a nucleotide inhibitor. In certain embodiments, the NS5B polymerase inhibitor is a 2′-C-methylnucleoside. In certain embodiments, the NS5B polymerase inhibitor is a 2′-F-2′-C-methylnucleoside. In certain embodiments, the NS5B polymerase inhibitor is a non-nucleoside inhibitor. In certain embodiments, the NS5B polymerase inhibitor is a benzofuran, benzothiadiazine, or thiophene.

In certain embodiments, the anti-HCV agent is an NS5A inhibitor. In certain embodiments, the anti-HCV agent is an NS5A inhibitor selected from daclatasvir (BMS-790052), BMS-824393, ledipasvir (GS-5885), GSK2336805, PPI-668, and mixtures thereof.

In certain embodiments, the drug-resistance of the HCV infection is caused by an HCV variant. In certain embodiments, the HCV variant contains an NS3 protein variant. In certain embodiments, the NS3 protein variant contains a mutation or deletion. In certain embodiments, the NS3 protein variant contains one or more mutations and/or deletions at the amino acid positions of 9, 16, 18, 23, 36, 39, 40, 41, 43, 54, 55, 65, 67, 70, 71, 80, 89, 109, 138, 155, 156, 162, 168, 170, 174, 176, 179, 260, and 489. In certain embodiments, the NS3 protein variant contains one or more mutations and/or deletions at the amino acid positions of 16, 23, 36, 39, 41, 43, 54, 55, 80, 89, 109, 138, 155, 156, 168, 170, 174, 176, 260, and 489. In certain embodiments, the NS3 protein variant contains one or more mutations and/or deletions at the amino acid positions of 36, 54, 155, 156, 168, and 170. In certain embodiments, the NS3 protein variant contains one, two, or more mutations and/or deletions, each independently selected from C16S, V23A, V36A, V36G, V36L, V36M, A39V, Q41R, F43C, F43I, F43S, F43V, T54A, T54S, V55A, Q80K, Q80G, Q80H, Q80L, Q80R, P89R, R109K, S138T, R155G, R155I, R155K, R155L, R155M, R155Q, R155S, R155T, E56G, E56I, E56S, E56T, E56V, D168A, D168E, D168G, D168H, D168I, D168N, D168T, D168V, D168Y, V170A, V170T, S174K, S174N, E176K, T260A, and S489L, provided that there is only one mutation or deletion at a given amino acid position in the NS3 protein variant. In certain embodiments, the NS3 protein variant contains one, two, or more mutations and/or deletions, each independently selected from R155K, E56S, E56T, D168V, and T260A, provided that there is only one mutation or deletion at a given amino acid position in the NS3 protein variant.

In certain embodiments, the HCV variant contains an NS4A protein variant. In certain embodiments, the NS4A protein variant contains a mutation or deletion. In certain embodiments, the NS4A protein variant contains a mutation at the amino acid position of 23. In certain embodiments, the NS4A protein variant contains the V23A mutation.

In certain embodiments, the HCV variant contains an NS4B protein variant. In certain embodiments, the NS4B protein variant contains a mutation or deletion. In certain embodiments, the NS4B protein variant contains a mutation at the amino acid position of 15. In certain embodiments, the NS4B protein variant contains the E15G mutation.

In certain embodiments, the HCV variant contains an NS5A protein variant. In certain embodiments, the NS5A protein variant contains a mutation or deletion. In certain embodiments, the NS5A protein variant contains one or more mutations and/or deletions at the amino acid positions of 23, 28, 30, 31, 32, 37, 54, 58, 63, and 93. In certain embodiments, the NS5A protein variant contains one or more mutations and/or deletions at the amino acid positions of 23, 24, 28, 30, 31, 32, 37, 54, 58, 63, 93, 295, 318, 320, 356, 404, and 442. In certain embodiments, the NS5A protein variant contains one or more mutations and/or deletions at the amino acid positions of 24, 28, 30, 31, 32, 54, 93, 295, and 318. In certain embodiments, the NS5A protein variant contains one, two, or more mutations and/or deletions, each independently selected from L23F, L28M, L28T, M28T, AQ30, Q30E, Q30H, Q30K, Q30R, ΔR30, R30E, R30Q, L31F, L31M, L31V, P32L, F37L, H54Y, Q54H, P58H, P58S, I63V, Y93C, Y93H, Y93N, and Y93S, provided that there is only one mutation or deletion at a given amino acid position in the NS5A protein variant. In certain embodiments, the NS5A protein variant contains one, two, or more mutations and/or deletions, each independently selected from L23F, K24E, L28M, L28T, M28T, AQ30, Q30E, Q30H, Q30K, Q30R, ΔR30, R30E, R30Q, L31F, L31M, L31V, P32L, F37L, H54Y, Q54H, P58H, P58S, I63V, Y93C, Y93H, Y93N, Y93S, E295G, R318W, D320E, R356Q, G404S, and E442G, provided that there is only one mutation or deletion at a given amino acid position in the NS5A protein variant. In certain embodiments, the NS5A protein variant contains one, two, or more mutations and/or deletions, each independently selected from L23F, K24E, L28M, L28T, AQ30, Q30E, Q30H, Q30K, Q30R, L31F, L31M, L31V, P32L, F37L, H54Y, Q54H, P58H, P58S, I63V, Y93C, Y93H, Y93N, Y93S, E295G, R318W, D320E, R356Q, G404S, and E442G, provided that there is only one mutation or deletion at a given amino acid position in the NS5A protein variant. In certain embodiments, the NS5A protein variant contains one, two, or more mutations and/or deletions, each independently selected from L23F, K24E, M28T, ΔR30, R30E, R30Q, L31F, L31M, L31V, P32L, F37L, H54Y, Q54H, P58H, P58S, I63V, Y93C, Y93H, Y93N, Y93S, E295G, R318W, D320E, R356Q, G404S, and E442G, provided that there is only one mutation or deletion at a given amino acid position in the NS5A protein variant. In certain embodiments, the NS5A protein variant contains one, two, or more mutations and/or deletions, each independently selected from K24E, M28T, Q30E, Q30H, Q30K, Q30R, L31F, L31M, L31V, P32L, Y93C, Y93H, Y93N, E295G, and R318W, provided that there is only one mutation or deletion at a given amino acid position in the NS5A protein variant.

In certain embodiments, the HCV variant contains an NS5B protein variant. In certain embodiments, the NS5B protein variant contains a mutation or deletion. In certain embodiments, the NS5B protein variant contains one or more mutations and/or deletions at the amino acid positions of 15, 95, 96, 142, 152, 156, 222, 223, 244, 282, 309, 310, 316, 320, 321, 326, 329, 333, 365, 411, 414, 415, 423, 445, 448, 451, 452, 495, 554, 558, and 559. In certain embodiments, the NS5B protein variant contains one or more mutations and/or deletions at the amino acid positions of 316, 414, and 423. In certain embodiments, the NS5B protein variant contains one, two, or more mutations and/or deletions, each independently selected from S15G, H95Q, H95R, S96T, N142T, G152E, P156L, R222Q, C223H, C223Y, D244N, S282T, Q309R, D310N, C316N, C316S, C316Y, L320I, V321I, S326G, T329I, A333E, S365A, S365T, N411S, M414I, M414L, M414T, F415Y, M423I, M423T, M423V, C445F, Y448H, C451R, Y452H, P495A, P495I, G554D, G554S, G558R, D559G, D559N, and D559S, provided that there is only one mutation or deletion at a given amino acid position in the NS5B protein variant. In certain embodiments, the NS5B protein variant contains one, two, or more mutations and/or deletions, each independently selected from C316Y, M414T, and M423T, provided that there is only one mutation or deletion at a given amino acid position in the NS5B protein variant.

In one embodiment, provided herein is a method for treating or preventing infection caused by or associated with a hepatitis C virus variant, comprising administering to a subject a therapeutically effective amount of a compound disclosed herein, e.g., a compound of any of Formulae disclosed herein, including Formulae I to XIV, IIIa to XIVa, IIIb to XIVb, IIIc to XIVc, IIId to XIVd, IIIe to XIVe, IA to IAe, IIA to IIAe, IB, IIB to IIBd, IIIB to IIIBd, IC to ICc, ID to IDd, and IE to IEc, including a single enantiomer, a racemic mixture, a diastereomer, a mixture of diastereomers, or an isotopic variant thereof; or a pharmaceutically acceptable salt or solvate thereof.

In another embodiment, provided herein is a method for treating, preventing, or ameliorating one or more symptoms of a liver disease or disorder caused by or associated with a hepatitis C virus variant, comprising administering to a subject a therapeutically effective amount of a compound disclosed herein, e.g., a compound of any of Formulae disclosed herein, including Formulae I to XIV, IIIa to XIVa, IIIb to XIVb, IIIc to XIVc, IIId to XIVd, IIIe to XIVe, IA to IAe, IIA to IIAe, IB, IIB to IIBd, IIIB to IIIBd, IC to ICc, ID to IDd, and IE to IEc, including a single enantiomer, a racemic mixture, a diastereomer, a mixture of diastereomers, or an isotopic variant thereof; or a pharmaceutically acceptable salt or solvate thereof.

In certain embodiments, the HCV variant contains an NS5A protein variant as described herein.

In one embodiment, provided herein is a method for treating or preventing infection caused by or associated with a hepatitis C virus containing an NS5A protein variant as described herein, comprising administering to a subject a therapeutically effective amount of a compound disclosed herein, e.g., a compound of any of Formulae disclosed herein, including Formulae I to XIV, IIIa to XIVa, IIIb to XIVb, IIIc to XIVc, IIId to XIVd, IIIe to XIVe, IA to IAe, IIA to IIAe, IB, IIB to IIBd, IIIB to IIIBd, IC to ICc, ID to IDd, and IE to IEc, including a single enantiomer, a racemic mixture, a diastereomer, a mixture of diastereomers, or an isotopic variant thereof; or a pharmaceutically acceptable salt or solvate thereof.

In another embodiment, provided herein is a method for treating, preventing, or ameliorating one or more symptoms of a liver disease or disorder caused by or associated with hepatitis C virus containing an NS5A protein variant as described herein, comprising administering to a subject a therapeutically effective amount of a compound disclosed herein, e.g., a compound of any of Formulae disclosed herein, including Formulae I to XIV, IIIa to XIVa, IIIb to XIVb, IIIc to XIVc, IIId to XIVd, IIIe to XIVe, IA to IAe, IIA to IIAe, IB, IIB to IIBd, IIIB to IIIBd, IC to ICc, ID to IDd, and IE to IEc, including a single enantiomer, a racemic mixture, a diastereomer, a mixture of diastereomers, or an isotopic variant thereof; or a pharmaceutically acceptable salt or solvate thereof.

In one embodiment, the subject is a mammal. In another embodiment, the subject is a human.

In one embodiment, provided herein is a method for inhibiting replication of a virus in a host, which comprises contacting the host with a therapeutically effective amount of a compound provided herein, e.g., a compound of any of Formulae disclosed herein, including Formulae I to XIV, IIIa to XIVa, IIIb to XIVb, IIIc to XIVc, IIId to XIVd, IIIe to XIVe, IA to IAe, IIA to IIAe, IB, IIB to IIBd, IIIB to IIIBd, IC to ICc, ID to IDd, and IE to IEc, including a single enantiomer, a racemic mixture, a diastereomer, a mixture of diastereomers, or an isotopic variant thereof; or a pharmaceutically acceptable salt or solvate thereof.

In certain embodiments, the virus is a hepatitis C virus. In certain embodiments, the virus is a drug-resistant hepatitis C virus. In certain embodiments, the virus is a hepatitis C virus variant.

In one embodiment, the hepatitis C virus is HCV genotype 1. In certain embodiments, the hepatitis C virus is HCV subtype 1a. In certain embodiments, the hepatitis C virus is HCV subtype 1b. In certain embodiments, the hepatitis C virus is HCV subtype 1c.

In another embodiment, the hepatitis C virus is HCV genotype 2. In certain embodiments, the hepatitis C virus is HCV subtype 2a. In certain embodiments, the hepatitis C virus is HCV subtype 2b. In certain embodiments, the hepatitis C virus is HCV subtype 2c.

In yet another embodiment, the hepatitis C virus is HCV genotype 3. In certain embodiments, the hepatitis C virus is HCV subtype 3a. In certain embodiments, the hepatitis C virus is HCV subtype 3b.

In yet another embodiment, the hepatitis C virus is HCV genotype 4. In certain embodiments, the hepatitis C virus is HCV subtype 4a. In certain embodiments, the hepatitis C virus is HCV subtype 4b. In certain embodiments, the hepatitis C virus is HCV subtype 4c. In certain embodiments, the hepatitis C virus is HCV subtype 4d. In certain embodiments, the hepatitis C virus is HCV subtype 4e.

In yet another embodiment, the hepatitis C virus is HCV genotype 5. In yet another embodiment, the hepatitis C virus is HCV subtype 5a.

In yet another embodiment, the hepatitis C virus is HCV genotype 6. In yet another embodiment, the hepatitis C virus is HCV subtype 6a.

In yet another embodiment, the hepatitis C virus is HCV genotype 7. In yet another embodiment, the hepatitis C virus is HCV subtype 7a.

In yet another embodiment, the hepatitis C virus is HCV genotype 8. In yet another embodiment, the hepatitis C virus is HCV subtype 8a. In yet another embodiment, the hepatitis C virus is HCV subtype 8b.

In yet another embodiment, the hepatitis C virus is HCV genotype 9. In yet another embodiment, the hepatitis C virus is HCV subtype 9a.

In yet another embodiment, the hepatitis C virus is HCV genotype 10. In yet another embodiment, the hepatitis C virus is HCV subtype 10a.

In still another embodiment, the hepatitis C virus is HCV genotype 11. In yet another embodiment, the hepatitis C virus is HCV subtype 11a.

In one embodiment, the HCV variant is a variant of HCV genotype 1. In certain embodiments, the HCV variant is a variant of HCV subtype 1a. In certain embodiments, the HCV variant is a variant of HCV subtype 1b. In certain embodiments, the HCV variant is a variant of HCV subtype 1c.

In certain embodiments, the HCV variant is a variant of HCV subtype 1a, which contains an NS5A protein variant. In certain embodiments, the NS5A protein variant contains a mutation or deletion. In certain embodiments, the NS5A protein variant contains one or more mutations and/or deletions at the amino acid positions of 28, 30, 31, 32, 54, and 93. In certain embodiments, the NS5A protein variant contains one or more mutations and/or deletions at the amino acid positions of 23, 24, 28, 30, 31, 32, 37, 54, 58, 63, 93, 295, 318, 320, 356, 404, and 442. In certain embodiments, the NS5A protein variant contains one or more mutations and/or deletions at the amino acid positions of 24, 28, 30, 31, 32, 54, 93, 295, and 318. In certain embodiments, the NS5A protein variant contains one, two, or more mutations and/or deletions, each independently selected from M28T, AQ30, Q30E, Q30H, Q30K, Q30R, L31F, L31M, L31V, P32L, H54Y, Y93C, Y93H, and Y93N, provided that there is only one mutation or deletion at a given amino acid position in the NS5A protein variant. In certain embodiments, the NS5A protein variant contains one, two, or more mutations and/or deletions, each independently selected from L23F, K24E, L28M, L28T, M28T, AQ30, Q30E, Q30H, Q30K, Q30R, ΔR30, R30E, R30Q, L31F, L31M, L31V, P32L, F37L, H54Y, Q54H, P58H, P58S, I63V, Y93C, Y93H, Y93N, Y93S, E295G, R318W, D320E, R356Q, G404S, and E442G, provided that there is only one mutation or deletion at a given amino acid position in the NS5A protein variant. In certain embodiments, the NS5A protein variant contains one, two, or more mutations and/or deletions, each independently selected from L23F, K24E, L28M, L28T, AQ30, Q30E, Q30H, Q30K, Q30R, L31F, L31M, L31V, P32L, F37L, H54Y, Q54H, P58H, P58S, I63V, Y93C, Y93H, Y93N, Y93S, E295G, R318W, D320E, R356Q, G404S, and E442G, provided that there is only one mutation or deletion at a given amino acid position in the NS5A protein variant. In certain embodiments, the NS5A protein variant contains one, two, or more mutations and/or deletions, each independently selected from L23F, K24E, M28T, ΔR30, R30E, R30Q, L31F, L31M, L31V, P32L, F37L, H54Y, Q54H, P58H, P58S, I63V, Y93C, Y93H, Y93N, Y93S, E295G, R318W, D320E, R356Q, G404S, and E442G, provided that there is only one mutation or deletion at a given amino acid position in the NS5A protein variant. In certain embodiments, the NS5A protein variant contains one, two, or more mutations and/or deletions, each independently selected from K24E, M28T, Q30E, Q30H, Q30K, Q30R, L31F, L31M, L31V, P32L, Y93C, Y93H, Y93N, E295G, and R318W, provided that there is only one mutation or deletion at a given amino acid position in the NS5A protein variant. In certain embodiments, the NS5A protein variant contains one or more mutations at the amino acid positions of 28, 30, 31, 32, and 93. In certain embodiments, the NS5A protein variant contains one, two, or more mutations, each independently selected from M28T, Q30E, Q30H, Q30K, Q30R, L31F, L31M, L31V, P32L, Y93C, Y93H, and Y93N, provided that there is only one mutation at a given amino acid position in the NS5A protein variant. In certain embodiments, the NS5A protein variant contains one or more mutations at the amino acid positions of 24, 28, 30, 31, 32, 93, 295, and 318. In certain embodiments, the NS5A protein variant contains one, two, or more mutations, each independently selected from K24E, M28T, Q30E, Q30H, Q30K, Q30R, L31F, L31M, L31V, P32L, Y93C, Y93H, Y93N, E295G, and R318W, provided that there is only one mutation at a given amino acid position in the NS5A protein variant.

In certain embodiments, the HCV variant is a variant of HCV subtype 1b, which contains an NS5A protein variant. In certain embodiments, the NS5A protein variant contains a mutation or deletion. In certain embodiments, the NS5A protein variant contains one or more mutations and/or deletions at the amino acid positions of 23, 28, 30, 31, 32, 37, 54, 58, 63, and 93. In certain embodiments, the NS5A protein variant contains one or more mutations and/or deletions at the amino acid positions of 23, 24, 28, 30, 31, 32, 37, 54, 58, 63, 93, 295, 318, 320, 356, 404, and 442. In certain embodiments, the NS5A protein variant contains one or more mutations and/or deletions at the amino acid positions of 24, 28, 30, 31, 32, 54, 93, 295, and 318. In certain embodiments, the NS5A protein variant contains one, two, or more mutations and/or deletions, each independently selected from L23F, L28M, L28T, ΔR30, R30E, R30Q, L31F, L31M, L31V, P32L, F37L, Q54H, P58H, P58S, I63V, Y93C, Y93H, Y93N, and Y93S, provided that there is only one mutation or deletion at a given amino acid position in the NS5A protein variant. In certain embodiments, the NS5A protein variant contains one, two, or more mutations and/or deletions, each independently selected from L23F, K24E, L28M, L28T, M28T, AQ30, Q30E, Q30H, Q30K, Q30R, ΔR30, R30E, R30Q, L31F, L31M, L31V, P32L, F37L, H54Y, Q54H, P58H, P58S, I63V, Y93C, Y93H, Y93N, Y93S, E295G, R318W, D320E, R356Q, G404S, and E442G, provided that there is only one mutation or deletion at a given amino acid position in the NS5A protein variant. In certain embodiments, the NS5A protein variant contains one, two, or more mutations and/or deletions, each independently selected from L23F, K24E, L28M, L28T, AQ30, Q30E, Q30H, Q30K, Q30R, L31F, L31M, L31V, P32L, F37L, H54Y, Q54H, P58H, P58S, I63V, Y93C, Y93H, Y93N, Y93S, E295G, R318W, D320E, R356Q, G404S, and E442G, provided that there is only one mutation or deletion at a given amino acid position in the NS5A protein variant. In certain embodiments, the NS5A protein variant contains one, two, or more mutations and/or deletions, each independently selected from L23F, K24E, M28T, ΔR30, R30E, R30Q, L31F, L31M, L31V, P32L, F37L, H54Y, Q54H, P58H, P58S, 163V, Y93C, Y93H, Y93N, Y93S, E295G, R318W, D320E, R356Q, G404S, and E442G, provided that there is only one mutation or deletion at a given amino acid position in the NS5A protein variant. In certain embodiments, the NS5A protein variant contains one, two, or more mutations and/or deletions, each independently selected from K24E, M28T, Q30E, Q30H, Q30K, Q30R, L31F, L31M, L31V, P32L, Y93C, Y93H, Y93N, E295G, and R318W, provided that there is only one mutation or deletion at a given amino acid position in the NS5A protein variant. In certain embodiments, the NS5A protein variant contains one or more mutations at the amino acid positions of 28, 30, 31, 32, and 93. In certain embodiments, the NS5A protein variant contains one, two, or more mutations, each independently selected from L28T, R30E, L31F, L31M, L31V, P32L, Y93C, Y93H, and Y93N, provided that there is only one mutation at a given amino acid position in the NS5A protein variant. In certain embodiments, the NS5A protein variant contains one or more mutations at the amino acid positions of 24, 28, 30, 31, 32, 93, 295, and 318. In certain embodiments, the NS5A protein variant contains one, two, or more mutations, each independently selected from K24E, M28T, Q30E, Q30H, Q30K, Q30R, L31F, L31M, L31V, P32L, Y93C, Y93H, Y93N, E295G, and R318W, provided that there is only one mutation at a given amino acid position in the NS5A protein variant.

In another embodiment, the HCV variant is a variant of HCV genotype 2. In certain embodiments, the HCV variant is a variant of HCV subtype 2a. In certain embodiments, the HCV variant is a variant of HCV subtype 2b. In certain embodiments, the HCV variant is a variant of HCV subtype 2c.

In yet another embodiment, the HCV variant is a variant of HCV genotype 3. In certain embodiments, the HCV variant is a variant of HCV subtype 3a. In certain embodiments, the HCV variant is a variant of HCV subtype 3b.

In yet another embodiment, the HCV variant is a variant of HCV genotype 4. In certain embodiments, the HCV variant is a variant of HCV subtype 4a. In certain embodiments, the HCV variant is a variant of HCV subtype 4b. In certain embodiments, the HCV variant is a variant of HCV subtype 4c. In certain embodiments, the HCV variant is a variant of HCV subtype 4d. In certain embodiments, the HCV variant is a variant of HCV subtype 4e.

In yet another embodiment, the HCV variant is a variant of HCV genotype 5. In yet another embodiment, the HCV variant is a variant of HCV subtype 5a.

In yet another embodiment, the HCV variant is a variant of HCV genotype 6. In yet another embodiment, the HCV variant is a variant of HCV subtype 6a.

In yet another embodiment, the HCV variant is a variant of HCV genotype 7. In yet another embodiment, the HCV variant is a variant of HCV subtype 7a.

In yet another embodiment, the HCV variant is a variant of HCV genotype 8. In yet another embodiment, the HCV variant is a variant of HCV subtype 8a. In yet another embodiment, the HCV variant is a variant of HCV subtype 8b.

In yet another embodiment, the HCV variant is a variant of HCV genotype 9. In yet another embodiment, the HCV variant is a variant of HCV subtype 9a.

In yet another embodiment, the HCV variant is a variant of HCV genotype 10. In yet another embodiment, the HCV variant is a variant of HCV subtype 10a.

In still another embodiment, the HCV variant is a variant of HCV genotype 11. In yet another embodiment, the HCV variant is a variant of HCV subtype 11a.

In certain embodiments, provided herein is a method for inhibiting replication of hepatitis C virus containing an NS5A protein variant in a host, which comprises administering to the host a therapeutically effective amount of a compound disclosed herein, e.g., a compound of any of Formulae disclosed herein, including Formulae I to XIV, IIIa to XIVa, IIIb to XIVb, IIIc to XIVc, IIId to XIVd, IIIe to XIVe, IA to IAe, IIA to IIAe, IB, IIB to IIBd, IIIB to IIIBd, IC to ICc, ID to IDd, and IE to IEc, including a single enantiomer, a racemic mixture, a diastereomer, a mixture of diastereomers, or an isotopic variant thereof; or a pharmaceutically acceptable salt or solvate thereof.

In one embodiment, the host is a cell. In another embodiment, the host is a human cell. In yet another embodiment, the host is a mammal. In still another embodiment, the host is human.

In certain embodiments, administration of a therapeutically effective amount of a compound provided herein (e.g., a compound of any of Formulae disclosed herein, including Formulae I to XIV, IIIa to XIVa, IIIb to XIVb, IIIc to XIVc, IIId to XIVd, IIIe to XIVe, IA to IAe, IIA to IIAe, IB, IIB to IIBd, IIIB to IIIBd, IC to ICc, ID to IDd, and IE to IEc, including a single enantiomer, a racemic mixture, a diastereomer, a mixture of diastereomers, or an isotopic variant thereof; or a pharmaceutically acceptable salt or solvate thereof) results in a 10%, 20%, 30%, 40%, 50%, 60%, 70%, 80%, 90%, 95%, 99% or more reduction in the replication of the virus relative to a subject without administration of the compound, as determined at 1 day, 2 days, 3 days, 4 days, 5 days, 10 days, 15 days, or 30 days after the administration by a method known in the art, e.g., determination of viral titer.

In certain embodiments, administration of a therapeutically effective amount of a compound provided herein (e.g., a compound of any of Formulae disclosed herein, including Formulae I to XIV, IIIa to XIVa, IIIb to XIVb, IIIc to XIVc, IIId to XIVd, IIIe to XIVe, IA to IAe, IIA to IIAe, IB, IIB to IIBd, IIIB to IIIBd, IC to ICc, ID to IDd, and IE to IEc, including a single enantiomer, a racemic mixture, a diastereomer, a mixture of diastereomers, or an isotopic variant thereof; or a pharmaceutically acceptable salt or solvate thereof) results in a 1, 2, 3, 4, 5, 10, 15, 20, 25, 50, 75, 100-fold or more reduction in the replication of the virus relative to a subject without administration of the compound, as determined at 1 day, 2 days, 3 days, 4 days, 5 days, 10 days, 15 days, or 30 days after the administration by a method known in the art.

In certain embodiments, administration of a therapeutically effective amount of a compound provided herein (e.g., a compound of any of Formulae disclosed herein, including Formulae I to XIV, IIIa to XIVa, IIIb to XIVb, IIIc to XIVc, IIId to XIVd, IIIe to XIVe, IA to IAe, IIA to IIAe, IB, IIB to IIBd, IIIB to IIIBd, IC to ICc, ID to IDd, and IE to IEc, including a single enantiomer, a racemic mixture, a diastereomer, a mixture of diastereomers, or an isotopic variant thereof; or a pharmaceutically acceptable salt or solvate thereof) results in a 10%, 20%, 30%, 40%, 50%, 60%, 70%, 80%, 90%, 95%, 99% or more reduction in the viral titer relative to a subject without administration of the compound, as determined at 1 day, 2 days, 3 days, 4 days, 5 days, 10 days, 15 days, or 30 days after the administration by a method known in the art.

In certain embodiments, administration of a therapeutically effective amount of a compound provided herein (e.g., a compound of any of Formulae disclosed herein, including Formulae I to XIV, IIIa to XIVa, IIIb to XIVb, IIIc to XIVc, IIId to XIVd, IIIe to XIVe, IA to IAe, IIA to IIAe, IB, IIB to IIBd, IIIB to IIIBd, IC to ICc, ID to IDd, and IE to IEc, including a single enantiomer, a racemic mixture, a diastereomer, a mixture of diastereomers, or an isotopic variant thereof; or a pharmaceutically acceptable salt or solvate thereof) results in a 1, 2, 3, 4, 5, 10, 15, 20, 25, 50, 75, 100 or more fold reduction in the viral titer relative to a subject without administration of the compound, as determined at 1 day, 2 days, 3 days, 4 days, 5 days, 10 days, 15 days, or 30 days after the administration by a method known in the art.

In certain embodiments, provided herein is a method for inhibiting the replication of an HCV virus, which comprises contacting the virus with a therapeutically effective amount of a compound provided herein, e.g., a compound of any of Formulae disclosed herein, including Formulae I to XIV, IIIa to XIVa, IIIb to XIVb, IIIc to XIVc, IIId to XIVd, IIIe to XIVe, IA to IAe, IIA to IIAe, IB, IIB to IIBd, IIIB to IIIBd, IC to ICc, ID to IDd, and IE to IEc, including a single enantiomer, a racemic mixture, a diastereomer, a mixture of diastereomers, or an isotopic variant thereof; or a pharmaceutically acceptable salt or solvate thereof.

In certain embodiments, the contacting of the virus with a therapeutically effective amount of a compound provided herein (e.g., a compound of any of Formulae disclosed herein, including Formulae I to XIV, IIIa to XIVa, IIIb to XIVb, IIIc to XIVc, IIId to XIVd, Ille to XIVe, IA to IAe, IIA to IIAe, IB, IIB to IIBd, IIIB to IIIBd, IC to ICc, ID to IDd, and IE to IEc, including a single enantiomer, a racemic mixture, a diastereomer, a mixture of diastereomers, or an isotopic variant thereof; or a pharmaceutically acceptable salt or solvate thereof) results in a 10%, 20%, 30%, 40%, 50%, 60%, 70%, 80%, 90%, 95%, 99% or more reduction in the virus titer relative to the virus without such contact, as determined at 1 day, 2 days, 3 days, 4 days, 5 days, 10 days, 15 days, or 30 days after the initial contact, by a method known in the art.

In certain embodiments, the contacting of the virus with a therapeutically effective amount of a compound provided herein (e.g., a compound of any of Formulae disclosed herein, including Formulae I to XIV, IIIa to XIVa, IIIb to XIVb, IIIc to XIVc, IIId to XIVd, IIIe to XIVe, IA to IAe, IIA to IIAe, IB, IIB to IIBd, IIIB to IIIBd, IC to ICc, ID to IDd, and IE to IEc, including a single enantiomer, a racemic mixture, a diastereomer, a mixture of diastereomers, or an isotopic variant thereof; or a pharmaceutically acceptable salt or solvate) results in a 1, 2, 3, 4, 5, 10, 15, 20, 25, 50, 75, 100 or more fold reduction in the viral titer relative to the virus without such contact, as determined at 1 day, 2 days, 3 days, 4 days, 5 days, 10 days, 15 days, or 30 days after the initial contact, by a method known in the art.

In still another embodiment, provided herein is a method for treating, preventing, or ameliorating one or more symptoms of a liver disease or disorder associated with an HCV infection, comprising administering to a subject a therapeutically effective amount of the compound provided herein, e.g., a compound of any of Formulae disclosed herein, including Formulae I to XIV, IIIa to XIVa, IIIb to XIVb, IIIc to XIVc, IIId to XIVd, IIIe to XIVe, IA to IAe, IIA to IIAe, IB, IIB to IIBd, IIIB to IIIBd, IC to ICc, ID to IDd, and IE to IEc, including a single enantiomer, a racemic mixture, a diastereomer, a mixture of diastereomers, or an isotopic variant thereof; or a pharmaceutically acceptable salt or solvate thereof. Non-limiting examples of diseases associated with HCV infection include chronic hepatitis, cirrhosis, hepatocarcinoma, or extra hepatic manifestation.

Depending on the condition, disorder, or disease, to be treated and the subject's condition, a compound provided herein may be administered by oral, parenteral (e.g., intramuscular, intraperitoneal, intravenous, intracerebroventricular (ICV), intracistemal injection or infusion, subcutaneous injection, or implant), inhalation, nasal, vaginal, rectal, sublingual, or topical (e.g., transdermal or local) routes of administration, and may be formulated, alone or together, in suitable dosage unit with pharmaceutically acceptable carriers, adjuvants and vehicles appropriate for each route of administration.

The dose may be in the form of one, two, three, four, five, six, or more sub-doses that are administered at appropriate intervals per day. The dose or sub-doses can be administered in the form of dosage units containing from about 0.1 to about 1,000 milligram, from about 0.1 to about 500 milligrams, or from 0.5 about to about 100 milligram active ingredient(s) per dosage unit, and if the condition of the patient requires, the dose can, by way of alternative, be administered as a continuous infusion.

In certain embodiments, an appropriate dosage level is about 0.01 to about 100 mg per kg patient body weight per day (mg/kg per day), about 0.01 to about 50 mg/kg per day, about 0.01 to about 25 mg/kg per day, or about 0.05 to about 10 mg/kg per day, which may be administered in single or multiple doses. A suitable dosage level may be about 0.01 to about 100 mg/kg per day, about 0.05 to about 50 mg/kg per day, or about 0.1 to about 10 mg/kg per day. Within this range the dosage may be about 0.01 to about 0.1, about 0.1 to about 1.0, about 1.0 to about 10, or about 10 to about 50 mg/kg per day.

### Combination Therapy

The compounds provided herein may also be combined or used in combination with other therapeutic agents useful in the treatment and/or prevention of an HCV infection.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a disease or disorder. A first therapy (e.g., a prophylactic or therapeutic agent such as a compound provided herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to the subject. Triple therapy is also contemplated herein.

As used herein, the term “synergistic” includes a combination of a compound provided herein and another therapy (e.g., a prophylactic or therapeutic agent) which has been or is currently being used to prevent, treat, or manage a condition, disorder, or disease, which is more effective than the additive effects of the therapies. A synergistic effect of a combination of therapies (e.g., a combination of prophylactic or therapeutic agents) permits the use of lower dosages of one or more of the therapies and/or less frequent administration of said therapies to a subject with a condition, disorder, or disease. The ability to utilize lower dosages of a therapy (e.g., a prophylactic or therapeutic agent) and/or to administer said therapy less frequently reduces the toxicity associated with the administration of said therapy to a subject without reducing the efficacy of said therapy in the prevention, treatment, or management of a condition, disorder, or disease). In addition, a synergistic effect can result in improved efficacy of agents in the prevention, treatment, or management of a condition, disorder, or disease. Finally, a synergistic effect of a combination of therapies (e.g., a combination of prophylactic or therapeutic agents) may avoid or reduce adverse or unwanted side effects associated with the use of either therapy alone.

The compound provided herein can be administered in combination or alternation with another therapeutic agent, such as an anti-HCV agent. In combination therapy, effective dosages of two or more agents are administered together, whereas in alternation or sequential-step therapy, an effective dosage of each agent is administered serially or sequentially. The dosages given will depend on absorption, inactivation, and excretion rates of the drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

It has been recognized that drug-resistant variants of HCV can emerge after prolonged treatment with an antiviral agent. Drug resistance most typically occurs due to the mutation of a gene that encodes for an enzyme used in viral replication. The efficacy of a drug against the viral infection can be prolonged, augmented, or restored by administering the compound in combination or alternation with a second, and perhaps third, antiviral compound that induces a different mutation from that caused by the principle drug. Alternatively, the pharmacokinetics, biodistribution, or other parameters of the drug can be altered by such combination or alternation therapy. In general, combination therapy is typically preferred over alternation therapy because it induces multiple simultaneous stresses on the virus.

In certain embodiments, the pharmaceutical compositions provided herein further comprise a second antiviral agent as described herein. In certain embodiments, the compound provided herein is combined with one or more agents selected from an interferon, ribavirin, amantadine, an interleukin, an NS3 protease inhibitor, a cysteine protease inhibitor, a phenanthrenequinone, a thiazolidine, a benzanilide, a helicase inhibitor, a polymerase inhibitor, a nucleotide analogue, a gliotoxin, a cerulenin, an antisense phosphorothioate oligodeoxynucleotide, an inhibitor of IRES-dependent translation, or a ribozyme. In one embodiment, the second antiviral agent is an interferon. In another embodiment, the interferon is selected from pegylated interferon alpha 2a, interferon alfacon-1, natural interferon, ALBUFERON®, interferon beta-1a, omega interferon, interferon alpha, interferon gamma, interferon tau, interferon delta, or interferon gamma-1b.

In certain embodiments, the compound provided herein is combined with an HCV protease inhibitor, including, but not limited to, BI 201335 (Boehringer Ingelheim); TMC 435 or TMC 435350 (Medivir/Tibotec); ITMN 191/R7227 (InterMune); MK 7009 (Merck); SCH 5034/SCH 503034/Boceprevir and SCH 900518/narlaprevir (Schering); VX950/telaprevir (Vertex); substrate-based NS3 protease inhibitors as disclosed in DE 19914474, WO 98/17679, WO 98/22496, WO 99/07734, and Attwood et al., Antiviral Chemistry and Chemotherapy 1999, 10, 259-273; non-substrate-based NS3 protease inhibitors, including 2,4,6-trihydroxy-3-nitro-benzamide derivatives (Sudo et al., Biochem. Biophys. Res. Commun. 1997, 238, 643-647), a phenanthrenequinone (Chu et al., Tetrahedron Letters 1996, 37, 7229-7232), RD3-4082, RD3-4078, SCH 68631, and SCH 351633 (Chu et al., Bioorganic and Medicinal Chemistry Letters 1999, 9, 1949-1952); and Eglin C, a potent serine protease inhibitor (Qasim et al., Biochemistry 1997, 36, 1598-1607).

Other suitable protease inhibitors for the treatment of HCV include those disclosed in, for example, U.S. Pat. No. 6,004,933, which discloses a class of cysteine protease inhibitors of HCV endopeptidase 2.

Additional hepatitis C virus NS3 protease inhibitors include those disclosed in, for example, Llinàs-Brunet et al., Bioorg. Med. Chem. Lett. 1998, 8, 1713-1718; Steinkühler et al., Biochemistry 1998, 37, 8899-8905; U.S. Pat. Nos. 5,538,865; 5,990,276; 6,143,715; 6,265,380; 6,323,180; 6,329,379; 6,410,531; 6,420,380; 6,534,523; 6,608,027; 6,642,204; 6,653,295; 6,727,366; 6,838,475; 6,846,802; 6,867,185; 6,869,964; 6,872,805; 6,878,722; 6,908,901; 6,911,428; 6,995,174; 7,012,066; 7,041,698; 7,091,184; 7,169,760; 7,176,208; 7,208,600; and 7,491,794; U.S. Pat. Appl. Publ. Nos.: 2002/0016294, 2002/0016442; 2002/0032175; 2002/0037998; 2004/0229777; 2005/0090450; 2005/0153877; 2005/176648; 2006/0046956; 2007/0021330; 2007/0021351; 2007/0049536; 2007/0054842; 2007/0060510; 2007/0060565; 2007/0072809; 2007/0078081; 2007/0078122; 2007/0093414; 2007/0093430; 2007/0099825; 2007/0099929; 2007/0105781, 2008/0152622, 2009/0035271, 2009/0035272, 2009/0047244, 2009/0111969, 2009/0111982, 2009/0123425, 2009/0130059, 2009/0148407, 2009/0156800, 2009/0169510, 2009/0175822, 2009/0180981, and 2009/0202480; U.S. patent application Ser. No. 12/365,127; and International Pat. Appl. Publ. Nos.: WO 98/17679; WO 98/22496; WO 99/07734; WO 00/09543; WO 00/59929; WO 02/08187; WO 02/08251; WO 02/08256; WO 02/08198; WO 02/48116; WO 02/48157; WO 02/48172; WO 02/60926; WO 03/53349; WO 03/64416; WO 03/64455; WO 03/64456; WO 03/66103; WO 03/99274; WO 03/99316; WO 2004/032827; WO 2004/043339; WO 2005/037214; WO 2005/037860; WO 2006/000085; WO 2006/119061; WO 2006/122188; WO 2007/001406; WO 2007/014925; WO 2007/014926; WO 2007/015824, WO 2007/056120, WO 2008/019289, WO 2008/021960, WO 2008/022006, WO 2008/086161, WO 2009/053828, WO 2009/058856, WO 2009/073713, WO 2009/073780, WO 2009/080542, WO 2009/082701, WO 2009/082697, and WO 2009/085978; the disclosure of each of which is incorporated herein by reference in its entirety.

Other protease inhibitors include thiazolidine derivatives, such as RD-1-6250, RD4 6205, and RD4 6193, which show relevant inhibition in a reverse-phase HPLC assay with an NS3/4A fusion protein and NS5A/5B substrate (Sudo et al., Antiviral Research 1996, 32, 9-18); and thiazolidines and benzanilides identified in Kakiuchi et al., FEBS Lett. 1998, 421, 217-220; and Takeshita et al., Analytical Biochemistry 1997, 247, 242-246.

Suitable helicase inhibitors include, but are not limited to, those disclosed in U.S. Pat. No. 5,633,358; and International Pat. Appl. Publ. No. WO 97/36554.

Suitable nucleotide polymerase inhibitors include, but are not limited to, gliotoxin (Ferrari et al., Journal of Virology 1999, 73, 1649-1654) and cerulenin (Lohmann et al., Virology 1998, 249, 108-118).

Suitable interfering RNA (iRNA) based antivirals include, but are not limited to, short interfering RNA (siRNA) based antivirals, such as Sirna-034 and those described in International Pat. Appl. Publ. Nos. WO/03/070750 and WO 2005/012525, and U.S. Pat. Appl. Publ. No. 2004/0209831.

Suitable antisense phosphorothioate oligodeoxynucleotides (S-ODN) complementary to sequence stretches in the 5′ non-coding region (NCR) of HCV virus include, but are not limited to those described in Alt et al., Hepatology 1995, 22, 707-717, and nucleotides 326-348 comprising the 3′ end of the NCR and nucleotides 371-388 located in the core coding region of HCV RNA (Alt et al., Archives of Virology 1997, 142, 589-599; and Galderisi et al., Journal of Cellular Physiology 1999, 181, 251-257);

Suitable inhibitors of IRES-dependent translation include, but are not limited to, those described in Japanese Pat. Appl. Publ. Nos.: JP 08268890 and JP 10101591.

Suitable ribozymes include those disclosed in, for example, U.S. Pat. Nos. 6,043,077; 5,869,253; and 5,610,054.

Suitable nucleoside analogs include, but are not limited to, the compounds described in U.S. Pat. Nos. 6,660,721; 6,777,395; 6,784,166; 6,846,810; 6,927,291; 7,094,770; 7,105,499; 7,125,855; and 7,202,224; U.S. Pat. Appl. Publ. Nos. 2004/0121980; 2005/0009737; 2005/0038240; and 2006/0040890; and International Pat. Appl. Publ. Nos: WO 99/43691; WO 01/32153; WO 01/60315; WO 01/79246; WO 01/90121, WO 01/92282, WO 02/18404; WO 02/32920, WO 02/48165, WO 02/057425; WO 02/057287; WO 2004/002422, WO 2004/002999, and WO 2004/003000.

Other miscellaneous compounds that can be used as second agents include, for example, 1-amino-alkylcyclohexanes (U.S. Pat. No. 6,034,134), alkyl lipids (U.S. Pat. No. 5,922,757), vitamin E and other antioxidants (U.S. Pat. No. 5,922,757), squalene, amantadine, bile acids (U.S. Pat. No. 5,846,964), N-(phosphonacetyl)-L-aspartic acid (U.S. Pat. No. 5,830,905), benzenedicarboxamides (U.S. Pat. No. 5,633,388), polyadenylic acid derivatives (U.S. Pat. No. 5,496,546), 2′,3′-dideoxyinosine (U.S. Pat. No. 5,026,687), benzimidazoles (U.S. Pat. No. 5,891,874), plant extracts (U.S. Pat. Nos. 5,725,859; 5,837,257; and 6,056,961), and piperidines (U.S. Pat. No. 5,830,905).

In certain embodiments, one or more compounds provided herein are administered in combination or alternation with an anti-hepatitis C virus interferon, including, but not limited to, INTRON® A (interferon alfa-2b), PEGASYS® (Peginterferon alfa-2a) ROFERON® A (recombinant interferon alfa-2a), INFERGEN® (interferon alfacon-1), and PEG-INTRON® (pegylated interferon alfa-2b). In one embodiment, the anti-hepatitis C virus interferon is INFERGEN®, IL-29 (PEG-Interferon lambda), R7025 (Maxy-alpha), BELEROFON®, oral interferon alpha, BLX-883 (LOCTERON®), omega interferon, MULTIFERON®, medusa interferon, ALBUFERON®, or REBIF®.

In certain embodiments, one or more compounds provided herein are administered in combination or alternation with an anti-hepatitis C virus polymerase inhibitor, such as ribavirin, viramidine, NM 283 (valopicitabine), PSI-6130, R1626, HCV-796, R7128, and those as disclosed in U.S. Pat. Appl. Publ. Nos. 2009/0081158 and 2009/0238790, the disclosure of each of which is incorporated herein by reference in its entirety.

In certain embodiments, the one or more compounds provided herein are administered in combination with ribavirin and an anti-hepatitis C virus interferon, such as INTRON® A (interferon alfa-2b), PEGASYS® (Peginterferon alfa-2a), ROFERON® A (recombinant interferon alfa-2a), INFERGEN® (interferon alfacon-1), and PEG-INTRON® (pegylated interferon alfa-2b),

In certain embodiments, one or more compounds provided herein are administered in combination or alternation with an anti-hepatitis C virus protease inhibitor, such as GS-9451, ITMN-191, SCH 503034, VX950 (telaprevir), and TMC 435.

In certain embodiments, one or more compounds provided herein are administered in combination or alternation with an anti-hepatitis C virus vaccine, including, but not limited to, TG4040, PEVIPRO™, CGI-5005, HCV/MF59, GV1001, IC41, and INNO0101 (E1).

In certain embodiments, one or more compounds provided herein are administered in combination or alternation with an anti-hepatitis C virus monoclonal antibody, such as AB68 and XTL-6865 (formerly HepX-C); or an anti-hepatitis C virus polyclonal antibody, such as cicavir.

In certain embodiments, one or more compounds provided herein are administered in combination or alternation with an anti-hepatitis C virus immunomodulator, such as ZADAXIN® (thymalfasin), NOV-205, and oglufanide.

In certain embodiments, one or more compounds provided herein are administered in combination or alternation with NEXAVAR®, doxorubicin, PI-88, amantadine, JBK-122, VGX-410C, MX-3253 (celgosivir), SUVUS® (BIVN-401 or virostat), PF-03491390 (formerly IDN-6556), G126270, UT-231B, DEBIO-025, EMZ702, ACH-0137171, MitoQ, ANA975, AVI-4065, bavituximab (tarvacin), ALINIA® (nitrazoxanide), GS-9620, and PYN17.

The compounds provided herein can also be administered in combination with other classes of compounds, including, but not limited to, (1) alpha-adrenergic agents; (2) antiarrhythmic agents; (3) anti-atherosclerotic agents, such as ACAT inhibitors; (4) antibiotics, such as anthracyclines, bleomycins, mitomycin, dactinomycin, and plicamycin; (5) anticancer agents and cytotoxic agents, e.g., alkylating agents, such as nitrogen mustards, alkyl sulfonates, nitrosoureas, ethylenimines, and triazenes; (6) anticoagulants, such as acenocoumarol, argatroban, bivalirudin, lepirudin, fondaparinux, heparin, phenindione, warfarin, and ximelagatran; (7) anti-diabetic agents, such as biguanides (e.g., metformin), glucosidase inhibitors (e.g., acarbose), insulins, meglitinides (e.g., repaglinide), sulfonylureas (e.g., glimepiride, glyburide, and glipizide), thiozolidinediones (e.g., troglitazone, rosiglitazone, and pioglitazone), and PPAR-gamma agonists; (8) antifungal agents, such as amorolfine, amphotericin B, anidulafungin, bifonazole, butenafine, butoconazole, caspofungin, ciclopirox, clotrimazole, econazole, fenticonazole, filipin, fluconazole, isoconazole, itraconazole, ketoconazole, micafungin, miconazole, naftifine, natamycin, nystatin, oxyconazole, ravuconazole, posaconazole, rimocidin, sertaconazole, sulconazole, terbinafine, terconazole, tioconazole, and voriconazole; (9) antiinflammatories, e.g., non-steroidal anti-inflammatory agents, such as aceclofenac, acemetacin, amoxiprin, aspirin, azapropazone, benorilate, bromfenac, carprofen, celecoxib, choline magnesium salicylate, diclofenac, diflunisal, etodolac, etoricoxib, faislamine, fenbufen, fenoprofen, flurbiprofen, ibuprofen, indometacin, ketoprofen, ketorolac, lornoxicam, loxoprofen, lumiracoxib, meclofenamic acid, mefenamic acid, meloxicam, metamizole, methyl salicylate, magnesium salicylate, nabumetone, naproxen, nimesulide, oxyphenbutazone, parecoxib, phenylbutazone, piroxicam, salicyl salicylate, sulindac, sulfinpyrazone, suprofen, tenoxicam, tiaprofenic acid, and tolmetin; (10) antimetabolites, such as folate antagonists, purine analogues, and pyrimidine analogues; (11) anti-platelet agents, such as GPIIb/IIIa blockers (e.g., abciximab, eptifibatide, and tirofiban), P2Y(AC) antagonists (e.g., clopidogrel, ticlopidine and CS-747), cilostazol, dipyridamole, and aspirin; (12) antiproliferatives, such as methotrexate, FK506 (tacrolimus), and mycophenolate mofetil; (13) anti-TNF antibodies or soluble TNF receptor, such as etanercept, rapamycin, and leflunimide; (14) aP2 inhibitors; (15) beta-adrenergic agents, such as carvedilol and metoprolol; (16) bile acid sequestrants, such as questran; (17) calcium channel blockers, such as amlodipine besylate; (18) chemotherapeutic agents; (19) cyclooxygenase-2 (COX-2) inhibitors, such as celecoxib and rofecoxib; (20) cyclosporins; (21) cytotoxic drugs, such as azathioprine and cyclophosphamide; (22) diuretics, such as chlorothiazide, hydrochlorothiazide, flumethiazide, hydroflumethiazide, bendroflumethiazide, methylchlorothiazide, trichloromethiazide, polythiazide, benzothiazide, ethacrynic acid, ticrynafen, chlorthalidone, furosenide, muzolimine, bumetanide, triamterene, amiloride, and spironolactone; (23) endothelin converting enzyme (ECE) inhibitors, such as phosphoramidon; (24) enzymes, such as L-asparaginase; (25) Factor VIIa Inhibitors and Factor Xa Inhibitors; (26) famesyl-protein transferase inhibitors; (27) fibrates; (28) growth factor inhibitors, such as modulators of PDGF activity; (29) growth hormone secretagogues; (30) HMG CoA reductase inhibitors, such as pravastatin, lovastatin, atorvastatin, simvastatin, NK-104 (a.k.a. itavastatin, nisvastatin, or nisbastatin), and ZD-4522 (also known as rosuvastatin, atavastatin, or visastatin); neutral endopeptidase (NEP) inhibitors; (31) hormonal agents, such as glucocorticoids (e.g., cortisone), estrogens/antiestrogens, androgens/antiandrogens, progestins, and luteinizing hormone-releasing hormone antagonists, and octreotide acetate; (32) immunosuppressants; (33) mineralocorticoid receptor antagonists, such as spironolactone and eplerenone; (34) microtubule-disruptor agents, such as ecteinascidins; (35) microtubule-stabilizing agents, such as pacitaxel, docetaxel, and epothilones A-F; (36) MTP Inhibitors; (37) niacin; (38) phosphodiesterase inhibitors, such as PDE III inhibitors (e.g., cilostazol) and PDE V inhibitors (e.g., sildenafil, tadalafil, and vardenafil); (39) plant-derived products, such as vinca alkaloids, epipodophyllotoxins, and taxanes; (40) platelet activating factor (PAF) antagonists; (41) platinum coordination complexes, such as cisplatin, satraplatin, and carboplatin; (42) potassium channel openers; (43) prenyl-protein transferase inhibitors; (44) protein tyrosine kinase inhibitors; (45) renin inhibitors; (46) squalene synthetase inhibitors; (47) steroids, such as aldosterone, beclometasone, betamethasone, deoxycorticosterone acetate, fludrocortisone, hydrocortisone (cortisol), prednisolone, prednisone, methylprednisolone, dexamethasone, and triamcinolone; (48) TNF-alpha inhibitors, such as tenidap; (49) thrombin inhibitors, such as hirudin; (50) thrombolytic agents, such as anistreplase, reteplase, tenecteplase, tissue plasminogen activator (tPA), recombinant tPA, streptokinase, urokinase, prourokinase, and anisoylated plasminogen streptokinase activator complex (APSAC); (51) thromboxane receptor antagonists, such as ifetroban; (52) topoisomerase inhibitors; (53) vasopeptidase inhibitors (dual NEP-ACE inhibitors), such as omapatrilat and gemopatrilat; and (54) other miscellaneous agents, such as, hydroxyurea, procarbazine, mitotane, hexamethylmelamine, and gold compounds.

The compounds provided herein can also be provided as an article of manufacture using packaging materials well known to those of skill in the art. See, e.g., U.S. Pat. Nos. 5,323,907; 5,052,558; and 5,033,252. Examples of pharmaceutical packaging materials include, but are not limited to, blister packs, bottles, tubes, inhalers, pumps, bags, vials, containers, syringes, and any packaging material suitable for a selected formulation and intended mode of administration and treatment.

Provided herein also are kits which, when used by the medical practitioner, can simplify the administration of appropriate amounts of active ingredients to a subject. In certain embodiments, the kit provided herein includes a container and a dosage form of a compound provided herein, e.g., a compound of any of Formulae disclosed herein, including Formulae I to XIV, IIIa to XIVa, IIIb to XIVb, IIIc to XIVc, IIId to XIVd, IIIe to XIVe, IA to IAe, IIA to IIAe, IB, IIB to IIBd, IIIB to IIIBd, IC to ICc, ID to IDd, and IE to IEc, including a single enantiomer, a racemic mixture, a diastereomer, a mixture of diastereomers, or an isotopic variant thereof; or a pharmaceutically acceptable salt or solvate thereof.

In certain embodiments, the kit includes a container comprising a dosage form of the compound provided herein, e.g., a compound of any of Formulae disclosed herein, including Formulae I to XIV, IIIa to XIVa, IIIb to XIVb, IIIc to XIVc, IIId to XIVd, IIIe to XIVe, IA to IAe, IIA to IIAe, IB, IIB to IIBd, IIIB to IIIBd, IC to ICc, ID to IDd, and IE to IEc, including a single enantiomer, a racemic mixture, a diastereomer, a mixture of diastereomers, or an isotopic variant thereof; or a pharmaceutically acceptable salt or solvate thereof, in a container comprising one or more other therapeutic agent(s) described herein.

Kits provided herein can further include devices that are used to administer the active ingredients. Examples of such devices include, but are not limited to, syringes, needle-less injectors drip bags, patches, and inhalers. The kits provided herein can also include condoms for administration of the active ingredients.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, Water for Injection USP, Sodium Chloride Injection, Ringer's Injection, Dextrose Injection, Dextrose and Sodium Chloride Injection, and Lactated Ringer's Injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

The disclosure will be further understood by the following non-limiting examples.

### EXAMPLES

As used herein, the symbols and conventions used in these processes, schemes and examples, regardless of whether a particular abbreviation is specifically defined, are consistent with those used in the contemporary scientific literature, for example, the Journal of the American Chemical Society or the Journal of Biological Chemistry. Specifically, but without limitation, the following abbreviations may be used in the examples and throughout the specification: g (grams); mg (milligrams); mL (milliliters); μL (microliters); L (liter); mM (millimolar); M (micromolar); Hz (Hertz); MHz (megahertz); mmol (millimoles); eq. (equivalent); hr or hrs (hours); min (minutes); MS (mass spectrometry); NMR (nuclear magnetic resonance); ESI (electrospray ionization); HPLC (high-performance liquid chromatography or high pressure liquid chromatography); ACN (acetonitrile); CDCl₃ (deuterated chloroform); DCM (dichloromethane); DMF (N,N-dimethylformamide); DMSO (dimethylsulfoxide); DMSO-d₆ (deuterated dimethylsulfoxide); EtOAc (ethyl acetate); Et₂O (diethyl ether); EtOH (ethanol); MeOH (methanol); PE (petroleum ether); THF (tetrahydrofuran); DIPEA (N,N-diisopropylethylamine); TEA (triethylamine); TFA (trifluoroacetic acid); BOP (benzotriazole-1-yl-oxy-tris-(dimethylamino)-phosphonium hexafluorophosphate); HATU (2-(7-aza-1H-benzotriazole-1-yl)-1,1,3,3-tetramethyluronium hexafluorophosphate); TBTU (O-(benzotriazol-1-yl)-N,N,N′,N′-tetramethyluronium tetrafluoroborate); DIPC (1,3-diisopropylcarbodiimide); Me (methyl); Et (ethyl); iPr, (isopropyl); tBu (tert-butyl); Boc (tert-butoxylcarbony); Bn (benzyl); Ph (phenyl); AcO (acetate); PdCl₂(dppf) ((1,1′-bis(diphenylphosphino)ferrocene) dichloropalladium(II)); and Pd118 (1,1′-bis(di-tert-butylphosphino)ferrocene palladium (II) dichloride).

For all of the following examples, standard work-up and purification methods known to those skilled in the art can be utilized. Unless otherwise indicated, all temperatures are expressed in ° C. (degrees Centigrade). All reactions conducted at room temperature unless otherwise noted. Synthetic methodologies herein are intended to exemplify the applicable chemistry through the use of specific examples and are not indicative of the scope of the disclosure.

### Example 1

### Synthesis of Hepatitis C Virus Inhibitor Compounds

The synthesis of hepatitis C virus inhibitor compounds is shown in Schemes 1-3.

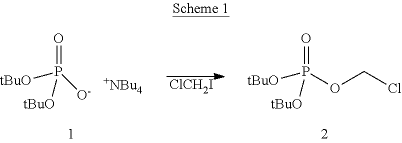 [SMILES: CC(C)(C)OP(=O)([O-])OC(C)(C)C ; "ClCH2I" ; CC(C)(C)OP(=O)(OCCl)OC(C)(C)C ; "+NBu4"]

Compound 2.

To chloroiodomethane 98% (53 mmol) was added dropwise a solution of tetra-n-butylammonium di-tert-butylphosphate (5.3 mmol) in benzene (1 mL/mmol). After stirred overnight at room temperature, the reaction mixture was concentrated under reduced pressure, and the oily residue was dissolved in diethyl ether and filtered. The filtrate was then washed with a saturated NaHCO₃ solution and brine, dried over sodium sulfate, filtered, and concentrated under reduced pressure. The crude residue was purified by flash chromatography on silica gel (PE/EtOAc: 0 to 100%) to afford di-tert-butyl (chloromethyl) phosphate 2 as a golden oil in 29% yield. ¹H NMR (CDCl₃, 400 MHz) δ (ppm): 5.64 (d, J=15.15, 2H), 1.51 (s, 18H).

Compound 2 was prepared by using an alternative method. To a solution of compound (1) (11.1 mmol) in benzene (0.9 mL/mmol) was added chloroiodomethane (111 mmol). The reaction mixture was stirred at room temperature for 20 hours in the dark. The crude mixture was concentrated under reduced pressure and the oily residue was dissolved in diethyl ether. The precipitate was filtered off and the organic layer was washed with a saturated NaHCO₃ solution and brine. The organic layer was dried over Na₂SO₄, concentrated under reduced pressure and purified by chromatography on silica (cyclohexane/EtOAc 9:1 then 8:2) to afford the expected compound as a colorless oil in 61% yield. ¹H NMR (CDCl₃, 400 MHz) δ (ppm) 5.64 (d, J=12 Hz, 2H), 1.51 (s, 18H).

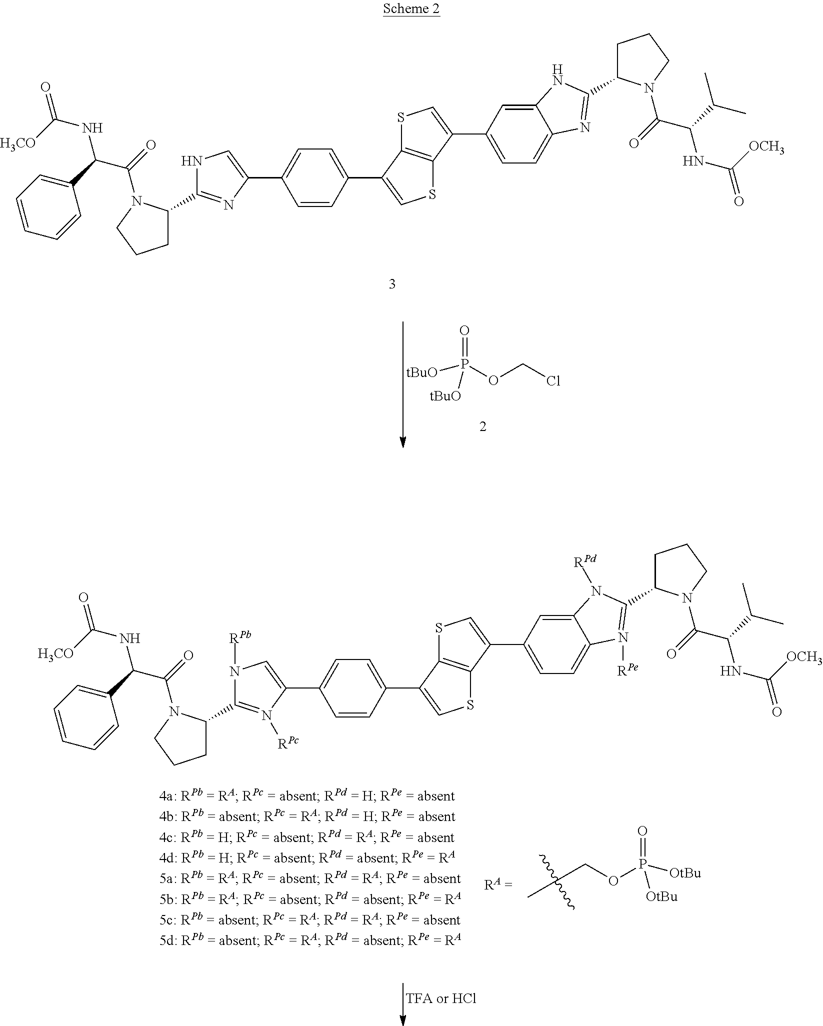

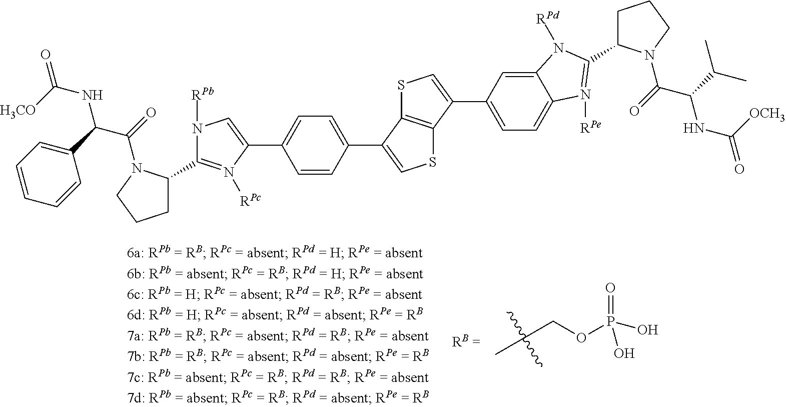

Compound 4.

To a solution of methyl N-[(1R)-2-[(2S)-2-[5-[4-[6-[2-[(2S)-1-[(2S)-2-(methoxycarbonylamino)-3-methyl-butanoyl]pyrrolidin-2-yl]-3H-benzimidazol-5-yl]thieno[3,2-b]thiophen-3-yl]phenyl]-1H-imidazol-2-yl]pyrrolidin-1-yl]-2-oxo-1-phenyl-ethyl]carbamate 3 (0.34 mmol) in N,N-dimethylformamide (30 mL/mmol) was added sodium hydride (60% in oil) (1.19 mmol) at room temperature. The resulting solution was stirred for 30 min at room temperature and a solution of di-tert-butyl (chloromethyl) phosphate 2 (0.51 mmol) in DMF (10 mL) was slowly added. After stirred overnight at room temperature, the reaction mixture was diluted with ethyl acetate and quenched with a saturated NaHCO₃ solution. The aqueous layer was extracted once with ethyl acetate. The combined organic layers were washed with brine, dried over sodium sulfate, and concentrated under reduced pressure. The crude residue was purified by flash chromatography on silica gel (DCM/methanol: 0 to 20%, and then DCM/methanol: 0 to 5%) to afford a mixture of isomers 4 as a yellow glue in 64% yield with 50% purity; MS (ESI) m/z=1108.2 (MH⁺).

Compound 6.

To a solution of the mixture of isomers 4 (0.22 mmol) in toluene (45 mL/mmol) and DCM (23 mL/mmol) was added trifluoroacetic acid (2.2 mmol). The reaction mixture was stirred 2 hrs at room temperature and then concentrated under reduced pressure. The crude residue was dissolved in a small amount of mixture water/TEAB (0.05M)/CH₃CN and then purified by RP-18 chromatography (TEAB 0.05M/CH₃CN: 60/40) to afford the expected mixture of isomers as a yellow solid in 18% yield. Additional purification by chiral preparative chromatography (Kromasil C 18 5μ 250*4.6 mm; H₂O/CH₃CN+0.1% HCOOH: 30 to 40%) to afford 2 major isomers.

Compound 6a: off-white solid; RT: 13.45 min; ¹H NMR (DMSO-d₆, 600 MHz) δ (ppm): 12.35 (brs, 1H), 8.08-8.00 (m, 3H), 7.96-7.93 (m, 1H), 7.89-7.88 (m, 2H), 7.82-7.81 (m, 2H), 7.71 (s, 1H), 7.65-7.61 (m, 3H), 7.41-7.40 (m, 2H), 7.38-7.36 (m, 2H), 7.33-7.32 (m, 1H), 7.29-7.28 (m, 1H), 6.98 (brs, 1H), 6.11 (brs, 1H), 5.65 (brs, 1H), 5.49-5.48 (m, 1H), 5.23-5.21 (m, 2H), 4.11-4.09 (m, 1H), 3.89-3.85 (m, 3H), 3.55 (s, 3H), 3.48 (s, 3H), 3.27 (m, 1H), 2.50 (m, 1H), 2.27-2.25 (m, 2H), 2.10-2.03 (m, 4H), 1.98-1.94 (m, 1H), 1.84 (m, 1H), 0.88-0.83 (m, 6H); ³¹P NMR (DMSO-d₆, 198 MHz) δ (ppm): −1.94 (s, 1P); MS (ESI) m/z=995.2 (MH⁺); solubility (microdisp)>0.13 mg/mL.

Compound 6b: off-white solid; RT: 15.48 min; ¹H NMR (CD₃OD, 600 MHz) δ (ppm): 8.03-8.01 (m, 1H), 7.86 (s, 2H), 7.84-7.83 (m, 1H), 7.82-7.81 (m, 1H), 7.80-7.77 (m, 2H), 7.49-7.48 (m, 2H), 7.43-7.41 (m, 2H), 7.39-7.37 (m, 1H), 7.07-6.93 (m, 1H), 6.32-6.29 (m, 1H), 6.10-6.07 (m, 1H), 5.54 (brs, 1H), 5.52-5.50 (m, 1H), 4.27-4.25 (m, 1H), 4.13-4.10 (brs, 1H), 4.03-4.00 (m, 1H), 3.99-3.96 (m, 1H), 3.68-3.66 (m, 3H), 3.652-3.650 (m, 3H), 3.58-3.56 (m, 1H), 3.33-3.32 (m, 1H), 2.64-2.60 (m, 1H), 2.40-2.36 (m, 1H), 2.34-2.30 (m, 1H), 2.28-2.25 (m, 1H), 2.20-2.18 (m, 1H), 2.17-2.12 (m, 1H), 2.03-1.98 (m, 1H), 1.62-1.59 (m, 2H), 1.33-1.32 (m, 1H), 0.92-0.83 (m, 6H); ³¹P NMR (DMSO-d₆, 198 MHz) δ (ppm): −1.86 (s, 1P); MS (ESI) m/z=995.2 (MH⁺); solubility (microdisp)>0.21 mg/mL.

Compounds 6 and 7 were obtained by using alternative methods.

Compounds 4c, 4d, 5a, and 5b.

A solution of compound 3 (1.13 mmol), compound 2 (1.69 mmol), K₂CO₃ (5.65 mmol) and KI (0.30 mmol) in acetone (88.5 mL/mmol) was stirred under reflux for 48 hours. The crude mixture was filtered over celite, concentrated under reduced pressure, and purified by 2 successive C-18 chromatographies (H₂O/CH₃CN+0.1% HCO₂H: 50 to 95%) to afford a mixture of partially deprotected monosubstituted compounds 4c and 4d as yellow solid in 45% yield, and a mixture of disubstituted compounds 5a and 5b as yellow solid in 21% yield.

Partially deprotected compounds 4c and 4d: MS (ESI) m/z=1052.0 (MH)⁺.

Compound 5a and 5b mixture: MS (ESI) m/z=1330.8 (MH)⁺.

Compounds 6c, 6d, 7a, and 7b.

A solution of mixture of compounds 4c and 4d (0.51 mmol) in HCl (4N in dioxane, 20 mL/mmol) was stirred 2 hours at room temperature and then concentrated under reduced pressure. The 2 isomers were purified by semi-preparative HPLC on HILIC column to afford the 2 expected pure compounds 6c and 6d and as yellow solids in 11% yield respectively (2 steps).

Compound 6c: ¹H NMR (DMSO-d₆, 600 MHz) δ (ppm) 11.98 (brs, 1H), 8.09 (s, 1H), 8.07-8.06 (m, 1H), 8.03-8.00 (m, 2H), 7.95-7.80 (m, 4H), 7.69 (d, J=8.60 Hz, 1H), 7.65 (d, J=8.60 Hz, 1H), 7.63 (s, 1H), 7.43-7.41 (m, 2H), 7.40-7.37 (m, 2H), 7.34-7.32 (m, 1H), 7.27-7.26 (m, 1H), 6.29-6.26 (m, 1H), 6.06-6.03 (m, 1H), 5.55-5.49 (m, 1H), 5.44-5.42 (m, 1H), 5.09 (brs, 1H), 4.06 (t, J=8.13 Hz, 1H), 3.94-3.88 (m, 2H), 3.83-3.81 (m, 1H), 3.57-3.54 (m, 6H), 3.18-3.15 (m, 1H), 2.35-2.29 (m, 2H), 2.15-2.12 (m, 1H), 2.04-1.96 (m, 4H), 1.85-1.82 (m, 2H), 0.86-0.78 (m, 6H); ³¹P NMR (DMSO-d₆, 243 MHz) δ (ppm) −1.92 (s, 1P); MS (ESI) m/z=995.7 (MH)⁺; Solubility (microdisp)=0.219 mg/mL.

Compound 6d: ¹H NMR (DMSO-d₆, 600 MHz) δ (ppm) 12.00-11.85 (m, 1H), 8.07-8.06 (m, 1H), 8.02-7.98 (m, 2H), 7.95 (s, 1H), 7.89-7.87 (m, 2H), 7.81-7.79 (m, 3H), 7.70-7.68 (m, 1H), 7.64-7.61 (m, 1H), 7.43-7.41 (m, 2H), 7.40-7.36 (m, 2H), 7.35-7.32 (m, 1H), 7.27-7.26 (m, 1H), 6.18-6.15 (m, 1H), 5.96-5.93 (m, 1H), 5.54-5.49 (m, 1H), 5.47-5.43 (m, 1H), 5.09-5.08 (m, 1H), 4.07 (t, J=8.71 Hz, 1H), 3.95-3.94 (m, 1H), 3.91-3.88 (m, 1H), 3.86-3.83 (m, 1H), 3.57-3.54 (m, 6H), 3.17-3.16 (m, 1H), 2.36-2.27 (m, 2H), 2.14-2.12 (m, 1H), 2.04-1.97 (m, 4H), 1.88-1.84 (m, 2H), 0.84-0.79 (m, 6H); ³¹P NMR (DMSO-d₆, 243 MHz) δ (ppm) −1.98 (s, 1P) RMN; MS (ESI) m/z=995.9 (MH)⁺; Solubility (microdisp)=0.260 mg/mL.

Compounds 7a and 7b. A mixture of disubstituted compounds 5a and 5b (0.29 mmol) in HCl (4N in dioxane, 34 mL/mmol) was stirred 2 hours at room temperature and then concentrated under reduced pressure. The 2 isomers were purified by semi-preparative HPLC to afford the mixture of 2 isomers 7a and 7b in 51% yield; ³¹P NMR (DMSO-d₆, 162 MHz) δ (ppm) −2.01; −1.93; −1.88; −1.84; MS (ESI) m/z=1106.6 (MH)⁺.

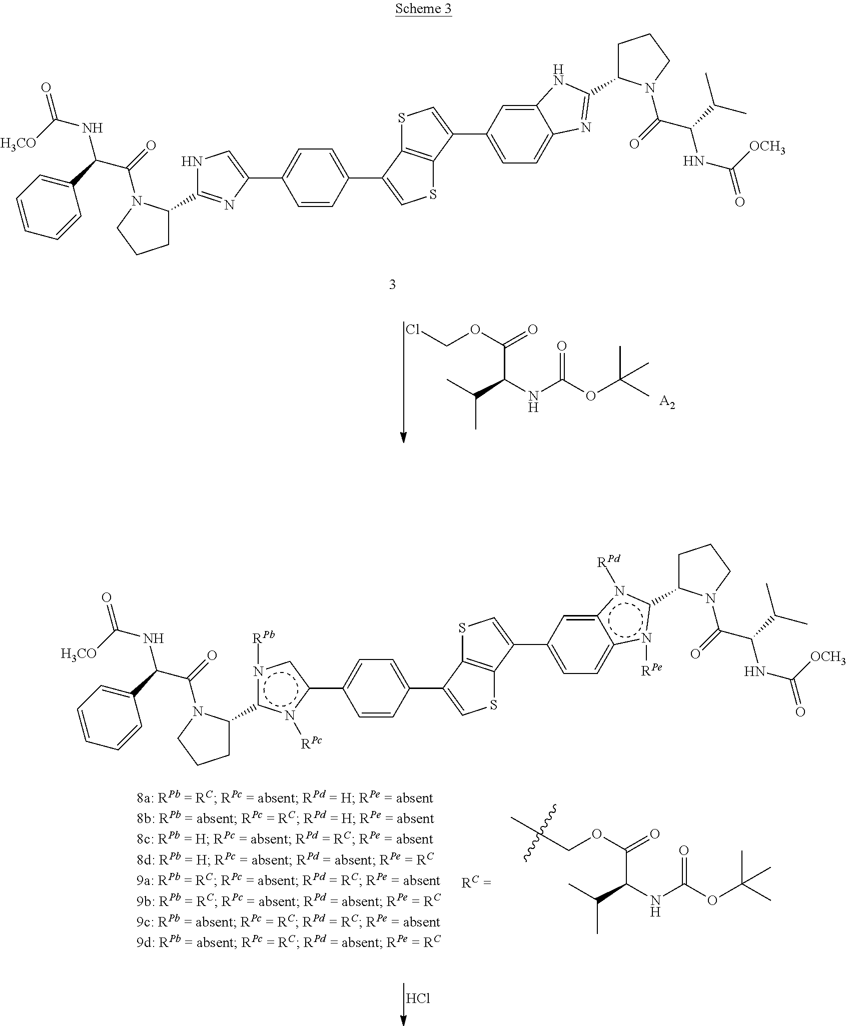

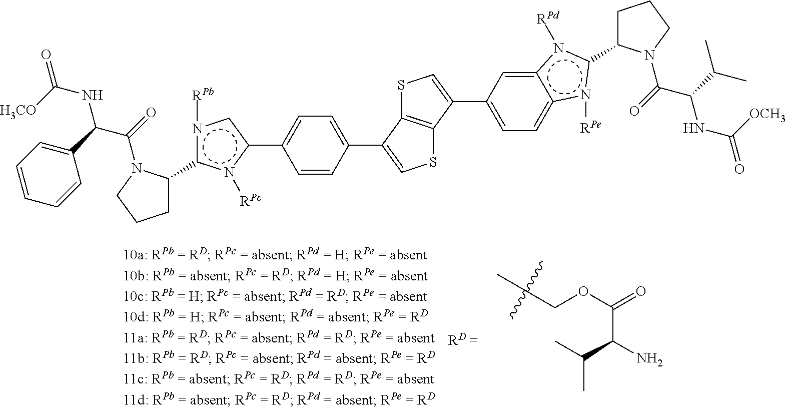

Compound A₂

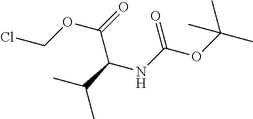 [SMILES: CC(C)[C@H](NC(=O)OC(C)(C)C)C(=O)OCCl]

To a solution of Boc-L-Valine (23.0 mmol), NaHCO₃ (92.0 mmol) and Bu₄NHSO₄ (2.3 mmol) in CH₂Cl₂ (1.5 mL/mmol) and H₂O (1.5 mL/mmol) was added, at 0° C., chloromethyl chlorosulfate (27.6 mmol). The reaction mixture was stirred 20 hours at room temperature and then extracted with CH₂Cl₂. Organic layer was washed with H₂O and brine, dried over Na₂SO₄ and concentrated under reduced pressure. The crude residue was purified by chromatography on silica (cyclohexane/EtOAc 10:0 then 9:1 then 8:2) to afford the expected intermediate as colorless oil in 92% yield. ¹H NMR (CDCl₃, 400 MHz) δ (ppm) 5.88-5.61 (m, 2H), 4.99-4.97 (m, 1H), 4.28-4.25 (m, 1H), 2.19-2.18 (m, 1H), 1.44 (s, 9H), 1.00-0.99 (d, 3H), 0.93-0.91 (d, 3H).

Compound 8 and 9. A solution of compound 3 (6.21 mmol), compound A₂ (9.32 mmol), K₂CO₃ (31.1 mmol) and KI (1.68 mmol) in acetone (100 mL/mmol) was stirred 21 hours under reflux. Acetone was removed under reduced pressure and the residue was dissolved in ethyl acetate and water. The solution was extracted with ethyl acetate and the organic layer was then dried over Na₂SO₄ and concentrated under reduced pressure. The crude residue was purified by chromatography on silica (EtOAc/methanol: 0 to 20%) to afford the mixture of dialkylated compounds 9a and 9b (1.71 g) as yellow solids, mixture of monoalkylated compounds 8d, 8c, and 8a (3.54 g) and starting material. (1.60 g). Mixture of monoalkylated compounds was next purified by C-18 flash chromatography (H₂O/CH₃CN+0.1% HCO₂H: 10 to 95%). Fractions were concentrated under reduced pressure to remove CH₃CN, and aqueous layers were extracted with EtOAc, dried over Na₂SO₄ and concentrated under reduced pressure to afford the mixture of compounds 8d and 8c (963 mg) and the compound 8a (518 mg).

Compounds 8d and 8c: MS (ESI) m/z=558.3 (MH₂)²⁺.

Compound 8a: MS (ESI) m/z=558.3 (MH₂)²⁺.

Compounds 9a and 9b: MS (ESI) m/z=672.9 (MH₂)²⁺.

Compound 10. A solution of mixture of monoalkylated compounds 8d and 8c (0.86 mmol) in HCl (4N in dioxane, 10 mL/mmol) was stirred 2 hours at room temperature and then concentrated under reduced pressure. The crude residue was purified by C-18 flash chromatography (H₂O/CH₃CN+0.1% HCO₂H: 10 to 45%) and semi-preparative HPLC (H₂O/CH₃CN+0.1% HCO₂H: 10 to 45%) to afford the monoalkylated compounds 10d and 10c as beige solids in 11% yield and 10% yield respectively.

Compound 10d: ¹H NMR (DMSO-d₆, 600 MHz) δ (ppm) 11.98-11.79 (m, 1H), 8.09-7.74 (m, 10H), 7.62-7.58 (m, 1H), 7.48-7.25 (m, 6H), 6.57 (d, J=11.79 Hz, 1H), 6.45 (d, J=11.79 Hz, 1H), 5.54-5.49 (m, 1H), 5.44-5.39 (m, 1H), 5.22-5.08 (m, 1H), 4.11-4.05 (m, 1H), 3.99-3.95 (m, 1H), 3.91-3.83 (m, 2H), 3.57-3.53 (m, 6H), 3.17-3.16 (m, 2H), 2.40-2.30 (m, 2H), 2.14-2.05 (m, 2H), 2.04-1.94 (m, 3H), 1.90-1.79 (m, 3H), 0.88-0.74 (m, 12H); MS (ESI) m/z=1015.0 (MH⁺).

Compound 10c: ¹H NMR (DMSO-d₆, 600 MHz) δ (ppm) 11.98-11.79 (m, 1H), 8.12-8.01 (m, 4H), 7.90-7.82 (m, 4H), 7.72-7.59 (m, 3H), 7.42-7.37 (m, 4H), 7.35-7.29 (m, 2H), 6.61 (d, J=11.51 Hz, 1H), 6.47 (d, J=11.51 Hz, 1H), 5.54-5.49 (m, 1H), 5.44-5.38 (m, 1H), 5.22-5.08 (m, 1H), 4.12-4.04 (m, 1H), 3.99-3.96 (m, 1H), 3.91-3.82 (m, 2H), 3.57-3.51 (m, 6H), 3.19-3.16 (m, 2H), 2.39-2.32 (m, 2H), 2.28-2.22 (m, 1H), 2.15-2.02 (m, 4H), 1.86-1.82 (m, 3H), 0.89-0.72 (m, 12H); MS (ESI) m/z=1014.7 (MH⁺).

Compound 10a.

A solution of monoalkylated compound 8a (0.19 mmol) in HCl (4N in dioxane, 20 mL/mmol) was stirred 1 hour at room temperature and then concentrated under reduced pressure. The crude residue was purified by C-18 flash chromatography (H₂O/CH₃CN+0.1% HCO₂H: 10 to 45%) to afford the monoalkylated compound 10a as yellow solid in 50% yield.

Compound 10a: ¹H NMR (DMSO-d₆, 600 MHz) δ (ppm) 12.35-12.32 (m, 1H), 8.10-8.02 (m, 3H), 7.94-7.80 (m, 4H), 7.76-7.74 (m, 1H), 7.69-7.61 (m, 3H), 7.42-7.29 (m, 6H), 6.49-6.41 (m, 1H), 6.18-6.06 (m, 1H), 5.50-5.49 (m, 1H), 5.21-5.16 (m, 2H), 4.11-4.04 (m, 1H), 3.89-3.86 (m, 3H), 3.58-3.55 (m, 3H), 3.49 (s, 3H), 3.30-3.26 (m, 2H), 2.55-2.54 (m, 1H), 2.28-2.22 (m, 2H), 2.10-2.01 (m, 4H), 1.98-1.85 (m, 3H), 0.88-0.80 (m, 12H); MS (ESI) m/z=1014.6 (MH⁺).

Compound 11. A solution of mixture of dialkylated compounds 9a and 9b (0.57 mmol) in HCl (4N in dioxane, 10 mL/mmol) was stirred 2 hours at room temperature and then concentrated under reduced pressure. The crude residue was purified by C-18 flash chromatography (H₂O/CH₃CN+0.1% HCO₂H: 10 to 45%) to afford a mixture of dialkylated compounds 11a and 11b as a beige solid in 43% yield.

Compound mixture 11a and 11b: MS (ESI) m/z=572.7 (MH₂)₂⁺.

### Example 2A

### HCV Replicon Assay

General procedure: Huh-7 cells containing HCV Con1 subgenomic replicon (GS4.1 cells) were grown in Dulbecco's Modified Eagle Medium (DMEM) supplemented with 10% fetal bovine serum (FBS), 2 mM L-glutamine, 110 mg/L sodium pyruvate, 1× non-essential amino acids, 100 U/mL penicillin-streptomycin, and 0.5 mg/mL G418 (Invitrogen). For dose-response testing, the cells were seeded in 96-well plates at 7.5×10³ cells/well in a volume of 50 μL, and incubated at 37° C./5% CO₂. Three hours after plating, 50 μL often 2-fold serial dilutions of compounds (highest concentration, 75 μM) were added, and cell cultures were incubated at 37° C./5% CO₂ in the presence of 0.5% DMSO. Alternatively, compounds were tested at a single concentration of 15 μM. In all cases, Huh-7 cells lacking the HCV replicon served as negative control. The cells were incubated in the presence of compounds for 72 hrs after which they were monitored for expression of the NS5A protein by enzyme-linked immunosorbent assay (ELISA). For this, the plates were then fixed for 1 min with acetone/methanol (1:1, v/v), washed twice with phosphate-buffered saline (PBS), 0.1% Tween 20, blocked for 1 hr at room temperature with TNE buffer containing 10% FBS and then incubated for 2 hr at 37° C. with the anti-NS5A mouse monoclonal antibody A-236 (ViroGen) diluted in the same buffer. After washing three times with PBS, 0.1% Tween 20, the cells were incubated 1 hr at 37° C. with anti-mouse immunoglobulin G-peroxidase conjugate in TNE, 10% FBS. After washing as described above, the reaction was developed with O-phenylenediamine (Zymed). The reaction was stopped after 30 min with 2 N H₂SO₄, and absorbance was read at 492 nm using Sunrise Tecan spectrophotometer. EC₅₀ values were determined from the % inhibition versus concentration data using a sigmoidal non-linear regression analysis based on four parameters with Tecan Magellan software. When screening at a single concentration, the results were expressed as % inhibition at 15 μM.

For cytotoxicity evaluation, GS4.1 cells were treated with compounds as described above and cellular viability was monitored using the Cell Titer 96 AQᵤₑₒᵤₛ One Solution Cell Proliferation Assay (Promega). CC₅₀ values were determined from the % cytotoxicity versus concentration data with Tecan Magellan software as described above.

### Example 2B

### Generation of HCV NS5A-Intergenotypic Stable Cell Lines for Genotypes 1a, 2a, 3a, and 4a

The nucleotide sequences of the NS5A region of genotype 2a (GenBank Accession #AB047639), genotype 3a (GenBank Accession #D17763), and genotype 4a (GenBank Accession#DQ418788) were synthesized by an outside vendor. The NS5A region of each of these genotypes included the first 11 amino acids of the protease recognition sequence of genotype 1b, as well as the last 10 amino acids of genotype 1b. The NS5A gene cassettes were excised with site specific restriction endonucleases and ligated into a ZS11-luciferase genotype 1b backbone (backbone contains the genotype 1b NS3 protease, NS4a, NS4b, and NS5b coding regions) with similarly cut restriction enzyme sites. Thus, the newly constructed plasmid contains a genotype 2α-, 3α- or 4α-specific NS5A gene within the 1b-replicon.

To generate the 1a-H77 NS5a intergenotypic plasmid, dual cut sites were inserted into the ZS11-lucifrease genotype 1b backbone that would bracket the NS5a region almost in its entirety. Using PCR and 1a-H77 specific primers also containing the corresponding restriction enzyme sites, the NS5a gene was amplified from the 1a-H77 replicon. The ZS11-luciferase genotype 1b backbone and the genotype 1a NS5A PCR products were restriction enzyme digested and then ligated using standard molecular cloning techniques. The newly constructed plasmid contains the genotype 1a-specific NS5a gene where as the backbone remains 1b as described herein.

These new intergenotypic plasmids were used to establish stable cell lines. RNA was generated from the NS5A intergenotypic plasmids and used in conjunction with a lipofectin reagent to transfect a cured Huh7 cell line. Transfected cells were selected for with G418. After selection has occurred the stable cell lines were propagated, tested for luciferase activity, and RT-PCR with genotype specific primers (either 1a, 2a, 3a, or 4a). Stable cell lines containing the intergenotypic replicon were then fully sequenced and analyzed for proper expression of NS3, NS5A and NS5B proteins.

Drug titration analysis was performed using the luciferase replicon assay described herein.

### Genotype 2a Infectious Virus Assay

The genotype 2a infectious virus assay measures the ability of a test compound to inhibit HCV replication in cell culture after 5 days of treatment at the time of HCV genotype 2a virus infection of a permissive human hepatoma cell line (HPC cells). The inhibition of HCV replication was measured by quantification of HCV core protein expression by an enzyme-linked immunosorbent assay (ELISA). Briefly, HPC cells were grown in DMEM containing glucose, L-glutamine and sodium pyruvate, 10% FBS, 100 IU/mL penicillin, 100 μg/mL streptomycin, 2 mM GlutaMAX, and non-essential amino acids. GlutaMAX was obtained from Invitrogen, Corp.; all other media reagents were obtained from Mediatech, Inc. For dose-response testing, ninety-six-well plates were seeded with HPC cells at a density of 2.5×10³ cells/well in a volume of 50 μL, and incubated at 37° C./5% CO₂. Three hours after plating, 50 μL of ten 5-fold serial dilutions of compound and 100 μL of genotype 2a virus were added, and cell cultures were incubated at 37° C./5% CO₂. In all cases, mock infected HPC cells served as negative control. At 16 hours post treatment and infection, the virus inoculum was removed by aspiration. The cultures were treated at the same final concentrations of drug diluted in media and incubated for 4 additional days at 37° C./5% CO₂. Subsequently, the core ELISA was performed as follows. The plates were fixed for 90 seconds with acetone/methanol (1:1, v/v), washed three times with KPL wash solution (KPL, Inc.), blocked for 1 hr at room temperature with TNE buffer containing 10% FBS and then incubated for 2 hr at 37° C. with the anti-HCV core mouse monoclonal antibody (Thermo Scientific) diluted in the same buffer. After washing three times with KPL wash solution, the cells were incubated for 1 hr at 37° C. with an anti-mouse immunoglobulin G-peroxidase conjugate in TNE/10% FBS. After washing as described above, the reaction was developed with O-phenylenediamine (Invitrogen). The reaction was stopped after 30 min with 2 N H₂SO₄, and absorbance was read at 490 nm in a Victor³V 1420 multilabel counter (Perkin Elmer) and EC₅₀ concentrations were determined using Microsoft Excel and XLfit 4.1 software.

For cytotoxicity evaluation, HPC cells were treated with compounds as described above in the absence of the genotype 2a virus and cellular viability was monitored using the Cell Titer 96 AQueous One Solution Cell Proliferation Assay (Promega). Plates were then read at 490 nm in a Victor³V 1420 multilabel counter (Perkin Elmer) and CC₅₀ concentrations were determined using Microsoft Excel and XLfit 4.1 software.

### Luciferase Replicon Assay

The HCV luciferase replicon assay measures the ability of a test compound to inhibit HCV replication in cell culture after 3 days of treatment in a human hepatoma cell line (Huh-7) bearing an HCV replicon containing a luciferase-neomycin phosphotransferase fusion gene. The inhibition of HCV replication was measured by quantification of luciferase protein expression. Briefly, Huh-7 cells containing either the HCV genotype 1a H77 strain or genotype 1b Con1 strain subgenomic luciferase replicon (H1α-luc or Zluc, respectively) were grown in DMEM containing glucose, L-glutamine and sodium pyruvate, 10% fetal bovine serum (FBS), 100 IU/mL penicillin, 100 μg/mL streptomycin, 2 mM GlutaMAX, non-essential amino acids and 0.25 (H1a-luc) or 0.5 (Zluc) mg/mL G418. GlutaMAX was obtained from Invitrogen, Corp.; all other media reagents were obtained from Mediatech, Inc. For dose-response testing, the cells were seeded in 96-well plates at 1×10⁴ (H1a-luc) or 7.5×10³ (Zluc) cells/well in a volume of 50 μL, and incubated at 37° C./5% CO₂. Three hours after plating, 50 μL of ten 5-fold serial dilutions of compound were added, and cell cultures were incubated at 37° C./5% CO₂ for 72 hours. In all cases, Huh-7 cells lacking the HCV replicon served as negative control. To assess luciferase expression, the media/compound was removed from the plates and ONE-glo Luciferase assay reagent (Promega) was added to each well. The assay plates were shaken for 3 minutes at room temperature and luciferase activity for each well was measured with a 1 sec read time on the Victor³V multilabel counter using a 700 nm cut-off filter (Perkin Elmer). EC₅₀ values were calculated from dose response curves from the resulting best-fit equations determined by Microsoft Excel and XLfit 4.1 software.

For cytotoxicity evaluation, H1a-luc or Zluc cells were treated with compounds as described above and cellular viability was monitored using the Cell Titer 96 AQueous One Solution Cell Proliferation Assay (Promega). Plates were then read at 490 nm in a Victor³V 1420 multilabel counter (Perkin Elmer) and CC₅₀ concentrations were determined using Microsoft Excel and XLfit 4.1 software.

### Example 2C

### Luciferase Replicon Transient Transfection Assay

General Procedure:

The luciferase replicon transient transfection assay measures the ability of a test compound to inhibit the replication of a transiently-transfected HCV luciferase-bearing wild-type or mutant replicon in cured human hepatoma cells (Huh7.5). The inhibition of HCV replication was measured by quantification of luciferase protein expression. This assay has been validated using a panel of genotype 1a and 1b replicons bearing mutations known to be associated with resistance to BMS-790052. Briefly, subconfluent Huh7.5 cells were electroporated with 10 μg of wild-type or mutant luciferase-bearing HCV replicon RNA. The cells were then seeded in 96-well opaque white plates at 3×10⁴ cells/well in 150 μL/well and incubated for 4 hrs at 37° C./5% CO₂. Ten 1:5 serial dilutions of each test compound were made in media (DMEM containing glucose, L-glutamine and sodium pyruvate, 10% fetal bovine serum, 100 IU/mL penicillin, 100 μg/mL streptomycin, 2 mM GlutaMAX, and 1×MEM non-essential amino acids (Mediatech, Inc. and Invitrogen Corp.)) at concentrations that were 4× higher than the final concentrations to be tested and 50 μL/well was added to the transfected cells. Untreated, mock-transfected cells served as a negative control of luciferase expression. The plates were incubated at 37° C./5% CO₂ for 4 days whereupon the media was removed and 50 μL of ONE-glo luciferase substrate (Promega) was added to each well. The plates were agitated on a rotating platform at room temperature for 3 min and read in a Victor³V microplate reader (Perkin-Elmer) using a 700 nm cut-off filter with a 1 sec read time. EC₅₀ values were calculated from dose response curves from the resulting best-fit equations determined by Microsoft Excel and XLfit 4.1 software.

To determine the replication capacity of each mutant relative to the wild-type parental replicon, transfected cells were plated on two plates and were not treated with compound. Luciferase activity was measured at time points of 4 hrs and 4 days after plating for each replicon. Replication capacity was calculated by dividing the day 4 CPS by the 4 hour CPS for each replicon and determining the percentage present for each mutant replicon relative to wild-type replicon values. The NS3, NS4B, and NS5B mutants were prepared and tested according to the methods described herein.

The compounds disclosed herein have EC₅₀ and CC₅₀ as follows:

| | HCV Replicon | HCV Replicon |
|---|---|---|
| Compound | CC₅₀ (μM) | EC₅₀ (μM) |
| 6c | >100 | + |
| 6d | >100 | ++ |
| 10c | >100 | +++ |
| 10a | >100 | +++ |

EC₅₀: 10⁻³ > + > 10⁻⁴ > ++ > 10⁻⁵ > +++

The examples set forth above are provided to give those of ordinary skill in the art with a complete disclosure and description of how to make and use the claimed embodiments, and are not intended to limit the scope of what is disclosed herein. Modifications that are obvious to persons of skill in the art are intended to be within the scope of the following claims. All publications, patents, and patent applications cited in this specification are incorporated herein by reference as if each such publication, patent or patent application were specifically and individually indicated to be incorporated herein by reference.

1. A compound of Formula IA: 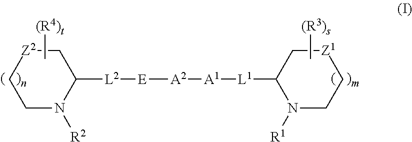
or a single enantiomer, a racemic mixture, a diastereomer, a mixture of diastereomers, or an isotopic variant thereof; or a pharmaceutically acceptable salt or solvate thereof;
A¹, A², and E are each independently (a) a bond; or (b) C₁₋₆ alkylene, C₂₋₆ alkenylene, C₂₋₆ alkynylene, C₂₋₂₀ cycloalkylene, C₆₋₂₀ arylene, heteroarylene; or heterocyclylene;
L¹ and L² are each independently (a) a bond; (b) C₁₋₆ alkylene, C₂₋₆ alkenylene, C₂₋₆ alkynylene, C₃₋₇ cycloalkylene, C₆₋₁₄ arylene, heteroarylene, or heterocyclylene; or (c) —C(O)—, —C(O)O—, —C(O)NR^{1a}—, —C(═NR^{1a})NR^{1c}, —O—, —OC(O)O—, —OC(O)NR^{1a}—, —OC(═NR^{1a})NR^{1c}—, —OP(O)(OR^{1a})—, —OP(O)(OR^{1a})O—, —NR^{1a}—, —NR^{1a}C(O)NR^{1c}—, —NR^{1a}C(═NR^{1b})NR^{1c}—, —NR^{1a}S(O)NR^{1c}—, —NR^{1a}S(O)₂NR^{1c}—, —S—, —S(O)—, —S(O)₂—, —S(O)NR^{1a}—, or —S(O)₂NR^{1a}—; wherein at least one of L¹ and L² is heteroarylene or heterocyclylene, which is substituted with —C₁₋₆ alkylene-OP(O)(OR^{P1})₂ (in one embodiment, —CH₂—OP(O)(OR^{P1})₂) or —C₁₋₆ alkylene-O-linked amino acid or a derivative thereof (in one embodiment, —CH₂—OC(O)C(R^{aa})₂NR^{1b}R^{1c});
Z¹ and Z² are each independently a bond, —O—, —S—, —S(O)—, —S(O₂)—, or —N(R^{N})—;
R¹ and R² are each independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (c) —C(O)R^{1a},
—C(O)CH(N(R^{1c})C(O)OR^{1b})R^{1a}, —C(O)OR^{1a}, —C(O)NR^{1b}R^{1c}, —C(NR^{1a})NR^{1b}R^{1c}, —P(O)(OR^{1a})R^{1d}, —CH₂P(O)(OR^{1a})R^{1d}, —S(O)R^{1a}, —S(O)₂R^{1a}, —S(O)NR^{1b}R^{1c}, or —S(O)₂NR^{1b}R^{1c}; (d) —C₁₋₆ alkylene-OP(O)(OR^{P1})₂, in one embodiment, —CH₂—OP(O)(OR^{P1})₂; or (e) —C₁₋₆ alkylene-O-linked amino acid or a derivative thereof, in one embodiment, —CH₂—OC(O)C(R^{aa})₂NR^{1b}R^{1c};
each R³ and R⁴ is independently (a) cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —SR^{1a}, —S(O)R^{1a}, —S(O)₂R^{1a}, —S(O)NR^{1b}R^{1c}, or —S(O)₂NR^{1b}R^{1c}; or
two R³ or two R⁴ that are attached to the same ring are linked together to form a bond, —O—, —NR^{N}—, —S—, C₁₋₆ alkylene, C₁₋₆ heteroalkylene, C₂₋₆ alkenylene, or C₂₋₆ heteroalkenylene;
each R^{N} is independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (c) —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —S(O)R^{1a}, —S(O)₂R^{1a}, —S(O)NR^{1b}R^{1c}, or —S(O)₂NR^{1b}R^{1c}; (d) —C₁₋₆ alkylene-OP(O)(OR^{P1})₂, in one embodiment, —CH₂—OP(O)(OR^{P1})₂; or (e) —C₁₋₆ alkylene-O-linked amino acid or a derivative thereof, in one embodiment, —CH₂—OC(O)C(R^{aa})₂NR^{1b}R^{1c};
each R^{P1} is independently (a) hydrogen; (b) a monovalent cation, in one embodiment, Na⁺ or K⁺, in another embodiment, Li⁺, Rb⁺, or Cs⁺; or (c) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or two R^{P1} together are a divalent cation, in one embodiment, Mg²⁺ or Ca²⁺;
each R^{1a}, R^{1b}, R^{1c}, and R^{1d} is independently hydrogen, C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or R^{1a} and R^{1c} together with the C and N atoms to which they are attached form heterocyclyl; or R^{1b} and R^{1c} together with the N atom to which they are attached form heterocyclyl;
each R^{aa} independently is a side chain of a naturally occurring or non-naturally occurring amino acid; in one embodiment, each R^{aa} independently is hydrogen, C₁₋₆ alkyl, heteroalkyl, C₆₋₁₄ aryl-C₁₆ alkyl and heteroaryl-C₁₆ alkyl;
each m and n is independently an integer of 1, 2, 3, or 4; and
each s and t is independently an integer of 0, 1, 2, 3, 4, 5, 6, or 7;
with the proviso that the compound is neither 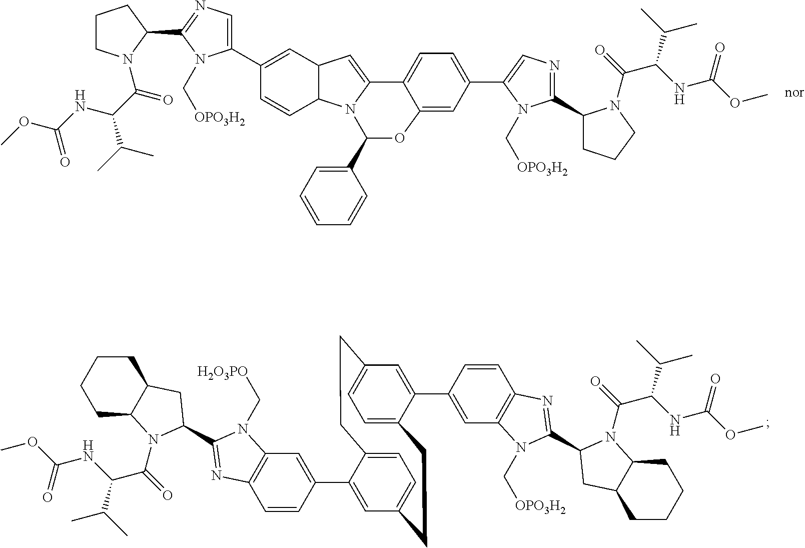
wherein each alkyl, alkylene, heteroalkylene, alkenyl, alkenylene, heteroalkenylene, alkynyl, alkynylene, cycloalkyl, cycloalkylene, aryl, arylene, aralkyl, heteroaryl, heteroarylene, heterocyclyl, and heterocyclylene is optionally substituted with one or more, in one embodiment, one, two, three, or four, substituents Q, where each Q is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is further optionally substituted with one or more, in one embodiment, one, two, three, or four, substituents Q^{a}; or (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OP(O)(OR^{e})₂, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —P(O)(OR^{a})R^{d}, —CH₂P(O)(OR^{a})R^{d}, —CH₂OP(O)(OR^{e})₂, —CH₂OC(O)C(R^{a})₂NR^{b}R^{c}, —SR^{a}, —S(O)R^{a}, —S(O)₂R^{a}, —S(O)NR^{b}R^{c}, or —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a}; and each R^{e} is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iv) two R^{e} together are a divalent cation (e.g., Mg²⁺ or Ca²⁺);
wherein each Q^{a} is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{f}, —C(O)OR^{f}, —C(O)NR^{g}R^{h}, —C(NR^{f})NR^{g}R^{h}, —OR^{f}, —OC(O)R^{f}, —OC(O)OR^{f}, —OC(O)NR^{g}R^{h}, —OC(═NR^{f})NR^{g}R^{h}, —OP(O)(ORⁿ)₂, —OS(O)R^{f}, —OS(O)₂R^{f}, —OS(O)NR^{g}R^{h}, —OS(O)₂NR^{g}R^{h}, —R^{g}R^{h}, —NR^{f}C(O)R^{k}, —NR^{f}C(O)OR^{k}, —NR^{f}C(O)NR^{g}R^{h}, —NR^{f}C(═NR^{k})NR^{g}R^{h}, —NR^{f}S(O)R^{k}, —NR^{f}S(O)₂R^{k}, —NR^{f}S(O)NR^{g}R^{h}, —NR^{f}S(O)₂NR^{g}R^{h}, —P(O)(OR^{f})R^{k}, —CH₂P(O)(OR^{f})R^{k}, —SR^{f}, —S(O)R^{f}, —S(O)₂R^{f}, —S(O)NR^{g}R^{h}, or —S(O)₂NR^{g}R^{h}; wherein each R^{f}, R^{g}, R^{h}, and R^{k} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{g} and R^{h} together with the N atom to which they are attached form heterocyclyl; and each Rⁿ is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iv) two Rⁿ together are a divalent cation (e.g., Mg²⁺ or Ca²⁺).

2. The compound of claim 1 having the structure of Formula II: 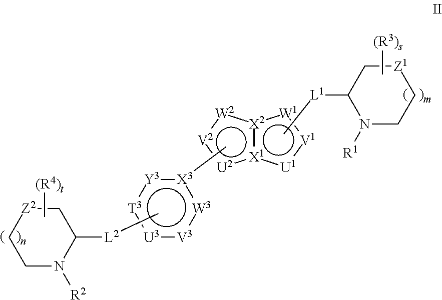
or a single enantiomer, a racemic mixture, a diastereomer, a mixture of diastereomers, or an isotopic variant thereof; or a pharmaceutically acceptable salt or solvate thereof; wherein:
T³ is a bond, C, N, O, S, CR⁷, or NR⁷;
U¹, U², U³, V¹, V², V³, W¹, W², W³, and Y³ are each independently C, N, O, S, CR⁷, or NR⁷;
X¹, X², and X³ are each independently C or N;
each R⁷ is independently (a) hydrogen, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q; (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —SR^{1a}, —S(O)R^{1a}, —S(O)₂R^{1a}, —S(O)NR^{1b}R^{1c}, or —S(O)₂NR^{1b}R^{1c}; (d) —C₁₋₆ alkylene-OP(O)(OR^{P1})₂, in one embodiment, —CH₂—OP(O)(OR^{P1})₂; or (e) —C₁₋₆ alkylene-O-linked amino acid or a derivative thereof, in one embodiment, —CH₂—OC(O)C(R^{aa})₂NR^{1b}R^{1c}.

3. (canceled)

4. The compound of claim 1 having the structure of Formula IVe: 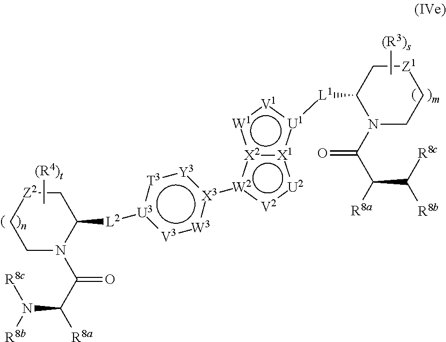
or a single enantiomer, a diastereomer, a mixture of diastereomers, or an isotopic variant thereof; or a pharmaceutically acceptable salt or solvate thereof.

5-40. (canceled)

41. A compound of Formula IA: 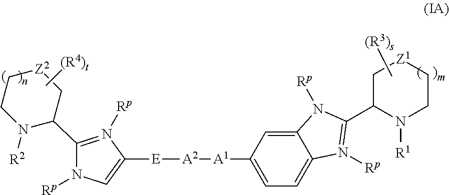
or a single enantiomer, a racemic mixture, a diastereomer, a mixture of diastereomers, or an isotopic variant thereof; or a pharmaceutically acceptable salt or solvate thereof;
A¹, A², and E are each independently (a) a bond; or (b) C₁₋₆ alkylene, C₂₋₆ alkenylene, C₂₋₆ alkynylene, C₂₋₂₀ cycloalkylene, C₆₋₂₀ arylene, heteroarylene; or heterocyclylene;
Z¹ and Z² are each independently a bond, —O—, —S—, —S(O)—, —S(O₂)—, or —N(R^{N})—;
R¹ and R² are each independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (c) —C(O)R^{1a}, —C(O)CH(N(R^{1c})C(O)OR^{1b})R^{1a}, —C(O)OR^{1a}, —C(O)NR^{1b}R^{1c}, —C(NR^{1a})NR^{1b}R^{1c}, —P(O)(OR^{1a})R^{1d}, —CH₂P(O)(OR^{1a})R^{1d}, —S(O)R^{1a}, —S(O)₂R^{1a}, —S(O)NR^{1b}R^{1c}, or —S(O)₂NR^{1b}R^{1c}; (d) —C₁₋₆ alkylene-OP(O)(OR^{P1})₂, in one embodiment, —CH₂—OP(O)(OR^{P1})₂; or (e) —C₁₋₆ alkylene-O-linked amino acid or a derivative thereof, in one embodiment, —CH₂—OC(O)C(R^{aa})₂NR^{1b}R^{1c};
each R³ and R⁴ is independently (a) cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{d}, —CH₂P(O)(OR^{1a})R^{1d}, —SR^{1a}, —S(O)R^{1a}, —S(O)₂R^{1a}, —S(O)NR^{1b}R^{1c}, or —S(O)₂NR^{1b}R^{1c}; or
two R³ or two R⁴ that are attached to the same ring are linked together to form a bond, —O—, —NR^{N}—, —S—, C₁₋₆ alkylene, C₁₋₆ heteroalkylene, C₂₋₆ alkenylene, or C₂₋₆ heteroalkenylene;
each R^{N} is independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (c) —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —S(O)R^{1a}, —S(O)₂R^{1a}, —S(O)NR^{1b}R^{1c}, or —S(O)₂NR^{1b}R^{1c}; (d) —C₁₋₆ alkylene-OP(O)(OR^{P1})₂, in one embodiment, —CH₂—OP(O)(OR^{P1})₂; or (e) —C₁₋₆ alkylene-O-linked amino acid or a derivative thereof, in one embodiment, —CH₂—OC(O)C(R^{aa})₂NR^{1b}R^{1c};
each R^{P} is independently absent, hydrogen, or —C₁₋₆ alkylene-OP(O)(OR^{P1})₂, in one embodiment, —CH₂—OP(O)(OR^{P1})₂; wherein, when the two R^{P} groups attached to the imidazolylene are neither absent nor hydrogen, the imidazolyene group carries a positive charge; and when the two R^{P} groups attached to the benzimidazolylene are neither absent nor hydrogen, the benzimidazolylene group carries a positive charge; and wherein at least one of the R^{P} groups is neither absent or hydrogen;
alternatively, each R^{P} is independently absent, hydrogen, —C₁₋₆ alkylene-OP(O)(OR^{P1})₂ (in one embodiment, —CH₂—OP(O)(OR^{P1})₂), or —C₁₋₆ alkylene-O-linked amino acid or a derivative thereof (in one embodiment, —CH₂—OC(O)C(R^{aa})₂NR^{1b}R^{1c}); wherein, when the two R^{P} groups attached to the imidazolylene are neither absent nor hydrogen, the imidazolyene group carries a positive charge; and when the two R^{P} groups attached to the benzimidazolylene are neither absent nor hydrogen, the benzimidazolylene group carries a positive charge; and wherein at least one of the R^{P} groups is neither absent or hydrogen;
each R^{P1} is independently (a) hydrogen; (b) a monovalent cation, in one embodiment, Na⁺ or K⁺, in another embodiment, Li⁺, Rb⁺, or Cs⁺; or (c) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or two R^{P1} together are a divalent cation, in one embodiment, Mg²⁺ or Ca²⁺;
each R^{1a}, R^{1b}, R^{1c}, and R^{1d} is independently hydrogen, C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or R^{1a} and R^{1c} together with the C and N atoms to which they are attached form heterocyclyl; or R^{1b} and R^{1c} together with the N atom to which they are attached form heterocyclyl;
each R^{aa} independently is a side chain of a naturally occurring or non-naturally occurring amino acid; in one embodiment, each R^{aa} independently is hydrogen, C₁₋₆ alkyl, heteroalkyl, C₆₋₁₄ aryl-C₁₋₆alkyl and heteroaryl-C₁₋₆alkyl;
each m and n is independently an integer of 1, 2, 3, or 4; and
each s and t is independently an integer of 0, 1, 2, 3, 4, 5, 6, or 7;
wherein each alkyl, alkylene, heteroalkylene, alkenyl, alkenylene, heteroalkenylene, alkynyl, alkynylene, cycloalkyl, cycloalkylene, aryl, arylene, aralkyl, heteroaryl, heteroarylene, imidazolylene, benzimidazolyl, heterocyclyl, and heterocyclylene is optionally substituted with one or more, in one embodiment, one, two, three, or four, substituents Q, where each Q is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is further optionally substituted with one or more, in one embodiment, one, two, three, or four, substituents Q^{a}; or (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OP(O)(OR^{e})₂, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —P(O)(OR^{a})R^{d}, —CH₂P(O)(OR^{a})R^{d}, —CH₂OP(O)(OR^{e})₂, —CH₂OC(O)C(R^{a})₂NR^{b}R^{c}, —SR^{a}, —S(O)R^{a}, —S(O)₂R^{a}, —S(O)NR^{b}R^{c}, or —S(O)₂NR^{b}R^{c}, wherein each R^{a}, R^{b}, R, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a}; and each R^{e} is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iv) two R^{e} together are a divalent cation (e.g., Mg²⁺ or Ca²⁺);
wherein each Q^{a} is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{f}, —C(O)OR^{f}, —C(O)NR^{g}R^{h}, —C(NR^{f})NR^{g}R^{h}, —OR^{f}, —OC(O)R^{f}, —OC(O)OR^{f}, —OC(O)NR^{g}R^{h}, —OC(═NR^{f})NR^{g}R^{h}, —OP(O)(ORⁿ)₂, —OS(O)R^{f}, —OS(O)₂R^{f}, —OS(O)NR^{g}R^{h}, —OS(o)₂NR^{g}R^{h}, —R^{g}R^{h}, —NR^{f}C(O)R^{k}, —NR^{f}C(O)OR^{k}, —NR^{f}C(O)NR^{g}R^{h}, —NR^{f}C(═NR^{k})NR^{g}R^{h}, —NR^{f}S(O)R^{k}, —NR^{f}S(O)₂R^{k}, —NR^{f}S(O)NR^{g}R^{h}, —NR^{f}S(O)₂NR^{g}R^{h}, —P(O)(OR^{f})R^{k}, —CH₂P(O)(OR^{f})R^{k}, —SR^{f}, —S(O)R^{f}, —S(O)₂R^{f}, —S(O)NR^{g}R^{h}, or —S(O)₂NR^{g}R^{h}; wherein each R^{f}, R^{g}, R^{h}, and R^{k} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{g} and R^{h} together with the N atom to which they are attached form heterocyclyl; and each Rⁿ is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iv) two Rⁿ together are a divalent cation (e.g., Mg²⁺ or Ca²⁺).

42-45. (canceled)

46. A compound of Formula IIA: 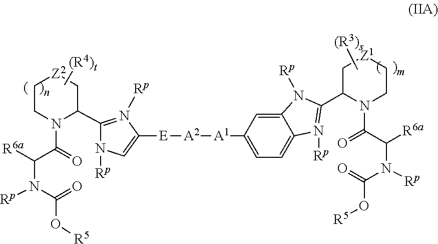
or a single enantiomer, a racemic mixture, a diastereomer, a mixture of diastereomers, or an isotopic variant thereof; or a pharmaceutically acceptable salt or solvate thereof; wherein:
A¹, A², and E are each independently (a) a bond; or (b) C₁₋₆ alkylene, C₂₋₆ alkenylene, C₂₋₆ alkynylene, C₂₋₂₀ cycloalkylene, C₆₋₂₀ arylene, heteroarylene; or heterocyclylene;
Z¹ and Z² are each independently a bond, —O—, —S—, —S(O)—, —S(O₂)—, or —N(R^{N})—;
each R³ and R⁴ is independently (a) cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —SR^{1a}, —S(O)R^{1a}, —S(O)₂R^{1a}, —S(O)NR^{1b}R^{1c}, or —S(O)₂NR^{1b}R^{1c}; or
two R³ or two R⁴ that are attached to the same ring are linked together to form a bond, —O—, —NR^{N}—, —S—, C₁₋₆ alkylene, C₁₋₆ heteroalkylene, C₂₋₆ alkenylene, or C₂₋₆ heteroalkenylene;
each R⁵ is independently C₁₋₆ alkyl, C₂₋₆ alkenyl, C₂₋₆ alkynyl, C₃₋₇cycloalkyl, C₆₋₁₄ aryl, C₇₋₁₅ aralkyl, heteroaryl, or heterocyclyl;
each R^{6a} is independently hydrogen, C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl;
each R^{N} is independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (c) —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{d}, —NR^{1a}C(O)OR^{d}, —NR^{1a}C(O)NR^{1b}R^{1c}, —NR^{1a}C(═NR^{1d})NR^{1b}R^{1c}, —NR^{1a}S(O)R^{d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —S(O)R^{1a}, —S(O)₂R^{1a}, —S(O)NR^{1b}R^{1c}, or —S(O)₂NR^{1b}R^{1c}; (d) —C₁₋₆ alkylene-OP(O)(OR^{P1})₂, in one embodiment, —CH₂—OP(O)(OR^{P1})₂; or (e) —C₁₋₆ alkylene-O-linked amino acid or a derivative thereof, in one embodiment, —CH₂—OC(O)C(R^{aa})₂NR^{1b}R^{1c};
each R^{P} is independently absent, hydrogen, —C₁₋₆alkylene-OP(O)(OR^{P1})₂ (in one embodiment, —CH₂—OP(O)(OR^{P1})₂) or —C₁₋₆ alkylene-O-linked amino acid or a derivative thereof (in one embodiment, —CH₂—OC(O)C(R^{aa})₂NR^{1b}R^{1c}); wherein, when the two R^{P} groups attached to the imidazolylene are neither absent nor hydrogen, the imidazolyene group carries a positive charge; and when the two R^{P} groups attached to the benzimidazolylene are neither absent nor hydrogen, the benzimidazolylene group carries a positive charge; with the proviso that at least one of the R^{P} groups is neither absent nor hydrogen;
each R^{P1} is independently (a) hydrogen; (b) a monovalent cation, in one embodiment, Na⁺ or K⁺, in another embodiment, Li⁺, Rb⁺, or Cs⁺; or (c) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or two R^{P1} together are a divalent cation, in one embodiment, Mg²⁺ or Ca²⁺;
each R^{1a}, R^{1b}, R^{1c}, and R^{1d} is independently hydrogen, C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or R^{1a} and R^{1c} together with the C and N atoms to which they are attached form heterocyclyl; or R^{1b} and R^{1c} together with the N atom to which they are attached form heterocyclyl;
each R^{aa} independently is a side chain of a naturally occurring or non-naturally occurring amino acid; in one embodiment, each R^{aa} independently is hydrogen, C₁₋₆ alkyl, heteroalkyl, C₆₋₁₄ aryl-C₁₋₆alkyl and heteroaryl-C₁₋₆alkyl;
each m and n is independently an integer of 1, 2, 3, or 4; and
each s and t is independently an integer of 0, 1, 2, 3, 4, 5, 6, or 7;
wherein each alkyl, alkylene, heteroalkylene, alkenyl, alkenylene,
heteroalkenylene, alkynyl, alkynylene, cycloalkyl, cycloalkylene, aryl, arylene, aralkyl, heteroarylene, heteroaryl, imidazolylene, heterocyclyl, and heterocyclylene is optionally substituted with one or more, in one embodiment, one, two, three, or four, substituents Q, where each Q is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is further optionally substituted with one or more, in one embodiment, one, two, three, or four, substituents Q^{a}; or (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OP(O)(OR^{e})₂, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR^{d})NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —P(O)(OR^{a})R^{d}, —CH₂P(O)(OR^{a})R^{d}, —CH₂OP(O)(OR^{e})₂, —CH₂OC(O)C(R^{a})₂NR^{b}R^{c}, —SR^{a}, —S(O)R^{a}, —S(O)₂R^{a}, —S(O)NR^{b}R^{c}, or —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R together with the N atom to which they are attached form heterocyclyl, optionally substituted with one or more, in one embodiment, one, two, three, or four, substituents Q^{a}; and each R^{e} is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iv) two R^{e} together are a divalent cation (e.g., Mg²⁺ or Ca²⁺);
wherein each Q^{a} is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{f}, —C(O)OR^{f}, —C(O)NR^{g}R^{h}, —C(NR^{f})NR^{g}R^{h}, —OR^{f}, —OC(O)R^{f}, —OC(O)OR^{f}, —OC(O)NR^{g}R^{h}, —OC(═NR^{f})NR^{g}R^{h}, —OP(O)(ORⁿ)₂, —OS(O)R^{f}, —OS(O)₂R^{f}, —OS(O)NR^{g}R^{h}, —OS(O)₂NR^{g}R^{h}, —NR^{g}R^{h}, —NR^{f}C(O)R^{k}, —NR^{f}C(O)OR^{k}, —NR^{f}C(O)NR^{g}R^{h}, —NR^{f}C(═NR^{k})NR^{g}R^{h}, —NR^{f}S(O)R^{k}, —NR^{f}S(O)₂R^{k}, —NR^{f}S(O)NR^{g}R^{h}, —NR^{f}S(O)₂NR^{g}R^{h}, —P(O)(OR^{f})R^{k}, —CH₂P(O)(OR^{f})R^{k}, —SR^{f}, —S(O)R^{f}, —S(O)₂R^{f}, —S(O)NR^{g}R^{h}, or —S(O)₂NR^{g}R^{h}; wherein each R^{f}, R^{g}, R^{h}, and R^{k} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{g} and R^{h} together with the N atom to which they are attached form heterocyclyl; and each Rⁿ is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iv) two Rⁿ together are a divalent cation (e.g., Mg²⁺ or Ca²⁺).

47. The compound of claim 46 having the structure of Formula IIAa: 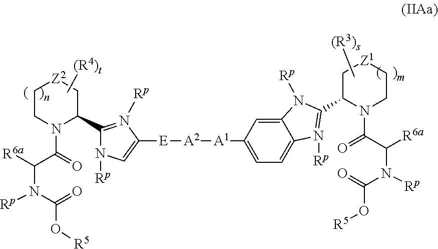
or a single enantiomer, a diastereomer, a mixture of diastereomers, or an isotopic variant thereof; or a pharmaceutically acceptable salt or solvate thereof.

48. The compound of claim 46 having the structure of Formula IIAb: 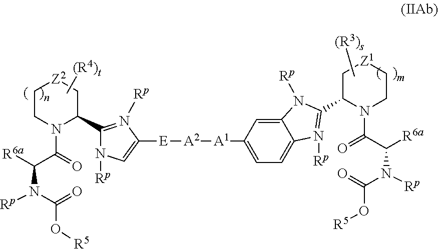
or a single enantiomer or an isotopic variant thereof; or a pharmaceutically acceptable salt or solvate thereof.

49. The compound of claim 46 having the structure of Formula IIAc: 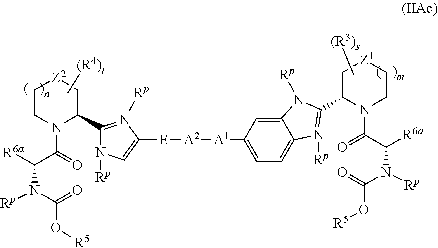
or a single enantiomer or an isotopic variant thereof; or a pharmaceutically acceptable salt or solvate thereof.

50-134. (canceled)

135. The compound of claim 46 having the structure of Formula IC: 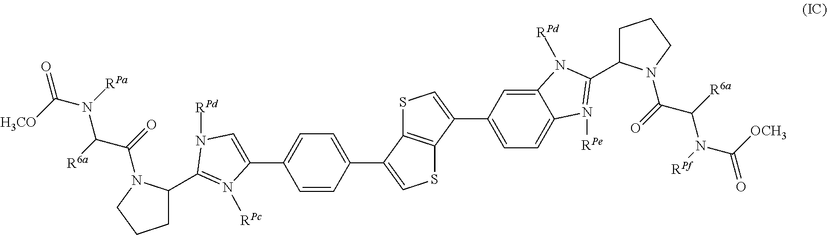
or a single enantiomer, a racemic mixture, a diastereomer, a mixture of diastereomers, or an isotopic variant thereof; or a pharmaceutically acceptable salt or solvate thereof; wherein R^{Pa}, R^{Pb}, R^{Pc}, R^{Pd}, R^{Pe}, and R^{Pf} are each independently absent, hydrogen, —C₁₋₆ alkylene-OP(O)(OR^{P1})₂ (in one embodiment, —CH₂—OP(O)(OR^{P})₂) or —C₁₋₆ alkylene-O-linked amino acid or a derivative thereof (in one embodiment, —CH₂—OC(O)C(R^{aa})₂NR^{1b}R^{1c}); wherein, when R^{Pb} and R^{Pc} are neither absent nor hydrogen, the imidazolyene group carries a positive charge; and when R^{Pd} and R^{Pe} are neither absent nor hydrogen, the benzimidazolylene group carries a positive charge; wherein at least one of R^{Pa}, R^{Pb}, R^{Pc}, R^{Pd}, R^{Pe}, and R^{Pf} is neither absent nor hydrogen; and wherein the alkylene is optionally substituted with one or more, in one embodiment, one, two, three, or four, substituents Q.

136-137. (canceled)

138. The compound of claim 46 having the structure of Formula ID: 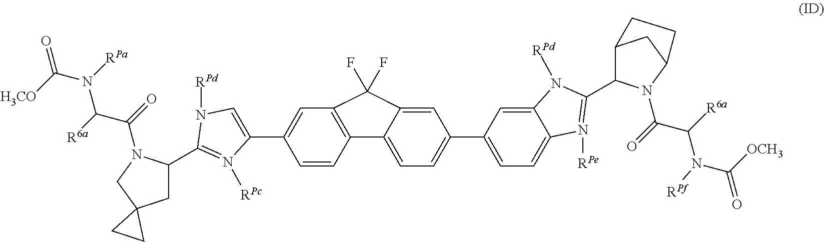
or a single enantiomer, a racemic mixture, a diastereomer, a mixture of diastereomers, or an isotopic variant thereof; or a pharmaceutically acceptable salt or solvate thereof; wherein R^{Pa}, R^{Pb}, R^{Pc}, R^{Pd}, R^{Pe}, and R^{Pf} are each independently absent, hydrogen, —C₁₋₆ alkylene-OP(O)(OR^{P1})₂ (in one embodiment, —CH₂—OP(O)(OR^{P1})₂) or —C₁₋₆ alkylene-O-linked amino acid or a derivative thereof (in one embodiment, —CH₂—OC(O)C(R^{aa})₂NR^{1b}R^{1c}); wherein, when R^{Pb} and R^{Pc} are neither absent nor hydrogen, the imidazolyene group carries a positive charge; and when R^{Pd} and R^{Pe} are neither absent nor hydrogen, the benzimidazolylene group carries a positive charge; and wherein at least one of R^{Pa}, R^{Pb}, R^{Pc}, R^{Pd}, R^{Pe}, and R^{Pf} is neither absent nor hydrogen; and wherein the alkylene is optionally substituted with one or more, in one embodiment, one, two, three, or four, substituents Q.

139-141. (canceled)

142. The compound of claim 1 having the structure of Formula IE: 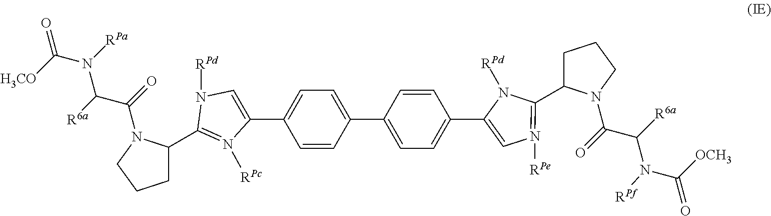
or a single enantiomer, a racemic mixture, a diastereomer, a mixture of diastereomers, or an isotopic variant thereof; or a pharmaceutically acceptable salt or solvate thereof; wherein R^{Pa}, R^{Pb}, R^{Pc}, R^{Pd}, R^{Pe}, and R^{Pf} are each independently absent, hydrogen, —C₁₋₆ alkylene-OP(O)(OR^{P1})₂ (in one embodiment, —CH₂—OP(O)(OR^{P1})₂) or —C₁₋₆ alkylene-O-linked amino acid or a derivative thereof (in one embodiment, —CH₂—OC(O)C(R^{aa})₂NR^{1b}R^{1c}); wherein, when R^{Pb} and R^{Pc} are neither absent nor hydrogen, the imidazolyene group carries a positive charge; and when R^{Pd} and R^{Pe} are neither absent nor hydrogen, the benzimidazolylene group carries a positive charge; and wherein at least one of R^{Pa}, R^{Pb}, R^{Pc}, R^{Pd}, R^{Pe}, and R^{Pf} is neither absent nor hydrogen; and wherein the alkylene is optionally substituted with one or more, in one embodiment, one, two, three, or four, substituents Q.

143. (canceled)

144. The compound of claim 1, selected from:

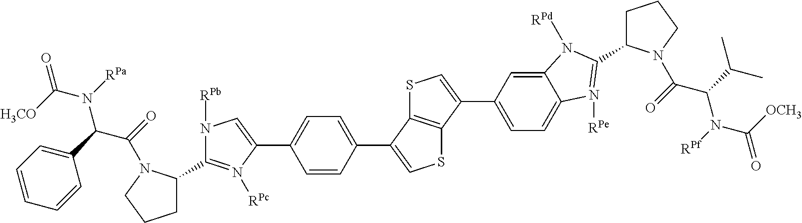

| Cmpd. | R^{Pa} | R^{Pb} | R^{Pc} | R^{Pd} | R^{Pe} | R^{Pf} |
|---|---|---|---|---|---|---|
| A1 | H | —CH₂O(O)P(ONa)₂ | Absent | H | Absent | H |
| A2 | H | H | Absent | —CH₂O(O)P(ONa)₂ | Absent | H |
| A3 | H | H | Absent | Absent | —CH₂O(O)P(ONa)₂ | H |
| A4 | H | Absent | —CH₂O(O)P(ONa)₂ | H | Absent | H |
| A5 | H | —CH₂O(O)P(ONa)₂ | Absent | Absent | —CH₂O(O)P(ONa)₂ | H |
| A6 | H | —CH₂O(O)P(ONa)₂ | Absent | —CH₂O(O)P(ONa)₂ | Absent | H |
| 7b | H | —CH₂O(O)P(OH)₂ | Absent | Absent | —CH₂O(O)P(OH)₂ | H |
| 6a | H | —CH₂O(O)P(OH)₂ | Absent | H | Absent | H |
| 6b | H | Absent | —CH₂O(O)P(OH)₂ | H | Absent | H |
| 6d | H | H | Absent | Absent | —CH₂O(O)P(OH)₂ | H |
| 6c | H | H | Absent | —CH₂O(O)P(OH)₂ | Absent | H |
| A12 | H | Absent | —CH₂O(O)P(ONa)₂ | Absent | —CH₂O(O)P(ONa)₂ | H |
| A13 | H | Absent | —CH₂O(O)P(ONa)₂ | —CH₂O(O)P(ONa)₂ | Absent | H |
| 7a | H | —CH₂O(O)P(OH)₂ | Absent | —CH₂O(O)P(OH)₂ | Absent | H |
| 7c | H | Absent | —CH₂O(O)P(OH)₂ | —CH₂O(O)P(OH)₂ | Absent | H |
| 7d | H | Absent | —CH₂O(O)P(OH)₂ | Absent | —CH₂O(O)P(OH)₂ | H |
| 10a | H | 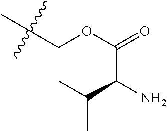 [SMILES: *COC(=O)[C@@H](N)C(C)C] | Absent | H | Absent | H |
| 10b | H | Absent | 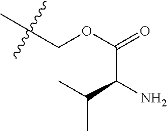 [SMILES: *COC(=O)[C@@H](N)C(C)C] | H | Absent | H |
| 10c | H | H | Absent | 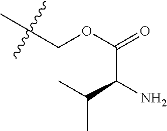 [SMILES: *COC(=O)[C@@H](N)C(C)C] | Absent | H |
| 10d | H | H | Absent | Absent | 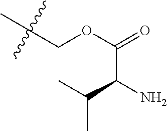 [SMILES: *COC(=O)[C@@H](N)C(C)C] | H |
| 11a | H | 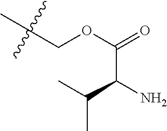 [SMILES: *COC(=O)[C@@H](N)C(C)C] | Absent | 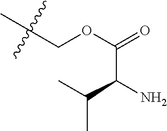 [SMILES: *COC(=O)[C@@H](N)C(C)C] | Absent | H |
| 11b | H | 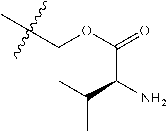 [SMILES: *COC(=O)[C@@H](N)C(C)C] | Absent | Absent | 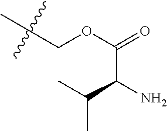 [SMILES: *COC(=O)[C@@H](N)C(C)C] | H |
| 11c | H | Absent | 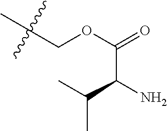 [SMILES: *COC(=O)[C@@H](N)C(C)C] | 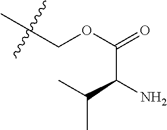 [SMILES: *COC(=O)[C@@H](N)C(C)C] | Absent | H |
| 11d | H | Absent | 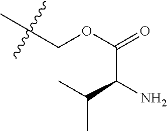 [SMILES: *COC(=O)[C@@H](N)C(C)C] | Absent | 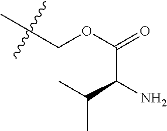 [SMILES: *COC(=O)[C@@H](N)C(C)C] | H |

and isotopic variants thereof; and pharmaceutically acceptable salts and solvates thereof .

145. A compound of Formula IB: 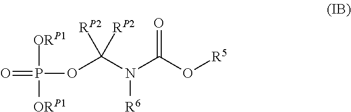
or a single enantiomer, a racemic mixture, a diastereomer, a mixture of diastereomers, or an isotopic variant thereof; or a pharmaceutically acceptable salt or solvate thereof;
R⁵ is C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl;
R⁶ is (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —CHR^{6a}C(O)R^{6b};
R^{6a} is hydrogen, C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl;
R^{6b} is: 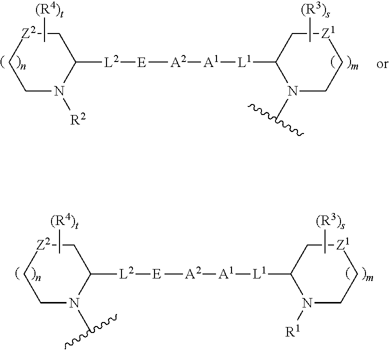
wherein:
A¹, A², and E are each independently (a) a bond; or (b) C₁₋₆ alkylene, C₂₋₆ alkenylene, C₂₋₆ alkynylene, C₂₋₂₀ cycloalkylene, C₆₋₂₀ arylene, heteroarylene; or heterocyclylene;
L¹ and L² are each independently (a) a bond; (b) C₁₋₆ alkylene, C₂₋₆ alkenylene, C₂₋₆ alkynylene, C₃₋₇ cycloalkylene, C₆₋₁₄ arylene, heteroarylene, or heterocyclylene; or (c) —C(O)—, —C(O)O—, —C(O)NR^{1a}—, —C(═NR^{1a})NR^{1c}—, —O—, —OC(O)O—, —OC(O)NR^{1a}—, —OC(═NR^{a})NR^{1c}—, —OP(O)(OR^{1a})—, —OP(O)(OR^{1a})O—, —NR^{1a}, —NR^{1a}C(O)NR^{1c}, —NR^{1a}C(═NR^{1b})NR^{1c}—, —NR^{1a}S(O)NR^{1c}—, —NR^{1a}S(O)₂NR^{1c}—, —S—, —S(O)—, —S(O)₂—, —S(O)NR^{1a}—, or —S(O)₂NR^{1a}—;
Z¹ and Z² are each independently a bond, —O—, —S—, —S(O)—, —S(O₂)—, or —N(R^{N})—;
R¹ and R² are each independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (c) —C(O)R^{1a}, —C(O)CH(N(R^{1c})C(O)OR^{1b})R^{1a}, —C(O)OR^{1a}, —C(O)NR^{1b}R^{1c}, —C(NR^{1a})NR^{1b}R^{1c}, —P(O)(OR^{1a})R^{1d}, —CH₂P(O)(OR^{1a})R^{1d}, —S(O)R^{1a}, —S(O)₂R^{1a}, —S(O)NR^{1b}R^{1c}, or —S(O)₂NR^{1b}R^{1c}; (d) —C₁₋₆ alkylene-OP(O)(OR^{P1})₂, in one embodiment, —CH₂—OP(O)(OR^{P1})₂; or (e) —C₁₋₆ alkylene-O-linked amino acid or a derivative thereof, in one embodiment, —CH₂—OC(O)C(R^{aa})₂NR^{1b}R^{1c};
each R³ and R⁴ is independently (a) cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{1a}, —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —NR^{1a}C(═NR^{1d})NR^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂R^{1b}R^{1c}, —P(O)(OR^{1a})R^{1d}, —CH₂P(O)(OR^{1a})R^{1d}, —SR^{1a}, —S(O)R^{1a}, —S(O)₂R^{1a}, —S(O)NR^{1b}R^{1c}, or —S(O)₂NR^{1b}R^{1c}; or
two R³ or two R⁴ that are attached to the same ring are linked together to form a bond, —O—, —NR^{N}—, —S—, C₁₋₆ alkylene, C₁₋₆ heteroalkylene, C₂₋₆ alkenylene, or C₂₋₆ heteroalkenylene;
each R^{N} is independently (a) hydrogen; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; (c) —C(O)OR^{1a}, —C(O)NR^{1b}R^{1c}, —C(NR^{1a})NR^{1b}R^{1c}, —OR^{1a}, —OC(O)R^{1a}, —OC(O)OR^{1a}, —OC(O)NR^{1b}R^{1c}, —OC(═NR^{1a})NR^{1b}R^{1c}, —OS(O)R^{1a}, —OS(O)₂R^{1a}, —OS(O)NR^{1b}R^{1c}, —OS(O)₂NR^{1b}R^{1c}, —NR^{1b}R^{1c}, —NR^{1a}C(O)R^{1d}, —NR^{1a}C(O)OR^{1d}, —NR^{1a}C(O)NR^{1b}R^{1c}, —R^{1a}C(═NR^{1d}R^{1b}R^{1c}, —NR^{1a}S(O)R^{1d}, —NR^{1a}S(O)₂R^{1d}, —NR^{1a}S(O)NR^{1b}R^{1c}, —NR^{1a}S(O)₂NR^{1b}R^{1c}, —P(O)(OR^{1a})R^{1d}, —CH₂P(O)(OR^{1a})R^{1d}, —S(O)R^{1a}, —S(O)₂R^{1a}, —S(O)NR^{1b}R^{1c}, or —S(O)₂NR^{1b}R^{1c}; (d) —C₁₋₆ alkylene-OP(O)(OR^{P1})₂, in one embodiment, —CH₂—OP(O)(OR^{P1})₂; or (e) —C₁₋₆ alkylene-O-linked amino acid or a derivative thereof, in one embodiment, —CH₂—OC(O)C(R^{aa})₂NR^{1b}R^{1c};
each m and n is independently an integer of 1, 2, 3, or 4; and
each s and t is independently an integer of 0, 1, 2, 3, 4, 5, 6, or 7;
each R^{P1} is independently (a) hydrogen; (b) a monovalent cation, in one embodiment, Na⁺ or K⁺, in another embodiment, Li⁺, Rb⁺, or Cs⁺; (c) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (d) two R^{P1} together are a divalent cation, in one embodiment, Mg²⁺ or Ca²⁺; and
each R^{P2} is independently (a) hydrogen, cyano, halo, or nitro; or (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl;
each R^{1a}, R^{1b}, R^{1c}, and R^{1d} is independently hydrogen, C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or R^{1a} and R^{1c} together with the C and N atoms to which they are attached form heterocyclyl; or R^{1b} and R^{1c} together with the N atom to which they are attached form heterocyclyl;
each R^{aa} independently is a side chain of a naturally occurring or non-naturally occurring amino acid; in one embodiment, each R^{aa} independently is hydrogen, C₁₋₆ alkyl, heteroalkyl, C₆₋₁₄ aryl-C₁₆ alkyl and heteroaryl-C₁₆ alkyl;
wherein each alkyl, alkylene, heteroalkylene, alkenyl, alkenylene, heteroalkenylene, alkynyl, alkynylene, cycloalkyl, cycloalkylene, aryl, arylene, aralkyl, heteroaryl, heteroarylene, heterocyclyl, and heterocyclylene is optionally substituted with one or more, in one embodiment, one, two, three, or four, substituents Q, where each Q is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is further optionally substituted with one or more, in one embodiment, one, two, three, or four, substituents Q^{a}; or (c) —C(O)R^{a}, —C(O)OR^{a}, —C(O)NR^{b}R^{c}, —C(NR^{a})NR^{b}R^{c}, —OR^{a}, —OC(O)R^{a}, —OC(O)OR^{a}, —OC(O)NR^{b}R^{c}, —OC(═NR^{a})NR^{b}R^{c}, —OP(O)(OR^{e})₂, —OS(O)R^{a}, —OS(O)₂R^{a}, —OS(O)NR^{b}R^{c}, —OS(O)₂NR^{b}R^{c}, —NR^{b}R^{c}, —NR^{a}C(O)R^{d}, —NR^{a}C(O)OR^{d}, —NR^{a}C(O)NR^{b}R^{c}, —NR^{a}C(═NR)NR^{b}R^{c}, —NR^{a}S(O)R^{d}, —NR^{a}S(O)₂R^{d}, —NR^{a}S(O)NR^{b}R^{c}, —NR^{a}S(O)₂NR^{b}R^{c}, —P(O)(OR^{a})R^{d}, —CH₂P(O)(OR^{a})R^{d}, —CH₂OP(O)(OR^{e})₂, —CH₂OC(O)C(R^{a})₂NR^{b}R^{c}, —SR^{a}, —S(O)R^{a}, —S(O)₂R^{a}, —S(O)NR^{b}R^{c}, or —S(O)₂NR^{b}R^{c}, wherein each R^{a}, R^{b}, R^{c}, and R^{d} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iii) R^{b} and R^{c} together with the N atom to which they are attached form heterocyclyl, optionally substituted with one or more, in one embodiment, one, two, three, or four, substituents Q^{a}; and each R^{e} is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl, each of which is optionally substituted with one or more, in one embodiment, one, two, three, or four, substituents Q^{a}; or (iv) two R^{e} together are a divalent cation (e.g., Mg²⁺ or Ca²⁺);
wherein each Q^{a} is independently selected from (a) oxo, cyano, halo, or nitro; (b) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (c) —C(O)R^{f}, —C(O)OR^{f}, —C(O)NR^{g}R^{h}, —C(NR^{f})NR^{g}R^{h}, —OR^{f}, —OC(O)R^{f}, —OC(O)OR^{f}, —OC(O)NR^{g}R^{h}, —OC(═NR^{f})NR^{g}R^{h}, —OP(O)(ORⁿ)₂, —OS(O)R^{f}, —OS(O)₂R^{f}, —OS(O)NR^{g}R^{h}, —OS(O)₂NR^{g}R^{h}, —NR^{g}R^{h}, —NR^{f}C(O)R^{k}, —NR^{f}C(O)OR^{k}, —NR^{f}C(O)NR^{g}R^{h} NR^{f}C(═NR^{k})NR^{g}R^{h}, —NR^{f}S(O)R^{k}, —NR^{f}S(O)₂R^{k}, —NR^{f}S(O)NR^{g}R^{h}, —NR^{f}S(O)₂NR^{g}R^{h}, —P(O)(OR^{f})R^{k}, —CH₂P(O)(OR^{f})R^{k}, —SR^{f}, —S(O)R, —S(O)₂R^{f}, —S(O)NR^{g}R^{h}, or —S(O)₂NR^{g}R^{h}; wherein each R^{f}, R^{g}, R^{h}, and R^{k} is independently (i) hydrogen; (ii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iii) R^{g} and R^{h} together with the N atom to which they are attached form heterocyclyl; and each Rⁿ is independently (i) hydrogen; (ii) a monovalent cation (e.g., Na⁺ or K⁺); (iii) C₁₋₆ alkyl, C₂₋₆ alkenyl, C₂₋₆ alkynyl, C₃₋₇ cycloalkyl, C₆₋₁₄ aryl, C₇₋₁₅ aralkyl, heteroaryl, or heterocyclyl; or (iv) two Rⁿ together are a divalent cation (e.g., Mg²⁺ or Ca²⁺).

146. The compound of claim 1 having the structure of Formula IIB:

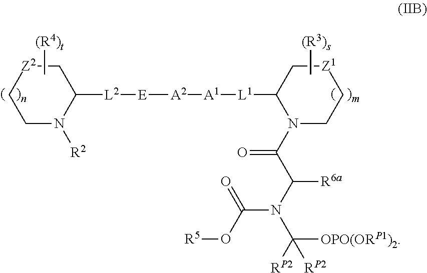

147. The compound of claim 1 having the structure of Formula IIIB:

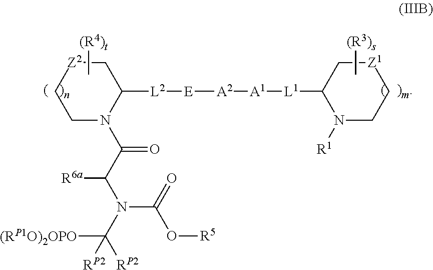

148. (canceled)

149. A pharmaceutical composition comprising the compound of claim 1, and one or more pharmaceutically acceptable carriers.

150. The pharmaceutical composition of claim 149, further comprising a second antiviral agent.

151-158. (canceled)

159. A method for treating or preventing an HCV infection in a subject, which comprises to the subject administering the compound of claim 1.

160. (canceled)

161. The method of claim 159, wherein the method comprises administering to the subject a second antiviral agent.

162-165. (canceled)

166. A method for inhibiting replication of a virus in a host, which comprises contacting the host with the compound of claim 1.

167-169. (canceled)
